(19) United States
(12) Patent Application Publication
(10) Pub. No.: US 2009/0017019 A1
(43) Pub. Date: Jan. 15, 2009

(54) METHODS AND COMPOSITIONS FOR MODULATING BMP-10 ACTIVITY

(75) Inventors: Kathleen M. Shields, Harvard, MA (US); Debra D. Pittman, Windham, NH (US); Jeffrey L. Feldman, Arlington, MA (US); Robert Martinez, Carlisle, MA (US); Christine Huard, Somerville, MA (US)

Correspondence Address: LOWRIE, LANDO & ANASTASI, LLP;W2023, ONE MAIN STREET, SUITE 1100, CAMBRIDGE, MA, 02142, US

(73) Assignee: Wyeth, Cambridge, MA (US)

(21) Appl. No.: 12/130,844

(22) Filed: May 30, 2008

Related U.S. Application Data
(60) Provisional application No. 60/932,815, filed on Jun. 1, 2007.

(51) Int. Cl. A61K 39/395; C12N 5/00; A61K 38/16; A61K 38/02; A61K 38/14; A61K 31/7052; G01N 33/68; G01N 33/53; C12Q 1/68; A61P 9/00; A61P 13/12

(52) U.S. Cl. 424/133.1; 435/375; 424/158.1; 514/12; 514/2; 424/178.1; 514/8; 514/44; 436/86; 436/94; 435/6; 435/7.21

(57) ABSTRACT

Methods and compositions for modulating cardiac, renal and vascular cell function and homeostasis using agonists and antagonists of BMP-10 are disclosed. In particular, methods for treating, preventing and/or diagnosing BMP-10-associated vascular, renal, fibrotic and cardiac conditions and/or disorders are disclosed. Screening methods for evaluating BMP-10 modulators, e.g., agonists and antagonists, are also disclosed.

### CROSS REFERENCE TO RELATED APPLICATIONS

This application claims priority to U.S. Ser. No. 60/932,815 filed on Jun. 1, 2007. The contents of the aforementioned application are hereby incorporated by reference in its entirety. This application also incorporates by reference the International Application filed with the U.S. Receiving Office on May 30, 2008, entitled “Methods and Compositions for Modulating BMP-10 Activity” and bearing attorney docket number W2023-7015WO.

### SEQUENCE LISTING

An electronic copy of the Sequence Listing in both pdf and txt formats is being submitted herewith.

### BACKGROUND

Bone morphogenic proteins (BMPs), named for their initial biological activity of inducing ectopic bone formation, belong to the transforming growth factor-beta (TGFβ) family. Other members of the TGFβ family include Growth Differentiation Factors (GDFs), Activins, Inhibins, and Nodal and Müllerian Inhibiting Substance (MIS) (Massague, J. (1998) Annu. Rev. Biochem. 67:753-791). TGFβ family members are expressed as pre-propeptides, which are proteolytically processed and secreted as cystine-knot cytokines. Most TGFβ family members bind to heteromeric complexes of type I and type II serine/threonine kinase receptors. In addition, the type III receptors (betaglycan and endoglin) act as co-receptors that can potentiate the signaling cascade (reviewed by Shi, Y and Massague, J. (2003) Cell 113:685-700). Upon ligand binding, the type II receptor phosphorylates and activates the type I receptor, also known as activin receptor-like kinase (ALK), which in turn phosphorylates a Smad protein (Smadt, Smad2, Smad3, Smad5 or Smad8). The phosphorylated Smad dimerizes with a common partner, Smad4, and this complex translocates to the nucleus where it regulates the transcription of target genes. The inhibitory Smad5, Smad6 and Smad7, can interrupt this signaling process (see Derynck, R. and Zhang, Y. E. (2003) Nature 425:577-584). TGFβ family members mediate a diverse spectrum of developmental and morphogenic effects, including cell proliferation, migration, and differentiation processes such as adipogenesis, myogenesis, chondrogenesis, cardiogenesis, hematopoiesis and epithelial cell differentiation (for review, see Ducy, P. and Karsenty, G. (2000) Kidney International 57:2207-2214; Nakayama et al. (2000)) Cell Mol. Life. Sci 57:943-956).

Bone Morphogenetic Protein 10 (BMP-10) was cloned in 1999 by Herbert Neuhaus, and has been studied for its biological effects on cardiac development (Neuhaus et al. (1999) Mechanism of Development 80:181-184). A typical cleavage site divides the 421 amino acid protein into a pro-region (309 amino acids) and a mature region of approximately 108 amino acid residues per monomer. The mature BMP-10 protein has a spatially conserved pattern of six to seven cysteines typically found in TGFβ family members. BMP-10 signals have been shown to mediate multiple steps of cardiac development, including cardiogenic induction, trabeculation of the embryonic heart and ventricular muscle cell lineage specification (see Schneider (2003) Cytokine Growth Factor Rev. 14:1-4).

Given the broad range of activities associated with TGFβ family members, and particularly BMP-10, the need still exists for identifying novel activities associated with, and modulators of, members of this family, such as BMP-10.

### SUMMARY

The present invention is based, at least in part, on the discovery that in vivo overexpression of bone morphogenic protein-10 (BMP-10) resulted in vascular dysplasia in animal models with a phenotype similar to Hereditary Hemorrhagic Telangiectasia (HHT). This finding implicates expression of BMP-10 in the regulation of cardiac and vascular homeostasis. To further support a role of BMP-10 in modulating vascular function, BMP-10 was shown to activate cell signaling events and gene expression in endothelial cells in vitro. In other embodiments, BMP-10 was shown to activate one or more signaling pathways and gene expression in renal cells in vitro (e.g., human primary renal proximal tubule epithelial cells). Applicants have further discovered an association of BMP-10 with fibrosis of various organs and tissues, including liver, lung, kidney and heart. Thus, the present invention provides, in part, methods and compositions for modulating BMP-10 function (e.g., cardiac and vascular homeostasis, renal function, and/or formation and/or accumulation of fibrous tissue) using agonists and antagonists of BMP-10. In particular, methods for treating, preventing and/or diagnosing BMP-10-associated conditions and/or disorders (e.g., BMP-10 associated vascular, cardiac, renal and/or fibrotic conditions and disorders) are disclosed. Screening methods for evaluating BMP-10 modulators, e.g., agonists and antagonists of BMP-10 function or expression, are also disclosed.

Accordingly, in one aspect, the invention features a method of modulating a function (e.g., one or more biological activities) of BMP-10 in a BMP-10-responsive cell, tissue and/or organ (e.g., a vascular (e.g., an endothelial, smooth muscle), renal, and/or cardiac, cell or tissue, or a fibrotic tissue or organ). The method includes contacting the BMP-10-responsive cell, tissue and/or organ with a BMP-10 modulator, e.g., an agonist or an antagonist of BMP-10 (e.g., an agonist or an antagonist of human mature or propeptide form of BMP-10) activity or expression, in an amount sufficient to modulate the function of the BMP-10 responsive cell, tissue and/or organ (or the biological activity of BMP-10 in the cell, tissue and/or organ). In one embodiment, the contacting step can be effected in vitro, e.g., in a cell lysate or in a reconstituted system. Alternatively, the subject method can be performed on cells in culture, e.g., in vitro or ex vivo. For example, cells (e.g., purified or recombinant cells) can be cultured in vitro and the contacting step can be effected by adding the BMP-10 modulator to the culture medium. In embodiments, the cells are previously or simultaneously exposed to BMP-10. Typically, the BMP-10-responsive cell is a mammalian cell, e.g., a human cell. In some embodiments, the BMP-10-responsive cell is an endothelial cell or population of cells (e.g., human umbilical vein endothelial cell (HUVECS), human aortic endothelial cells (HAECS)); a vascular smooth muscle cell or population thereof; a cardiac cell (e.g., a cardiomyocyte) or a population thereof; or a renal cell (e.g., a human renal proximal tubule cell) or a population thereof. In other embodiment, the BMP-10 tissue is an endothelial, vascular, cardiac, renal, or a fibrotic tissue or organ (e.g., a fibrotic or fibrous tissue of the liver, lung, peritoneum, kidney or heart). In other embodiments, the method can be performed on cells (e.g., BMP-10-responsive cells) present in a subject, e.g., as part of an in vivo (e.g., therapeutic or prophylactic) protocol, or in an animal subject (e.g., an in vivo animal model, such as a cardiovascular ischemic model or a genetically modified model, e.g., an animal model having a mutation in a BMP receptor (BMPR2) or an NKX2-5 deficient animal; or a fibrotic animal model). In embodiments, the subject has elevated expression or activity of BMP-10.

For in vivo methods, the BMP-10 modulator, alone or in combination with another agent, can be administered to a subject, e.g., a mammal, suffering from a BMP-10-associated vascular, cardiac, renal or fibrotic condition and/or disorder, in an amount sufficient to modulate, BMP-10 function (e.g., one or more BMP-10 activities) in the subject.

In some embodiments, the amount or dosage of the BMP-10 modulator, e.g., antagonist, administered can be determined, e.g., prior to administration to the subject, by testing in vitro or ex vivo the amount of BMP-10 antagonist required to modulate, e.g., decrease or inhibit, one or more of BMP-10 biological activities (e.g., one or more of the BMP-10 activities described herein). The in vivo method can, optionally, include the step(s) of identifying (e.g., evaluating, diagnosing, screening, and/or selecting) a subject at risk of having, or having, one or more symptoms associated with the disorder or condition.

In embodiments where inhibition, reduction or otherwise diminution of one or more BMP-10 biological activities is desired, the BMP-10 responsive cell and/or tissue is contacted with a BMP-10 antagonist, e.g., by administering the BMP-10 antagonist to the subject. In one embodiment, the BMP-10 antagonist interacts with, e.g., binds to, BMP-10 or a BMP-10 receptor (also individually referred to herein as a “BMP-10 antagonist” and “BMP-10 receptor antagonist,” respectively), and reduces or inhibits one or more of BMP-10 and/or BMP-10 receptor activities. Typically, the BMP-10 or the BMP-10 receptor antagonized is a mammalian, e.g., human, BMP-10 or BMP-10 receptor (or a functional variant thereof). In embodiments, the BMP-10 antagonized includes a mature BMP-10 sequence (e.g., a mature BMP-10 sequence comprising an amino acid sequence of about amino acids 314 to 424, or a portion, of the human BMP-10 amino acid sequence shown in FIG. 2 (SEQ ID NO:2), or a sequence substantially homologous thereto; or encoded by a nucleotide sequence shown in FIG. 1 (SEQ ID NO:1), or a sequence substantially homologous thereto). In other embodiments, the BMP-10 antagonized includes a propeptide BMP-10 sequence (e.g., a propeptide BMP-10 sequence comprising an amino acid sequence of about amino acids 22 to 424, or a portion, of the human BMP-10 amino acid sequence shown in FIG. 2 (SEQ ID NO:2), or a sequence substantially homologous thereto; or encoded by a nucleotide sequence shown in FIG. 1 (SEQ ID NO:1), or a sequence substantially homologous thereto). In embodiments, the BMP-10 receptor antagonized is an endoglin, or an activin receptor-like kinase (ALK)-1, -3, or -6 (e.g., an ALK-1, or -3 comprising an amino acid sequence identical to a mammalian, e.g., a human, ALK-1 amino acid sequence shown in FIG. 3D (SEQ ID NO:4), or a encoded by a nucleic acid comprising a nucleotide sequence shown in FIGS. 3A-3C (SEQ ID NO:3); or a human ALK-3 comprising an amino acid sequence shown in FIG. 4C (SEQ ID NO:6), or encoded by a nucleic acid comprising a nucleotide sequence shown in FIGS. 4A-4B (SEQ ID NO:5); or a sequence substantially homologous thereto).

Typical antagonists bind to BMP-10 or the BMP-10 receptor with high affinity, e.g., with an affinity constant of at least about 10⁷ M⁻¹, typically about 10⁸ M⁻¹, and more typically, about 10⁹ M⁻¹ to 10¹⁰ M⁻¹ or stronger; and reduce and/or inhibit one or more BMP-10 biological activities in a BMP-10 responsive cell, tissue and/or organ (e.g., a vascular (e.g., an endothelial, smooth muscle), and/or cardiac, cell or tissue, or fibrotic tissue or organ). Exemplary BMP-10 activities that can be inhibited or reduced using the methods and compositions of the invention include, but are not limited to, one or more of the following: (i) phosphorylation of a Smad protein (e.g., phosphorylation of Smad 1, 5 and/or 8); (ii) induction of gene expression of myostatin, endoglin and/or an inhibitory Smad (e.g., induction of expression of Smad 6 and/or 7); (iii) increased expression of one or more pro-angiogenic genes (e.g., VEGF, ID1 and ID2); (iv) decreased expression of Ras-related protein-1a (Rap1a); (v) modulation of, e.g., increase or decrease, expression of one or more genes in response to BMP-10 stimulation of endothelial or renal cells in vitro or in vivo identified in FIGS. 22-28; (vi) increased serum levels of stromal-derived differentiation factor (SDF-1) and/or matrix metallopeptidase 9 (MMP-9); and/or (vii) increased abnormalities in blood vessels, such as vascular dysplasia, hemorrhaging, telangiectasias, and/or arteriovenous malformations.

In one embodiment, the BMP-10 antagonist is an antibody molecule against BMP-10 or a BMP-10 receptor. The antibody molecule can be a monoclonal or single specificity antibody, or an antigen-binding fragment thereof (e.g., an Fab, F(ab′)₂, Fv, a single chain Fv fragment, a single domain antibody, a diabody (dAb), a bivalent or bispecific antibody or fragment thereof, a single domain variant thereof, or a camelid or shark antibody) that binds to BMP-10 or a BMP-10 receptor, e.g., a mammalian (e.g., human, BMP-10 or BMP-10 receptor (or a functional variant thereof)). In embodiments, the antibody molecule binds to mature BMP-10 (e.g., a mature human BMP-10 as described herein). Typically, the antibody molecule is a human, humanized, chimeric, camelid, shark or in vitro generated antibody to human BMP-10 or human BMP-10 receptor polypeptide (or functional fragment thereof, e.g., an antibody fragment as described herein). Typically, the antibody inhibits, reduces or neutralizes one or more activities of BMP-10 or a BMP-10 receptor (e.g., one or more biological activities of BMP-10 as described herein).

In one embodiment, the antibody molecule binds to a mature BMP-10 polypeptide (e.g., about amino acids 314 to 424, or an epitope comprising fragments thereof, e.g., about amino acids 314 to 325, 325 to 335, 335 to 345, 345 to 355, 355 to 365, 365 to 375, 375 to 385, 385 to 395, 395 to 405, 405 to 415, and 415 to 424, of FIG. 2 (SEQ ID NO:2)), and inhibits, reduces or neutralizes one or more activities of BMP-10. In another embodiment, the antibody molecule binds to a BMP-10 propeptide (e.g., about amino acids 22 to 424, or an epitope comprising a fragment thereof (e.g., about amino acids 22 to 313, 22 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, of FIG. 2 (SEQ ID NO:2)), and inhibits, reduces or neutralizes one or more activities of BMP-10. In yet other embodiments, the antibody molecule binds to a cleavage site in the BMP-10 propeptide, e.g., binds to an epitope located at about amino acids 21-22 or 313-314 of SEQ ID NO:2, and inhibits, reduces or neutralizes one or more activities of BMP-10. In embodiments, the antibody molecule binds to a BMP-10 receptor, e.g., endoglin, e.g., a human endoglin; or an activin receptor-like kinase (ALK)-1, -3, or -6 (e.g., an ALK-1, or -3 comprising an amino acid sequence identical to a mammalian, e.g., human, ALK-1 and -3 as shown in FIG. 3D (SEQ ID NO:4) and FIG. 4C (SEQ ID NO:6), respectively), or a sequence substantially homologous thereto, and inhibits, reduces or neutralizes one or more activities of BMP-10.

The antibody molecule can be full-length (e.g., can include at least one, and typically two, complete heavy chains, and at least one, and typically two, complete light chains) or can include an antigen-binding fragment (e.g., a Fab, F(ab′)₂, Fv, a single chain Fv fragment, or a single domain antibody or fragment thereof). In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function).

In other embodiments, the BMP-10 antagonist is a full length, or a fragment of a BMP-10 receptor polypeptide, e.g., an inhibitory BMP-10 binding domain of a BMP-10 receptor polypeptide. For example, the antagonist can be a soluble form of a BMP-10 receptor (e.g., a soluble form of mammalian (e.g., human) ALK-1, -3 or -6 comprising a BMP-10 binding domain; e.g., a soluble form of an extracellular domain of mammalian (e.g., human) ALK-1, -3 or -6). For example, the BMP-10 antagonist can include about amino acids 22 to 118 of human ALK-1 (FIG. 3D; SEQ ID NO:4); or about amino acids 24 to 152 of human ALK3 (FIG. 4C; SEQ ID NO:6). In other embodiments, the BMP-10 antagonist is a soluble form of a mammalian (e.g., human) activin receptor (e.g., activin receptor IIB (ActRIIB), e.g., including about amino acids 17 to 133 of FIG. 5B; SEQ ID NO:18).

In yet other embodiments, the BMP-10 antagonist is a BMP-10 antagonistic propeptide (e.g., a truncated or variant form of BMP-10 (e.g., a truncated or variant form of the propeptide region of human BMP-10 comprising about amino acids 22 to 313 of FIG. 2 (SEQ ID NO:2) or a portion or variant thereof) that is capable of forming an inhibitory complex with mature BMP-10).

A soluble form of a BMP-10 receptor or a BMP-10 antagonistic propeptide can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. The fusion proteins may additionally include a linker sequence joining the first moiety, e.g., a soluble BMP-10 receptor or BMP-10 propeptide, to the second moiety. In other embodiments, additional amino acid sequences can be added to the N- or C-terminus of the fusion protein to facilitate expression, steric flexibility, detection and/or isolation or purification. For example, a soluble form of a BMP-10 receptor or a BMP-10 antagonistic propeptide can be fused to a heavy chain constant region of the various isotypes, including: IgG1, IgG2, IgG3, IgG4, IgM, IgA1, IgA2, IgD, and IgE). Typically, the fusion protein can include the extracellular domain of a human BMP-10 receptor, or a BMP-10 propeptide (or a sequence homologous thereto), and, e.g., fused to, a human immunoglobulin Fc chain, e.g., human IgG (e.g., human IgG1 or human IgG2, or a mutated form thereof). The Fc sequence can be mutated at one or more amino acids to reduce effector cell function, Fc receptor binding and/or complement activity. An exemplary fusion protein that includes the amino acid sequence from about amino acids 17 to 133 of ActRIIB fused to a human IgG1 Fc is shown in FIG. 5B; SEQ ID NO:18).

It will be understood that the antibody molecules and soluble or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, among others.

In yet another embodiment, the BMP-10 antagonist is a binding domain fusion variant, or a small molecule. Binding domain fusion variants provide an example of a variant molecule that typically includes a binding domain polypeptide that is fused or otherwise connected to a hinge or hinge-acting region polypeptide, which in turn is fused or otherwise connected to a region comprising one or more native or engineered constant regions from a heavy chain, other than CH1, for example, the CH2 and CH3 regions of IgG and IgA, or the CH3 and CH4 regions of IgE. Typically, the binding domain fusion variant or small molecule will bind to a mammalian, e.g., human, BMP-10 or a BMP-10 receptor with an affinity of at least about 10⁷ M⁻¹, typically about 10⁸ M⁻¹, and more typically, about 10⁹ M⁻¹ to 10¹⁰ M⁻¹ or stronger; and reduce and/or inhibit one or more BMP-10 biological activities as described herein. In embodiments, the binding domain fusion variant, or small molecule, binds to a mature BMP-10 sequence (e.g., about amino acids 314 to 424, or a fragments thereof, e.g., about amino acids 314 to 325, 325 to 335, 335 to 345, 345 to 355, 355 to 365, 365 to 375, 375 to 385, 385 to 395, 395 to 405, 405 to 415, and 415 to 424, of FIG. 2 (SEQ ID NO:2)), and inhibits, reduces or neutralizes one or more activities of BMP-10. In another embodiment, the binding domain fusion variant, or small molecule, binds to a BMP-10 propeptide (e.g., about amino acids 22 to 424, or a fragment thereof (e.g., about amino acids 22 to 313, 22 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 314, of FIG. 2 (SEQ ID NO:2)), and inhibits, reduces or neutralizes one or more activities of BMP-10. In yet other embodiments, the binding domain fusion variant, or small molecule, binds to a cleavage site in the BMP-10 propeptide, e.g., binds to an region located at about amino acids 21-22 or 313-314 of SEQ ID NO:2, and inhibits, reduces or neutralizes one or more activities of BMP-10. In embodiments, the binding domain fusion variant, or small molecule, binds to a BMP-10 receptor, e.g., an endoglin, e.g., a human endoglin; or an activin receptor-like kinase (ALK)-1, -3, or -6 (e.g., an ALK-1, or -3 comprising an amino acid sequence identical to a mammalian, e.g., human, ALK-1 and -3 as shown in FIG. 3D (SEQ ID NO:4) and FIG. 4C (SEQ ID NO:6), respectively), or a sequence substantially homologous thereto, and inhibits, reduces or neutralizes one or more activities of BMP-10.

In yet another embodiment, the BMP-10 antagonist is a KL-4 Surfactant (lucinactant) or a variant thereof. For example, the BMP-10 antagonist can be an engineered version of natural human lung surfactant, e.g., a KL-4 protein-like substance that is designed to closely mimic the attributes of human surfactant protein B (SP-B). In other embodiments, the BMP-10 antagonist has an amino acid sequence of a naturally occurring BMP-10 antagonist, or a sequence substantially homologous thereto. For example, the BMP-10 antagonist can have an amino acid sequence of a naturally-occurring BMP-10 antagonist chosen from a uterine sensitization-associated gene 1 (USAG-1), sclerostin, noggin, chordin, gremlin or twisted gastrulation, or a variant or fragment thereof.

In another embodiment, the BMP-10 antagonist inhibits the expression of nucleic acid encoding a BMP-10 or a BMP-10 receptor. Examples of such BMP-10 antagonists include nucleic acid molecules, for example, antisense molecules, ribozymes, RNAi, triple helix molecules that hybridize to a nucleic acid encoding a BMP-10 or BMP-10 receptor, or a transcription regulatory region, and blocks or reduces mRNA expression of BMP-10 or a BMP-10 receptor.

In in vivo embodiments, the BMP-10 antagonist can be administered to a subject, at risk of, or having, a BMP-10-associated disorder and/or condition. The subject can be a mammal, e.g., a human, suffering from, for example, a BMP-10-associated vascular, renal, fibrotic or cardiac condition and/or disorder. For example, the subject is a mammal (e.g., a human patient) suffering from a vascular disorder or condition characterized by endothelial cell dysfunction; e.g., a disorder or condition chosen from one or more of: Hereditary Hemorrhagic Telangiectasia (HHT); nephritic syndrome, nephropathy (e.g., diabetic nephropathy), retinopathy (e.g., diabetic retinopathy), neovascular glaucoma and other diabetic vascular conditions; stroke, atherosclerosis, arteriosclerosis, peripheral artery disease, hypertension (e.g., pulmonary hypertension), pulmonary disease, hyperlipidemia, thrombosis and/or restenosis. BMP-10 antagonists can also be used to treat or prevent neoplastic and non-neoplastic vascular disorders characterized by undesirable or excessive endothelial cell proliferation or neovascularization. As shown in the appended examples, administration of BMP-10 increased the expression of pro-angiogenic genes (e.g., VEGF, ID1 and ID2) in endothelial cells in culture, and increased expression of SDF-1b and MMP-9 in vivo. Thus, antagonism of BMP-10 induction of these genes and proteins may be used to inhibit or decrease angiogenesis and vascularization, and thus be useful in treating or preventing neoplastic and non-neoplastic disorders chosen from one or more of: neoplasms, e.g., solid malignant tumors, such as colorectal, gastric, breast, lung, kidney, esophageal and liver carcinomas; and other cancerous conditions, such as retinoblastomas, glioblastomas, astrocytomas, neuroblastomas, among others; as well as non-neoplastic conditions such as rheumatoid arthritis, psoriasis and other chronic inflammatory conditions, corneal and tissue transplantation, among others.

In other embodiments, the subject treated with the BMP-10 antagonist suffers from a BMP-10 associated cardiac disorder or condition. Cardiac or heart disorders can be characterized by any kind of cardiac dysfunction involving, e.g., inadequate blood supply to the heart; irregularities in the heart rhythm; and/or defective conduction of impulses from the atria to the ventricles of the heart. Examples of cardiac disorders include, but are not limited to, congenital heart disease, cardiomyopathy (e.g., dilated, hypertrophic, restrictive cardiomyopathy), congestive heart failure, and myocardial infarction.

In other embodiments, the subject treated with the BMP-10 antagonist suffers from a fibrotic disorder or condition. Applicants have shown herein that addition of BMP-10 to human renal proximal tubule epithelial cells induces expression of pro-fibrotic genes (FIGS. 24-25). Addition of BMP-10 to renal cells in culture also stimulates renal epithelial cells; the activation of which is blocked by BMP-10 receptor antagonists (FIGS. 19-20). These results indicate that antagonism of BMP-10 can be useful in treating or preventing fibrotic disorders or conditions. Fibrotic disorders or conditions can be characterized by aberrant and/or excessive formation or accumulation of fibrous material (e.g., extracellular matrix) or tissue. In embodiments, a fibrous connective tissue replaces normal tissue (e.g., normal smooth muscle or other normal organ tissue). The formation of fibrous tissue can result from a reparative or reactive process. Fibrosis conditions or disorders include, but are not limited to, fibroproliferative disorders that are associated with vascular diseases, such as cardiac disease, cerebral disease, and peripheral vascular disease, as well as in tissues and organ systems, including the heart, skin, kidney, lung, peritoneum, gut and liver (as disclosed in, e.g., Wynn, Nature Reviews 4:583-594 (2004), incorporated herein by reference). Exemplary disorders that can be treated include, but are not limited to, kidney fibrosis, including, but not limited to, nephropathies associated with injury/fibrosis, e.g., chronic nephropathies associated with diabetes (e.g., diabetic nephropathy), lupus, scleroderma, glomerular nephritis, focal segmental glomerular sclerosis, and IgA nephropathy; lung or pulmonary fibrosis, e.g., idiopathic pulmonary fibrosis, radiation induced fibrosis, chronic obstructive pulmonary disease (COPD), scleroderma, and chronic asthma; gut fibrosis, e.g., scleroderma, and radiation induced gut fibrosis; liver fibrosis, e.g., cirrhosis, alcohol induced liver fibrosis, biliary duct injury, primary biliary cirrhosis, infection or viral induced liver fibrosis, congenital hepatic fibrosis and autoimmune hepatitis; and other fibrotic conditions, such as cystic fibrosis, endomyocardial fibrosis, mediastinal fibrosis, pleural fibrosis, sarcoidosis, scleroderma, spinal cord injury/fibrosis, myelofibrosis, vascular restenosis and atherosclerosis.

The BMP-10 antagonist can be administered to the subject alone, or in combination with one or more agents or therapeutic modalities, e.g., therapeutic agents, which are useful for treating BMP-10 associated vascular, renal, fibrotic or cardiac disorders and/or conditions. In one embodiment, the second agent or therapeutic modality is chosen from one or more of: angioplasty, beta blockers, anti-hypertensives, cardiotonics, anti-thrombotics, vasodilators, hormone antagonists, endothelin antagonists, calcium channel blockers, phosphodiesterase inhibitors, angiotensin type 2 antagonists, cytokine blockers/inhibitors, statins, anti-inflammatory agents, among others.

In embodiments where an increase in BMP-10 function, is desired, the BMP-10 responsive cell, tissue and/or organ (e.g., the vascular (e.g., an endothelial, smooth muscle), renal, and/or cardiac, cell or tissue) is contacted with a BMP-10 agonist, e.g., by administering the BMP-10 agonist to a subject. Examples of BMP-10 agonists include a BMP-10 protein or a functionally active fragment, peptide, or variant thereof (e.g., a mammalian, e.g., human, BMP-10 (e.g., mature BMP-10 as described herein or a sequence substantially homologous thereto); or a nucleic acid encoding the BMP-10 protein or functionally active fragment or variant thereof (e.g., a nucleic acid that includes the nucleotide sequence shown in FIG. 1 (SEQ ID NO:1) (or a portion or variant thereof). The BMP-10 agonist can be used alone or functionally linked (e.g., by chemical coupling, genetic or polypeptide fusion, non-covalent association or otherwise) to a second moiety, e.g., an immunoglobulin Fc domain, serum albumin, as described herein. In other embodiments, the BMP-10 agonist binds to a BMP-10 receptor, e.g., a receptor as described herein, and increases the receptor activity. For example, the BMP-10 agonist can be an antibody molecule, a binding domain fusion variant, or any other agent that binds to a BMP-10 receptor and stimulates one or more activities.

In embodiments, the BMP-10 agonist is used to expand the growth and/or differentiation of cells in culture or ex vivo. For example, stem cells can be obtained from a subject, e.g., a patient having a BMP-10-associated disorder and contacted with the BMP-10 agonist, thereby expanding the stem cell population. The expanded stem cells can then be re-introduced into the subject.

In embodiments, the BMP-10 agonist is administered to a subject. The subject can be a mammal, e.g., a human, suffering from, for example, a disorder characterized by underactive or disrupted BMP-10 function (e.g., a disorder characterized by underactive or disrupted vascular or cardiac cell proliferation and/or activity). For example, the BMP-10 agonist can be used to treat or prevent a disorder or condition following endothelial cell injury, e.g., after an ischemia attack or microvascular angiopathy. In some embodiments, the amount or dosage of the BMP-10 agonist administered can be determined, e.g., prior to administration to the subject, by testing in vitro or ex vivo the amount of BMP-10 agonist required to increase or stimulate one or more of the aforesaid BMP-10 biological activities. The in vivo method can, optionally, include the step(s) of identifying (e.g., evaluating, diagnosing, screening, and/or selecting) a subject at risk of having, or having, one or more symptoms associated with the disorder or condition.

In yet another aspect, the invention features a method of treating or preventing (e.g., curing, suppressing, ameliorating, delaying or preventing the onset of, or preventing recurrence or relapse of) a BMP-10-associated condition and/or disorder (e.g., a BMP-10-associated vascular, renal, cardiac or fibrotic condition and/or disorder), in a subject. The method includes administering to the subject a BMP-10 antagonist (e.g., a BMP-10 antagonist as described herein), in an amount sufficient to inhibit or reduce one or more BMP-10 biological activities in a BMP-10-responsive cell and/or tissue, e.g., a vascular (e.g., an endothelial, smooth muscle), renal, and/or cardiac, cell or tissue, or a fibrotic tissue (e.g., a biological activity as described herein), thereby treating or preventing the disorder or condition. The subject can be a mammal, e.g., a human, suffering from, for example, a BMP-10-associated condition and/or disorder as described herein. In some embodiments, the amount or dosage of the BMP-10 antagonist administered can be determined, e.g., prior to administration to the subject, by testing in vitro or ex vivo the amount of BMP-10 antagonist required to inhibit or reduce one or more of the aforesaid BMP-10 biological activities. The method can, optionally, include the step(s) of identifying (e.g., evaluating, diagnosing, screening, and/or selecting) a subject at risk of having, or having, one or more symptoms associated with the BMP-10-associated condition and/or disorder (e.g., a vascular, renal, cardiac or fibrotic condition and/or disorder as described herein).

The BMP-10 antagonist can be administered to the subject alone, or in combination with one or more agents or therapeutic modalities, e.g., therapeutic agents, which are useful for treating BMP-10 associated (e.g., vascular, renal, cardiac or fibrotic) disorders or conditions. In one embodiment, the second agent or therapeutic modality is a chosen from one or more of: tumor necrosis factor inhibitors; anti-fibroblast growth factor (FGF) antibodies; hepatocyte growth factors (e.g., in the treatment of diabetic nephropathy and other renal indications); antibodies capable of inhibiting or neutralizing the coagulant activities of tissue growth factor, protein C or protein S; anti-neoplastic agents (e.g., alkylating agents, folic acid antagonists, anti-metabolites, 5-fluorouracil, purine nucleosides, among others; angioplasty; beta blockers; anti-hypertensives; cardiotonics; anti-thrombotics; vasodilators; hormone antagonists; endothelin antagonists; calcium channel blockers; phosphodiesterase inhibitors; angiotensin type 2 antagonists, cytokine blockers/inhibitors, statins and/or anti-inflammatory agents.

In yet another aspect, the invention features a method of treating or preventing (e.g., curing, suppressing, ameliorating, delaying or preventing the onset of, or preventing recurrence or relapse of) a disorder characterized by underactive or disrupted BMP-10-responsive cell proliferation and/or activity. For example, the BMP-10 agonist can be used to treat or prevent a disorder or condition following endothelial cell injury, e.g., after an ischemia attack or microvascular angiopathy. The method includes administering to the subject a BMP-10 agonist (e.g., a BMP-10 agonist as described herein), in an amount sufficient to increase or stimulate one or more BMP-10 biological activities in an endothelial cell and/or vascular tissue (e.g., a biological activity as described herein), thereby treating or preventing the disorder or condition. The subject can be a mammal, e.g., a human, suffering from, for example, the disorder or condition. In some embodiments, the amount or dosage of the BMP-10 agonist administered can be determined, e.g., prior to administration to the subject, by testing in vitro or ex vivo the amount of BMP-10 agonist required to increase or stimulate one or more of the aforesaid BMP-10 biological activities. The method can, optionally, include the step(s) of identifying (e.g., evaluating, diagnosing, screening, and/or selecting) a subject at risk of having, or having, one or more symptoms associated with the condition and/or disorder.

In another aspect, the invention provides BMP-10 modulators, e.g., agonists or antagonists of BMP-10 expression and/or activity, in a vascular (e.g., an endothelial, smooth muscle), renal, and/or cardiac, cell or tissue, or a fibrotic tissue. For example, BMP-10 antagonists (e.g., the anti-BMP-10 or anti-BMP-10 receptor antibodies, BMP-10 propeptides, soluble BMP-10 receptors (e.g., BMP-10 receptors); or the BMP-10 agonists, can be identified and/or generated using the methods disclosed herein. Compositions, e.g., pharmaceutical compositions, that include the BMP-10 modulators are also disclosed. It is noted that the compositions, e.g., pharmaceutical compositions, may additionally include a second therapeutic agent, e.g., a second therapeutic agent as described herein.

Packaged pharmaceutical compositions that include the BMP-10 modulators for use in treating a vascular and/or cardiac disorder or condition described herein are also encompassed by the present invention. Optionally, the packaged pharmaceutical composition is labeled and/or contains some instructions for use in treating a vascular, renal, fibrotic and/or cardiac disorder or condition described herein.

In another aspect, the invention features BMP-10 binding agents, e.g., antibody molecules, binding domain fusion variants, antisense nucleic molecules which interact with, or more preferably specifically bind to BMP-10 polypeptides or fragments thereof, or nucleic acids encoding BMP-10. In one embodiment, the antibody molecules or the binding domain fusion variants bind to a mammalian, e.g., human, BMP-10 polypeptide or a fragment thereof. In one embodiment, the antibody molecule binds to an epitope located on: a pre-pro BMP-10 polypeptide (e.g., about amino acids 1 to 424 of FIG. 2 (SEQ ID NO:2)); a mature BMP-10 polypeptide (e.g., about amino acids 314 to 424, 314 to 325, 325 to 335, 335 to 345, 345 to 355, 355 to 365, 365 to 375, 375 to 385, 385 to 395, 395 to 405, 405 to 415, 415 to 424, of FIG. 2 (SEQ ID NO:2); a BMP-10 signal sequence (e.g., about amino acids 1 to 21 of FIG. 2 (SEQ ID NO:2)); and/or a BMP-10 propeptide region (e.g., about amino acids 22 to 424, 22 to 313, 22 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, of FIG. 2 (SEQ ID NO:2)). In one embodiment, the antibody molecules or binding domain fusion variants modulate, e.g., agonize or antagonize, one or more BMP-10-associated activities, e.g., an activity as described herein.

In one aspect, the invention features a method of providing an antibody molecule or a binding domain fusion variant that specifically binds to a human BMP-10 protein. The method includes: providing a human BMP-10 protein or fragment thereof (e.g., an antigen that comprises at least a portion of the BMP-10 protein as described herein); obtaining an antibody molecule or binding domain fusion variant that specifically binds to the human BMP-10 protein or fragment thereof; and evaluating if the antibody molecule or binding domain fusion variant specifically binds to the human BMP-10 protein, or evaluating efficacy of the antibody molecule or binding domain fusion variant in modulating, e.g., inhibiting, the activity of the human BMP-10 protein. The method can further include administering the antibody molecule to a subject, e.g., a human or non-human animal.

In another aspect, the invention features a method of evaluating, diagnosing, and/or monitoring the progression of, a BMP-10 associated disorder, e.g., a vascular and/or cardiac disorder (e.g., a disorder as described herein) in a test sample. The method includes evaluating the expression or activity of a nucleic acid or polypeptide chosen from BMP-10 or a BMP-10-associated gene, such that, a difference in the level of the nucleic acid or polypeptide relative to a reference sample, e.g., a sample obtained from normal subject or prior to treatment, is indicative of the presence or progression of the disorder. In embodiments, the BMP-10-associated nucleic acid or polypeptide is characterized by altered expression in response to BMP-10. Exemplary BMP-10-associated genes include, but are not limited to, GDF-8, GDF-10, endoglin, inhibitory Smad (e.g., Smad 6 and/or 7); and pro-angiogenic genes (e.g., VEGF, ID1 and ID2). In certain embodiments, an increase in the level of BMP-10 or a BMP-10-associated gene in the test sample, relative to a reference sample, is associated with the diagnosis of BMP-10 disorder where antagonism of BMP-10 function is desirable (e.g., a vascular, renal, fibrotic and/or cardiac disorder as described herein). In other embodiments, a decrease in the level of a BMP-10 or a BMP-10-associated gene in the test sample, relative to a reference sample, is associated with the diagnosis of BMP-10 vascular and/or cardiac disorder where agonism of BMP-10 function is desirable.

In one embodiment, the evaluating step occurs in vitro or ex vivo. For example, a sample, e.g., a serum sample, is obtained from the subject.

In another embodiment, the evaluating step occurs in vivo. For example, by administering to the subject a detectably labeled agent that interacts with the BMP-10, or BMP-10 associated, nucleic acid or polypeptide, such that a signal is generated relative to the level of activity or expression of the nucleic acid or polypeptide.

In yet another aspect, the invention provides a method, or an assay, for identifying a compound, e.g., a test compound, that modulates endothelial cell or vascular function The method, or the assay, includes: (i) (optionally) providing or identifying a test agent that interacts with, e.g., binds to, BMP-10 or a BMP-10 receptor; and/or (ii) evaluating a change in an activity of an endothelial cell and/or vascular tissue in the presence of the test agent, relative to a reference, e.g., a reference sample.

The test compound can be an antibody molecule; peptide; a soluble BMP-10 receptor or a fusion thereof; a binding domain fusion variant; a small molecule, e.g., a member of a combinatorial or natural product library; a nucleic acid; an antisense molecule; a ribozyme; an RNAi; a triple helix molecule; or any combination thereof. In one embodiment, the test compound modulates (e.g., decreases or increases) the activity or expression of a BMP-10 or a BMP-10 receptor polypeptide or nucleic acid. For example, the expression of the BMP-10 or a BMP-10 receptor nuclei acid can be modulated by e.g., altering mRNA transcription, mRNA stability, etc.

In embodiments, the evaluating step includes contacting one or more of: a BMP-10 or BMP-10 receptor polypeptide (e.g., a BMP-10 or BMP-10 receptor as described herein), or a nucleic acid encoding the BMP-10 or BMP-10 receptor, with the test compound; and evaluating a change in one or more activities of the BMP-10 or the BMP-10 receptor polypeptide or nucleic acid, in the presence of the test compound, relative to a predetermined level, e.g., a control sample without the test compound. The contacting step can be effected in vitro (in cultured cells, e.g., HUVECS or HAECS, or a reconstituted system) or in vivo (e.g., by administering the test compound to a non-human subject, e.g., an animal model having a mutation in a BMPR2 or a NKX2-5 gene). The contacting step(s) and/or the administration of the test compound can be repeated.

In embodiments, the change in an activity of the endothelial cell and/or vascular tissue is evaluated by measuring a change, in the presence of the test compound, relative to a reference, e.g., a reference sample (e.g., a control sample not exposed to the test compound), in one or more of: (i) phosphorylation of a Smad protein (e.g., phosphorylation of Smad 1, 5 and/or 8); (ii) gene expression of myostatin, endoglin and/or an inhibitory Smad (e.g., induction of expression of Smad 6 and/or 7); (iii) expression of one or more pro-angiogenic genes (e.g., VEGF, ID1 and ID2); (iv) expression of Ras-related protein-1a (Rap1a); (v) expression of one or more genes in response to BMP-10 stimulation of endothelial cells in vitro or in vivo identified in FIGS. 22-28; (vi) serum levels of stromal-derived differentiation factor (SDF-1) and/or matrix metallopeptidase 9 (MMP-9); and/or (vii) abnormalities in blood vessels, such as vascular dysplasia, hemorrhaging, telangiectasias, and/or arteriovenous malformations. In embodiments, a decrease in one or more of (i)-(iii) and (vi), and an increase in (iv), is indicative of an antagonist of BMP-10 function and thus, a candidate for treatment of a vascular and/or cardiac disorder where BMP-10 antagonism is desirable. In other embodiments, an increase in one or more of (i)-(iii) and (vi), and a decrease in (iv) is indicative of an agonist of BMP-10 function and thus, a candidate for treatment of a vascular and/or cardiac disorder where BMP-10 agonism is desirable.

In certain embodiments, an interaction between the test compound, the BMP-10 or the BMP-10 receptor is evaluated. In embodiments, such interaction can be evaluated by detecting a change in the formation and/or stability of the complex between the test compound and BMP-10 and/or BMP-10 receptor can be determined by detecting one or more of: a change in the binding or physical formation of the complex itself, e.g., by biochemical detection, affinity based detection (e.g., Western blot, affinity columns), immunoprecipitation, fluorescence resonance energy transfer (FRET)-based assays, spectrophotometric means (e.g., circular dichroism, absorbance, and other measurements of solution properties); a change, e.g., increase or decrease, in signal transduction, e.g., phosphorylation of Smads and/or transcription activity of a BMP-10-associated gene; a change, e.g., increase or decrease, in serum levels of stromal-derived differentiation factor (SDF-1) and/or matrix metallopeptidase 9 (MMP-9); and/or (vii) a change, e.g., increase or decrease in abnormalities in blood vessels, such as vascular dysplasia, hemorrhaging, telangiectasias, and/or arteriovenous malformations.

In one embodiment, the test compound is identified and re-tested in the same or a different assay. For example, a test compound is identified in an in vitro or cell-free system, and re-tested in an animal model or a cell-based assay. Any order or combination of assays can be used. For example, a high throughput assay can be used in combination with an animal model or tissue culture.

In other embodiments, the method, or assay, includes providing a step based on proximity-dependent signal generation, e.g., a two-hybrid assay that includes a first fusion protein (e.g., a fusion protein comprising a BMP-10 portion), and a second fusion protein (e.g., a fusion protein comprising a BMP-10 receptor), contacting the two-hybrid assay with a test compound, under conditions wherein said two hybrid assay detects a change in the formation and/or stability of the complex, e.g., the formation of the complex initiates transcription activation of a reporter gene.

In yet another aspect, the invention provides a host cell comprising one or more nucleic acids encoding one or more of the BMP-10 or BMP-10 receptor polypeptide constituents of the complex disclosed herein.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

All publications, patent applications, patents, and other references mentioned herein are incorporated by reference in their entirety.

Other features, objects, and advantages of the invention will be apparent from the description and drawings, and from the claims.

### DESCRIPTION OF DRAWINGS

FIG. 1 shows a nucleic acid sequence encoding a human BMP-10 preproprotein (SEQ ID NO: 1, 1584 bp).

FIG. 2 shows a human BMP-10 preproprotein amino acid sequence (SEQ ID NO: 2, 424 aa).

FIGS. 3A-3C shows a nucleic acid sequence encoding a human Activin A Receptor Type-II like 1 precursor protein (also referred to herein as “ALK1”) (SEQ ID NO: 3, 4263 bp).

FIG. 3D shows a human Activin A Receptor Type-II like 1 precursor protein (ALK1) amino acid sequence (SEQ ID NO: 4, 503 aa).

FIGS. 4A-4B shows a nucleic acid sequence encoding a human BMP-10 Receptor Type 1A precursor protein (also referred to herein as “ALK3”) (SEQ ID NO: 5, 3631 bp).

FIG. 4C shows a human BMP-10 Receptor Type 1A precursor protein (ALK3) amino acid sequence (SEQ ID NO: 6, 532 aa).

FIG. 5A shows a nucleic acid sequence encoding a human ActRIIb-Fc fusion protein (SEQ ID NO 17). The nucleic acid sequence encoding Gp1b signal sequence is underlined. The nucleic acid sequence encoding about amino acids 17 to 133 corresponding to human ActRIIb extracellular domain is fused to the sequence encoding a human IgG1 Fc. The nucleic acid sequence encoding human IgG1 Fc is shown in italic.

FIG. 5B shows a human ActRIIb-Fc fusion protein amino acid sequence (SEQ ID NO: 18). The Gp1b signal peptide sequence is underlined. The amino acid sequence from about amino acids 17 to 133 corresponding to human ActRIIb extracellular domain is fused to a human IgG1 Fc. The IgG1 Fc amino acid sequence is shown in italic.

FIGS. 6A-6B shows a nucleic acid sequence encoding a human SMAD family member 6 (SMAD6) protein (SEQ ID NO: 16, 2912 bp).

FIG. 7A shows a Northern blot analysis of human BMP-10 mRNA expression in human adult tissues.

FIG. 7B shows an interpretation guide for a multiple human tissue mRNA expression dot blot probed with human BMP-10.

FIG. 7C shows the results of a multiple human tissue expression mRNA dot blot probed with human BMP-10.

FIG. 8 shows a gross dissection of whole animals after BMP-10 overexpression in mice.

FIG. 9 shows a mouse brain dissection of an AdBMP-10 mouse after BMP-10 overexpression in mice.

FIG. 10 shows in vivo histopathological studies of brains of AdBMP-10 and control mice.

FIG. 11 shows in vivo histopathological studies of livers of AdBMP-10 and control mice.

FIG. 12 shows terminal body weights for AdBMP-10 and control mice.

FIG. 13 shows terminal organ weights for AdBMP-10 and control mice spleen, liver and thymus.

FIGS. 14A-14B shows data for serum chemistry changes for AST, ALP, and ALT in AdBMP-10 and control mice.

FIGS. 15A-15B shows differential serum chemistry studies of AdBMP-10 and control mice.

FIG. 16 shows an immunoblot demonstrating a time course of BMP-10 activation of endothelial cells in vitro via R-Smad 1, 5, 8 phosphorylation.

FIG. 17 shows quantitative PCR results demonstrating BMP-10 induction of inhibitory Smads 6 and 7 in endothelial cells in vitro.

FIG. 18 shows an immunoblot demonstrating the presence of BMP-10 in conditioned media, as probed with anti-BMP-10 pro antibody.

FIG. 19 shows an immunoblot demonstrating BMP-10 activation of renal epithelial cells in vitro via R-Smad 1, 5, 8 phosphorylation.

FIG. 20 shows an immunoblot demonstrating inhibition of BMP-10 signaling in renal epithelia cells by soluble ALK1 and soluble ActRIIB.

FIG. 21 shows an immunoblot demonstrating BMP-10 activation of mouse fibroblast cells in vitro via R-Smad 1, 5, 8 phosphorylation at various stages of adipocyte differentiation.

FIG. 22 shows a global gene expression analysis of differential gene expression in response to thirty minutes of BMP-10 treatment of both human umbilical vein endothelial cells and aortic endothelial cells (HUVECS and HUAECS) in vitro.

FIGS. 23A-23D shows a global gene expression analysis of differential gene expression in response to sixty minutes of BMP-10 treatment of both human umbilical vein endothelial cells and aortic endothelial cells (HUVECS and HUAECS) in vitro.

FIGS. 24A-24D shows a global gene expression analysis of differential gene expression in response to 5 hours of BMP-10 treatment of human renal proximal tubule epithelial cells in vitro.

FIGS. 25A-25E shows a global gene expression analysis of differential gene expression in response to 17 hours of BMP-10 treatment of human renal proximal tubule epithelial cells in vitro.

FIGS. 26A-26B shows a global gene expression analysis of differential gene expression in heart tissue of AdBMP-10 mice on day 3 post-injection.

FIGS. 26C-26L shows a global gene expression analysis of differential gene expression in heart tissue of AdBMP-10 mice on day 7 post-injection.

FIGS. 27A-27D shows a global gene expression analysis of differential gene expression in muscle tissue of AdBMP-10 mice on day 3 post-injection.

FIGS. 27E-27T shows a global gene expression analysis of differential gene expression in muscle tissue of AdBMP-10 mice on day 7 post-injection.

FIGS. 28A-28D shows a global gene expression analysis of differential gene expression in fat tissue of AdBMP-10 mice on day 3 post-injection.

FIG. 29 shows ELISA assay results demonstrating SDF-1 and MMP9 serum changes in AdBMP-10 mice.

Like reference symbols in the various drawings indicate like elements.

### DETAILED DESCRIPTION

The present invention is based, at least in part, on the discovery that in vivo overexpression of bone morphogenic protein-10 (BMP-10) resulted in profound subcutaneous vascular dysplasia in the brain and liver in animal models with a phenotype similar to Hereditary Hemorrhagic Telangiectasia (HHT). Animals overexpressing BMP-10 also presented a number of physiological abnormalities consistent with a function of BMP-10 in regulating vascular homeostasis, including widespread congestion and scattered hemorrhages, and enlarged blood vessels in the brain, liver and lung. Transcriptional profiling studies in response to BMP-10 overexpression in mice showed widespread changes in gene expression of regulators of muscle growth, such as myostatin, upregulation of pro-angiogenic proteins such as VEGF and ID1, and inhibitory Smads in fat and the heart. These results are consistent with upregulation of BMP-10 signal transduction cascade.

To further support a role of BMP-10 in modulating cardiac and vascular function, BMP-10 was shown to activate cell signaling events and gene expression in primary human aortic endothelial cells and primary human umbilical vein endothelial cells. For example, activation of BMP-10 signalling in these cells was shown to increase phosphorylation of Smads (e.g., Smad 1, 5 and 8) and induce expression of the inhibitory Smad 6 and 7.

In other embodiments, BMP-10 was shown to activate one or more signaling pathways and gene expression in renal cells in vitro (e.g., human primary renal proximal tubule epithelial cells). Applicants have further discovered an association of BMP-10 with fibrosis of various organs and tissues, including liver, lung, kidney and heart.

Thus, the present invention provides methods and compositions for modulating vascular (e.g., endothelial, smooth muscle), renal and/or cardiac cell function using BMP-10 agonists and antagonists. In particular, methods for treating, preventing and/or diagnosing BMP-10-associated vascular, renal, fibrotic and/or cardiac conditions and/or disorders are disclosed. Screening methods for evaluating BMP-10 modulators, e.g., agonists and antagonists, are also disclosed.

In order that the present invention may be more readily understood, certain terms are first defined. Additional definitions are set forth throughout the detailed description.

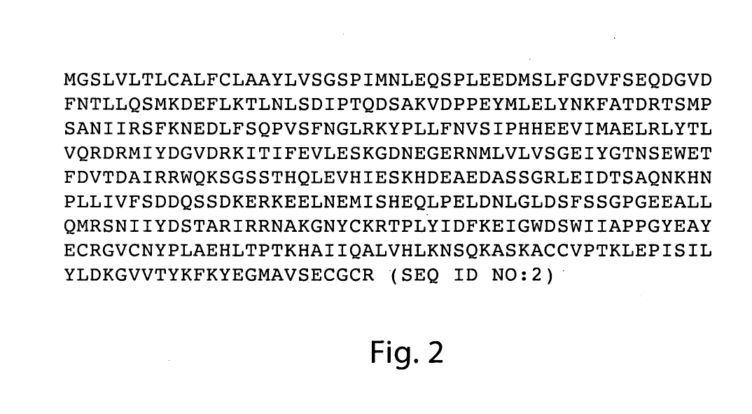

Members of the TGFβ superfamily, e.g., BMP-10, are generally encoded as a large precursor, referred to herein as “pre-propeptide,” that contains a signal sequence at its N-terminus (e.g., about amino acids 1 to 21 of human BMP-10 (FIG. 2; SEQ ID NO:2)) followed by a di-basic amino acid cleavage site and a propeptide, which, in turn, is followed by another dibasic amino acid cleavage site and a mature domain. Thus, a propeptide or prodomain is the portion that is N-terminal to the mature domain and C-terminal to the signal peptide. For example, human BMP-10 has a cleavage site that divides the 424 amino acid protein into a propeptide region (e.g., about 291 amino acids from amino acids 22 to 313 of FIG. 2 (SEQ ID NO:2)) and a mature region of approximately 110 amino acid residues per monomer (e.g., from about amino acids 314 to 424 of FIG. 2 (SEQ ID NO:2)). The mature BMP-10 protein has a spatially conserved pattern of six to seven cysteines typically found in TGFβ family members. The mature BMP-10 region is typically highly conserved among species.

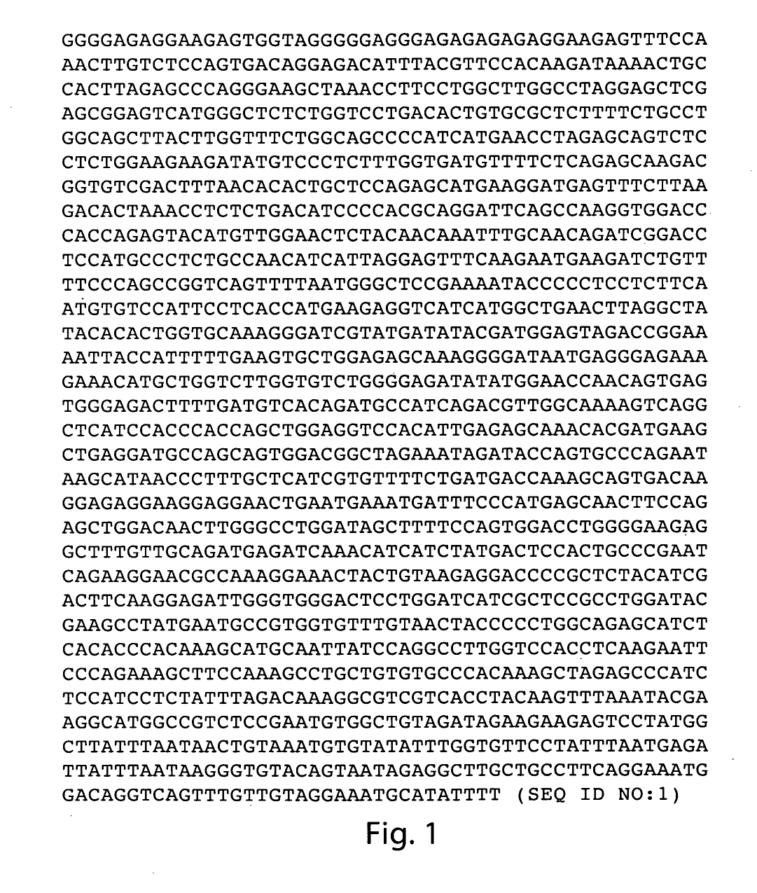

The term “Bone Morphogenic Protein-10” or “BMP-10” refers to a member of the transforming growth factor-beta (TGFβ) family from any species (typically of mammalian, e.g., murine, or human or non-human primate origin), as well as any variants thereof (including mutants, fragments and peptidomimetic forms) that retain a BMP-10 activity (e.g., which is capable of interacting with, e.g., binding to, BMP-10 receptor (typically of mammalian, e.g., murine or human BMP-10 receptor)). Typically, the BMP-10 has a biological activity as described herein and one of the following features: (i) an amino acid sequence of a naturally occurring mammalian BMP-10 polypeptide or a fragment thereof (e.g., a mature or propeptide form of BMP-10), e.g., an amino acid sequence shown as FIG. 2 (SEQ ID NO:2) (human) or a fragment thereof (e.g., about amino acids 314 to 424 of FIG. 2 (SEQ ID NO:2) (mature)); (ii) an amino acid sequence substantially homologous to, e.g., at least 85%, 90%, 95%, 98%, 99% homologous to, an amino acid sequence shown as FIG. 2 (SEQ ID NO:2) (human) or a fragment thereof (e.g., about amino acids 314 to 424 of FIG. 2 (SEQ ID NO:2) (mature)); (iii) an amino acid sequence which is encoded by a naturally occurring mammalian BMP-10 nucleotide sequence or a fragment thereof (e.g., FIG. 1 (SEQ ID NO:1) (human) or a fragment thereof); (iv) an amino acid sequence encoded by a nucleotide sequence which is substantially homologous to, e.g., at least 85%, 90%, 95%, 98%, 99% homologous to, a nucleotide sequence shown as FIG. 1 (SEQ ID NO:1) (human) or a fragment thereof; (v) an amino acid sequence encoded by a nucleotide sequence degenerate to a naturally occurring BMP-10 nucleotide sequence or a fragment thereof, e.g., FIG. 1 (SEQ ID NO:1) (human) or a fragment thereof; or (vi) a nucleotide sequence that hybridizes to one of the foregoing nucleotide sequence sequences under stringent conditions, e.g., highly stringent conditions.

The term “BMP-10 propeptide” is used to refer to polypeptides comprising any naturally occurring propeptide of a BMP-10 family member, as well as any variants thereof (including mutants, fragments and peptidomimetic forms) that retain a useful activity. In some embodiments, the BMP-10 propeptides disclosed herein are used as antagonists of a mature BMP-10. As the term is used herein, BMP-10 propeptides include fragments, functional variants, and modified forms of BMP-10 propeptides. In one embodiment, the BMP-10 propeptide includes the amino acid sequence of amino acids 22 to 313 of FIG. 2 (SEQ ID NO:2), or a portion thereof, but does not include the full length mature BMP-10 sequence. The BMP-10 propeptide can contain fewer than 50, 40, 30, 20, 10 or 5 amino acids of its cognate mature domain. Functional variants of a BMP-10 propeptide may be characterized by, for example, binding to mature BMP-10 protein and/or the ability to competitively inhibit the binding of BMP-10 to a BMP-10 receptor.

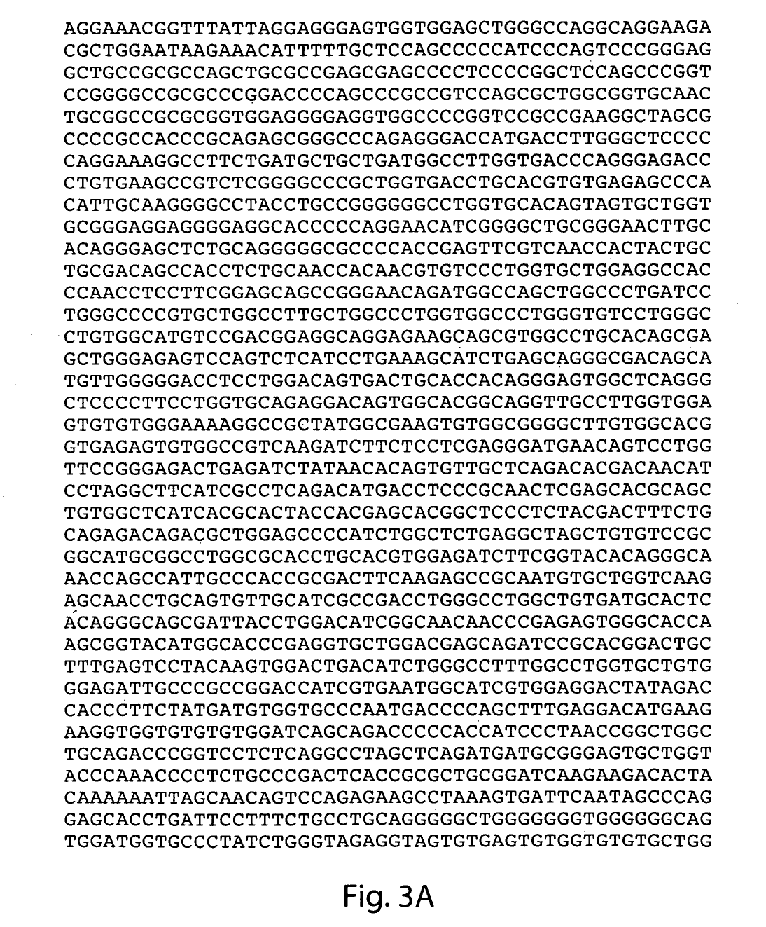

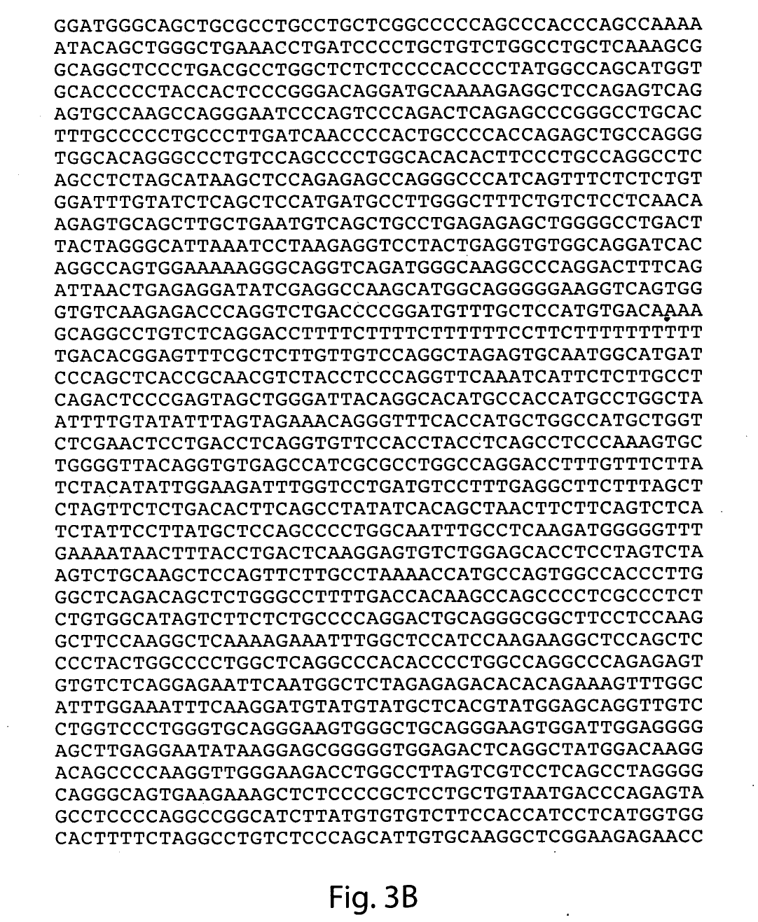

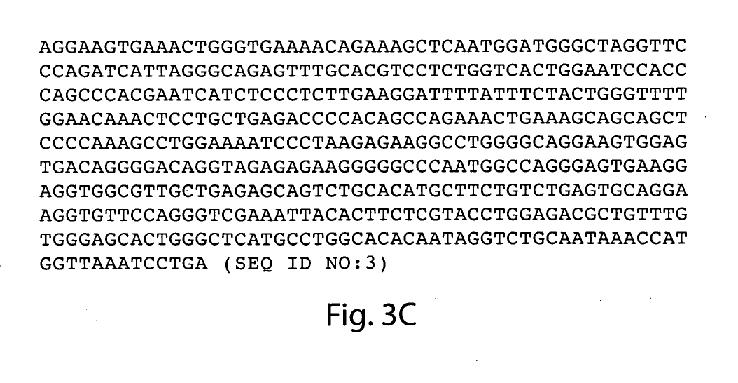

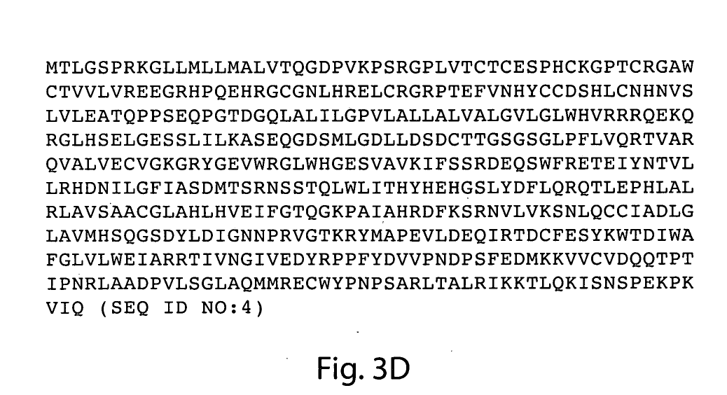

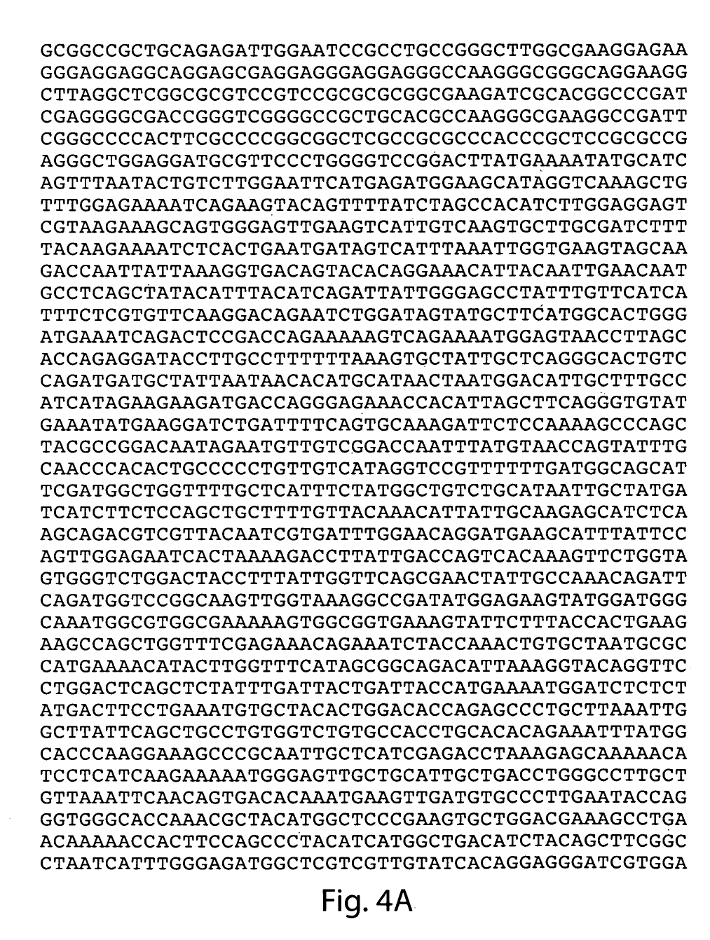

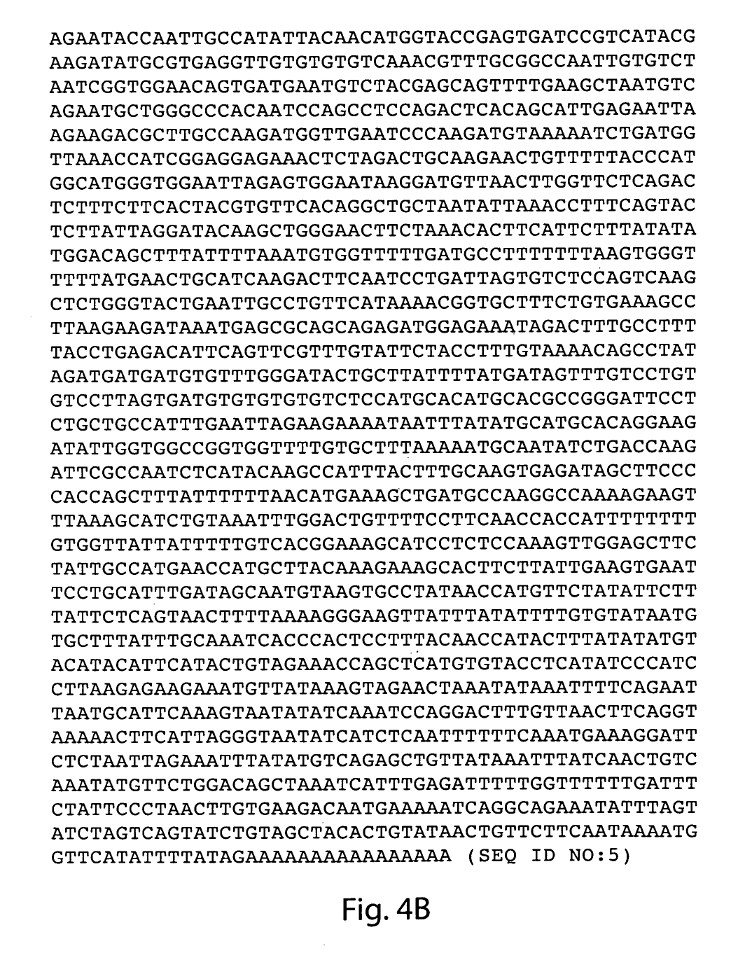

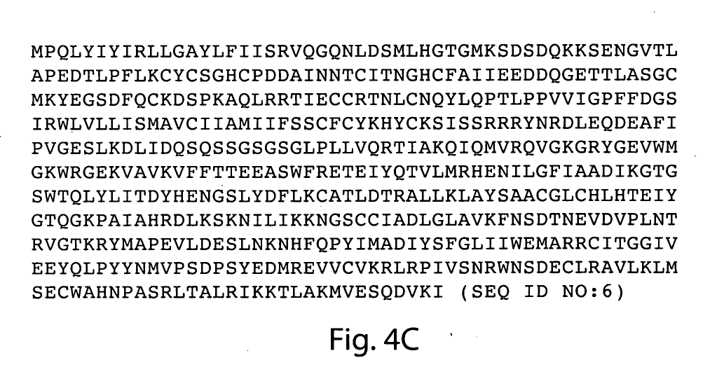

As used herein, the term “Bone Morphogenic Protein-10 receptor” or “BMP-10 receptor” refers to a receptor from any species that is capable of binding BMP-10 and transducing a signal in a cell, e.g., an endothelial cell. Examples of BMP-10 receptors include endoglin, Activin A Receptor Type-II like 1 precursor protein (also referred to herein as “ALK1,” the nucleotide and amino acid sequence of which is depicted herein as FIGS. 3A-3D; SEQ ID NO: 3 and SEQ ID NO: 4, respectively), and human BMP-10 Receptor Type 1A precursor protein (also referred to herein as “ALK3,” the nucleotide and amino acid sequence of which are depicted herein as FIGS. 4A-4C; SEQ ID NO: 5 and SEQ ID NO:6, respectively). Activation of a BMP-10 receptor typically results in phosphorylation of a Smad protein (e.g., phosphorylation of one or more of Smadt, Smad2, Smad3, Smad5 or Smad8), and gene transcription events.

Typically, the BMP-10 receptor has a biological activity as described herein and/or one of the following features: (i) an amino acid sequence of a naturally occurring mammalian BMP-10 receptor polypeptide or a fragment thereof, e.g., an amino acid sequence shown as FIG. 3D (SEQ ID NO:4) (human ALK-1), or FIG. 4C (SEQ ID NO:6) (human ALK3), or a fragment thereof (e.g., about amino acids 22-118 of FIG. 3D, SEQ ID NO:4 or about amino acids 24 to 152 of FIG. 4C (SEQ ID NO:6) (extracellular domain)); (ii) an amino acid sequence substantially homologous to, e.g., at least 85%, 90%, 95%, 98%, 99% homologous to, an amino acid sequence shown as FIG. 3D (SEQ ID NO:4) (human ALK-1), or FIG. 4C (SEQ ID NO:6) (human ALK3), or a fragment thereof (e.g., about amino acids 22-118 of FIG. 3D, SEQ ID NO:4 or about amino acids 24 to 152 of FIG. 4C (SEQ ID NO:6) (extracellular domain)); (iii) an amino acid sequence which is encoded by a naturally occurring mammalian BMP-10 receptor nucleotide sequence or a fragment thereof (e.g., FIGS. 3A-3C (SEQ ID NO:3) (human ALK1) or FIGS. 4A-4B (SEQ ID NO:5 (human ALK3), or a fragment thereof); (iv) an amino acid sequence encoded by a nucleotide sequence which is substantially homologous to, e.g., at least 85%, 90%, 95%, 98%, 99% homologous to, a nucleotide sequence shown as FIGS. 3A-3C (SEQ ID NO:3) (human ALK1) or FIGS. 4A-4B (SEQ ID NO:5 (human ALK3); (v) an amino acid sequence encoded by a nucleotide sequence degenerate to a naturally occurring BMP-10 nucleotide sequence or a fragment thereof, e.g., FIGS. 3A-3C (SEQ ID NO:3) (human ALK1) or FIGS. 4A-4B (SEQ ID NO:5 (human ALK3); or (vi) a nucleotide sequence that hybridizes to one of the foregoing nucleotide sequence sequences under stringent conditions, e.g., highly stringent conditions.

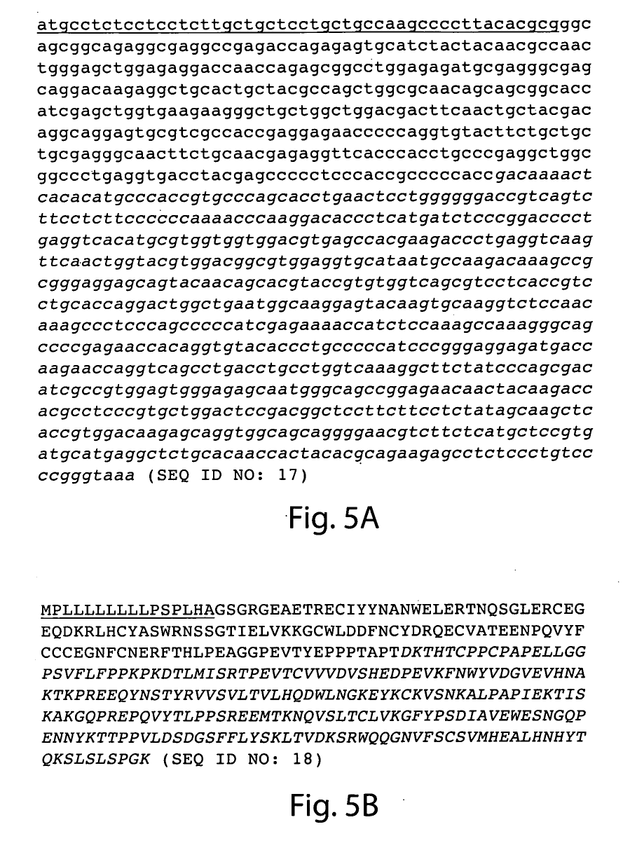

As used herein, a “soluble BMP-10 receptor polypeptide” is a BMP-10 receptor polypeptide incapable of anchoring itself in a membrane. Such soluble polypeptides include, for example, a BMP-10 receptor polypeptide as described herein that lack a sufficient portion of their membrane spanning domain to anchor the polypeptide or are modified such that the membrane spanning domain is non-functional. Typically, the soluble BMP-10 receptor polypeptide retains the ability of binding to BMP-10. E.g., a soluble fragment of a BMP-10 receptor polypeptide (e.g., a fragment of a BMP-10 receptor comprising the extracellular domain of human ALK-1 or ALK-including an amino acid sequence from about amino acids 22-118 of FIG. 3D (SEQ ID NO:4); about amino acids 24 to 152 of FIG. 4C (SEQ ID NO:6); or a soluble fragment of an activin receptor IIB (ActRIIB), e.g., including about amino acids 17 to 133 of FIG. 5B; SEQ ID NO:18. A soluble BMP-10 receptor polypeptide can additionally include, e.g., be fused to, a second moiety, e.g., a polypeptide (e.g., an immunoglobulin chain, a GST, Lex-A or MBP polypeptide sequence). For example, a fusion protein can includes at least a fragment of a BMP-10 receptor polypeptide, which is capable of binding BMP-10, fused to a second moiety, e.g., a polypeptide (e.g., an immunoglobulin chain, an Fc fragment, a heavy chain constant regions of the various isotypes, including: IgG1, IgG2, IgG3, IgG4, IgM, IgA1, IgA2, IgD, and IgE).

The phrase “a biological activity of” a BMP-10 receptor polypeptide refers to one or more of the biological activities of the corresponding mature BMP-10 protein, including, but not limited to, (1) interacting with, e.g., binding to, an BMP-10 polypeptide (e.g., a human mature BMP-10 polypeptide); (2) stimulating phosphorylation and/or activation of Smad proteins (e.g., phosphorylation of one or more of Smad1, Smad2, Smad3, Smad5 or Smad8); (3) increase or decreased transcription of a BMP-10-associated gene; and/or (4) modulating, e.g., stimulating or decreasing, proliferation, differentiation of endothelian and cardiac cells.

The methods and compositions of the present invention encompass BMP-10 and BMP-10 receptor polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to SEQ ID NO:2, SEQ ID NO:4 or SEQ ID NO:6 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to SEQ ID NO:1, 3, or 5 are termed substantially identical.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to BMP-10/BMP-10 receptor nucleic acid (SEQ ID NO:1) molecules of the invention. BLAST protein searches can be performed with the XBLAST program, score=50, wordlength=3 to obtain amino acid sequences homologous to BMP-10/BMP-10 receptor (SEQ ID NO:1) protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the BMP-10/BMP-10 receptor agonists and antagonists of the present invention may have additional conservative or non-essential amino acid substitutions, which do not have a substantial effect on their functions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Various aspects of the invention are described in further detail below.

### I. Modulation of BMP-10-Associated Cell Function

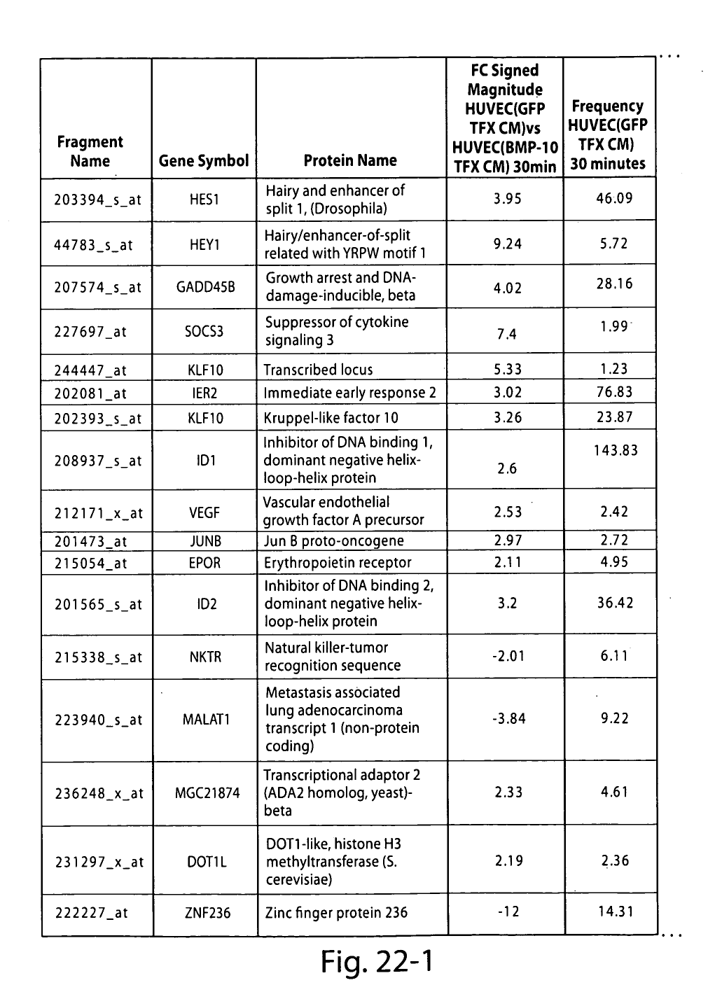

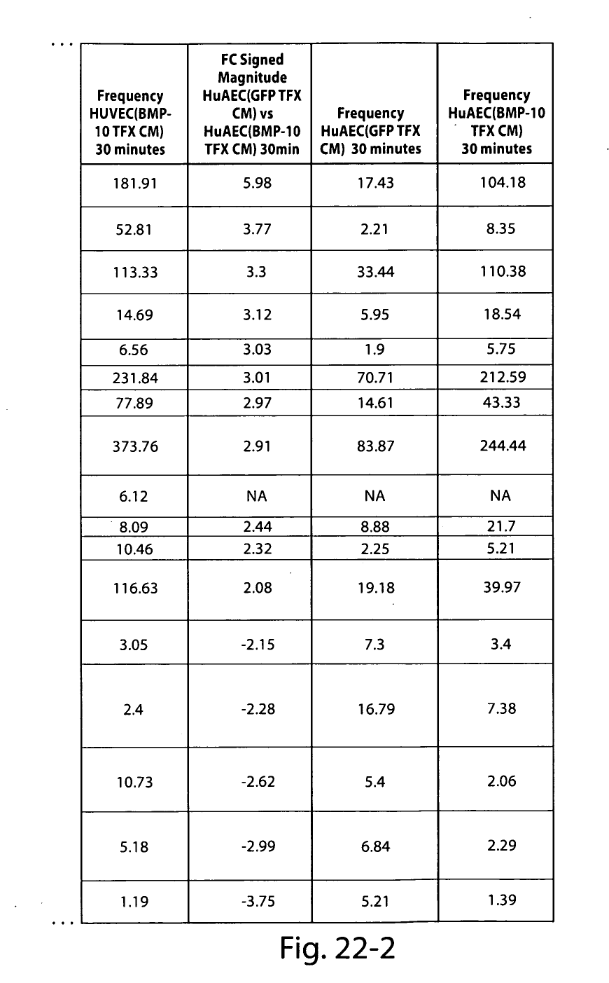

The invention provides methods for modulating a BMP-10 function (e.g., by modulating one or more biological activities of BMP-10 in a cardiac, renal and/or vascular cell and/or tissue, or fibrotic tissue or organ). The method includes contacting the cell and/or tissue with a BMP-10 modulator, e.g., an agonist or an antagonist of BMP-10 (e.g., an agonist or an antagonist of human mature BMP-10 or BMP-10 pro-peptide) activity or expression, in an amount sufficient to modulate (e.g., increase or decrease), the function of the BMP-10-responsive cell and/or tissue (or the biological activity of BMP-10 in the cell or tissue). The term “BMP-10-responsive cell and/or tissue” refers to any cell and/or tissue capable of transducing a signal, e.g., phosphorylation, gene expression, in response to BMP-10. In embodiments, the BMP-10-responsive cell is a vascular cell and/or tissue, such as an endothelial and/or smooth muscle cell or tissue. In other embodiments, the BMP-1 responsive cell and/or tissue includes cardiac cell and/or tissue, such as a cardiomyocyte. In other embodiments, the BMP-10 responsive cell and/or tissue includes a renal cell or tissue. In yet other embodiments, the BMP-10 responsive cell includes a fibrotic tissue or organ (e.g., a fibrotic skin, kidney, lung, gut, liver, peritoneum or heart). Exemplary BMP-10 activities that can be modulated using the methods and compositions of the invention include, but not limited to, one or more of the following: (i) phosphorylation of a Smad protein (e.g., phosphorylation of Smad 1, 5 and/or 8); (ii) induction of gene expression of myostatin, endoglin and/or an inhibitory Smad (e.g., induction of expression of Smad 6 and/or 7); (iii) increased expression of pro-angiogenic genes (e.g., VEGF, ID1 and ID2); (iv) decreased expression of Ras-related protein-1a (Rap1a); (v) modulation of, e.g., increase or decrease, expression of one or more genes in response to BMP-10 stimulation of endothelial cells in vitro or in vivo identified in FIGS. 22-28; (vi) increased serum levels of stromal-derived differentiation factor (SDF-1) and/or matrix metallopeptidase 9 (MMP-9); and/or (vii) increased abnormalities in blood vessels, such as vascular dysplasia, hemorrhaging, telangiectasias, and/or arteriovenous malformations.

The methods of the invention can be performed on cells (e.g., cardiomyocytes, endothelial and/or smooth muscle cells) present in a subject, e.g., as part of an in vivo (e.g., therapeutic or prophylactic) protocol, or in an animal subject (e.g., an in vivo animal model, such as a cardiovascular ischemic model or a genetically modified model, e.g., an animal model having a mutation in a BMP receptor (BMPR2) or an NKX2-5 deficient animal). For in vivo methods, the BMP-10 modulator, alone or in combination with another agent, can be administered to a subject, e.g., a mammal, suffering from a BMP-10-associated (e.g., vascular or cardiac) condition and/or disorder, in an amount sufficient to modulate, BMP-10 function, and/or one or more BMP-10 activities in the subject.

In embodiments, the BMP-10 modulator is administered in a therapeutically effective amount. As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of a BMP-10-agonist or antagonist may be administered either alone or in combination with other therapies such as treatments. When co-administered with one or more agents, a BMP-10- and/or BMP-10 receptor-modulator may be administered either simultaneously with the second agent, or sequentially. If administered sequentially, the attending physician will decide on the appropriate sequence of administering a BMP-10/BMP-10 receptor-agonist or antagonist in combination with other agents. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

In embodiments where inhibition, reduction or otherwise diminution of one of more BMP-10 biological activities is desired, a BMP-10-responsive cell, e.g., a vascular (e.g., endothelial, smooth muscle), and/or cardiac, cell and/or tissue is contacted with a BMP-10 antagonist, e.g., by administering the BMP-10 antagonist to the subject. In one embodiment, the BMP-10 antagonist interacts with, e.g., binds to, BMP-10 or a BMP-10 receptor (also individually referred to herein as a “BMP-10 antagonist” and “BMP-10 receptor antagonist,” respectively), and reduces or inhibits one or more of BMP-10 and/or BMP-10 receptor activities. Typical antagonists bind to BMP-10 or the BMP-10 receptor with high affinity, e.g., with an affinity constant of at least about 10⁷ M⁻¹, typically about 10⁸ M⁻¹, and more typically, about 10⁹ M⁻¹ to 10¹⁰ M⁻¹ or stronger; and reduce and/or inhibit one or more BMP-10 biological activities in an endothelial cell and/or vascular tissue. As used herein, a “BMP-10 antagonist” or a “BMP-10 receptor antagonist,” that is useful in the method of the invention, refers to an agent which reduces, inhibits or otherwise diminishes one or biological activities of a BMP-10/BMP-10 receptor polypeptide. Typically, the antagonist interacts with, e.g., binds to, a BMP-10/BMP-10 receptor polypeptide. Antagonism using a BMP-10/BMP-10 receptor antagonist does not necessarily indicate a total elimination of the BMP-10/BMP-10 receptor polypeptide biological activity.

Thus, the invention provides methods of treating or preventing (e.g., curing, suppressing, ameliorating, delaying or preventing the onset of, or preventing recurrence or relapse of) a BMP-10-associated (e.g., vascular or cardiac) condition and/or disorder, in a subject. The method includes administering to the subject a BMP-10 antagonist (e.g., a BMP-10 antagonist as described herein), in an amount sufficient to inhibit or reduce one or more BMP-10 biological activities in the vascular and/or cardiac cell and/or tissue (e.g., a biological activity as described herein), thereby treating or preventing the disorder or condition.

The subject to whom the BMP-10 antagonist is administered can be a mammal, e.g., a human, suffering from, for example, a condition and/or disorder characterized by aberrant vascular, e.g., endothelial and/or smooth muscle) cell activity, referred to herein as a “BMP-10-associated vascular condition and/or disorder.” For example, the subject can be a mammal (e.g., a human patient) suffering from a disorder or condition chosen from one or more of: Hereditary Hemorrhagic Telangiectasia (HHT); nephritic syndrome and nephropathy (e.g., diabetic nephropathy), retinopathy (e.g., diabetic retinopathy), neovascular glaucoma and other diabetic vascular conditions; stroke, atherosclerosis, arteriosclerosis, peripheral artery disease, hypertension (e.g., pulmonary hypertension), hyperlipidemia, thrombosis, restenosis, rheumatoid arthritis, psoriasis, hemangiomas, thyroid hyperplasias (including Grave's disease), corneal and other tissue transplantation, and chronic inflammation and retrolental fibroplasias. The antagonists are also useful for the treatment of disorders characterized by undesirable excessive vascular permeability, such as edema associated with brain tumors, ascites associated with malignancies, Meigs' syndrome, lung inflammation, nephrotic syndrome, pericardial effusion (such as that associated with pericarditis), and pleural effusion.

For example, Applicants have discovered that overexpression of BMP-10 in mice causes a phenotype similar to hereditary hemorrhagic telangiectasia (HHT). HHT, also known as Rendu-Osler disease, is an autosomal dominant vascular dysplasia that affects about 1 in 10,000 people (Johnson, D. W. et al. (1996) Nat. Genet. 13:189-195). The clinical abnormalities in HHT are typically caused by direct arteriovenous connections without an intervening capillary bed. The resulting telangiectases occur in the oral cavity (e.g., lips and tongue), in the nose, and the fingertips. Larger arteriovenous malformations (AVMs) can also be found in the lung, brain and liver (Marchuk, D. A. et al. (2003) Hum. Mol. Genet. 12:R97-R112). The majority of the HHT cases are caused by mutations in either Endoglin (ENG) or ALK1 genes. Recently mutations in SMAD4 have also been described in cases with combined juvenile polyposis and HHT syndromes (Gallione, C. J. et al. (2006) J. Med. Genet. 43:793-797). Each of the three genes implicated in HHT (ENG, ACVRL1 and SMAD4) encode receptors or signaling molecules from the TGFβ family. Thus, the appended Examples, in combination with the literature, support the use of antagonists of BMP-10 activity to treat HHT.

The BMP-10 antagonists can also be useful in the treatment of various neoplastic disorders. Neoplasms and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, and Meigs' syndrome.

The BMP-10 antagonists can also be useful in the treatment of fibrotic conditions or disorders. Fibrosis conditions are pathological conditions that are characterized by the abnormal and/or excessive accumulation of fibrotic material (e.g., extracellular matrix) following tissue damage. Fibrosis conditions include fibroproliferative disorders that are associated with vascular diseases, such as cardiac disease, cerebral disease, and peripheral vascular disease, as well as in many tissues and organ systems, including the skin, kidney, lung, gut and liver. (Wynn, Nature Reviews 4:583-594 (2004)). Although fibrosis conditions are a diverse group of pathologies, it is believed that for most fibrosis conditions, the general mechanisms leading to fibrotic tissue accumulation have many elements in common.

Exemplary fibrosis conditions include, but are not limited to: (i) Lung or pulmonary fibrosis, e.g., idiopathic pulmonary fibrosis, radiation induced fibrosis, chronic obstructive pulmonary disease (COPD), scleroderma, pulmonary fibrosis, chronic asthma, silicosis, asbestos-induced pulmonary fibrosis, acute lung injury and acute respiratory distress (including bacterial pneumonia induced, trauma induced, viral pneumonia induced, ventilator induced, non-pulmonary sepsis induced, and aspiration induced); (ii) kidney fibrosis, including, but not limited to, nephropathies associated with injury/fibrosis, e.g., chronic nephropathies associated with diabetes (e.g., diabetic nephropathy), lupus, scleroderma, glomerular nephritis, focal segmental glomerular sclerosis, and IgA nephropathy; (iii) gut fibrosis, e.g., scleroderma, and radiation induced gut fibrosis; (iv) liver fibrosis, e.g., cirrhosis, alcohol induced liver fibrosis, non-alcoholic steatohepatitis (NASH), biliary duct injury, primary biliary cirrhosis, infection or viral induced liver fibrosis (e.g., chronic HCV infection), and autoimmune hepatitis; (v) head and neck fibrosis, e.g., radiation induced; (vi) corneal scarring, e.g., laser surgery, corneal transplant, and trabeculectomy; (vii) hypertrophic scarring and keloids, e.g., burn induced and surgical; and (viii) other fibrotic diseases, e.g., sarcoidosis, scleroderma, spinal cord injury/fibrosis, myelofibrosis, vascular restenosis, atherosclerosis, Wegener's granulomatosis, mixed connective tissue disease and Peyronie's disease.

Several assay systems and animal models are known in the art for evaluating the effects of the BMP-10 modulators (see e.g., Wynn, Nature Reviews 4:583-594 (2004); Phan and Kunkel (1992) Exp. Lung Res., 18: 29-43; WO2000/064944, the contents of which are hereby incorporated by reference). One exemplary model for pulmonary fibrosis is the bleomycin (BL)-rodent models. This model is appealing because it produces a characteristic fibrosis symptoms with many of the components of human disease, and because BL-induced pulmonary fibrosis is a well-recognized adverse effect in human chemotherapy. Intratracheal (IT) instillation of BL in rodents has been widely used for studying mechanisms of fibrogenesis and for screening potentially desirable antifibrotic compounds. Although the initial cause of BL-induced pulmonary toxicity is attributed to the generation of reactive oxygen species (ROS) once it binds to iron and DNA, the process leading to the final manifestation of pulmonary fibrosis involves release of various inflammatory mediators (Giri and Wang., Comments Toxicol., 3: 145-176 (1989)). The pathogenesis of BL-induced lung injury is initially characterized by edema, hemorrhage, and a cellular infiltrate predominated by neutrophils and macrophages. An excess accumulation of the inflammatory leukocytes in vascular, interstitial and alveolar spaces of the lung could inflict vascular- and parenchymal injury by the generation of ROS and proteolytic enzymes.

According to another embodiment of the invention, the effectiveness of the antagonist in preventing or treating disease may be improved by administering the antagonist serially or in combination with another agent that is effective for those purposes, such as tumor necrosis factor (TNF) inhibitors; an antibody capable of inhibiting or neutralizing the angiogenic activity of acidic or basic fibroblast growth factor (FGF); hepatocyte growth factor (HGF) (TGFB) (e.g., in the treatment of diabetic nephropathy and other renal indications); an antibody capable of inhibiting or neutralizing the coagulant activities of tissue factor, protein C, or protein S (see Esmon, et al., PCT Patent Publication No. WO 91/01753, published 21 Feb. 1991), or one or more conventional therapeutic agents such as, for example, alkylating agents, folic acid antagonists, anti-metabolites of nucleic acid metabolism, antibiotics, pyrimidine analogs, 5-fluorouracil, purine nucleosides, amines, amino acids, triazol nucleosides, or corticosteroids. Such other agents may be present in the composition being administered or may be administered separately. Also, the antagonist is suitably administered serially or in combination with radiological treatments, whether involving irradiation or administration of radioactive substances.

In other embodiments, BMP-10 antagonists can be used to treat a subject that suffers from a BMP-10 associated cardiac disorder or condition. Cardiac or heart disorders can be characterized by any kind of cardiac dysfunction involving, e.g., inadequate blood supply to the heart; irregularities in the heart rhythm; and/or defective conduction of impulses from the atria to the ventricles of the heart. Examples of cardiac disorders include, but are not limited to, congenital heart disease, cardiomyopathy (e.g., dilated, hypertrophic, restrictive cardiomyopathy), congestive heart failure, and myocardial infarction. The BMP-10 antagonist can be administered to the subject alone, or in combination with one or more agents or therapeutic modalities, e.g., therapeutic agents, which are useful for treating BMP-10 associated vascular or cardiac disorders and/or conditions. In one embodiment, the second agent or therapeutic modality is a chosen from one or more of: angioplasty, beta blockers, anti-hypertensives, cardiotonics, anti-thrombotics, vasodilators, hormone antagonists, endothelin antagonists, calcium channel blockers, phosphodiesterase inhibitors, angiotensin type 2 antagonists and/or cytokine blockers/inhibitors.

In embodiments where an increase in endothelial cell or vascular function, is desired, the endothelial cell or vascular tissue is contacted with a BMP-10 agonist, e.g., by administering the BMP-10 agonist to a subject. As used herein, a “BMP-10 agonist” or a “BMP-10 receptor agonist,” that is useful in the method of the invention, refers to an agent which potentiates, induces or otherwise enhances one or biological activities of a BMP-10/BMP-10 receptor polypeptide. Typically, the agonist interacts with, e.g., binds to, a BMP-10 receptor polypeptide and activates one or more BMP-10 receptor activities. Examples of BMP-10 agonists include a BMP-10 protein or a functionally active fragment, peptide, or variant thereof (e.g., a mammalian, e.g., human, BMP-10 (e.g., mature BMP-10 as described herein or a sequence substantially homologous thereto); or a nucleic acid encoding the BMP-10 protein or functionally active fragment or variant thereof (e.g., a nucleic acid that includes the nucleotide sequence shown in FIG. 1 (SEQ ID NO:1) (or a portion or variant thereof). The BMP-10 agonist can be used alone or functionally linked (e.g., by chemical coupling, genetic or polypeptide fusion, non-covalent association or otherwise) to a second moiety, e.g., an immunoglobulin Fc domain, serum albumin, as described herein. In other embodiments, the BMP-10 agonist binds to a BMP-10 receptor, e.g., a receptor as described herein, and increases the receptor activity. For example, the BMP-10 agonist can be an antibody molecule, a binding domain fusion variant, or any other agent that binds to a BMP-10 receptor and stimulates one or more activities.

The subject treated with the BMP-10 agonist(s) can be a mammal, e.g., a human, suffering from, for example, a disorder characterized by underactive or disrupted BMP-10 cell proliferation and/or activity. For example, the BMP-10 agonist can be used to treat or prevent a disorder or condition following endothelial cell injury, e.g., after an ischemia attack or microvascular angiopathy. Thus, methods of treating or preventing (e.g., curing, suppressing, ameliorating, delaying or preventing the onset of, or preventing recurrence or relapse of) a disorder characterized by characterized by underactive or disrupted BMP-10-responsive cell proliferation and/or activity are disclosed. For example, the BMP-10 agonist can be used to treat or prevent a disorder or condition following endothelial cell injury, e.g., after an ischemia attack or microvascular angiopathy. The method includes administering to the subject a BMP-10 agonist (e.g., a BMP-10 agonist as described herein), in an amount sufficient to increase or stimulate one or more BMP-10 biological activities in an endothelial cell and/or vascular tissue (e.g., a biological activity as described herein), thereby treating or preventing the disorder or condition. The subject can be a mammal, e.g., a human, suffering from, for example, the disorder or condition.

The BMP-10/BMP-10 receptor agonists and antagonists (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

When a therapeutically effective amount of a BMP-10/BMP-10 receptor-agonist or antagonist is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the binding agent, and preferably from about 1 to 50% the binding agent.

When a therapeutically effective amount of a BMP-10/BMP-10 receptor-agonist or antagonist is administered by intravenous, cutaneous or subcutaneous injection, binding agent will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to binding agent an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additive known to those of skill in the art.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In those embodiments where the antagonist is a biologic, e.g., an antibody molecule, soluble receptor fusion, about 1 μg/kg to 15 mg/kg of antagonist is an initial candidate dosage for administration to the patient depending on the type and severity of the disease, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic tumor imaging.

The duration of therapy using the pharmaceutical composition of the present invention will vary, depending on the severity of the disease being treated and the condition and potential idiosyncratic response of each individual patient. It is contemplated that the duration of each application of the BMP-10/BMP-10 receptor-agonist or antagonist will be in the range of 12 to 24 hours of continuous intravenous administration. Ultimately the attending physician will decide on the appropriate duration of intravenous therapy using the pharmaceutical composition of the present invention.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### II. BMP-10 Agonists and Antagonists

### Antibody Molecules

In certain embodiments, the BMP-10 agonists/antagonists are antibody molecules against BMP-10 or a BMP-10 receptor. In embodiments, the antibody molecule is a monoclonal or single specificity antibody, or an antigen-binding fragment thereof (e.g., an Fab, F(ab′)₂, Fv, a single chain Fv fragment, or a camelid variant) that binds to BMP-10 or a BMP-10 receptor, e.g., a mammalian (e.g., human, BMP-10 or BMP-10 receptor (or a functional variant thereof)). In embodiments, the antibody molecule binds to mature BMP-10 (e.g., a mature human BMP-10 as described herein).

Typically, the antibody molecule is a human, humanized, chimeric, camelid, or in vitro generated antibody to human BMP-10 or human BMP-10 receptor polypeptide (or functional fragment thereof). Typically, the antibody inhibits, reduces or neutralizes one or more activities of BMP-10 or a BMP-10 receptor (e.g., one or more biological activities of BMP-10 as described herein). In one embodiment, the antibody molecule binds to a mature BMP-10 polypeptide (e.g., about amino acids 314 to 424, or an epitope comprising fragments thereof, e.g., about amino acids 314 to 325, 325 to 335, 335 to 345, 345 to 355, 355 to 365, 365 to 375, 375 to 385, 385 to 395, 395 to 405, 405 to 415, and 415 to 424, of FIG. 2 (SEQ ID NO:2)). In other embodiments, the antibody molecule binds to a BMP-10 propeptide (e.g., about amino acids 22 to 424, or an epitope comprising a fragment thereof (e.g., about amino acids 22 to 313, 22 to 30, to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, of FIG. 2 (SEQ ID NO:2)). In yet other embodiments, the antibody molecule binds to a cleavage site in the BMP-10 propeptide, e.g., binds to an epitope located at about amino acids 21-22 or 313-314 of SEQ ID NO:2. In embodiments, the antibody molecule binds to a BMP-10 receptor, e.g., endoglin, e.g., a human endoglin; or an activin receptor-like kinase (ALK)-1, -3, or -6 (e.g., an ALK-1, or -3 comprising an amino acid sequence identical to a mammalian, e.g., human, ALK-1 and -3 as shown in FIG. 3D (SEQ ID NO:4) and FIG. 4C (SEQ ID NO:6), respectively), or a sequence substantially homologous thereto.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present invention can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof, Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibodies of the present invention can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the invention, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies derived from a variable region of NAR (“IgNARs”) are described in WO 03/014161 and Streltsov (2005) Protein Sci. 14:2901-2909. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to BMP-10/BMP-10 receptor, or an epitope thereof. With respect to proteins (or protein mimetics), the antigen-binding site typically includes one or more loops (of at least four amino acids or amino acid mimics) that form an interface that binds to BMP-10/BMP-10 receptor. Typically, the antigen-binding site of an antibody molecule includes at least one or two CDRs, or more typically at least three, four, five or six CDRs.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The anti-BMP-10/BMP-10 receptor antibody can be a polyclonal or a monoclonal antibody. In other embodiments, the antibody can be recombinantly produced, e.g., produced by phage display or by combinatorial methods.

Phage display and combinatorial methods for generating anti-BMP-10/BMP-10 receptor antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-BMP-10/BMP-10 receptor antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-BMP-10/BMP-10 receptor antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to a BMP-10/BMP-10 receptor or a fragment thereof. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are known to those skilled in the art and, for example, may be obtained from a hybridoma producing an antibody against a BMP-10/BMP-10 receptor polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In one embodiment, an antibody can be made by immunizing with purified BMP-10/BMP-10 receptor antigen, or a fragment thereof, e.g., a fragment described herein, membrane associated antigen, tissue, e.g., crude tissue preparations, whole cells, preferably living cells, lysed cells, or cell fractions, e.g., membrane fractions.

The anti-BMP-10/BMP-10 receptor antibody can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target BMP-10/BMP-10 receptor protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

An anti-BMP-10/BMP-10 receptor antibody (e.g., monoclonal antibody) can be used to isolate BMP-10/BMP-10 receptor by standard techniques, such as affinity chromatography or immunoprecipitation. Moreover, an anti-BMP-10/BMP-10 receptor antibody can be used to detect BMP-10/BMP-10 receptor protein (e.g., in a cellular lysate or cell supernatant) in order to evaluate the abundance and pattern of expression of the protein. Anti-BMP-10/BMP-10 receptor antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid which encodes an anti-BMP-10/BMP-10 receptor antibody, e.g., an anti-BMP-10/BMP-10 receptor antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-BMP-10/BMP-10 receptor antibody, e.g., and antibody described herein, and method of using said cells to make a BMP-10/BMP-10 receptor antibody.

Also featured are nucleic acids encoding the BMP-10/BMP-10 receptor sequence and variants thereof. The polypeptide can be used to provide a BMP-10/BMP-10 receptor binding agent that binds BMP-10/BMP-10 receptor, and optionally, also a BMP-10 from another species.

In one aspect, the invention features a method of providing a target binding molecule that specifically binds to BMP-10/BMP-10 receptor. For example, the target binding molecule is an antibody molecule. The method includes: providing a target protein that comprises at least a portion of non-human protein, the portion being homologous to (at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining a binding agent that specifically binds to the antigen; and evaluating efficacy of the binding agent in modulating activity of the target protein. The method can further include administering the binding agent (e.g., antibody molecule) or a derivative (e.g., a humanized antibody molecule) to a human subject. In one embodiment, the target protein is BMP-10/BMP-10 receptor.

In one embodiment, the step of obtaining comprises using a protein expression library, as described above. For example, the library displays antibody molecules such as Fab's of scFv's. In one embodiment, the step of obtaining comprises immunizing an animal using the antigen as an immunogen. For example, the animal can be a rodent, e.g., a mouse or rat. The animal can be a transgenic animal.

### BMP-10 Propeptides

In yet other embodiments, the BMP-10 antagonist is a BMP-10 antagonistic propeptide (e.g., a truncated or variant form of BMP-10 (e.g., a truncated or variant form of the propeptide region of human BMP-10 comprising about amino acids 22 to 313 of FIG. 2 (SEQ ID NO:2)) that is capable of forming an inhibitory complex with mature BMP-10). Methods for generating BMP-10 propeptides and variants thereof are known in the art and are disclosed in US 2006/0024783, the contents of which are incorporated by reference herein.

### Soluble BMP-10 Receptors

A soluble form of a BMP-10 receptor or a BMP-10 antagonistic propeptide can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. As used herein, a “fusion protein” refers to a protein containing two or more operably associated, e.g., linked, moieties, e.g., protein moieties. Typically, the moieties are covalently associated. The moieties can be directly associate, or connected via a spacer or linker.

The fusion proteins may additionally include a linker sequence joining the first moiety, e.g., a soluble BMP-10 receptor or BMP-10 propeptide, to the second moiety. For example, the fusion protein can include a peptide linker, e.g., a peptide linker of about 4 to 20, more preferably, 5 to 10, amino acids in length; the peptide linker is 8 amino acids in length. Each of the amino acids in the peptide linker is selected from the group consisting of Gly, Ser, Asn, Thr and Ala; the peptide linker includes a Gly-Ser element. In other embodiments, the fusion protein includes a peptide linker and the peptide linker includes a sequence having the formula (Ser-Gly-Gly-Gly-Gly) y wherein y is 1, 2, 3, 4, 5, 6, 7, or 8 (SEQ ID NO:19).

In other embodiments, additional amino acid sequences can be added to the N- or C-terminus of the fusion protein to facilitate expression, detection and/or isolation or purification. For example, BMP-10 receptor fusion protein may be linked to one or more additional moieties, e.g., GST, His6 tag (SEQ ID NO:20), FLAG tag. For example, the fusion protein may additionally be linked to a GST fusion protein in which the fusion protein sequences are fused to the C-terminus of the GST (i.e., glutathione S-transferase) sequences. Such fusion proteins can facilitate the purification of the BMP-10 receptor fusion protein.

In another embodiment, the fusion protein is includes a heterologous signal sequence (i.e., a polypeptide sequence that is not present in a polypeptide encoded by a BMP-10 receptor nucleic acid) at its N-terminus. For example, the native BMP-10 receptor signal sequence can be removed and replaced with a signal sequence from another protein. In certain host cells (e.g., mammalian host cells), expression and/or secretion of BMP-10 receptor can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that encode a fusion moiety (e.g., an Fc region of an immunoglobulin heavy chain). A BMP-10 receptor encoding nucleic acid can be cloned into such an expression vector such that the fusion moiety is linked in-frame to the immunoglobulin protein.

In some embodiments, BMP-10 receptor fusion polypeptides exist as oligomers, such as dimers or trimers.

In other embodiments, the BMP-10 receptor polypeptide moiety is provided as a variant BMP-10 receptor polypeptide having a mutation in the naturally-occurring BMP-10 receptor sequence (wild type) that results in higher affinity (relative to the non-mutated sequence) binding of the BMP-10 receptor polypeptide to BMP-10.

In other embodiments, the BMP-10 receptor polypeptide moiety is provided as a variant BMP-10 receptor polypeptide having mutations in the naturally-occurring BMP-10 receptor polypeptide sequence (wild type) that results in a BMP-10 receptor sequence more resistant to proteolysis (relative to the non-mutated sequence).

In some embodiments, the first polypeptide includes full-length BMP-10 receptor polypeptide. Alternatively, the first polypeptide comprise less than full-length BMP-10 receptor polypeptide. For example, the antagonist can be a soluble form of a BMP-10 receptor (e.g., a soluble form of mammalian (e.g., human) endoglin, ALK-1, -3 or -6 comprising a BMP-10 binding domain; e.g., a soluble form of an extracellular domain of mammalian (e.g., human) ALK-1, -3 or -6). For example, the BMP-10 antagonist can include about amino acids 22 to 118 of human ALK-1 (FIG. 3D; SEQ ID NO:4); about amino acids 24 to 152 of human ALK3 (FIG. 4C; SEQ ID NO:6); or a soluble fragment of an activin receptor IIB (ActRIIB), e.g., including about amino acids 17 to 133 of FIG. 5B; SEQ ID NO:18.

In other embodiments, additional amino acid sequences can be added to the N- or C-terminus of the fusion protein to facilitate expression, steric flexibility, detection and/or isolation or purification. The second polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second BMP-10 receptor polypeptide. In embodiments, the second polypeptide includes at least a region of an immunoglobulin polypeptide. Immunoglobulin fusion polypeptide are known in the art and are described in e.g., U.S. Pat. Nos. 5,516,964; 5,225,538; 5,428,130; 5,514,582; 5,714,147; and 5,455,165. For example, a soluble form of a BMP-10 receptor or a BMP-10 antagonistic propeptide can be fused to a heavy chain constant region of the various isotypes, including: IgG1, IgG2, IgG3, IgG4, IgM, IgA1, IgA2, IgD, and IgE). Typically, the fusion protein can include the extracellular domain of a human BMP-10 receptor, or a BMP-10 propeptide (or a sequence homologous thereto), and, e.g., fused to, a human immunoglobulin Fc chain, e.g., human IgG (e.g., human IgG1 or human IgG2, or a mutated form thereof).

The Fc sequence can be mutated at one or more amino acids to reduce effector cell function, Fc receptor binding and/or complement activity. Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would reduce or eliminate these functions. For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In embodiments, the second polypeptide has less effector function that the effector function of a Fc region of a wild-type immunoglobulin heavy chain. Fc effector function includes for example, Fc receptor binding, complement fixation and T cell depleting activity (see for example, U.S. Pat. No. 6,136,310). Methods for assaying T cell depleting activity, Fc effector function, and antibody stability are known in the art. In one embodiment, the second polypeptide has low or no detectable affinity for the Fc receptor. In an alternative embodiment, the second polypeptide has low or no detectable affinity for complement protein C1q.

It will be understood that the antibody molecules and soluble receptor or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, among others.

### Binding Domain Fusion Variants

In yet another embodiment, the BMP-10 agonist/antagonist is a binding domain fusion variant, or a small molecule. Binding domain fusion variants provide an example of a variant molecule that typically includes a binding domain polypeptide that is fused or otherwise connected to a hinge or hinge-acting region polypeptide, which in turn is fused or otherwise connected to a region comprising one or more native or engineered constant regions from a heavy chain, other than CH1, for example, the CH2 and CH3 regions of IgG and IgA, or the CH3 and CH4 regions of IgE (see e.g., U.S. Ser. No. 05/0136049 by Ledbetter, J. et al. for a more complete description). The binding domain-fusion protein can further include a region that includes a native or engineered heavy chain CH2 constant region polypeptide (or CH3 in the case of a construct derived in whole or in part from IgE) that is fused or otherwise connected to the hinge region polypeptide and a native or engineered heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-fusion proteins are capable of at least one activity selected from the group consisting of fusion protein-dependent cell-mediated cytotoxicity, complement fixation, and/or binding to a target, for example, a BMP-10/BMP-10 receptor.

Typically, the binding domain fusion variant or small molecule will bind to a mammalian, e.g., human, BMP-10 or a BMP-10 receptor with an affinity of at least about 10⁷ M⁻¹, typically about 10⁸ M⁻¹, and more typically, about 10⁹ M⁻¹ to 10¹⁰ M⁻¹ or stronger; and reduce and/or inhibit one or more BMP-10 biological activities as described herein. In embodiments, the binding domain fusion variant, or small molecule, binds to a mature BMP-10 sequence (e.g., a mature BMP-10 sequence comprising an amino acid sequence of about amino acids 314 to 424, 314 to 325, 325 to 335, 335 to 345, 345 to 355, 355 to 365, 365 to 375, 375 to 385, 385 to 395, 395 to 405, 405 to 415, 415 to 424, of human BMP-10 as shown in FIG. 2; SEQ ID NO:2)), or a sequence substantially homologous thereto, and inhibits, reduces or neutralizes one or more activities of BMP-10. In embodiments, the BMP-10 receptor is an activin receptor-like kinase (ALK)-1, -3, or -6 (e.g., an ALK-1, or -3 comprising an amino acid sequence identical to a mammalian, e.g., human, ALK-1 and -3 as shown in FIG. 3D (SEQ ID NO:4) and FIG. 4C (SEQ ID NO:6), respectively), or a sequence substantially homologous thereto, and inhibits, reduces or neutralizes one or more activities of BMP-10.

### Other BMP-10 Antagonists

In another embodiment, the BMP-10 antagonist is a KL-4 Surfactant (lucinactant) or a variant thereof. For example, the BMP-10 antagonist can be an engineered version of natural human lung surfactant, e.g., a KL-4 protein-like substance that is designed to closely mimic the attributes of human surfactant protein B (SP-B).

In other embodiments, the BMO-10 antagonist is a naturally-occurring antagonists, or a functional fragment or variant thereof. Examples of naturally-occurring BMP-10 antagonists include USAG-1, sclerostin, chordin, twisted gastrulation, endoglin and other antagonists described in Yanagita, M. (2005) Cytokine & Growth Factors Reviews 16:309-317, the contents of which are hereby incorporated by reference.

In yet another embodiment, the BMP-10 antagonist inhibits the expression of nucleic acid encoding a BMP-10 or a BMP-10 receptor. Examples of such BMP-10 antagonists include nucleic acid molecules, for example, antisense molecules, ribozymes, RNAi, triple helix molecules that hybridize to a nucleic acid encoding a BMP-10 or BMP-10 receptor, or a transcription regulatory region, and blocks or reduces mRNA expression of BMP-10 or a BMP-10 receptor.

In embodiments, nucleic acid antagonists are used to decrease expression of an endogenous gene encoding BMP-10/BMP-10 receptor. In one embodiment, the nucleic acid antagonist is an siRNA that targets mRNA encoding BMP-10/BMP-10 receptor. Other types of antagonistic nucleic acids can also be used, e.g., a dsRNA, a ribozyme, a triple-helix former, or an antisense nucleic acid. Accordingly, isolated nucleic acid molecules that are nucleic acid inhibitors, e.g., antisense, RNAi, to a BMP-10/BMP-10 receptor-encoding nucleic acid molecule are provided.

An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire BMP-10/BMP-10 receptor coding strand, or to only a portion thereof. In another embodiment, the antisense nucleic acid molecule is antisense to a “noncoding region” of the coding strand of a nucleotide sequence encoding BMP-10/BMP-10 receptor (e.g., the 5′ and 3′ untranslated regions). Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding BMP-10/BMP-10 receptor. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂) alkylaminocytosines and N⁴,N⁴—(C₁-C₁₂) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂) alkylaminopurines and N⁶,N⁶—(C₁-C₁₂) dialkylaminopurines, including N⁶-methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like. Descriptions of other types of nucleic acid agents are also available. See, e.g., U.S. Pat. Nos. 4,987,071; 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher (1992) Bioassays 14:807-15.

The antisense nucleic acid molecules of the invention are typically administered to a subject (e.g., by direct injection at a tissue site), or generated in situ such that they hybridize with or bind to cellular mRNA and/or genomic DNA encoding a BMP-10/BMP-10 receptor protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

In still another embodiment, an antisense nucleic acid of the invention is a ribozyme. A ribozyme having specificity for a BMP-10/BMP-10 receptor-encoding nucleic acid can include one or more sequences complementary to the nucleotide sequence of a BMP-10/BMP-10 receptor cDNA disclosed herein (i.e., SEQ ID NO:1 or SEQ ID NO:3), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a BMP-10/BMP-10 receptor-encoding mRNA. See, e.g., Cech et al. U.S. Pat. No. 4,987,071; and Cech et al. U.S. Pat. No. 5,116,742. Alternatively, BMP-10/BMP-10 receptor mRNA can be used to select a catalytic RNA having a specific ribonuclease activity from a pool of RNA molecules. See, e.g., Bartel, D. and Szostak, J. W. (1993) Science 261:1411-1418.

BMP-10/BMP-10 receptor gene expression can be inhibited by targeting nucleotide sequences complementary to the regulatory region of the BMP-10/BMP-10 receptor (e.g., the BMP-10/BMP-10 receptor promoter and/or enhancers) to form triple helical structures that prevent transcription of the BMP-10/BMP-10 receptor gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A BMP-10/BMP-10 receptor nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of BMP-10/BMP-10 receptor nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of BMP-10/BMP-10 receptor nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; WO88/09810) or the blood-brain barrier (see, e.g., WO 89/10134). In addition, oligonucleotides can be modified with hybridization-triggered cleavage agents (see, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### III. Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a BMP-10/BMP-10 receptor nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., BMP-10/BMP-10 receptor proteins, mutant forms of BMP-10/BMP-10 receptor proteins, fusion proteins, and the like).

The term “recombinant host cell” (or simply “host cell”), as used herein, is intended to refer to a cell into which a recombinant expression vector has been introduced. It should be understood that such terms are intended to refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein.

The recombinant expression vectors of the invention can be designed for expression of BMP-10/BMP-10 receptor proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in BMP-10/BMP-10 receptor activity assays, (e.g., direct assays or competitive assays described in detail below), or to generate antibodies specific for BMP-10/BMP-10 receptor proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The BMP-10/BMP-10 receptor expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a BMP-10/BMP-10 receptor nucleic acid molecule within a recombinant expression vector or a BMP-10/BMP-10 receptor nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a BMP-10/BMP-10 receptor protein can be expressed in bacterial cells (such as E. coli), insect cells, yeast or mammalian cells (such as Chinese hamster ovary cells (CHO) or COS cells e.g., COS-7 cells, CV-1 origin SV40 cells; Gluzman (1981) Cell 23:175-182). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a BMP-10/BMP-10 receptor protein. Accordingly, the invention further provides methods for producing a BMP-10/BMP-10 receptor protein using the host cells of the invention. In one embodiment, the method includes culturing the host cell of the invention (into which a recombinant expression vector encoding a BMP-10/BMP-10 receptor protein has been introduced) in a suitable medium such that a BMP-10/BMP-10 receptor protein is produced. In another embodiment, the method further includes isolating a BMP-10/BMP-10 receptor protein from the medium or the host cell.

In another aspect, the invention features, a cell or purified preparation of cells which include a BMP-10/BMP-10 receptor transgene, or which otherwise misexpress BMP-10/BMP-10 receptor. The cell preparation can consist of human or non-human cells, e.g., rodent cells, e.g., mouse or rat cells, rabbit cells, or pig cells. In preferred embodiments, the cell or cells include a BMP-10/BMP-10 receptor transgene, e.g., a heterologous form of a BMP-10/BMP-10 receptor, e.g., a gene derived from humans (in the case of a non-human cell). The BMP-10/BMP-10 receptor transgene can be misexpressed, e.g., overexpressed or underexpressed. In other preferred embodiments, the cell or cells include a gene that mis-expresses an endogenous BMP-10/BMP-10 receptor, e.g., a gene the expression of which is disrupted, e.g., a knockout. Such cells can serve as a model for studying disorders that are related to mutated or mis-expressed BMP-10/BMP-10 receptor alleles or for use in drug screening.

Also provided are cells, preferably human cells, e.g., fibroblast cells, in which an endogenous BMP-10/BMP-10 receptor is under the control of a regulatory sequence that does not normally control the expression of the endogenous BMP-10/BMP-10 receptor gene. The expression characteristics of an endogenous gene within a cell, e.g., a cell line or microorganism, can be modified by inserting a heterologous DNA regulatory element into the genome of the cell such that the inserted regulatory element is operably linked to the endogenous BMP-10/BMP-10 receptor gene. For example, an endogenous BMP-10/BMP-10 receptor gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a BMP-10/BMP-10 receptor polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Production of BMP-10/BMP-10 receptor polypeptide can be regulated in the subject by administering an agent (e.g., a steroid hormone) to the subject. In another preferred embodiment, the implanted recombinant cells express and secrete an antibody specific for a BMP-10/BMP-10 receptor polypeptide. The antibody can be any antibody or any antibody derivative described herein.

### IV. Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to BMP-10/BMP-10 receptor proteins, have a stimulatory or inhibitory effect on, for example, BMP-10/BMP-10 receptor expression or BMP-10/BMP-10 receptor activity, or have a stimulatory or inhibitory effect on, for example, the expression or activity of a BMP-10/BMP-10 receptor substrate. Compounds thus identified can be used to modulate the activity of target gene products (e.g., BMP-10/BMP-10 receptor genes) in a therapeutic protocol, to elaborate the biological function of the target gene product, or to identify compounds that disrupt normal target gene interactions. In one embodiment, the invention provides assays for screening candidate or test compounds that bind to or modulate an activity of a BMP-10/BMP-10 receptor protein or polypeptide or a biologically active portion thereof.

Accordingly, the invention provides a method, or an assay, for identifying a compound, e.g., a test compound, that modulates endothelial cell or vascular function The method, or the assay, includes: (i) (optionally) providing or identifying a test agent that interacts with, e.g., binds to, BMP-10 or a BMP-10 receptor; and/or (ii) evaluating a change in an activity of a BMP-10-responsive cell (e.g., a vascular and/or cardiac cell and/or tissue) in the presence of the test agent, relative to a reference, e.g., a reference sample.

The test compound or agent can be an antibody molecule; peptide; a soluble BMP-10 receptor or a fusion thereof; a binding domain fusion variant; a small molecule, e.g., a member of a combinatorial or natural product library; a nucleic acid; an antisense molecule; a ribozyme; an RNAi; a triple helix molecule; or any combination thereof. In one embodiment, the test compound modulates (e.g., decreases or increases) the activity or expression of a BMP-10 or a BMP-10 receptor polypeptide or nucleic acid. For example, the expression of the BMP-10 or a BMP-10 receptor nuclei acid can be modulated by e.g., altering mRNA transcription, mRNA stability, etc.

In embodiments, the evaluating step includes contacting one or more of: a BMP-10 or BMP-10 receptor polypeptide (e.g., a BMP-10 or BMP-10 receptor as described herein), or a nucleic acid encoding the BMP-10 or BMP-10 receptor, with the test compound; and evaluating a change in one or more activities of the BMP-10 or the BMP-10 receptor polypeptide or nucleic acid, in the presence of the test compound, relative to a predetermined level, e.g., a control sample without the test compound. The contacting step can be effected in vitro (in cultured cells, e.g., HUVECS or HAECS, or a reconstituted system) or in vivo (e.g., by administering the test compound to a non-human subject, e.g., an animal model having a mutation in a BMPR2 or a NKX2-5 gene). The contacting step(s) and/or the administration of the test compound can be repeated.

In embodiments, the change in an activity of the BMP-10 responsive cell (e.g., the vascular and/or cardiac cell and/or tissue) is evaluated by measuring a change, in the presence of the test compound, relative to a reference, e.g., a reference sample (e.g., a control sample not exposed to the test compound), in one or more of: (i) phosphorylation of a Smad protein (e.g., phosphorylation of Smad 1, 5 and/or 8); (ii) gene expression of myostatin, endoglin and/or an inhibitory Smad (e.g., induction of expression of Smad 6 and/or 7); (iii) expression of pro-angiogenic genes (e.g., VEGF, ID1 and ID2); (iv) expression of Ras-related protein-1a (Rap1a); (v) expression of one or more genes in response to BMP-10 stimulation of endothelial cells in vitro or in vivo identified in FIGS. 22-28; (vi) serum levels of stromal-derived differentiation factor (SDF-1) and/or matrix metallopeptidase 9 (MMP-9); and/or (vii) abnormalities in blood vessels, such as vascular dysplasia, hemorrhaging, telangiectasias, and/or arteriovenous malformations. In embodiments, a decrease in one or more of (i)-(iii) and (vi), and an increase in (iv), is indicative of an antagonist of BMP-10 function and thus, a candidate for treatment of a vascular and/or cardiac disorder where BMP-10 antagonism is desirable. In other embodiments, an increase in one or more of (i)-(iii) and (vi), and a decrease in (iv) is indicative of an agonist of BMP-10 function and thus, a candidate for treatment of a vascular and/or cardiac disorder where BMP-10 agonism is desirable.

In certain embodiments, an interaction between the test compound, the BMP-10 or the BMP-10 receptor is evaluated. In embodiments, such interaction can be evaluated by detecting a change in the formation and/or stability of the complex between the test compound and BMP-10 and/or BMP-10 receptor can be determined by detecting one or more of: a change in the binding or physical formation of the complex itself, e.g., by biochemical detection, affinity based detection (e.g., Western blot, affinity columns), immunoprecipitation, fluorescence resonance energy transfer (FRET)-based assays, spectrophotometric means (e.g., circular dichroism, absorbance, and other measurements of solution properties); a change, e.g., increase or decrease, in signal transduction, e.g., phosphorylation of Smads and/or transcription activity of a BMP-10-associated gene; a change, e.g., increase or decrease, in serum levels of stromal-derived differentiation factor (SDF-1) and/or matrix metallopeptidase 9 (MMP-9); and/or (vii) a change, e.g., increase or decrease in abnormalities in blood vessels, such as vascular dysplasia, hemorrhaging, telangiectasias, and/or arteriovenous malformations.

In one embodiment, the test compound is identified and re-tested in the same or a different assay. For example, a test compound is identified in an in vitro or cell-free system, and re-tested in an animal model or a cell-based assay. Any order or combination of assays can be used. For example, a high throughput assay can be used in combination with an animal model or tissue culture.

In other embodiments, the method, or assay, includes providing a step based on proximity-dependent signal generation, e.g., a two-hybrid assay that includes a first fusion protein (e.g., a fusion protein comprising a BMP-10 portion), and a second fusion protein (e.g., a fusion protein comprising a BMP-10 receptor), contacting the two-hybrid assay with a test compound, under conditions wherein said two hybrid assay detects a change in the formation and/or stability of the complex, e.g., the formation of the complex initiates transcription activation of a reporter gene.

In one non-limiting example, the three-dimensional structure of the active site of BMP-10 is determined by crystallizing the complex formed by the enzyme and a known inhibitor. Rational drug design is then used to identify new test agents by making alterations in the structure of a known inhibitor or by designing small molecule compounds that bind to the active site of the enzyme. Similarly, the skilled artisan would recognize that rational drug design could also be used to design antagonists of BMP-10 receptor, which would also be useful in modulating the production of BMP-10/BMP-10 receptor.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a BMP-10/BMP-10 receptor protein or biologically active portion thereof is contacted with a test compound, and the ability of the test compound to modulate BMP-10/BMP-10 receptor activity is determined. Determining the ability of the test compound to modulate BMP-10/BMP-10 receptor activity can be accomplished by monitoring, for example, Smad protein phosphorylation and/or transcription activity. The cell, for example, can be of mammalian origin, e.g., human (e.g., HUVECS or HAECS).

The ability of the test compound to modulate BMP-10/BMP-10 receptor binding to a compound, e.g., a BMP-10/BMP-10 receptor substrate, or to bind to BMP-10/BMP-10 receptor can also be evaluated. This can be accomplished, for example, by coupling the compound, e.g., the substrate, with a radioisotope or enzymatic label such that binding of the compound, e.g., the substrate, to BMP-10/BMP-10 receptor can be determined by detecting the labeled compound, e.g., substrate, in a complex. Alternatively, BMP-10/BMP-10 receptor could be coupled with a radioisotope or enzymatic label to monitor the ability of a test compound to modulate BMP-10/BMP-10 receptor binding to a BMP-10/BMP-10 receptor substrate in a complex. For example, compounds (e.g., BMP-10/BMP-10 receptor substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a BMP-10/BMP-10 receptor substrate) to interact with BMP-10/BMP-10 receptor with or without the labeling of any of the interactants can be evaluated. For example, a microphysiometer can be used to detect the interaction of a compound with BMP-10/BMP-10 receptor without the labeling of either the compound or the BMP-10/BMP-10 receptor. McConnell, H. M. et al. Science 257:1906-1912, 1992. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and BMP-10/BMP-10 receptor.

In yet another embodiment, a cell-free assay is provided in which a BMP-10/BMP-10 receptor protein or biologically active portion thereof is contacted with a test compound and the ability of the test compound to bind to the BMP-10/BMP-10 receptor protein or biologically active portion thereof is evaluated. Preferred biologically active portions of the BMP-10/BMP-10 receptor proteins to be used in assays of the present invention include fragments which participate in interactions with non-BMP-10/BMP-10 receptor molecules, e.g., fragments with high surface probability scores.

Soluble and/or membrane-bound forms of isolated proteins (e.g., BMP-10/BMP-10 receptor proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected. The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means known in the art (e.g., using a fluorimeter).

In another embodiment, determining the ability of the BMP-10/BMP-10 receptor protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either BMP-10/BMP-10 receptor, an anti-BMP-10/BMP-10 receptor antibody or its target molecule to facilitate separation of complexed from uncomplexed forms of one or both of the proteins, as well as to accommodate automation of the assay. Binding of a test compound to a BMP-10/BMP-10 receptor protein, or interaction of a BMP-10/BMP-10 receptor protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/BMP-10/BMP-10 receptor fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or BMP-10/BMP-10 receptor protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of BMP-10/BMP-10 receptor binding or activity determined using standard techniques.

Other techniques for immobilizing either a BMP-10/BMP-10 receptor protein or a target molecule on matrices include using conjugation of biotin and streptavidin. Biotinylated BMP-10/BMP-10 receptor protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with BMP-10/BMP-10 receptor protein or target molecules but which do not interfere with binding of the BMP-10/BMP-10 receptor protein to its target molecule. Such antibodies can be derivatized to the wells of the plate, and unbound target or BMP-10/BMP-10 receptor protein trapped in the wells by antibody conjugation. Methods for detecting such complexes, in addition to those described above for the GST-immobilized complexes, include immunodetection of complexes using antibodies reactive with the BMP-10/BMP-0 receptor protein or target molecule, as well as enzyme-linked assays which rely on detecting an enzymatic activity associated with the BMP-10/BMP-10 receptor protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N.H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the BMP-10/BMP-10 receptor protein or biologically active portion thereof with a known compound which binds BMP-10/BMP-10 receptor to form an assay mixture, contacting the assay mixture with a test compound, and determining the ability of the test compound to interact with a BMP-10/BMP-10 receptor protein, wherein determining the ability of the test compound to interact with a BMP-10/BMP-10 receptor protein includes determining the ability of the test compound to preferentially bind to BMP-10/BMP-10 receptor or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the BMP-10/BMP-10 receptor genes herein identified. In an alternative embodiment, the invention provides methods for determining the ability of the test compound to modulate the activity of a BMP-10/BMP-10 receptor protein through modulation of the activity of a downstream effector of a BMP-10/BMP-10 receptor target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the BMP-10/BMP-10 receptor proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with BMP-10/BMP-10 receptor (“BMP-10/BMP-10 receptor-binding proteins” or “BMP-10/BMP-10 receptor-bp”) and are involved in BMP-10/BMP-10 receptor activity. Such BMP-10/BMP-10 receptor-bps can be activators or inhibitors of signals by the BMP-10/BMP-10 receptor proteins or BMP-10/BMP-10 receptor targets as, for example, downstream elements of a BMP-10/BMP-10 receptor-mediated signaling pathway.

The two-hybrid system is based on the modular nature of most transcription factors, which consist of separable DNA-binding and activation domains. Briefly, the assay utilizes two different DNA constructs. In one construct, the gene that codes for a BMP-10/BMP-10 receptor protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: BMP-10/BMP-10 receptor protein can be the fused to the activator domain.) If the “bait” and the “prey” proteins are able to interact, in vivo, forming a BMP-10/BMP-10 receptor-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the BMP-10/BMP-10 receptor protein.

In another embodiment, modulators of BMP-10/BMP-10 receptor expression are identified. For example, a cell or cell free mixture is contacted with a candidate compound and the expression of BMP-10/BMP-10 receptor mRNA or protein evaluated relative to the level of expression of BMP-10/BMP-10 receptor mRNA or protein in the absence of the candidate compound. When expression of BMP-10/BMP-10 receptor mRNA or protein is greater in the presence of the candidate compound than in its absence, the candidate compound is identified as a stimulator of BMP-10/BMP-10 receptor mRNA or protein expression. Alternatively, when expression of BMP-10/BMP-10 receptor mRNA or protein is less (statistically significantly less) in the presence of the candidate compound than in its absence, the candidate compound is identified as an inhibitor of BMP-10/BMP-10 receptor mRNA or protein expression. The level of BMP-10/BMP-10 receptor mRNA or protein expression can be determined by methods described herein for detecting BMP-10/BMP-10 receptor mRNA or protein.

In another aspect, the invention pertains to a combination of two or more of the assays described herein. For example, a modulating agent can be identified using a cell-based or a cell free assay, and the ability of the agent to modulate the activity of a BMP-10/BMP-10 receptor protein can be confirmed in vivo, e.g., in an animal such as an animal model for inflammatory lung disease, such asthma.

This invention further pertains to novel agents identified by the above-described screening assays. Accordingly, it is within the scope of this invention to further use an agent identified as described herein (e.g., a BMP-10/BMP-10 receptor modulating agent, an antisense BMP-10/BMP-10 receptor nucleic acid molecule, a BMP-10/BMP-10 receptor-specific antibody, or a BMP-10/BMP-10 receptor-binding partner) in an appropriate animal model to determine the efficacy, toxicity, side effects, or mechanism of action, of treatment with such an agent. Furthermore, novel agents identified by the above-described screening assays can be used for treatments as described herein.

The screening methods of the invention are performed either in vitro (for example by monitoring BMP-10/BMP-10 receptor activity in a cell-based assay or in an enzymatic activity assay) or in vivo (for example by monitoring BMP-10/BMP-10 receptor activity or expression in tissue samples such as BAL after administering a test agent to a mammal). Exemplary mammals include without limitation, human, mouse, rat, and dog.

The invention also provides non-human transgenic animals. Such animals are useful for studying the function and/or activity of a BMP-10/BMP-10 receptor protein and for identifying and/or evaluating modulators of BMP-10/BMP-10 receptor activity.

As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous BMP-10/BMP-10 receptor gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a BMP-10/BMP-10 receptor protein to particular cells. A transgenic founder animal can be identified based upon the presence of a BMP-10/BMP-10 receptor transgene in its genome and/or expression of BMP-10/BMP-10 receptor mRNA in tissues or cells of the animals. A transgenic founder animal can then be used to breed additional animals carrying the transgene. Moreover, transgenic animals carrying a transgene encoding a BMP-10/BMP-10 receptor protein can further be bred to other transgenic animals carrying other transgenes.

BMP-10/BMP-10 receptor proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

### V. Diagnostic and Prognostic Assays

In another aspect, the invention provides methods for evaluating, diagnosing, and/or monitoring the progression of, a BMP-10 associated vascular and/or cardiac disorder (e.g., a disorder as described herein) in a test sample. The method includes evaluating the expression or activity of a nucleic acid or polypeptide chosen from BMP-10 or a BMP-10-associated gene, such that, a difference in the level of the nucleic acid or polypeptide relative to a reference sample, e.g., a sample obtained from normal subject or prior to treatment, is indicative of the presence or progression of the disorder. In embodiments, the BMP-10-associated nucleic acid or polypeptide is characterized by altered expression in response to BMP-10. Exemplary BMP-10-associated genes include, but are not limited to, GDF-8, GDF-10, endoglin, inhibitory Smad (e.g., Smad 6 and/or 7); and pro-angiogenic genes (e.g., VEGF, ID1 and ID2). In certain embodiments, an increase in the level of BMP-10 or a BMP-10-associated gene in the test sample, relative to a reference sample, is associated with the diagnosis of BMP-10 vascular and/or cardiac disorder where antagonism of BMP-10 function is desirable (e.g., a vascular and/or cardiac disorder as described herein). In other embodiments, a decrease in the level of a BMP-10 or a BMP-10-associated gene in the test sample, relative to a reference sample, is associated with the diagnosis of BMP-10 vascular and/or cardiac disorder where agonism of BMP-10 function is desirable.

In one embodiment, the evaluating step occurs in vitro or ex vivo. For example, a sample, e.g., a serum sample, is obtained from the subject.

In another embodiment, the evaluating step occurs in vivo. For example, by administering to the subject a detectably labeled agent that interacts with the BMP-10, or BMP-10 associated, nucleic acid or polypeptide, such that a signal is generated relative to the level of activity or expression of the nucleic acid or polypeptide.

### Expression Monitoring and Profiling.

The presence, level, or absence of BMP-10/BMP-10 receptor protein or nucleic acid in a biological sample can be evaluated by obtaining a biological sample from a test subject and contacting the biological sample with a compound or an agent capable of detecting BMP-10/BMP-10 receptor protein or nucleic acid (e.g., mRNA, genomic DNA) that encodes BMP-10/BMP-10 receptor protein such that the presence of BMP-10/BMP-10 receptor protein or nucleic acid is detected in the biological sample. The term “biological sample” includes tissues, cells and biological fluids isolated from a subject, as well as tissues, cells and fluids present within a subject. A preferred biological sample is serum. The level of expression of the BMP-10/BMP-10 receptor gene can be measured in a number of ways, including, but not limited to: measuring the mRNA encoded by the BMP-10/BMP-10 receptor genes; measuring the amount of protein encoded by the BMP-10/BMP-10 receptor genes; or measuring the activity of the protein encoded by the BMP-10/BMP-10 receptor genes.

The level of mRNA corresponding to the BMP-10/BMP-10 receptor gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length BMP-10/BMP-10 receptor nucleic acid, such as the nucleic acid of SEQ ID NO:1, or a portion thereof, such as an oligonucleotide of at least 7, 15, 30, 50, 100, 250 or 500 nucleotides in length and sufficient to specifically hybridize under stringent conditions to BMP-10/BMP-10 receptor mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the BMP-10/BMP-10 receptor genes.

The level of mRNA in a sample that is encoded by one of BMP-10/BMP-10 receptor can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al. U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the BMP-10/BMP-10 receptor gene being analyzed.

In another embodiment, the methods further contacting a control sample with a compound or agent capable of detecting BMP-10/BMP-10 receptor mRNA, or genomic DNA, and comparing the presence of BMP-10/BMP-10 receptor mRNA or genomic DNA in the control sample with the presence of BMP-10/BMP-10 receptor mRNA or genomic DNA in the test sample. In still another embodiment, serial analysis of gene expression, as described in U.S. Pat. No. 5,695,937, is used to detect BMP-10/BMP-10 receptor transcript levels.

A variety of methods can be used to determine the level of protein encoded by BMP-10/BMP-10 receptor. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect BMP-10/BMP-10 receptor protein in a biological sample in vitro as well as in vivo. In vitro techniques for detection of BMP-10/BMP-10 receptor protein include enzyme linked immunosorbent assays (ELISAs), immunoprecipitations, immunofluorescence, enzyme immunoassay (EIA), radioimmunoassay (RIA), and Western blot analysis. In vivo techniques for detection of BMP-10/BMP-10 receptor protein include introducing into a subject a labeled anti-BMP-10/BMP-10 receptor antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-BMP-10/BMP-10 receptor antibody positioned on an antibody array (as described below). The sample can be detected, e.g., with avidin coupled to a fluorescent label.

In another embodiment, the methods further include contacting the control sample with a compound or agent capable of detecting BMP-10/BMP-10 receptor protein, and comparing the presence of BMP-10/BMP-10 receptor protein in the control sample with the presence of BMP-10/BMP-10 receptor protein in the test sample.

The invention also includes kits for detecting the presence of BMP-10/BMP-10 receptor in a biological sample. For example, the kit can include a compound or agent capable of detecting BMP-10/BMP-10 receptor protein or mRNA in a biological sample; and a standard. The compound or agent can be packaged in a suitable container. The kit can further comprise instructions for using the kit to detect BMP-10/BMP-10 receptor protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted BMP-10/BMP-10 receptor expression or activity. As used herein, the term “unwanted” includes an unwanted phenomenon involved in a biological response such as airway inflammation.

In one embodiment, a disease or disorder associated with aberrant or unwanted BMP-10/BMP-10 receptor expression or activity is identified. A test sample is obtained from a subject and BMP-10/BMP-10 receptor protein or nucleic acid (e.g., mRNA or genomic DNA) is evaluated, wherein the level, e.g., the presence or absence, of BMP-10/BMP-10 receptor protein or nucleic acid is diagnostic for a subject having or at risk of developing a disease or disorder associated with aberrant or unwanted BMP-10/BMP-10 receptor expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted BMP-10/BMP-10 receptor expression or activity. For example, such methods can be used to determine whether a subject can be effectively treated with an agent to antagonize or otherwise inhibit BMP-10/BMP-10 receptor expression or activity.

In another aspect, the invention features a computer medium having a plurality of digitally encoded data records. Each data record includes a value representing the level of expression of BMP-10/BMP-10 receptor in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than BMP-10/BMP-10 receptor (e.g., other genes associated with a BMP-10/BMP-10 receptor-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of BMP-10/BMP-10 receptor expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a airway inflammatory disorder in a subject wherein an increase in BMP-10/BMP-10 receptor expression or activity is an indication that the subject has or is disposed to having a chronic airway inflammatory disorder, including, for example, asthma. The method can be used to monitor a treatment for asthma in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of BMP-10/BMP-10 receptor expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of BMP-10/BMP-10 receptor expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of BMP-10/BMP-10 receptor expression.

The Examples that follow are set forth to aid in the understanding of the inventions but are not intended to, and should not be construed to, limit its scope in any way.

### EXAMPLE 1

### mRNA Expression Analysis of Human BMP-10 in Adult Human and Mouse Tissues

Experiment A. Northern Blot Analysis of BMP-10 mRNA Transcripts in Human Adult Tissues

### Methods.

Northern blot analysis of BMP-10 mRNA expression was performed using manufacturer's recommendations (Clontech). A radiolabeled probe for human BMP-10 representing the entire coding region was used to assess expression of BMP-10 mRNA in human heart, skeletal muscle, colon, thymus, spleen, kidney, liver, small intestine, placenta, lung, and peripheral blood lymphocyte tissues, prepared by the manufacturer.

Results.

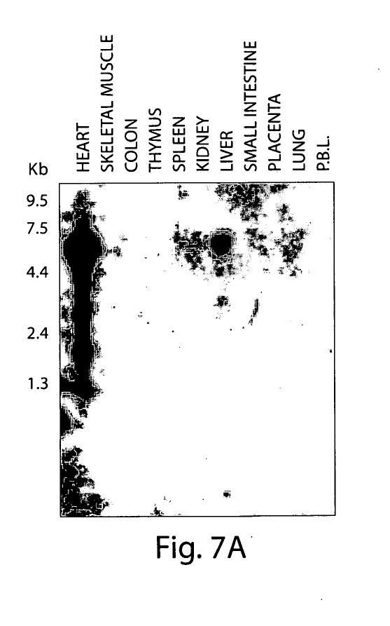

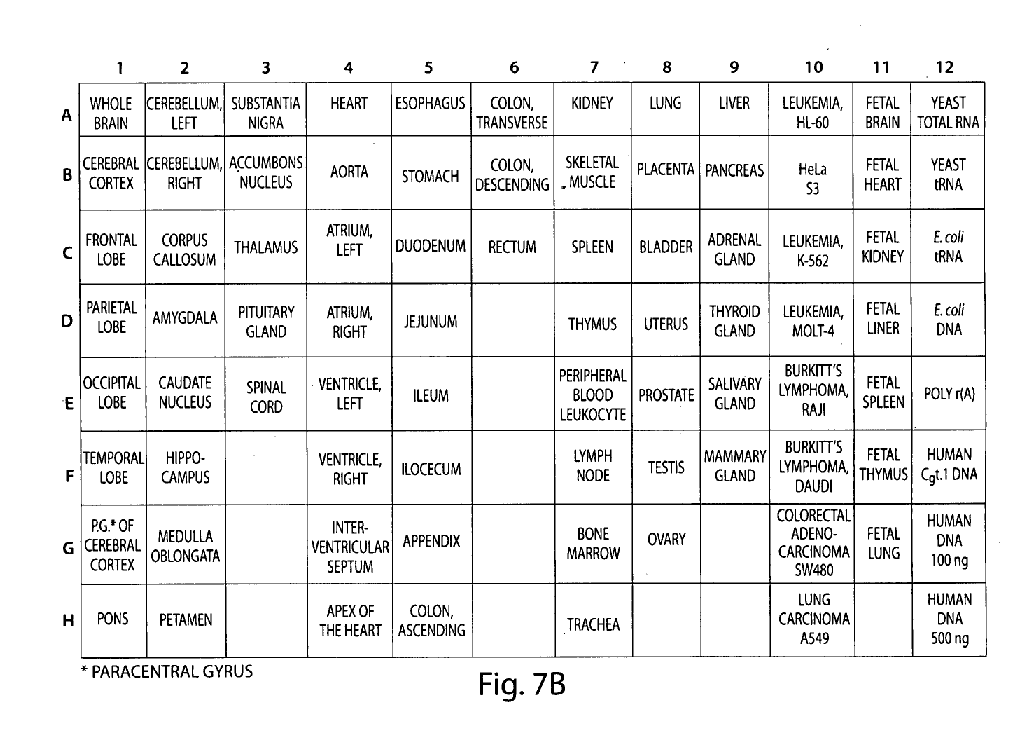

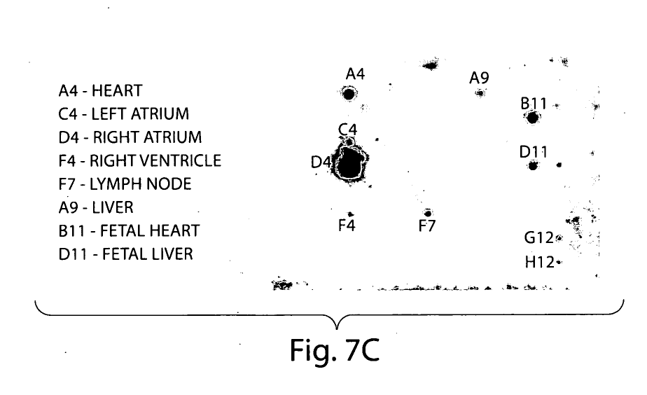

BMP-10 mRNA expression was detected in human adult heart, kidney and liver tissues, as shown in the Northern blot in FIG. 7A.

### Experiment B. BMP-10 Expression in Adult Human Tissues

Methods.

Detection of BMP-10 mRNA in human adult tissues was performed using multiple tissue northern dot blots (Clontech cat #636806). Radiolabeled BMP-10 cDNA probe representing the entire coding region was generated following manufacturer's directions for random-primer radioactive labeling. Hybridization was performed following manufacturer's recommendations.

Results.

In addition to the high levels of BMP-10 mRNA expression found in adult human heart tissue, other adult human tissues showed significant mRNA expression, notably lymph node tissue and liver tissues (FIGS. 7B and 7C).

### EXAMPLE 2

### In Vivo Observations and Pathology of Human AdBMP-10 Overexpression in Mice

Experiment A. Gross In Vivo Observations of Mice Injected with AdBMP-10

Methods

Recombinant adenovirus was constructed as follows. The Adori 1-2 human BMP-10 (hBMP-10) vector was derived by digesting pED6dpc-hBMP-10, and ligating the 1.27 kb hBMP1-0 cDNA fragment into adenovirus vector Adori 1-2. The construct was verified by extensive restriction digestion analysis and sequencing of the cDNA insert within the plasmid (AdBMP-10). An adenovirus vector encoding green fluorescent protein (GFP) (AdGFP), secreted alkaline phosphatase (SEAP) (AdSEAP) or Beta-galactosidase (B-gal) (Ad B-gal) were also used. Expression of the hBMP-10 cDNA, B-gal, and GFP are driven from cytomegalovirus (CMV) immediate early promoter and enhancer.

Replication-defective, E1 and E3 deleted recombinant, type 5 adenovirus was generated by homologous recombination in human embryonic kidney 293 cells (ATCC, Rockville, Md.). Recombinant adenovirus was amplified on 293 cells and the virus was released from infected cells by three cycles of freeze thawing. The virus was further purified by two cesium chloride centrifugation gradients and dialyzed against phosphate buffer saline (PBS) pH 7.2 at 4° C. Following dialysis, glycerol was added to a concentration of 10% and the virus was stored at −80° C. until use. Virus concentration, expressed in particles/ml, was determined by measuring the optical density at 260 nm. Endotoxin levels were measured using Limulus Amebocyte Lysate kit (BioWhittaker, Walkersville, Md.) and were below the detection limits of the assay. The virus was further characterized by PCR amplification of the insert using vector specific primers, and sequencing the ends of the PCR product. Expression of the hBMP-10 was verified by metabolically labeling transduced 293 cells using ³⁵S-methionine/cysteine and analysis of the conditioned medium by sodium dodecyl sulfate (SDS)-polyacrylamide gel electrophoresis ((PAGE)Novex, Invitrogen Life Technologies, Carlsbad, Calif.). Heterologous expression of BMP-10 was further confirmed by Western immunoblot analysis after transfection of HEK 293 cells, as described in Example 3.

A single dose of 5×10¹⁰ particles of recombinant adenovirus encoding hBMP-10 was injected into the tail vein of female C57BL/6J mice, age 7-8 weeks. Control mice received an adenovirus encoding either B-gal or GFP, or were injected with PBS/10% glycerol as the buffer control. Mice from each experimental group were euthanized at scheduled time points post injection and terminal body weights were obtained. Blood was collected via the retro-orbital sinus and differential counts were performed on blood smears. For hematological and serum chemistry analysis blood was collected by cardiac puncture. Macroscopic analysis was performed on all animals and selected tissues were weighed. Tissue was harvested, fixed in 10% neutral buffered formalin, trimmed, embedded in paraffin, sectioned and stained with hematoxylin and eosin for histopathology. Tissues for RNA analysis were snap frozen in liquid nitrogen and stored at −80° C. prior to analysis.

Results

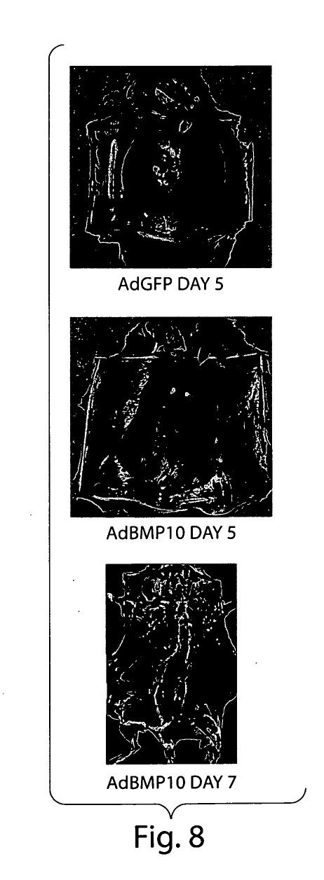

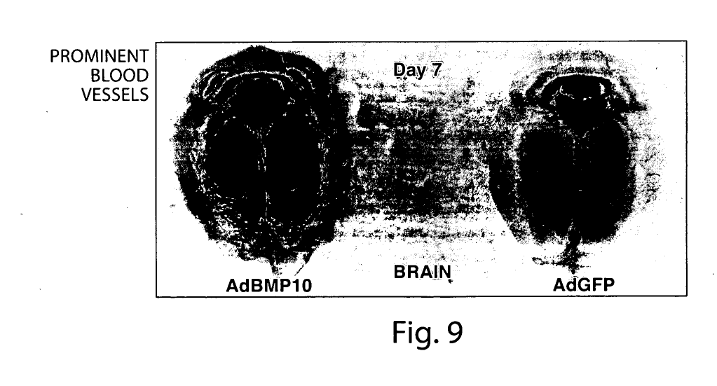

The effects of overexpression of BMP-10 were obvious by day 3, and mice were moribund by day 7. Due to pronounced debilitation and emaciation all animals were analyzed on or before day 7. Vascular dysplasia was evidenced by a pronounced increase in subcutaneous blood vessels observed in gross, in the brain, and in the liver. A whole animal view of a representative control mouse, AdGFP day 5 is shown in FIG. 8. In comparison, a representative AdBMP-10 day 5 mouse and a representative AdBMP-10 day 7 mouse had marked red discoloration of numerous organs (fat, brain, and lung), subcutaneous reddening (vascular dysplasia), general wasting, and decreased body fat (FIG. 8). The brain and liver, when studied in closer detail, exhibited other signs of vascular dysplasia in response to BMP-10 overexpression. FIG. 9 shows the prominent blood vessels in the brain of a representative AdBMP-10 day 7 mouse, the control AdGFP day 7 mouse brain is shown for comparison.

### Experiment B. Gross Histopathological Analyses of In Vivo Changes in AdBMP-10 Mice

### Methods

Histopathological Methods were as Described Above and/or Standard Procedure.

### Results.

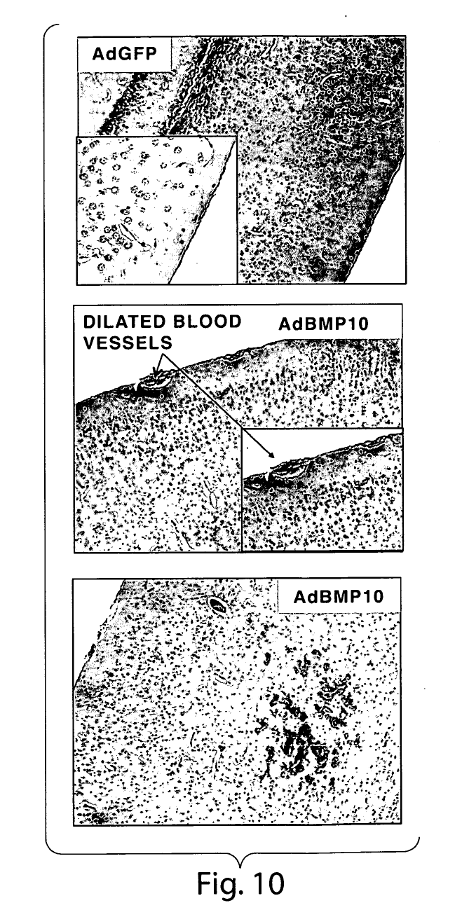

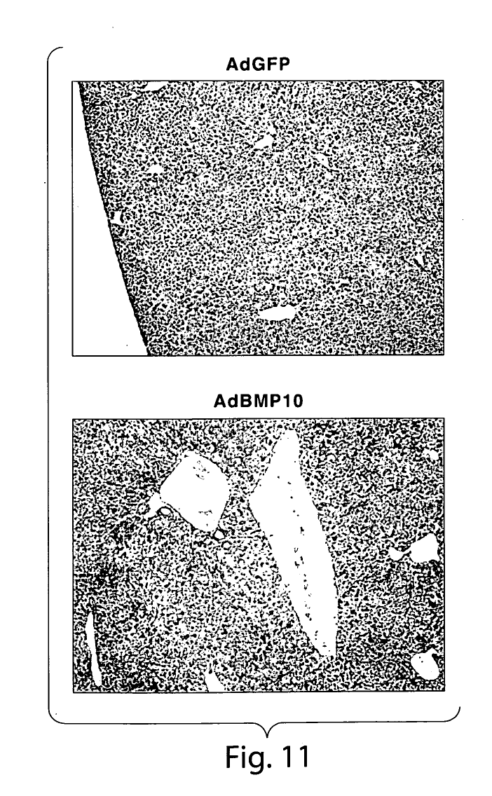

Histopathological analyses revealed dilated blood vessels, widespread congestion and scattered hemorrhages. A histopathological analysis of a representative AdBMP-10 Day 7 mouse is shown in FIG. 10, in which congestion, hemorrhages, and mild necroses can be seen. The livers of representative AdBMP-10 and AdGFP control mice, both day 7, are shown in FIG. 11. The liver of the AdBMP-10 mouse has multifocal areas of enlarged hepatocytes and coagulative necrosis.

### Experiment C. Organ and Body Weights of AdBMP-10 Mice Compared to Control Mice

### Methods.

Terminal body weights of AdBMP-10 mice were measured on day 3 and day 7 after injection with a single dose of 5×10¹⁰ particles of AdBMP-10, and compared to control mice injected with buffer, or injected with an adenovirus encoding GFP. Organ weights (spleen, liver, thymus) of AdBMP-10 mice were measured 7 and 14 days after an injection with a single dose of 5×10¹⁰ particles of AdBMP-10, or a control injection of buffer or AdB-gal. All AdBMP-10 mice had to be sacrificed by day 7 due to pronounced debilitation and emaciation.

### Results.

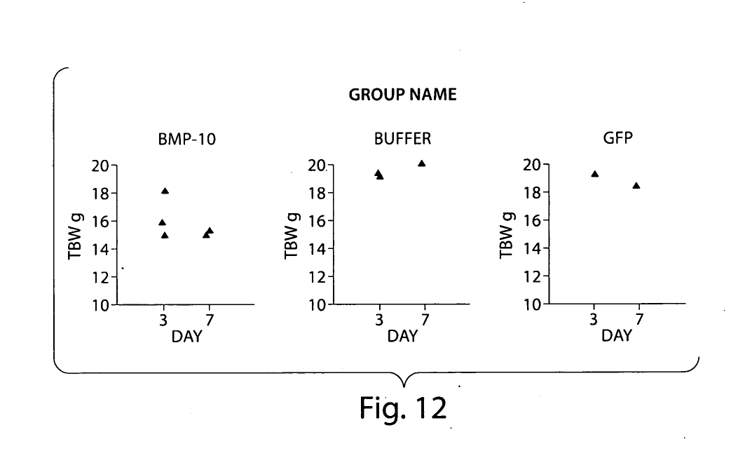

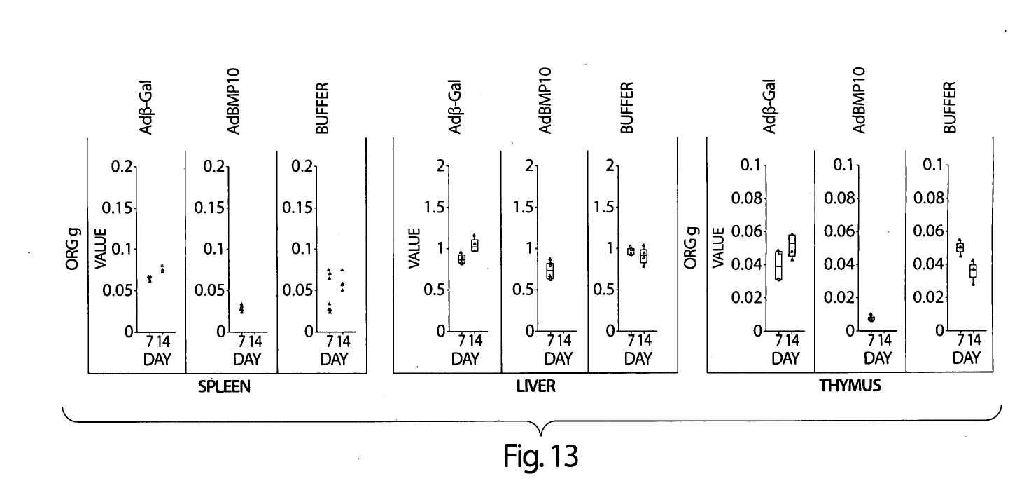

The distribution of terminal body weights (gms) by group for AdBMP-10 and control mice on day 3 and day 7 after injection is shown in FIG. 12. There was a dramatic decrease in the terminal body weight of AdBMP-10 mice by day 7, compared to the buffer and AdGFP controls. The distribution of organ weights for spleen, liver, and thymus for AdBMP-10 mice on day 7, and control AdB-gal and buffer mice on day 7 and day 14 is shown in FIG. 13. The spleen, liver, and thymus of AdBMP-10 mice all show marked decrease in mass compared to the AdB-gal and buffer control mice.

### Experiment D. Further Pathological Analysis of Cellular Changes and Serum Chemistry in AdBMP-10 Mice.

### Methods.

A single dose of 5×10¹⁰ particles of a replication deficient recombinant adenovirus encoding human BMP-10 (AdBMP-10) or a control virus (AdB-gal, or secreted alkaline phosphatase, SEAP) was injected intravenously into C57BL/6 mice, age 7-8 weeks, as described above. Serum chemistry was analyzed on day 4.

### Results.

Most changes considered to be associated with gene expression were characteristic of pronounced debilitation. These changes included the gross observations of emaciation, and atrophy of the thymus, the spleen, and occasionally the liver, the decrease in terminal body weight and in absolute and/or relative (to body) liver, spleen and thymus weights (methods and results described above and already shown in FIGS. 8-13, inclusively).

The microscopic correlates of the observed gross changes were atrophy of the abdominal and subcutaneous fat and atrophy of the thymus. Further microscopic indications of debilitation were found in the lymph node (lymphoid depletion) and stomach (atrophy of the glandular mucosa with single cell necrosis). Congestion was found in several organs (abdominal and subcutaneous fat, femur, and intestine) and correlated to the discolorations observed in these organs at the gross level.

In all animals there was a minimal to moderate multi-focal liver necrosis. This change was random in distribution and characterized by small areas of coagulation necrosis, approximately the size of one fourth to two third of a lobule. Some of the necrotic areas were composed of swollen hydropic necrotic hepatocytes associated with mild influx of granulocytes (vacuolar degeneration and necrosis). These areas were usually sharply demarcated. The pathophysiology of this necrotic phenomenon was not apparent. This change is not typically associated with debilitation in mice. The consistent occurrence of this change in all animals from the AdBMP-10 group strongly suggests an association with gene expression.

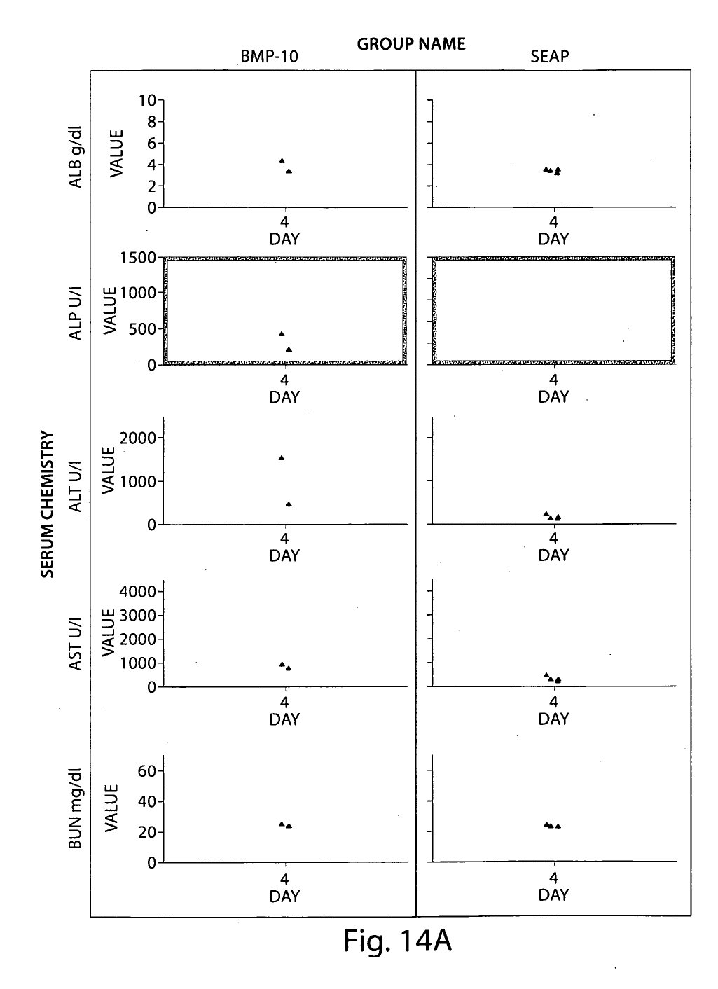

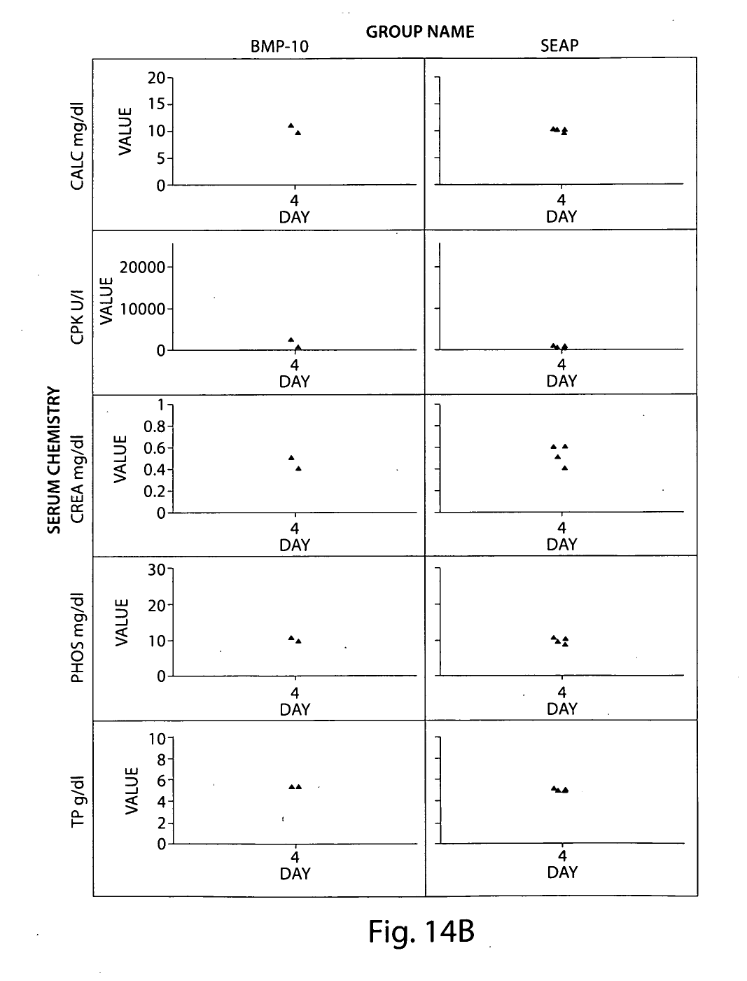

There was statistically significant and dramatic increase in aspartate aminotransferase (AST), alanine aminotransferase (ALT) and alkaline phosphatase (ALP) on day 4 compared to the SEAP group (FIGS. 14A-14B). All other hematology and clinical chemistry data variations were considered to be of no relevance to gene expression as they were of minimal amplitude, were lacking corresponding microscopic hepatic changes were consistent over time (data not shown). Other microscopic hepatic changes were consistent with lesions associated with the viral system and were associated with a characteristic serum chemistry profile.

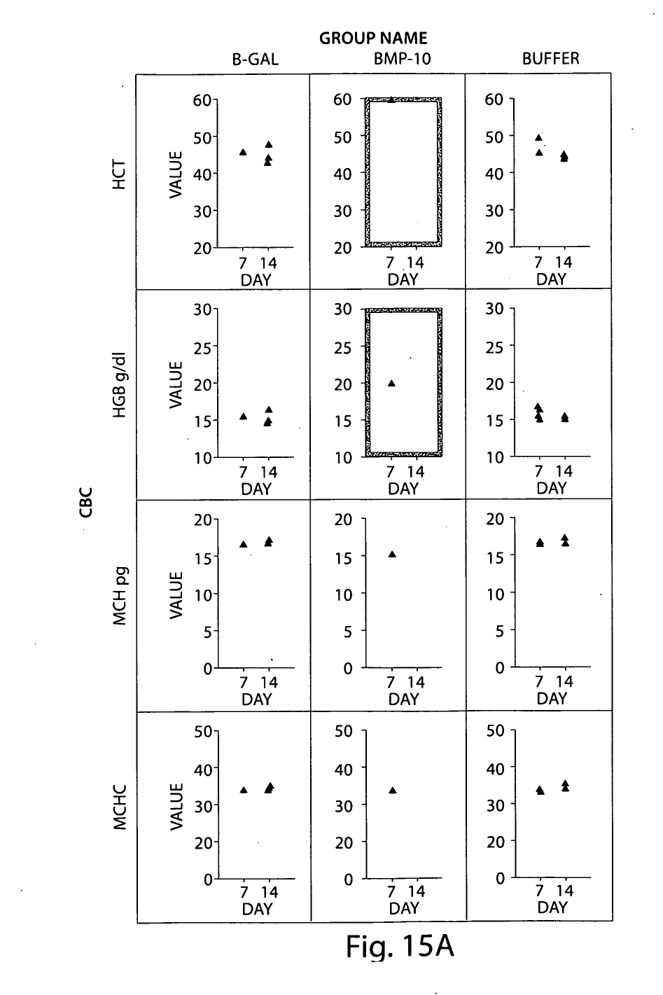

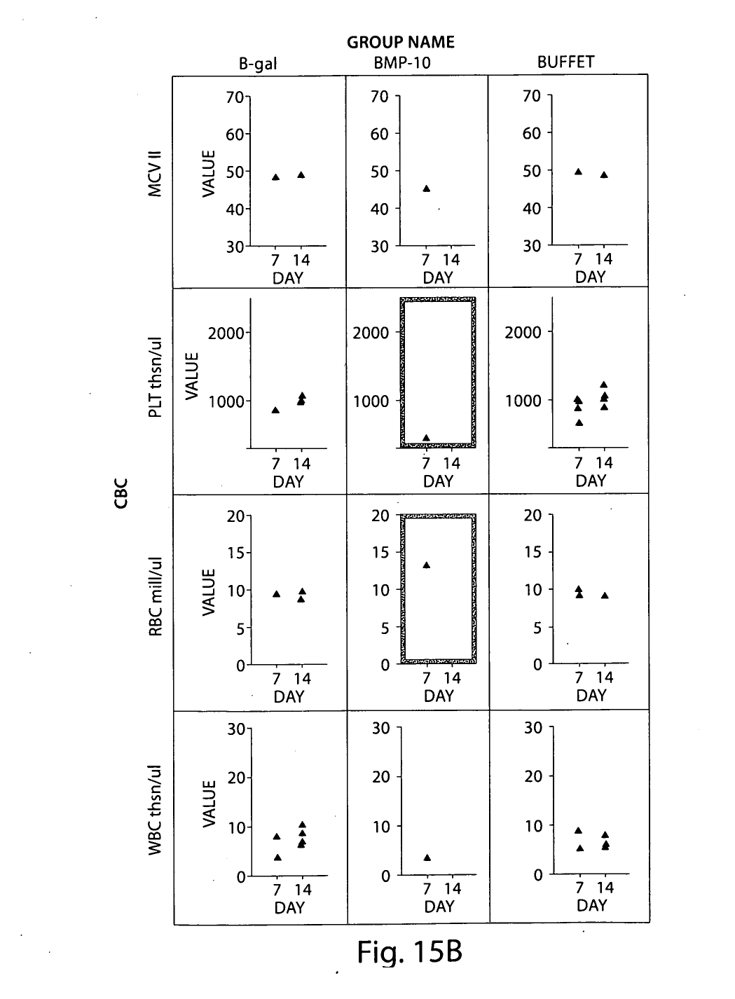

Certain cellular and serum chemistry changes in AdBMP-10 mice were found to be statistically significant compared to mice injected with control virus. These were increase hematocrit, increased hemoglobin, increased red blood cells, and decreased platelets (FIGS. 15A-15B).

Changes considered to be related to test gene expression were found in the AdBMP-10 group and consisted in the pronounced debilitation and emaciation in all AdBMP-10 treated animals. This change was responsible for the premature sacrifice of all AdBMP-10 treated animals on day 7. In addition, there was multifocal random hydropic degeneration and coagulation necrosis in the liver in all BMP-10 animals. Congestion was found in several organs (abdominal and subcutaneous fat, femur, and intestine) and correlated to the discolorations observed in these organs at the gross level.

### Experiment E. Continuing Study of Morphologic Effects of BMP-10 Expression in AdBMP-10 Mice

### Methods.

Two groups of C57BL/6 mice were treated, one with AdBMP-10 and one with AdSEAP as a control. The route of administration was via intravenous injection of AdBMP-10 or adenovirus encoding SEAP as previously described. Wet tissue for histologic preparation and microscopic evaluation were prepared using standard techniques. Statistically analyzed organ weight data, clinical chemistry data, hematology data and terminal body weight data were supplied by the sponsor for analysis in this report.

### Results.

All macroscopic and microscopic lesions observed in the AdSEAP control mice were consistent with changes commonly observed in mice.

Most changes considered to be associated with ectopic expression of BMP-10 were characteristic of pronounced debilitation with emaciation and dehydration. These changes included the gross observations of emaciation, atrophy of the thymus, pancreas and occasionally the liver and the spleen, the decrease in terminal body weight, and in absolute and/or relative (to body) liver, spleen and thymus weights. The most striking microscopic correlates were moderate atrophy of the thymus and mild to moderate zymogen granules depletion in the pancreas. Minimal to moderate multifocal liver necrosis was found in two animals and correlated with the gross observation of liver discoloration. The necrosis was characterized by scattered foci of acute coagulation necrosis preferentially distributed in the mid-zonal area and occasionally encompassing the centrilobular area. This change was not homogeneously distributed throughout the liver and some areas of the liver were spared. The cause of the liver necrosis was not apparent. Microscopic liver changes typically associated with viral exposure were reduced, particularly the mitogenic response. The gross observation of red discoloration at the pylorus found in all animals correlated with hemorrhage in the wall of the proximal duodenum in three animals. The hemorrhage was located in the lamina propria, submucosa (in the stroma associated with Bruner's glands), and in the muscular wall, particularly in the inner muscular layer. The inner muscular layer appeared segmentally effaced by the hemorrhage and segmentally necrotic in one animal. Occasional fibrin thrombi were present in dilated vessels of the submucosa (in association with Bruner's glands). The cause for this duodenal transmural hemorrhage with slight muscular necrosis was not apparent. The morphology of this change is indicative of localized vascular compromise and is reminiscent, although not pathognomonic of ischemic changes observed with intussusception.

Hematology and serum chemistry values showed marked polycythemia (increased red blood cells (RBC), hemoglobin (Hb), and hematocrit (HCT)). In the absence of erythroid hyperplasia in the bone marrow and spleen, the polycythemia is indicative of pronounced dehydration. The slight decrease in MCV may also be related to dehydration. The cause for the slight decrease in MCH is unclear. Platelets were moderately decreased, possibly as a result of moderate consumption at the site of duodenal hemorrhage. AST and ALT were moderately increased when compared to the SEAP group. The magnitude of this increase remained within the limits of what is commonly associated with virus vector-related liver damage. The cause for the absence of similar variations in liver enzymes in the SEAP group is unclear considering that microscopic changes indicative of virus exposure were present in that group. Dehydration is typically associated with an increase in albumin and total protein. The absence of albumin increase and the relatively low increase in total protein (TP) might result from liver compromise or less likely could be due to protein loss.

In the absence of changes indicative of renal insufficiency or enteropathy, the clustering of the findings above described would suggest that debilitation with emaciation and dehydration could be related to decrease food and water intakes (pancreatic zymogen granule depletion, enlarged gall bladder). The liver and/or duodenal microscopic changes might be associated with the onset of this syndrome.

Statistically and/or biologically significant findings are shown in Table 1 below. Abbreviations are as follows: RBC, Red Blood Cell Count; Hb, Hemoglobin; HCT, Hematocrit; MCV, Mean Cell Volume; MCH; Mean Cell Hemoglobin; AST, Aspartate Aminotransferase; ALT, Alanine Aminotransferase; ALP, Alkaline Phosphatase; TP, Total Protein; Plat, Platelets.

TABLE 1

| Parameter | Day | Statistically and/or Biologically Significant Findings |
|---|---|---|
| Body Weight (% from control) | 4 | ↓ (76%) |
| Organ Weight (Ab) | 4 | ↓ Liver, ↓ Spleen, ↓ Thymus |
| % Organ/BW Ratio | 4 | ↓ Liver, ↓ Spleen, ↓ Thymus |
| Hematology | 4 | ↑ RBC, ↑ Hb, ↑ HCT, ↓MCV, ↓MCH, ↓ Plat |
| Clinical Chemistry | 4 | ↑ AST, ↑ ALT, ↓ ALP, ↑ TP |

In summary, the following notable histopathological changes were found in AdBMP-10 mice compared to control mice. Hemorrhages in the wall of the proximal duodenum were found, and correlated with the gross observation of red discoloration at the pylorus. Fibrin thrombi were present in dilated vessels of the submucosa, in association with findings in Brunner's glands of the duodenum. Duodenal transmural hemorrhage with slight muscular necrosis was observed, indicative of localized vascular compromise. The livers of AdBMP-10 mice have a mottled appearance, resulting from focal necrosis caused by scattered hemorrhaging.

In summary, AdBMP-10 mice were observed to have vascular dysplasia, as evidenced by pronounced subcutaneous vascular dysplasia, widespread congestion and scattered hemorrhages, and enlarged blood vessels in the brain, liver, and lung. AdBMP-10 mice were debilitated and died by day 7. There was a marked decrease in body weight, with loss of subcutaneous and visceral fat. Lymphoid atrophy was observed, in addition to thrombocytopenia.

### EXAMPLE 3

### BMP-10 Activation of Signaling in Human Primary Endothelial Cells

Endothelial cells play an important role in vascular homeostasis. The ability of BMP-10 to activate signaling through receptor serine/threonine kinases was studied in human primary endothelial cells. Cell responsiveness to BMP-10 treatment was analyzed by assessing the phosphorylation state of receptor Smads (R-Smad) in addition to using quantitative PCR to determine expression levels of the inhibitory receptor Smads after BMP-10 treatment.

### Experiment A. Activation of R-Smad 1, 5, 8 in HUVECs and HUAECs.

### Methods.

BMP-10- and GFP-conditioned media were generated as follows. Human embryonic kidney cells (HEK293) were plated at a density of 1×10⁶ cells/p100 tissue culture dish in 10 mls of Dulbecco's modified Eagle's media (DMEM)+10% heat inactivated fetal bovine serum+200 mM glutamine. Plates were incubated at 37° C., 5% CO2 for 24 hours. Cells were transfected with 10 ug of AdBMP-10 plasmid or AdGFP plasmid using fugene, a lipid based transfection method. Plates were returned to incubator for 18 hours and then washed 1× with DMEM+200 mM glutamine. Media was removed and replaced with 6 ml of DMEM+200 mM glutamine. Plates were incubated for an additional 48 hours. Media was removed and centrifuged for 5 minutes at 300 rpm to remove floating cells and transferred to a clean tube. Conditioned media containing BMP-10 or GFP was analyzed under non-denaturing conditions on an SDS-polyacrylamide gel and transferred to a nitrocellulose membrane. Immunodetection of BMP-10 was performed using a BMP-10 specific antibody (Orbigen, Catalog Number PAB-10450) according to manufacturer's directions. (Experiment C, below).

Human umbilical vein endothelial cells (HUVEC) (Lonza Catalog NumberCC-2517) and human aortic endothelial cells (HUAEC) (Lonza Catalog NumberCC-2520) were cultured according to manufacturers directions in EGM complete media (Lonza Catalog Number CC-3024) in p60 tissue culture dish and incubated at 37° C., 5% CO2, when they reached a confluence of ˜70-80% the complete growth media was removed and cells were washed 1× with basal media (Lonza Catalog Number CC-3129)+0.1% delipidized BSA (BD Bioscience Catalog Number 354331) and 3.6 ml basal media with 0.1% delipidized BSA was added and plates were incubated for 4 hours. After 4 hours either 400 ul of BMP-10 conditioned media or 400 ul GFP (control) conditioned media was added to the cells. Plates were returned to the incubator for 15, 30, 60 or 120 minutes. At the various time points stated, plates were removed from the incubator, media was removed and cells were washed 1× with 3 ml of cold PBS. PBS was removed and cells were lysed with 300 ul cell lysis buffer (Cell Signaling Technology Catalog Number 9803) plus proteinase inhibitors for the immunoblot samples. Cell lysates were separated by SDS polyacrylamide gel electrophoresis and transferred to a nitrocellulose membrane. Immunodetection of phosphorylated Smad 1, 5, 8 was performed using phospho Smad 1, 5, 8 specific antibodies (Cell Signaling Technology Catalog Number 9511) according to manufacturer's directions.

### Results.

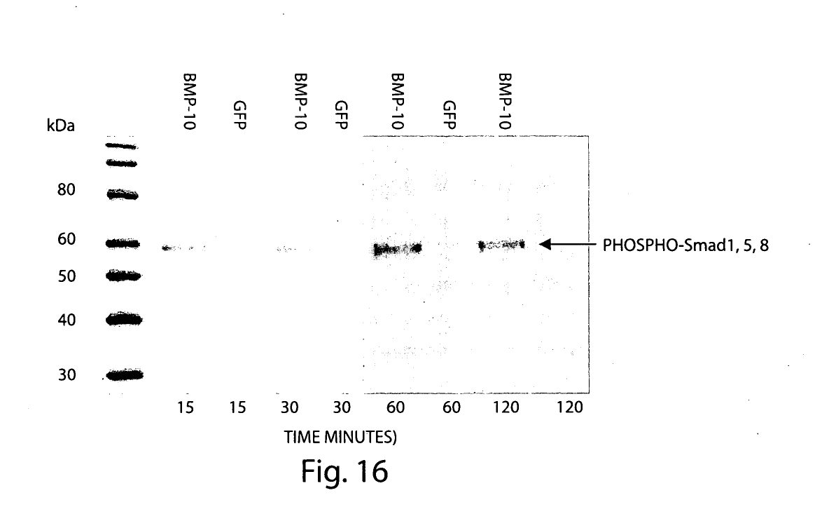

BMP-10-conditioned media clearly elicited phosphorylation of R-Smad 1, 5, and 8 as shown in the immunoblot in FIG. 16. No Smad phosphorylation was elicited in HUVECs by GFP-conditioned medium. Thus, results showed an activation of the BMP signaling pathway that is specific for endothelial cells treated with BMP-10-containing conditioned medium.

Experiment B. Smad 6, 7 mRNA Expression in Response to BMP-10 Conditioned Media.

### Methods.

Conditioned media containing BMP-10 or GFP was prepared essentially as described in the above experiment, except that after the various times (30, 60, 120 minutes) of treatment with BMP-10-conditioned medium, the HUVECs were lysed in buffer suitable for total RNA recovery for analysis of RNA using a quantitative polymerase chain reactions assay (TaqMan®) or global gene expression analysis using oligonucleotide arrays (latter experiment is described below in Example 8). Quantitative PCR experiments were performed to measure induction of mRNA for the inhibitory Smads 6 and 7, and validated the gene expression profiling results of Example 8. Analysis of human SMAD6, SMAD7 and GAPDH (glyceraldehyde-3-phosphate dehydrogenase) mRNA levels were performed using TaqMan® EZ RT-PCR Core Reagents (Applied Biosystems) according to manufacturer's recommendations. Fold change determinations were calculated using the standard curve method and samples were normalized to GAPDH as described by manufacturer.

Primers used were as follows.

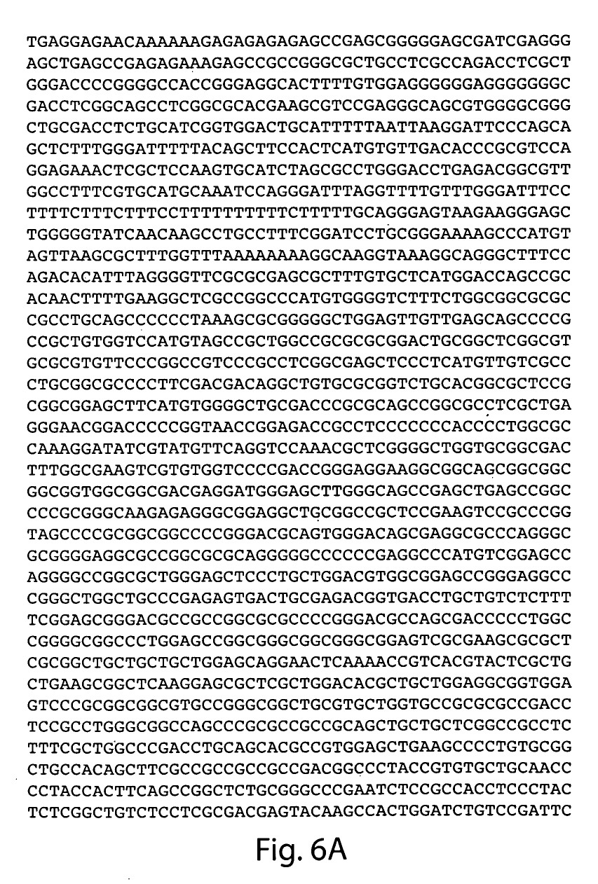

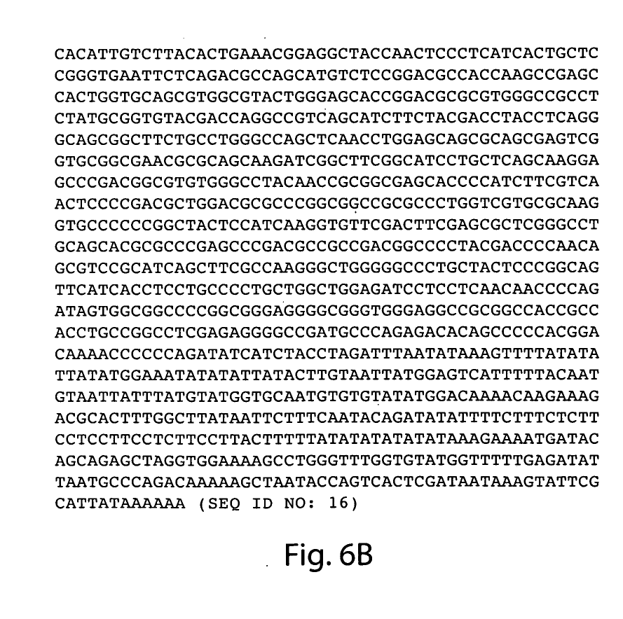

Human SMAD6 (FIG. 6A-6B, SEQ ID NO:16, Accession
number NM_005585)
Forward primer

(SEQ ID NO:7)

5′-GCCACTGGATCTGTCCGATT-3′
Reverse primer

(SEQ ID NO:8)

5′-CACCCGGAGCAGTGATGAG-3′
Probe

(SEQ ID NO:9)

5′-FAM-ACATTGTCTTACACTGAAACGGAGGCTACCAACT-TAMRA-3′
Human SMAD7 (Hayashi et al. (1997) Cell 89:1165-
1173)
Forward

(SEQ ID NO:10)

5′-CAGAAGGTGCGGAGCAAAAT-3′
(TM = 59)
Reverse

(SEQ ID NO:11)

5′-TGTACACCCACACACCATCCA-3′
(TM = 59)
Probe

(SEQ ID NO:12)

5′-FAM-CTGCGGCATCCAGCTGACGC-TAMRA-3′
Human GAPDH:
Forward primer

(SEQ ID NO:13)

5′-CCACATCGCTCAGACACCAT-3′
Reverse Primer

(SEQ ID NO:14)

GCGCCCAATACGACCAAA
Probe

(SEQ ID NO:15)

5′-FAM-CGTTGACTCCGACCTTCACCTTCCC-TAMRA-3′

### Results.

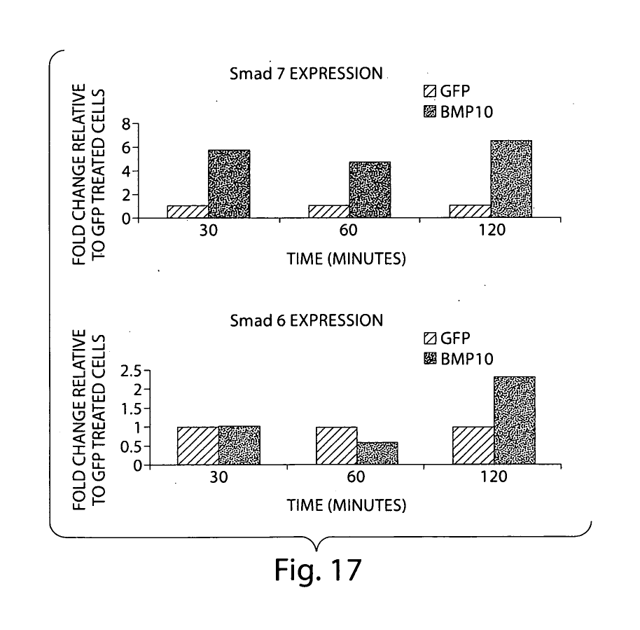

The time course study of the induction of the inhibitory Smads 6 and 7 mRNA can be seen in FIG. 17. As compared to the GFP-treated cells, expression of Smad 7 was induced 4 to 6 fold, and Smad 6 was expressed two-fold after 120 minutes.

Experiment C. Presence of BMP-10 in Conditioned Media Confirmed by antiBMP-10pro Antibody Immunoblot.

### Methods.

Conditioned media from the HEK293 cells transfected with either AdBMP-10 or AdGFP was generated as described in Experiment A, above. Samples of conditioned media were separated by sodium dodecyl sulfate (SDS)-PAGE, transferred to nitrocellulose, and probed with an anti-BMP-10pro primary antibody, followed by standard immunodetection techniques.

### Results

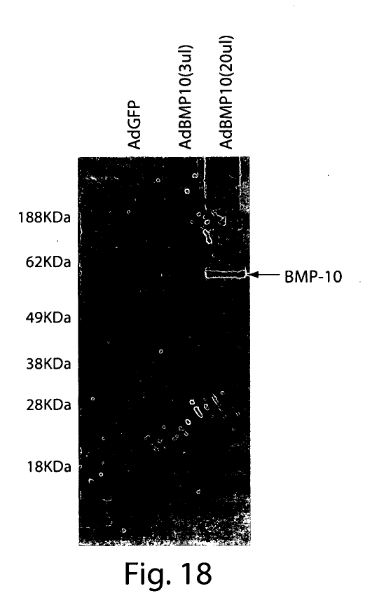

The presence of human BMP-10 protein in the conditioned media as detected by immunoblot analysis is shown in FIG. 18. The BMP-10 has migrated with the expected molecular weight in a SDS-PAGE denaturing gel of approximately 60 kDa.

### EXAMPLE 4

### BMP-10 Activation of Signaling in Human Primary Renal Proximal Tubule Epithelial Cells

The ability of BMP-10 to activate signaling through receptor serine/threonine kinases was studied in human primary renal epithelial cells. Cell responsiveness to BMP-10 treatment was analyzed by assessing the phosphorylation state of BMP receptor Smads (R-Smads 1, 5, 8).

### Activation of R-Smad 1, 5, 8 in RPTECs.

### Methods.

Human primary renal proximal tubule epithelial cells (RPTEC) (Lonza Catalog Number CC-2517) were cultured according to manufacturers directions in renal epithelial growth media (REGM) with added growth supplements (Lonza Catalog Number CC-3190) in 60 mm tissue culture plates and incubated at 37° C., 5% CO2, when they reached a confluence of ˜70-80% the complete growth media was removed and cells were washed 1× with renal epithelial basal media, REBM (Lonza Catalog Number CC-3191)+0.1% delipidized BSA (BD Bioscience Catalog Number 354331) and 4 ml rREBM with 0.1% delipidized BSA was added and plates were incubated for 3 hours. After 3 hours cells were treated with either mature recombinant BMP-10 protein at a final concentration of 100 ng/ml (R&D Systems Catalog Number 2926-BP) or phosphate buffered saline (PBS). Plates were returned to the incubator for 60 minutes. After 60 minutes, media was removed and cells were washed 1× with 3 ml of cold PBS. The PBS was removed and the cells were lysed with 300 ul cell lysis buffer (Cell Signaling Technology Catalog Number 9803) plus proteinase inhibitors (Roche Applied Science Catalog Number 11697498001) for the protein analysis using a Western immunoblot. Cell lysates were separated by sodium dodecyl sulfate-polyacrylamide gel electrophoresis and transferred to a nitrocellulose membrane. Immunodetection of phosphorylated Smad 1, 5, 8 was performed using phosphoSmad 1, 5, 8 specific antibodies (Cell Signaling Technology Catalog Number 9511) according to manufacturer's directions. Immunodetection of β-actin (Cell Signaling Technology Catalog Number 4967) was used to normalize sample protein loading.

### Results.

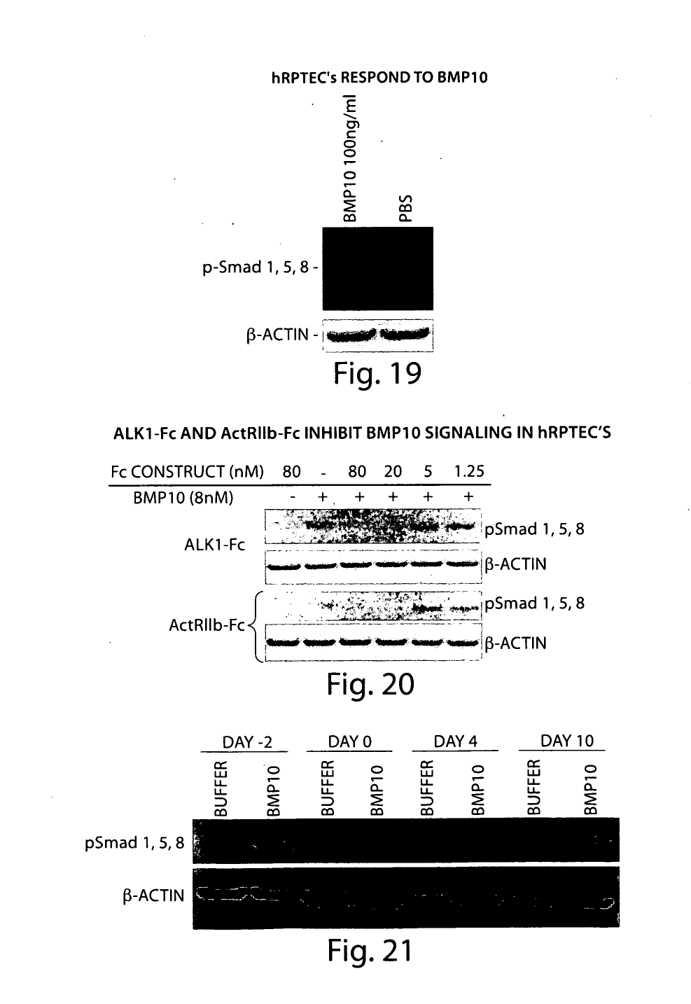

BMP-10 treatment clearly elicited phosphorylation of R-Smad 1, 5, and 8 as shown in the immunoblot in FIG. 19. No Smad phosphorylation was elicited in hRPTECs by PBS treatment. Thus, results showed an activation of the BMP signaling pathway that is specific for renal proximal tubule epithelial cells treated with BMP-10 protein.

### EXAMPLE 5

### Inhibition of BMP-10 Signaling in Human Primary Renal Proximal Tubule Epithelial Cells using Soluble Activin Receptor-Like Kinase 1 (ALK1) and Soluble Activin ReceptorIIb (ActRIIb)

ALK1 is a type I receptor for transforming growth factor-β (TGF-β) family proteins. It was previously reported that BMP-10 signaling in human umbilical vein endothelial cells could be blocked by soluble ALK1 (David et al. (2007) Blood 109:1953-1961). We tested the ability of both soluble ALK1 and ActRIIb to block the phosphorylation of BMP receptor Smads (R-Smads 1, 5, 8) in primary renal proximal tubule cells.

### Inhibition of R-Smad 1, 5, 8 in RPTECs Using Soluble ALK-1 and Soluble ActRIIb.

### Methods.

Human primary renal proximal tubule epithelial cells (RPTEC) (Lonza Catalog Number CC-2517) were cultured according to manufacturers directions in REGM media with added growth supplements (Lonza Catalog Number CC-3190) in 60 mm tissue culture plates and incubated at 37° C., 5% CO2, when they reached a confluence of ˜70-80% the complete growth media was removed and cells were washed 1× with renal epithelial basal media, REBM (Lonza Catalog Number CC-3191)+0.1% delipidized BSA (BD Bioscience Catalog Number 354331) and 4 ml REBM with 0.1% delipidized BSA was added and plates were incubated for 3 hours. After 3 hours of serum starvation, media was removed and replaced with 4 ml of serum free basal media containing mature recombinant BMP-10 protein at a concentration of 100 ng/ml (R&D Systems Catalog Number 2926-BP) that had been pre-incubated for 45 minutes at 37° C. with varying concentrations (20, 10, 2.5 and 1.25 fold molar excess compared to BMP-10 (8 nM)) of either, soluble ALK-1 (R&D Systems Catalog Number 370-AL, amino acid residues 1-118 of the extracellular domain fused to Fc region of human IgG1₁), soluble ActRIIb (Extracellular domain fused to human IgG1) or soluble ALK-3 (R&D Systems Catalog Number 315-BR, amino acid residues 1-152 of the extracellular domain fused to Fc region of human IgG1₁) Controls included BMP-10 treatment alone and Fc constructs at a 10 fold molar excess alone. After 60 minutes, media was removed and cells were washed 1× with 3 ml of cold PBS. PBS was removed and cells were lysed with 300 ul cell lysis buffer (Cell Signaling Technology Catalog Number 9803) plus proteinase inhibitors (Roche Applied Science Catalog Number 11697498001) for phospho-protein analysis. Cell lysates were separated by sodium dodecyl sulfate (SDS)polyacrylamide gel electrophoresis and transferred to a nitrocellulose membrane. Immunodetection of phosphorylated Smad 1, 5, 8 was performed using phospho-Smad 1, 5, 8 (p-Smad 1, 5, 8) specific antibodies (Cell Signaling Technology Catalog Number 9511) according to manufacturer's directions. Immunodetection of β-actin (Cell Signaling Technology Catalog Number 4967) was used to normalize sample protein loading.

### Results

Soluble ALK1 or soluble ActRIIB treatment clearly inhibited the phosphorylation of R-Smad 1, 5, and 8 elicited by BMP-10 as shown in the immunoblot in FIG. 20. Thus, results showed an inhibition of the BMP signaling pathway that is specific for renal proximal tubule epithelial cells treated with BMP-10.

### EXAMPLE 6

### BMP-10 Activates 3T3-L1 Cells at Various Stages of Adipocyte Differentiation

### Methods.

### Adipocyte Differentiation Protocol

The mouse fibroblast cell line, 3T3-L1 (ATCC, Catalog Number CL-173) was grown to confluency in Dulbecco's modified Eagle's medium (DMEM) supplemented with 10% (v/v) fetal bovine serum (FBS) in 6-well culture dishes (day −2 of differentiation) and incubated at 37° C., 10% CO₂. After two days of post confluency (day 0 of differentiation) cells were stimulated with differentiation induction media (DMEM supplemented with 10% (v/v) FBS, 1 μM dexamethasone (Sigma), 0.5 mM isobutylmethylxanthine (Sigma), 1 μg/ml insulin (NovoNordisk). After two days (day 2 of differentiation) induction media is replaced with insulin media (DMEM supplemented with 10% (v/v) FBS (fetal bovine serum), insulin (1 μg/ml). Two days later (day 4 of differentiation) insulin media is removed and replaced with DMEM supplemented with 10% (v/v) FBS and cells are fed every two days until full differentiation is achieved by day 10.

### BMP-10 Treatment of 3T3-L1 Cells During Adipocyte Differentiation

3T3-L1 cells were treated with BMP-10 (100 ng/ml) at various stages of adipocyte differentiation (day −2, 0, 2, 4 and 10) as follows: media was removed and cells were rinsed with serum free DMEM (DMEM supplemented with 0.1% delipidized BSA (BD Bioscience Catalog Number 354331)). 3 ml of serum free DMEM was added to each well of a 6-well plate and cells were incubated at 37° C., 10% CO₂ for four hours. Following incubation in serum free media, 3 μl of mature recombinant BMP-10 protein at a concentration of 100 ng/μl (R&D Systems Catalog Number 2926-BP) or 3 μl of PBS was added to cells and plates were returned to incubator. One hour after treatment media was removed and cells were washed 1× with 3 ml of cold PBS. PBS was removed and cells were lysed with 300 μl cell lysis buffer (Cell Signaling Technology Catalog Number 9803) plus proteinase inhibitors (Roche Applied Science Catalog Number 11697498001) for protein analysis using Western immunoblot. Cell lysates were separated by SDS polyacrylamide gel electrophoresis and transferred to a nitrocellulose membrane. Immunodetection of phosphorylated Smad 1, 5, 8 was performed using phospho-Smad 1, 5, 8 specific antibodies (Cell Signaling Technology Catalog Number 9511) according to manufacturer's directions. Immunodetection of β-actin (Cell Signaling Technology Catalog Number 4967) was used to normalize sample protein loading.

### Results.

BMP-10 treatment clearly elicited phosphorylation of R-Smad 1, 5, and 8 at various stages of adipocyte differentiation as shown in the immunoblot in FIG. 21. No Smad phosphorylation was elicited in 3T3-L1 cells by PBS treatment. Thus, results showed an activation of the BMP signaling pathway that is specific for fibroblast cells treated with BMP-10 protein prior to and during adipocyte differentiation.

### EXAMPLE 7

### Immunohistochemistry (IHC) Analysis of Human BMP-10 Protein Expression in Human and Mouse Tissues

### Experiment A. Immunohistochemistry Analysis of BMP-10 Protein Expression in Human Normal and Disease Tissues.

### Methods.

### Peptide Selection and Antibody Production:

The sequence for BMP-10-LP9168 was analyzed by the algorithm of Hopp and Woods to determine potential peptides for synthesis and antibody production. The peptides were then BLASTed against the Swissprot database to determine uniqueness and to help predict the specificity of the resulting antibodies. Peptide DKGVVTYKFKYE (SEQ ID NO:21) was selected and synthesized, and rabbit polyclonal antisera were generated. In order to allow for peptide conjugation to the carrier protein, a cysteine residue was added to the N-terminus of the peptide. The third bleeds were subjected to peptide affinity purification, and the resulting antisera were then used as primary antibodies in immunohistochemistry experiments.

### Antibody Titration Protocol and Positive Control Study Results:

Antibody titration experiments were conducted with antibody BMP-10-LP9168 (rabbit polyclonal) to establish concentrations that would result in minimal background and maximal detection of signal. Serial dilutions were performed at 20 ug/ml, 10 ug/ml, 5 ug/ml, and 2.5 ug/ml. The serial dilution study demonstrated the highest signal-to-noise ratios at a concentration of 2.5 ug/ml on paraffin-embedded, formalin-fixed tissues. This concentration was used for the study. Antibody BMP-10-LP9168 was used as the primary antibody, and the principal detection system consisted of a Vector anti-rabbit secondary (BA-1000) and a Vector ABC-AP kit (AK-5000) with a Vector Red substrate kit (SK-5100), which was used to produce a fuchsia-colored deposit.

Tissues were also stained with a positive control antibody (CD31) to ensure that the tissue antigens were preserved and accessible for immunohistochemical analysis. Only tissues that stained positive for CD31 were selected for the remainder of the study. The negative control consisted of performing the entire immunohistochemistry procedure on adjacent sections in the absence of primary antibody.

Slides were imaged with a DVC 1310C digital camera coupled to a Nikon microscope. Images were stored as TIFF files using Adobe Photoshop.

### Results.

Antibody LP9168 to bone morphogenic protein 10 (BMP-10) was evaluated on an extensive series of normal human tissues, as well as matched samples of selected normal and diabetic tissues, normal and atherosclerotic coronary arteries, normal and ischemic heart, and normal lung and lung from patients with pulmonary hypertension. Because of the similarity of BMP-10 to other proteins, this is believed to be a secreted target.

This antibody showed predominantly cytoplasmic staining, although nuclear staining and membranous staining were also rarely present. The quality of staining was satisfactory, with good distinction between positive and negative cell types.

In normal tissues, the most prominent staining was identified in placental cytotrophoblasts, breast epithelium, adrenal medulla, inflammatory subsets, ovarian follicles, testicular Leydig cells, spermatocytic precursors, and endometrium. Positive staining was also identified in colonic surface epithelium, prostatic epithelium, intestinal epithelium, gastric epithelium, pancreatic acini, and urothelium. Peripheral nerves, ganglion cells, gastrointestinal neuroendocrine cells, and pancreatic islet cell subsets were positive. Focal positivity was identified in squamous epithelium, hepatocytes, bile ducts, and neurons in the brain.

In the diseased tissues, there were no significant alterations in staining when compared to normal tissues.

Report sections followed by “[NTS2]” indicate samples that were part of the Normal Tissue Screen II panel. Report sections followed by “[TIIDSI]” indicate samples that were part of the Type II Diabetic Screen I panel. Report sections followed by “[Single Tissue]” indicate samples that were individual tissue sections.

| | | |
|---|---|---|
| Cell/Zone | Intensity | Localization |

Breast [NTS2]
Sample 1: This sample of normal breast was obtained at surgery
from an 18-year-old female.

| | | |
|---|---|---|
| epithelium | 3 | C, M |
| stroma | 0 | |

Sample 2: This sample of normal breast was obtained at surgery
from a 21-year-old female.

| | | |
|---|---|---|
| epithelium | 3 | C, M |
| stroma | 0 | |

Colon [NTS2]
Sample 1: This sample of normal colon was obtained at surgery
from a 54-year-old male.

| | | |
|---|---|---|
| epithelium | 2 (occasional) | C |
| smooth muscle cells | 0 | |
| inflammatory cells | 2-3 (many) | C |
| ganglion cells | (NI) | |

Sample 2: This sample of normal colon was obtained at surgery
from a 71-year-old female.
Neuroendocrine cells were positive.

| | | |
|---|---|---|
| epithelium | 1 (many) | C |
| smooth muscle cells | 0 | |
| inflammatory cells | 2-3 (occasional) | C |
| ganglion cells | (NI) | |
| others | 2-3 (rare) | C |

Lung [NTS2]
Sample 1: This sample of normal lung was obtained from an
81-year-old female who died of an abdominal aortic aneurysm.
Inflammatory cells were positive.

| | | |
|---|---|---|
| airway | 2-3 (many) | C |
| pneumocytes | 2 (occasional) | C |
| alveolar macrophages | 2 (occasional) | C |
| others | 3 (occasional) | C |

Sample 2: This sample of normal lung was obtained at surgery
from a 66-year-old male.
Peripheral nerves were positive.

| | | |
|---|---|---|
| airway | 2 (many) | C |
| pneumocytes | 2 (occasional) | C |
| alveolar macrophages | 1-2 | C |
| others | 2 | C |

Ovary [NTS2]
Sample 1: This sample of normal ovary was obtained at surgery
from a female patient of unknown age.

| | | |
|---|---|---|
| follicle | 3 | C |
| stroma | 1-2 | C |

Sample 2: This sample of normal ovary was obtained at surgery
from a female patient of unknown age.

| | | |
|---|---|---|
| follicle | 3 (many) | C |
| stroma | 1-2 | C |

Pancreas [NTS2]
Sample 1: This sample of normal pancreas was obtained from
a 16-year-old female who died of a self-inflicted gunshot wound.

| | | |
|---|---|---|
| acinar epithelium | 2-3 (many) | C, M |
| islets of Langerhans | 1-2 | C |
| ductal epithelium | 0 | |

Sample 2: This sample of normal pancreas was obtained at autopsy
from an 82-year-old male.

| | | |
|---|---|---|
| acinar epithelium | 2-3 (many) | C, M |
| islets of Langerhans | 2-3 (occasional) | C |
| ductal epithelium | 0 (most) | |

Prostate [NTS2]
Sample 1: This sample of normal prostate was obtained at surgery
from a 62-year-old male.

| | | |
|---|---|---|
| glandular epithelium | 3 | C, N |
| fibromuscular stroma | 0 | |

Sample 2: This sample of normal prostate was obtained from a
28-year-old male who died of head trauma.

| | | |
|---|---|---|
| glandular epithelium | 2 | C, N |
| fibromuscular stroma | 0 | |

Skin [NTS2]
Sample 1: This sample of normal skin was obtained at surgery from a
32-year-old female.

| | | |
|---|---|---|
| epidermis | 1-2 (many) | C, N |
| dermis | 0 | |

Sample 2: This sample of normal skin was obtained from a
38-year-old male who died of carbon monoxide poisoning.

| | | |
|---|---|---|
| epidermis | 1-2 (many) | C |
| dermis | 0 | |

Adrenal [NTS2]
Sample 1: This sample of normal adrenal was obtained from a
73-year-old female who died of coronary arteriosclerosis with
organizing thrombosis.

| | | |
|---|---|---|
| cortex | 2 (occasional) | C |
| medulla | 3 | C, M |

Sample 2: This sample of normal adrenal was obtained from a
73-year-old female who died of pancreatic adenocarcinoma.

| | | |
|---|---|---|
| cortex | 2 (occasional) | C |
| medulla | 3 | C, M |

Bladder [NTS2]
Sample 1: This sample of normal bladder was obtained from an
80-year-old female who died of an intracerebral hemorrhage.

| | | |
|---|---|---|
| urothelium | 2 | C |
| smooth muscle cells | 0 | |

Sample 2: This sample of normal bladder was obtained from an
82-year-old female who died of chronic obstructive pulmonary disease.

| | | |
|---|---|---|
| urothelium | 2 | C, M |
| smooth muscle cells | 0 | |

Brain, Cortex [NTS2]
Sample 1: This sample of normal cortex was obtained from a 54-year-old
female who died of an acute myocardial infarct with probable arrhythmia.

| | | |
|---|---|---|
| neurons | 1-2 (occasional) | C |
| glia | 0 | |

Sample 2: This sample of normal cortex was obtained from an 87-year-old
male who died of renal failure.

| | | |
|---|---|---|
| neurons | 1 (occasional) | C, N |
| glia | 0 | |

Heart [NTS2]
Sample 1: This sample of normal heart was obtained from a 59-year-old
male who died of sepsis and pulmonary edema.
Endothelium was positive. Granular interstitial staining of possible
nerve twiglets was occasionally identified.

| | |
|---|---|
| cardiac myocytes | 0 |

Sample 2: This sample of normal heart was obtained from a
17-year-old male who died of trauma.

| | |
|---|---|
| cardiac myocytes | 0 |

Liver [NTS2]
Sample 1: This sample of normal liver was obtained from a
46-year-old male who died of an intracranial hemorrhage.

| | | |
|---|---|---|
| hepatocytes | 2 | C, N |
| bile duct epithelium | 1-2 | C, N |
| Kupffer cells | 0 | |

Sample 2: This sample of normal liver was obtained from a
14-year-old male who died of trauma.

| | | |
|---|---|---|
| hepatocytes | 2 (many) | C, N |
| bile duct epithelium | 1-2 | C, N |
| Kupffer cells | 0 | |

Skeletal Muscle [NTS2]
Sample 1: This sample of normal skeletal muscle was obtained
from a 62-year-old male with brain carcinoma and diabetes.

| | |
|---|---|
| myocytes | 0 (most) |

Sample 2: This sample of normal skeletal muscle was obtained
at autopsy from a 79-year-old female with diabetes.

| | |
|---|---|
| myocytes | 0 |

Placenta [NTS2]
Sample 1: This sample of normal placenta was obtained at surgery
from a 33-year-old female.

| | | |
|---|---|---|
| trophoblasts | 2-3 (many) | C, M, N |
| stroma | 1 (occasional) | C, N |

Sample 2: This sample of normal placenta was obtained at
delivery from a 34-year-old female.

| | | |
|---|---|---|
| trophoblasts | 3 (many) | C |
| stroma | 0 (most) | |

Small Intestine [NTS2]
Sample 1: This sample of normal small intestine was obtained
from an 18-year-old male who died of trauma.

| | | |
|---|---|---|
| epithelium | 2-3 (many) | C |
| smooth muscle cells | 0 | |
| inflammatory cells | 2 (many) | C |
| ganglion cells | 2 | C |

Sample 2: This sample of normal small intestine was obtained
from a 59-year-old female.
Neuroendocrine cells were positive.

| | | |
|---|---|---|
| epithelium | 2-3 (many) | C |
| smooth muscle cells | 0 | |
| inflammatory cells | 2 (many) | C |
| ganglion cells | 2 | C |
| others | 4 (occasional) | C |

Spleen [NTS2]
Sample 1: This sample of normal spleen was obtained at
splenectomy from a 77-year-old female.

| | | |
|---|---|---|
| white pulp | 2 (many) | C |
| red pulp | 2 (occasional) | C |

Sample 2: This sample of normal spleen was obtained from an
83-year-old male who died of cerebral arteriosclerosis.

| | | |
|---|---|---|
| white pulp | 2 (many) | C |
| red pulp | 2 (occasional) | C |

Stomach [NTS2]
Sample 1: This sample of normal stomach was obtained from a
62-year-old male who died of a myocardial infarct.

| | | |
|---|---|---|
| epithelium | 2-3 (many) | C |
| smooth muscle cells | 0 | |

Sample 2: This sample of normal stomach was obtained from a
33-year-old male who died of trauma associated with a
motor-vehicle accident.
Ganglion cells and peripheral nerves were positive.

| | | |
|---|---|---|
| epithelium | 2 (many) | C, N |
| smooth muscle cells | 0 | |
| others | 2 | C |

Testis [NTS2]
Sample 1: This sample of normal testis was obtained at surgery
from a 67-year-old male with prostate carcinoma.

| | | |
|---|---|---|
| seminiferous epithelium | 2-3 | C, M |
| Leydig cells | 2-3 | C, N |

Sample 2: This sample of normal testis was obtained from a
62-year-old male who died of a myocardial infarct.

| | | |
|---|---|---|
| seminiferous epithelium | 2 (many) | C |
| Leydig cells | 2-3 | C |

Thymus [NTS2]
Sample 1: This sample of normal thymus was obtained at surgery
from a 28-year-old female.

| | | |
|---|---|---|
| lymphocytes | 2 (many) | C |
| epithelium | 2 (many) | C |

Sample 2: This sample of normal thymus was obtained from a
36-week-old female fetus who died of pulmonary insufficiency
due to diaphragmatic herniation.

| | | |
|---|---|---|
| lymphocytes | 2 (many) | C |
| epithelium | 1 (many) | C |

Thyroid [NTS2]
Sample 1: This sample of normal thyroid was obtained from a
76-year-old male who died of a ruptured esophagus with mediastinitis.

| | | |
|---|---|---|
| follicular epithelium | 2 (occasional) | C |

Sample 2: This sample of normal thyroid was obtained from a
78-year-old female who died of a pulmonary embolism.

| | | |
|---|---|---|
| follicular epithelium | 1-2 (occasional) | C |

Tonsil [NTS2]
Sample 1: This sample of normal tonsil was obtained at tonsillectomy
from a 20-year-old male.

| | | |
|---|---|---|
| epithelium | 2 (occasional) | C |
| lymphocytes | 2 (many) | C |

Sample 2: This sample of normal tonsil was obtained at surgery
from a 27 -year-old male.

| | | |
|---|---|---|
| epithelium | (NI) | |
| lymphocytes | 2-3 (many) | C |

Uterus [NTS2]
Sample 1: This sample of normal uterus was obtained at surgery from
a 36-year-old female.

| | | |
|---|---|---|
| endometrium | 3 (many) | C |
| myometrium | 2 | C |

Sample 2: This sample of normal uterus was obtained at surgery from
a 42-year-old female.

| | | |
|---|---|---|
| endometrium | 3 (many) | C |
| myometrium | 1 | C |

Kidney, Medulla [NTS2]
Sample 1: This sample of normal kidney was obtained at autopsy from a
78-year-old female.

| | | |
|---|---|---|
| renal tubular epithelium | 2 (many) | C, N |

Sample 2: This sample of normal kidney was obtained at surgery from
a 57-year-old female with a renal mass.

| | | |
|---|---|---|
| renal tubular epithelium | 2 (many) | C, M |

Kidney, Cortex [NTS2]
Sample 1: This sample of normal kidney was obtained at surgery from a
68-year-old female.

| | | |
|---|---|---|
| glomeruli | 0 | |
| renal tubular epithelium | 2-3 (many) | C, M |

Sample 2: This sample of normal kidney was obtained at nephrectomy
from a 67-year-old male.

| | | |
|---|---|---|
| glomeruli | 0 | |
| renal tubular epithelium | 2-3 (many) | C |

Heart [Single Tissue]
Sample 1: This sample of normal heart was obtained from an
86-year-old male who died of a necrotic bowel.

| | |
|---|---|
| cardiac myocytes | 0 |

Sample 2: This sample of normal heart was obtained from a
58-year-old female who died of gastrointestinal bleeding.
Endothelium was positive. Granular interstitial staining of
possible nerve twiglets was occasionally identified.

| | | |
|---|---|---|
| cardiac myocytes | 1 | C |
| others | 2-3 (rare) | C |

Sample 3: This sample of normal heart was obtained from a
60-year-old male who died of non-small cell lung carcinoma.

| | | |
|---|---|---|
| cardiac myocytes | 1-2 (many) | C |

Artery [Single Tissue]
Sample 1: This sample of normal artery was obtained from an
84-year-old female with hypertension who died of chronic obstructive
pulmonary disease.

| | |
|---|---|
| endothelium | 0 |
| smooth muscle cells | 0 |

Sample 2: This sample of normal artery was obtained from an
82-year-old female who died of chronic obstructive pulmonary disease.

| | |
|---|---|
| endothelium | 0 |
| smooth muscle cells | 0 |

Sample 3: This sample of normal artery was obtained from a 17-year-old
female who died of trauma.

| | |
|---|---|
| endothelium | 0 |
| smooth muscle cells | 0 |

Artery, Atherosclerosis [Single Tissue]
Sample 1: This sample of artery was obtained at autopsy from a
57-year-old female.

| | |
|---|---|
| endothelium | 0 |
| plaque | 0 |
| smooth muscle cells | 0 |
| inflammatory cells | 0 |

Sample 2: This sample of artery was obtained from a
79-year-old male who died of peritonitis secondary to bowel ischemia.

| | |
|---|---|
| endothelium | 0 |
| plaque | 0 |
| smooth muscle cells | 0 |
| inflammatory cells | 0 |

Sample 3: This sample of artery was obtained at autopsy
from a 74-year-old male.

| | | |
|---|---|---|
| endothelium | 0 | |
| plaque | 0 | |
| smooth muscle cells | 0 | |
| inflammatory cells | 1-2 (rare) | C |

Adipose, Subcutaneous, Non-Diabetic [TIIDS1]
Sample 1: This sample of adipose was obtained from a 75-year-old male.

| | |
|---|---|
| adipocytes | 0 |

Sample 2: This sample of adipose was obtained at breast reduction from a
59-year-old female with macromastia.

| | |
|---|---|
| adipocytes | 0 |

Adipose, Visceral, Non-Diabetic [TIIDS1]
Sample 1: This sample of adipose was obtained at autopsy from an
83-year-old female.

| | |
|---|---|
| adipocytes | 0 |

Sample 2: This sample of adipose was obtained at autopsy from a
71-year-old male.
Serum and macrophages were positive.

| | | |
|---|---|---|
| adipocytes | 0 | |
| others | 1-2 | C, E |

Colon, Non-Diabetic [TIIDS1]
Sample 1: This sample of normal colon was obtained at hemicolectomy
from a 66-year-old female with metastatic colon carcinoma.

| | | |
|---|---|---|
| epithelium | 2-3 (many) | C |
| smooth muscle cells | 0 | |
| inflammatory cells | 2-3 (many) | C, N |
| ganglion cells | 2 | |

Sample 2: This sample of normal colon was obtained at hemicolectomy
from a 75-year-old female with colon cancer.

| | | |
|---|---|---|
| epithelium | 2-3 (occasional) | C, N |
| smooth muscle cells | 0 | |
| inflammatory cells | 2-3 (many) | C |
| ganglion cells | 2-3 | C |

Heart, Non-Diabetic [TIIDS1]
Sample 1: This sample of normal heart was obtained from a 75-year-old
female who died of lung cancer.
Granular interstitial staining was present, possibly
representing nerve twiglets.

| | | |
|---|---|---|
| cardiac myocytes | 0 | |
| others | 2 | C |

Sample 2: This sample of normal heart was obtained at autopsy from
a patient of unknown age and sex.

| | |
|---|---|
| cardiac myocytes | 0 |

Kidney, Cortex, Non-Diabetic [TIIDS1]
Sample 1: This sample of normal kidney was obtained from a 68-year-old
male who died of metastatic pancreatic carcinoma.

| | | |
|---|---|---|
| glomeruli | 2 (rare) | N |
| renal tubular epithelium | 2-3 (many) | C |

Sample 2: This sample of normal kidney was obtained at autopsy from
a 52-year-old female.

| | | |
|---|---|---|
| glomeruli | 0 | |
| renal tubular epithelium | 2-3 (many) | C |

Kidney, Medulla, Non-Diabetic [TIIDS1]
Sample 1: This sample of normal kidney was obtained at autopsy from a
45-year-old male.
Urothelium was positive.

| | | |
|---|---|---|
| renal tubular epithelium | 2-3 (many) | C, M |
| others | 2-3 | C, M |

Sample 2: This sample of normal kidney was obtained from a
68-year-old male who died of metastatic pancreatic carcinoma.

| | | |
|---|---|---|
| renal tubular epithelium | 2-3 (many) | C, N |

Liver, Non-Diabetic [TIIDS1]
Sample 1: This sample of normal liver was obtained from a 75-year-old
male who died of congestive heart failure.

| | | |
|---|---|---|
| hepatocytes | 1-2 | C, N |
| bile duct epithelium | 2 (many) | C, N |
| Kupffer cells | 0 | |

Sample 2: This sample of normal liver was obtained at autopsy
from a 76-year-old female who died of diverticulosis.

| | | |
|---|---|---|
| hepatocytes | 1-2 | C |
| bile duct epithelium | 1-2 | C |
| Kupffer cells | 0 | |

Lung, Non-Diabetic [TIIDS1]
Sample 1: This sample of normal lung was obtained at autopsy from a
54-year-old female with BMI = 20.4.

| | | |
|---|---|---|
| airway | (NI) | |
| pneumocytes | 0 | |
| alveolar macrophages | 2 (occasional) | C |

Sample 2: This sample of normal lung was obtained at autopsy
from a 58-year-old female.

| | |
|---|---|
| airway | 0 |
| pneumocytes | 0 |
| alveolar macrophages | 0 |

Pancreas, Non-Diabetic [TIIDS1]
Sample 1: This sample of normal pancreas was obtained at autopsy from
a 41-year-old female who died of metastatic malignant melanoma.

| | | |
|---|---|---|
| acinar epithelium | 2-3 | C, M |
| islets of Langerhans | 2-3 | C |
| ductal epithelium | 1-2 (occasional) | C |

Sample 2: This sample of normal pancreas was obtained from an
84-year-old male who died of emphysema.

| | | |
|---|---|---|
| acinar epithelium | 2 | C |
| islets of Langerhans | 3 (many) | C |
| ductal epithelium | 0 | |

Skeletal Muscle, Non-Diabetic [TIIDS1]
Sample 1: This sample of normal skeletal muscle was obtained at autopsy
from a 45-year-old male.

| | |
|---|---|
| myocytes | 0 |

Sample 2: This sample of normal skeletal muscle was obtained from a
16-year-old female who died of a self-inflicted gunshot wound

| | |
|---|---|
| myocytes | 0 |

Adipose, Subcutaneous, Diabetic [TIIDS1]
Sample 1: This sample of adipose was obtained at leg amputation from a
76-year-old male with Type II diabetes.

| | |
|---|---|
| adipocytes | 0 |

Sample 2: This sample of adipose was obtained from a patient of
unknown age and sex.

| | |
|---|---|
| adipocytes | 0 |

Adipose, Visceral, Diabetic [TIIDS1]
Sample 1: This sample of adipose was obtained at autopsy from a patient
of unknown age and sex.

| | |
|---|---|
| adipocytes | 0 |

Sample 2: This sample of adipose was obtained from an 86-year-old
male with BMI = 25.6.

| | |
|---|---|
| adipocytes | 0 |

Colon, Diabetic [TIIDS1]
Sample 1: This sample of colon was obtained at rectal resection from a
75-year-old male with rectal adenocarcinoma.

| | | |
|---|---|---|
| epithelium | 3 (most) | C |
| smooth muscle cells | 0 | |
| inflammatory cells | 3-4 (many) | C |
| ganglion cells | 2 | C |

Sample 2: This sample of colon was obtained at left hemicolectomy
from a 64-year-old female with colonic adenocarcinoma.

| | | |
|---|---|---|
| epithelium | 3 (most) | C |
| smooth muscle cells | 0 | |
| inflammatory cells | 3-4 (many) | C |
| ganglion cells | 2-3 | C |

Heart, Diabetic [TIIDS1]
Sample 1: This sample of heart was obtained at autopsy from an
80-year-old female with BMI = 25.3.

| | |
|---|---|
| cardiac myocytes | 1 (rare) |

Sample 2: This sample of heart was obtained at autopsy from
a 65-year-old male with BMI = 25.9.
Interstitial fibers (possible nerve twiglets) were positive.

| | |
|---|---|
| cardiac myocytes | 0 |
| others | 2 (occasional) |

Kidney, Cortex, Diabetic [TIIDS1]
Sample 1: This sample of kidney was obtained at autopsy from a patient
of unknown age and sex.

| | | |
|---|---|---|
| glomeruli | 2 (rare) | N |
| renal tubular epithelium | 2-3 (many) | C, N |

Sample 2: This sample of kidney was obtained at autopsy from a
patient of unknown age and sex with BMI = 19.8.

| | | |
|---|---|---|
| glomeruli | 0 | |
| renal tubular epithelium | 2 | C, N |

Kidney, Medulla, Diabetic [TIIDS1]
Sample 1: This sample of kidney was obtained at autopsy from a patient
of unknown age and sex.

| | | |
|---|---|---|
| renal tubular epithelium | 2 (many) | C, N |

Sample 2: This sample of kidney was obtained at autopsy from a
patient of unknown age and sex with BMI = 19.8.

| | | |
|---|---|---|
| renal tubular epithelium | 2-3 (many) | C, M |

Liver, Diabetic [TIIDS1]
Sample 1: This sample of liver was obtained at autopsy from a patient
of unknown age and sex with BMI = 19.8.

| | | |
|---|---|---|
| hepatocytes | 1-2 | C |
| bile duct epithelium | 1-2 | C, N |
| Kupffer cells | 0 | |

Sample 2: This sample of liver was obtained at autopsy from a
57-year-old patient of unknown sex.

| | | |
|---|---|---|
| hepatocytes | 1 (many) | C |
| bile duct epithelium | 1-2 | C, N |
| Kupffer cells | 0 | |

Lung, Diabetic [TIIDS1]
Sample 1: This sample of lung was obtained at autopsy from a patient
of unknown age and sex.

| | |
|---|---|
| airway | 0 |
| pneumocytes | 0 |
| alveolar macrophages | 0 |

Sample 2: This sample of lung was obtained at autopsy from an
88-year-old male who died of heart failure.

| | | |
|---|---|---|
| airway | 1-2 (many) | C |
| pneumocytes | 0 | |
| alveolar macrophages | 1-2 (occasional) | C |

Pancreas, Diabetic [TIIDS1]
Sample 1: This sample of pancreas was obtained at autopsy from a
patient of unknown age and sex.

| | | |
|---|---|---|
| acinar epithelium | 2 | C |
| islets of Langerhans | 2-3 | C |
| ductal epithelium | 1-2 | C |

Sample 2: This sample of pancreas was obtained at autopsy
from a patient of unknown age and sex.

| | | |
|---|---|---|
| acinar epithelium | 2-3 (many) | C, M |
| islets of Langerhans | 2 (many) | C |
| ductal epithelium | 0 | |

Skeletal Muscle, Diabetic [TIIDS1]
Sample 1: This sample of skeletal muscle was obtained from a
62-year-old male with brain carcinoma and diabetes.

| | |
|---|---|
| myocytes | 0 |

Sample 2: This sample of skeletal muscle was obtained from a
patient of unknown age and sex.

| | | |
|---|---|---|
| myocytes | 1-2 (rare) | C |

Heart, Ischemia [Single Tissue]
Sample 1: This sample of heart was obtained at autopsy from an
89-year-old female who died of congestive heart failure.
Macrophages, Purkinje fibers, and endothelium were positive.

| | | |
|---|---|---|
| cardiac myocytes | 1-2 (occasional) | C |
| others | 2-3 | C, N |

Sample 2: This sample of heart was obtained at autopsy from a
patient of unknown age and sex.

| | | |
|---|---|---|
| cardiac myocytes | 1 (rare) | C |
| others | 2 (occasional) | C |

Sample 3: This sample of heart was obtained at autopsy from a
72-year-old male who died of congestive heart failure.

| | | |
|---|---|---|
| cardiac myocytes | 1 (rare) | C |

Lung [Single Tissue]
Sample 1: This sample of normal lung was obtained at autopsy from a
75-year-old male who died of a myocardial infarct.

| | |
|---|---|
| airway | 0 |
| pneumocytes | 0 |
| alveolar macrophages | 0 |

Sample 2: This sample of normal lung was obtained at autopsy
from a 29-year-old female.

| | | |
|---|---|---|
| airway | 1 (rare) | C |
| pneumocytes | 0 | |
| alveolar macrophages | 0 | |

Sample 3: This sample of normal lung was obtained at autopsy
from a 73-year-old female.

| | | |
|---|---|---|
| airway | 2 (occasional) | C |
| pneumocytes | 0 | |
| alveolar macrophages | 2 (occasional) | C |

Lung, Hypertension [Single Tissue]
Sample 1: This sample of lung was obtained from a 92-year-old female
who died of congestive heart failure.

| | | |
|---|---|---|
| airway | 1 (occasional) | C |
| pneumocytes | 2 (rare) | C |
| alveolar macrophages | 2 (occasional) | C |

Sample 2: This sample of lung was obtained at autopsy from
a 53-year-old female.

| | | |
|---|---|---|
| airway | 1 (rare) | C |
| pneumocytes | 2 (occasional) | C |
| alveolar macrophages | 1 (occasional) | C |

Sample 3: This sample of lung was obtained at autopsy from
a 46-year-old female.

| | | |
|---|---|---|
| airway | 1 (occasional) | C |
| pneumocytes | 0 | |
| alveolar macrophages | 0 | |

Legend for Cellular Localization of Staining
Cytoplasm (C),
Extracellular (E),
Membrane (M),
Nuclear (N),
Perinuclear (P)

### Experiment B. Immunohistochemistry Analysis of BMP-10 Protein Expression in Mouse Tissues.

### Methods.

Immunohistochemistry methods were as described above and/or standard procedure.

### Results

In this study, antibody LP9168 to bone morphogenic protein 10 (BMP-10) was evaluated on a series of selected mouse tissues. BMP-10 is a novel TGF-beta family member that has been identified in mouse heart and is believed to be involved in fetal heart development. Because of the structural similarity of BMP-10 to other proteins, this is believed to be a secreted target. In the mouse tissues, most staining was cytoplasmic, but occasional nuclear and extracellular staining was also identified. The distribution of staining was compatible with the limited published data available for this target.

The most prominent staining was identified in cardiomyocytes, lymphocyte subsets, thyroid epithelium, and pancreatic islets. Focal prominent staining was also present in seminiferous precursors and endometrium. Other positive cell types included urothelium, ganglion cells, hepatocytes, and renal tubular epithelium. Focal positivity was present in skin adnexal structures, neurons, and epithelium lining the gut (stomach, intestine, and colon). Occasional weak staining was present in pancreatic acini, respiratory epithelium, and alveolar macrophages.

EXAMPLE 8
In vitro Global Gene Expression Analysis of Human Endothelial
Cells Treated with Human BMP-10-Conditioned Media.

| | | |
|---|---|---|
| Cell/Zone | Intensity | Localization |

Mouse Bladder
Sample 1: This sample of bladder was obtained from a mouse.

| | | |
|---|---|---|
| urothelium | 2 | C |
| smooth muscle cells | 0 | |

Sample 2: This sample of bladder was obtained from a mouse.
Serum was positive.

| | | |
|---|---|---|
| urothelium | 2 (occasional) | C |
| smooth muscle cells | 0 | |
| others | 2 (occasional) | E |

Mouse Brain, Cerebellum
Sample 1: This sample of brain was obtained from a mouse.
Neuropil was positive.

| | | |
|---|---|---|
| neurons | 1 (occasional) | C |
| glia | 0 | |

Sample 2: This sample of brain was obtained from a mouse.

| | | |
|---|---|---|
| neurons | 2 (many) | C |
| glia | 1 (occasional) | C |

Mouse Brain, Cerebral Cortex
Sample 1: This sample of brain was obtained from a mouse.

| | | |
|---|---|---|
| neurons | 3 (occasional) | C |
| glia | 0 (most) | |

Sample 2: This sample of brain was obtained from a mouse.

| | | |
|---|---|---|
| neurons | 2 (occasional) | C |
| glia | 0 | |

Mouse Colon
Sample 1: This sample of colon was obtained from a mouse.

| | | |
|---|---|---|
| epithelium | 2 (occasional) | C |
| smooth muscle cells | 0 | |
| inflammatory cells | 3 (many) | C |
| ganglion cells | 2 | C |

Sample 2: This sample of colon was obtained from a mouse.
Serum was positive. Staining in epithelium was in surface epithelium

| | | |
|---|---|---|
| epithelium | 2-3 (many) | C |
| smooth muscle cells | 0 | |
| inflammatory cells | 2 (occasional) | C, N |
| ganglion cells | 2 | C |
| others | 3 (occasional) | E |

Mouse Heart
Sample 1: This sample of heart was obtained from a mouse.
Serum was positive.

| | | |
|---|---|---|
| cardiac myocytes | 2-3 (many) | C, N |
| others | 3 (occasional) | E |

Sample 2: This sample of heart was obtained from a mouse.

| | | |
|---|---|---|
| cardiac myocytes | 2-3 (many) | C, N |

Mouse Kidney
Sample 1: This sample of kidney was obtained from a mouse.

| | | |
|---|---|---|
| glomeruli | 0 | |
| renal tubular epithelium | 2 (many) | C |

Sample 2: This sample of kidney was obtained from a mouse.

| | | |
|---|---|---|
| glomeruli | 0 | |
| renal tubular epithelium | 3 | C, M |

Mouse Liver
Sample 1: This sample of liver was obtained from a mouse.

| | | |
|---|---|---|
| hepatocytes | 2 | C |
| bile duct epithelium | 0 | |
| Kupffer cells | 0 | |

Sample 2: This sample of liver was obtained from a mouse.
Serum was positive.

| | | |
|---|---|---|
| hepatocytes | 2-3 | C |
| bile duct epithelium | 1-2 | C |
| Kupffer cells | 0 (most) | |
| others | 3 (occasional) | E |

Mouse Lung
Sample 1: This sample of lung was obtained from a mouse.

| | | |
|---|---|---|
| airway | 1 (rare) | C |
| pneumocytes | 1-2 (occasional) | C |
| alveolar macrophages | 2 (occasional) | C |

Sample 2: This sample of lung was obtained from a mouse.
Inflammatory cells, surfactant, and serum were positive.

| | | |
|---|---|---|
| airway | 2 (many) | C |
| pneumocytes | 1 (many) | C |
| alveolar macrophages | 2 (many)-3 (occasional) | C |
| others | 2-3 | E |

Mouse Pancreas
Sample 1: This sample of pancreas was obtained from a mouse.

| | | |
|---|---|---|
| acinar epithelium | 1 | C |
| islets of Langerhans | 2-3 | C |
| ductal epithelium | 1 (occasional) | C, M |

Sample 2: This sample of pancreas was obtained from a mouse.
Serum was positive.

| | | |
|---|---|---|
| acinar epithelium | 1-2 | C |
| islets of Langerhans | 3 | C |
| ductal epithelium | 1 | C, E |
| others | 2 | E |

Mouse Skeletal Muscle
Sample 1: This sample of skeletal muscle was obtained from a mouse.

| | | |
|---|---|---|
| myocytes | 1 (rare) | C |

Sample 2: This sample of skeletal muscle was obtained from a mouse.

| | | |
|---|---|---|
| myocytes | 1 | C |

Mouse Skin
Sample 1: This sample of skin was obtained from a mouse.

| | | |
|---|---|---|
| epidermis | 1 (occasional) | C |
| dermis | 2 (occasional) | C |

Sample 2: This sample of skin was obtained from a mouse.

| | | |
|---|---|---|
| epidermis | 2 (many) | C, N |
| dermis | 2-3 (many) | C |

Mouse Small Intestine
Sample 1: This sample of small intestine was obtained from a mouse.

| | | |
|---|---|---|
| epithelium | 2 (many) | C |
| smooth muscle cells | 0 | |
| inflammatory cells | 1-2 (occasional) | C |
| ganglion cells | 2 | C |

Sample 2: This sample of small intestine was obtained from a mouse.

| | | |
|---|---|---|
| epithelium | 2-3 (many) | C |
| smooth muscle cells | 0 | |
| inflammatory cells | 2 (occasional) | C |
| ganglion cells | 2 (occasional) | C |

Mouse Spleen
Sample 1: This sample of spleen was obtained from a mouse.

| | | |
|---|---|---|
| white pulp | 1-2 (occasional) | C |
| red pulp | 2 (occasional) | C |

Sample 2: This sample of spleen was obtained from a mouse.

| | | |
|---|---|---|
| white pulp | 1 | C |
| red pulp | 1 (occasional) | C |

Mouse Stomach
Sample 1: This sample of stomach was obtained from a mouse.

| | | |
|---|---|---|
| epithelium | 2 (many) | C |
| smooth muscle cells | 0 | |

Sample 2: This sample of stomach was obtained from a mouse.

| | | |
|---|---|---|
| epithelium | 2-3 (many) | C |
| smooth muscle cells | 0 | |

Mouse Testis
Sample 1: This sample of testis was obtained from a mouse.

| | | |
|---|---|---|
| seminiferous epithelium | 1-2 | C, M, N |
| Leydig cells | 2 (many) | C |

Sample 2: This sample of testis was obtained from a mouse.

| | | |
|---|---|---|
| seminiferous epithelium | 3-4 (many) | C, E |
| Leydig cells | 0 | |

Mouse Thymus
Sample 1: This sample of thymus was obtained from a mouse.

| | | |
|---|---|---|
| lymphocytes | 2-3 (occasional) | C |
| epithelium | 0 | |

Sample 2: This sample of thymus was obtained from a mouse.

| | | |
|---|---|---|
| lymphocytes | 2 (many) | C |
| epithelium | 0 (most) | |

Mouse Thyroid
Sample 1: This sample of thyroid was obtained from a mouse.

| | | |
|---|---|---|
| follicular epithelium | 3 (many) | C |

Sample 2: This sample of thyroid was obtained from a mouse.

| | | |
|---|---|---|
| follicular epithelium | 3 (many) | C |

Mouse Uterus
Sample 1: This sample of uterus was obtained from a mouse.

| | | |
|---|---|---|
| endometrium | 1-2 | C, M |
| myometrium | 0 | |

Sample 2: This sample of uterus was obtained from a mouse.

| | | |
|---|---|---|
| endometrium | 3 (many) | C, N |
| myometrium | 1 | C |

Legend for Cellular Localization of Staining
Cytoplasm (C),
Extracellular (E),
Membrane (M),
Nuclear (N),
Perinuclear (P)

Experiment A. Genes Differentially Expressed in Human Aortic Endothelial Cells (HUAECs) Treated with BMP-10 Versus Control Treatment.

### Methods:

Conditioned media from the HEK293 cells transfected with either AdBMP-10 or AdGFP was generated as described in Experiment 3, above. An aliquot of CM was reserved and frozen for use in a BMP-10 western blot to confirm the presence of expressed BMP-10 in the conditioned media, as described in Example 3.

Human aortic endothelial cells were treated with conditioned media as described in Example 3. Cells were lysed for RNA at 30 minutes and 60 minutes for use in transcriptional profiling. Total RNA was isolated from cells using the RNeasy minikit (Qiagen, Hidden, Germany). Analysis and quantification of RNA was done as described below in Example 5 for RNA isolated from mouse tissues. First and second strand cDNA synthesis and generation of biotin labeled cRNA probes was done according to manufacturers directions (Affymetrix, Inc). Hybridization to oligonucleotide arrays (GeneChip Human Genome U133_plus 2.0) and data analysis was performed as described below in Example 10 for mouse tissues.

### Results:

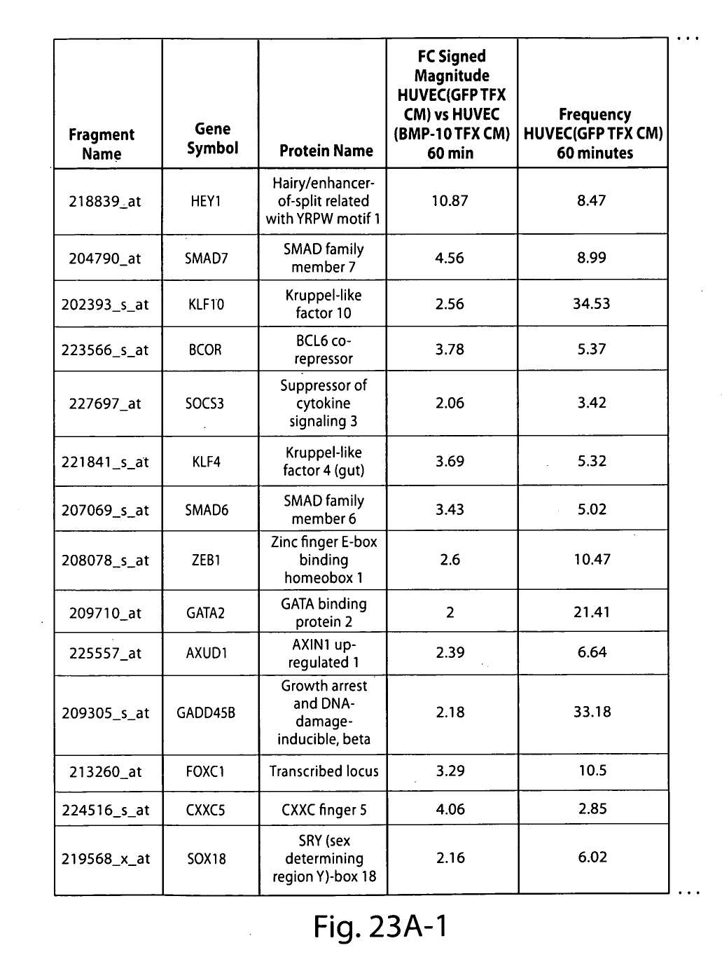

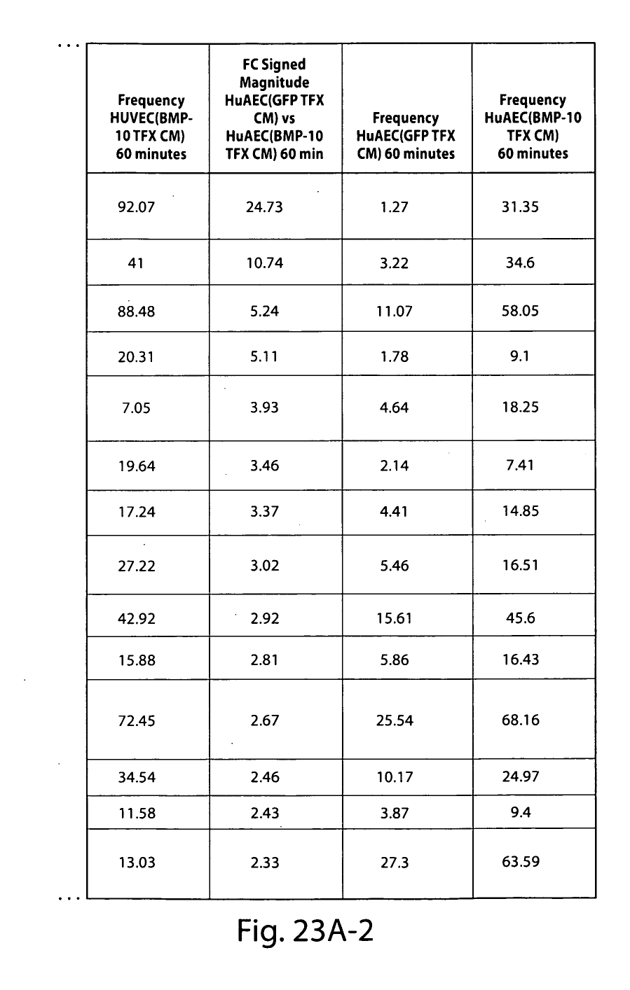

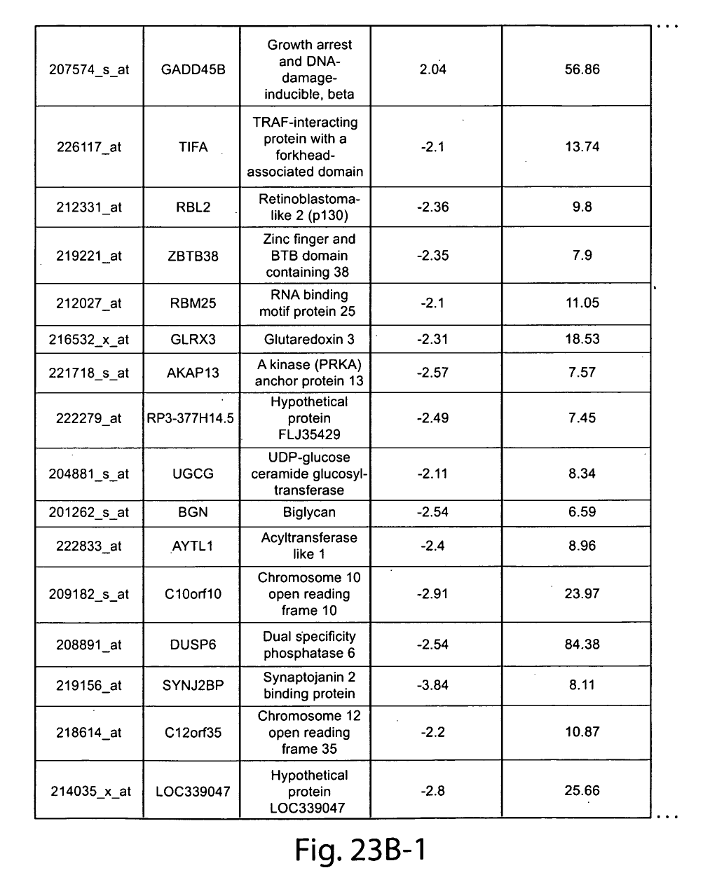

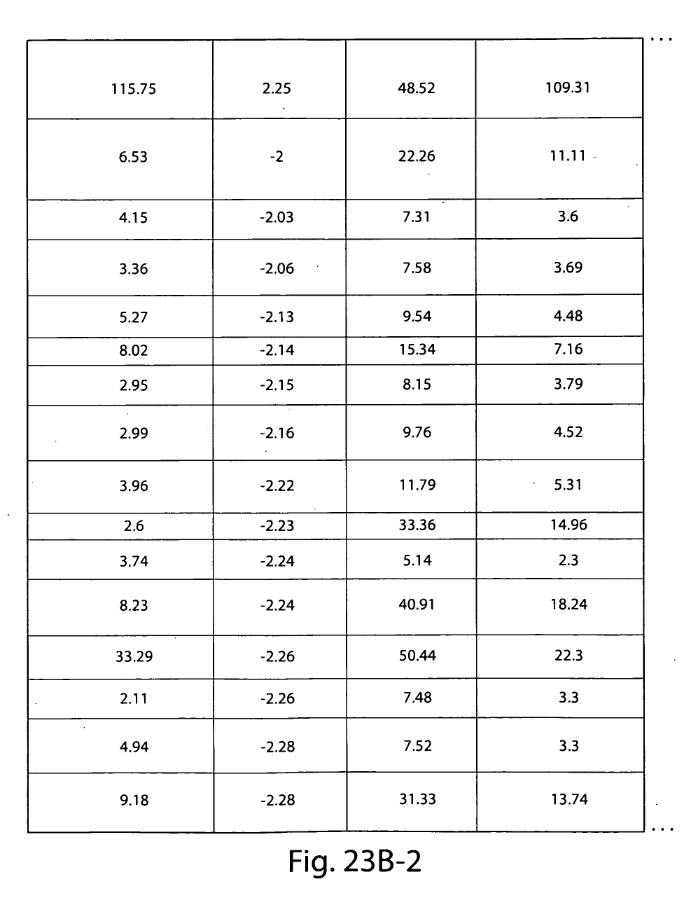

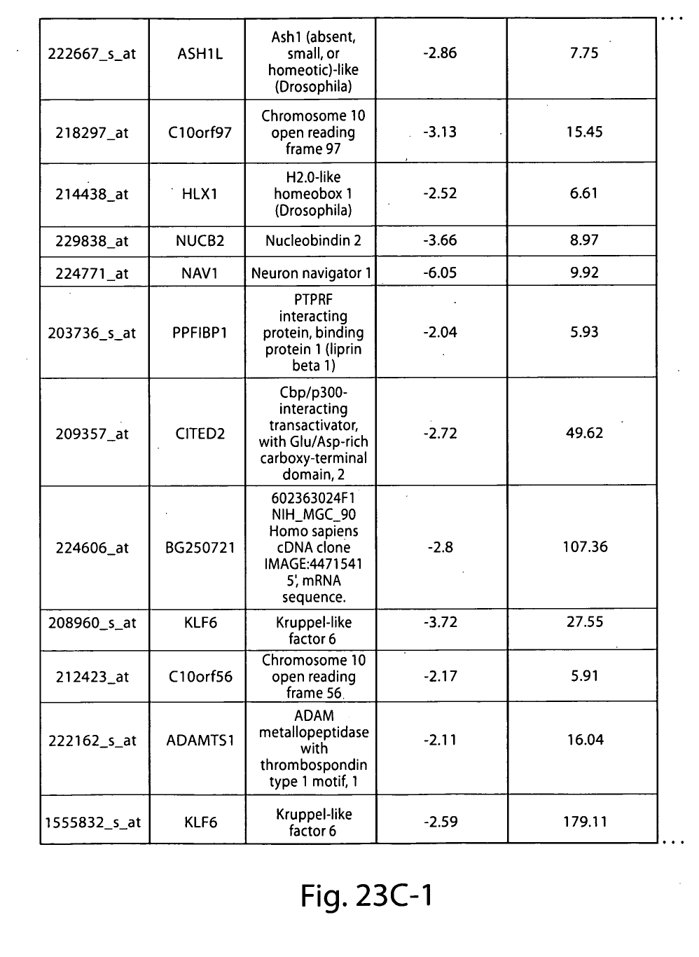

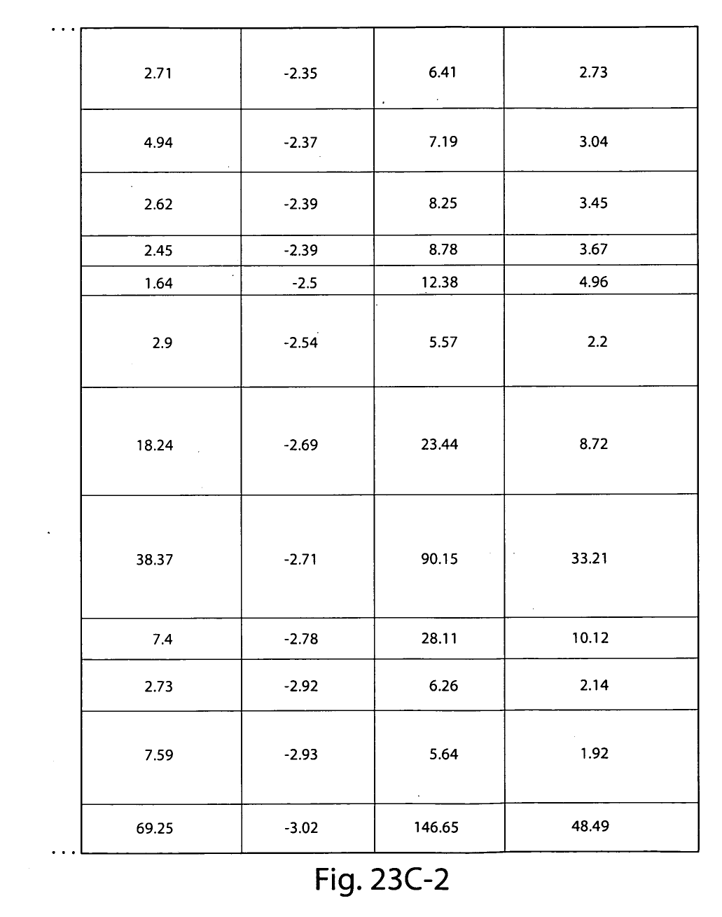

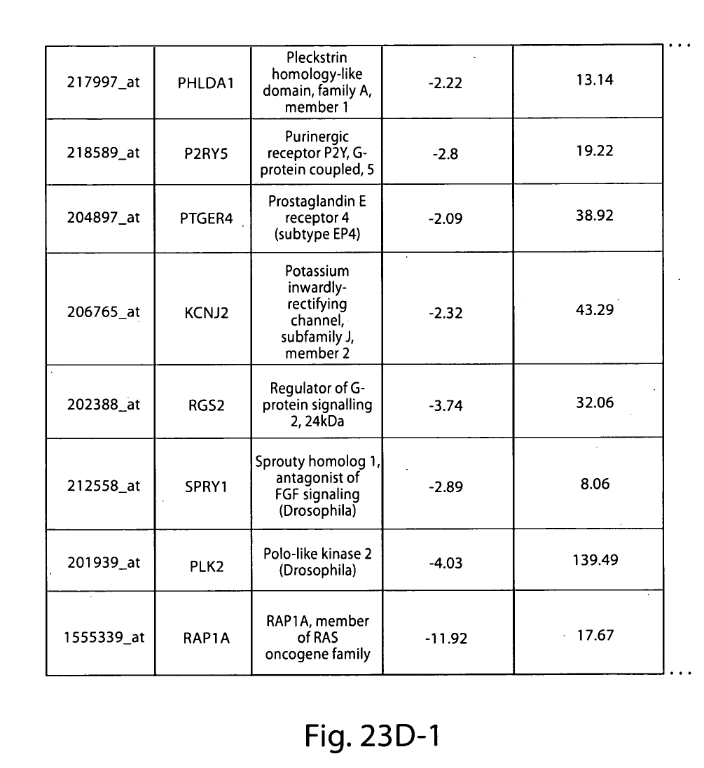

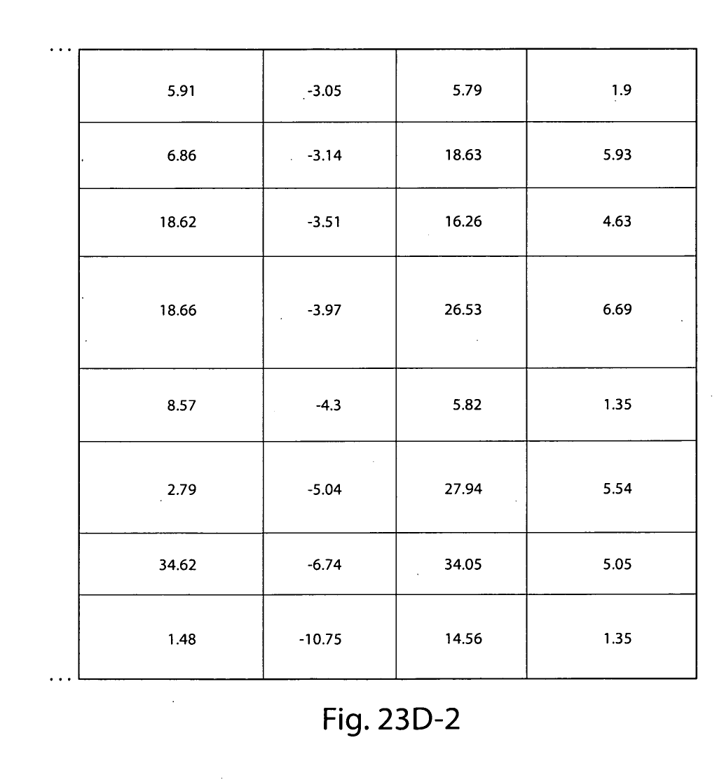

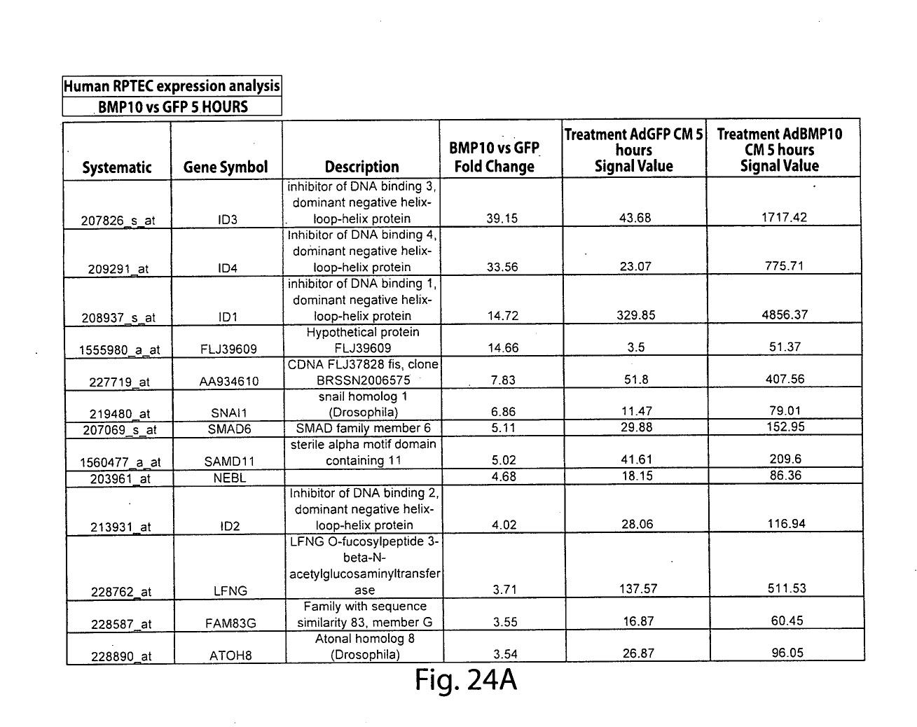

Raw fluorescent intensity values were collected and reduced as described. Transcripts determined to be increasing in expression had to have a “Present” (“P”) call (determined by the GEDS processing), a minimum frequency of 5 ppm (based upon comparison to a standard curve incorporated into the hybridization sample), and a minimum change in expression of 2 fold relative to the buffer control sample. Genes determined to be decreasing with treatment had to be called “P” and have a minimum frequency of 5 ppm in the buffer control sample, and had to have a maximum change in expression of −2 fold in the treated sample. Results of the global gene expression analysis of mRNA from both HUVEC and HUAECs treated for 30 minutes with BMP-10 can be seen in FIG. 22; results from both HUVEC and HUAECs treated for 60 minutes can be seen in FIGS. 23A-23D.

Experiment B. Genes Differentially Expressed in Human Umbilical Vein Endothelial Cells Treated with AdBMP-10 Versus GFP

### Methods.

Conditioned media from the HEK293 cells transfected with either AdBMP-10 or AdGFP was generated as previously described in Experiment 3. Conditioned media was collected 48 hours post transfection. An aliquot of CM was reserved and frozen for use in a BMP-10 western immunoblot analysis to confirm the presence of expressed BMP-10 in the conditioned media, as described in Example 3.

Human umbilical vein endothelial cells were treated with AdBMP-10- or AdGFP-conditioned media as described in Example 3. Cells were lysed for RNA at 30 minutes and at 60 minutes for use in global gene expression analysis, as described in the above experiment.

### Results:

Raw fluorescent intensity values were collected and reduced as described. Transcripts determined to be increasing in expression had to have a “Present” (“P”) call (determined by the GEDS processing), a minimum frequency of 5 ppm (based upon comparison to a standard curve incorporated into the hybridization sample), and a minimum change in expression of 2 fold relative to the buffer control sample. Genes determined to be decreasing with treatment had to be called “P” and have a minimum frequency of 5 ppm in the buffer control sample, and had to have a maximum change in expression of −2 fold in the treated sample. Results of the global gene expression analysis of mRNA from both HUVEC and HUAECs treated for 30 minutes with BMP-10 can be seen in FIG. 22; results from both HUVEC and HUAECs treated for 60 minutes can be seen in FIGS. 23A-23D.

### EXAMPLE 9

### Gene Expression Analysis of Human Renal Proximal Tubule Epithelial Cells Overexpressing BMP-10

### Methods.

This example shows gene expression analysis of human renal proximal tubule epithelial cells transduced with either AdBMP-10 or AdGFP (control) at time points: 5 and 17 hours. Conditioned media (CM) from the HEK293 cells transduced with either AdBMP-10 or AdGFP was generated as described in Example 3, above. An aliquot of CM was reserved and frozen for use in a BMP-10 western immunoblot analysis to confirm the presence of expressed BMP-10 in the conditioned media, as described in Example 3. Human primary renal proximal tubule epithelial cells (RPTEC) (Lonza Catalog Number CC-2517) were cultured according to manufacturers directions in REGM media with added growth supplements (Lonza Catalog Number CC-3190) in 60 mm tissue culture plates and incubated at 37° C., 5% CO2, when they reached a confluence of ˜70-80% the complete growth media was removed and cells were washed 1× with basal media (Lonza Catalog Number CC-3191)+0.1% delipidized BSA (BD Bioscience Catalog Number 354331) and 4 ml basal media with 0.1% delipidized BSA was added and plates were incubated for 3 hours. After 3 hours cells were treated with either AdBMP-10 or AdGFP containing CM diluted 1:10 in basal media with 0.1% delipidized BSA. Cells were lysed for RNA at 5 hours and 17 hours after treatment and total RNA was isolated from cells using the RNeasy minikit (Qiagen, Hidden, Germany). Analysis and quantification of RNA was done as described in Example 5 for RNA isolated from mouse tissues. First and second strand cDNA synthesis and generation of biotin labeled cRNA probes was done according to manufacturers directions (Affymetrix, Inc). Hybridization to oligonucleotide arrays (GeneChip Human Genome U133_plus 2.0) and data analysis was performed as described in Example 10 for mouse tissues.

### Results.

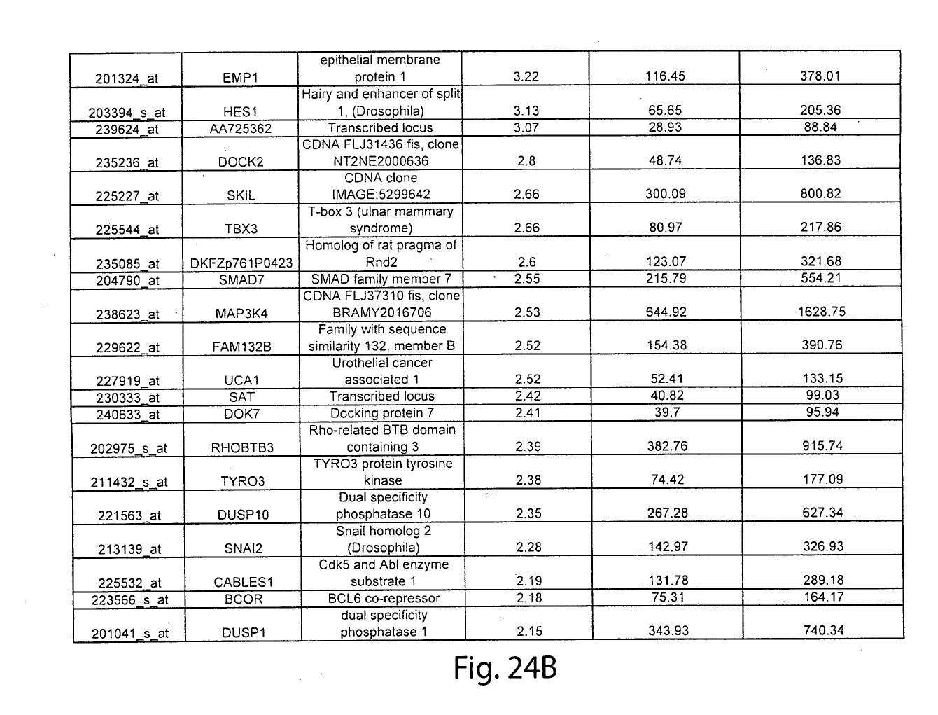

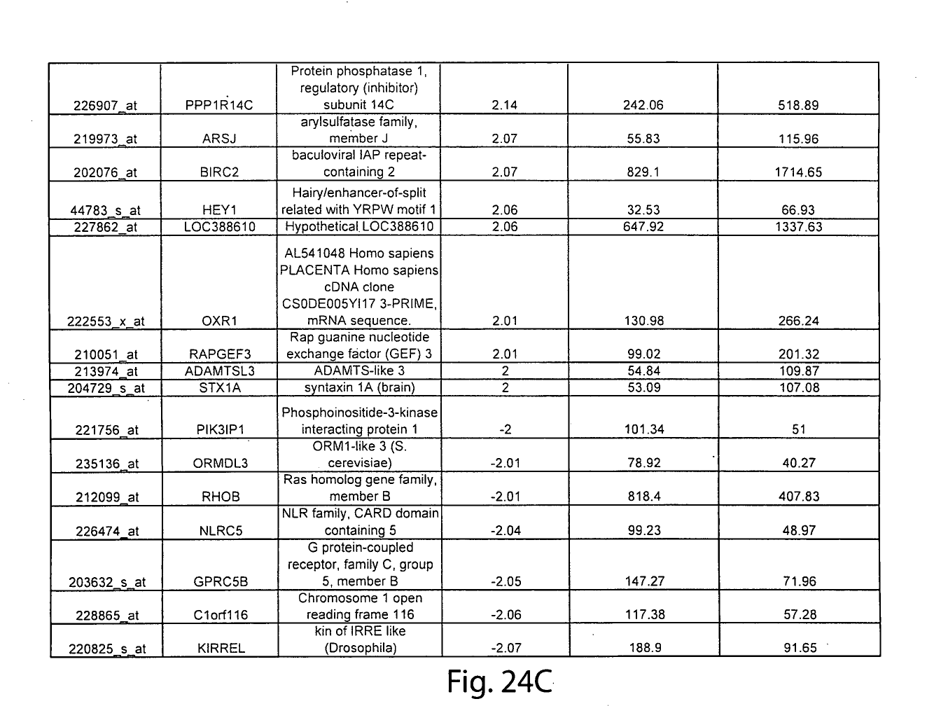

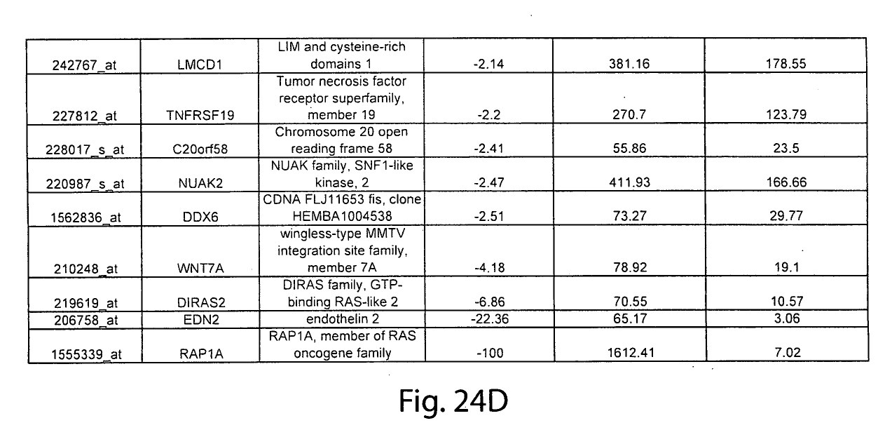

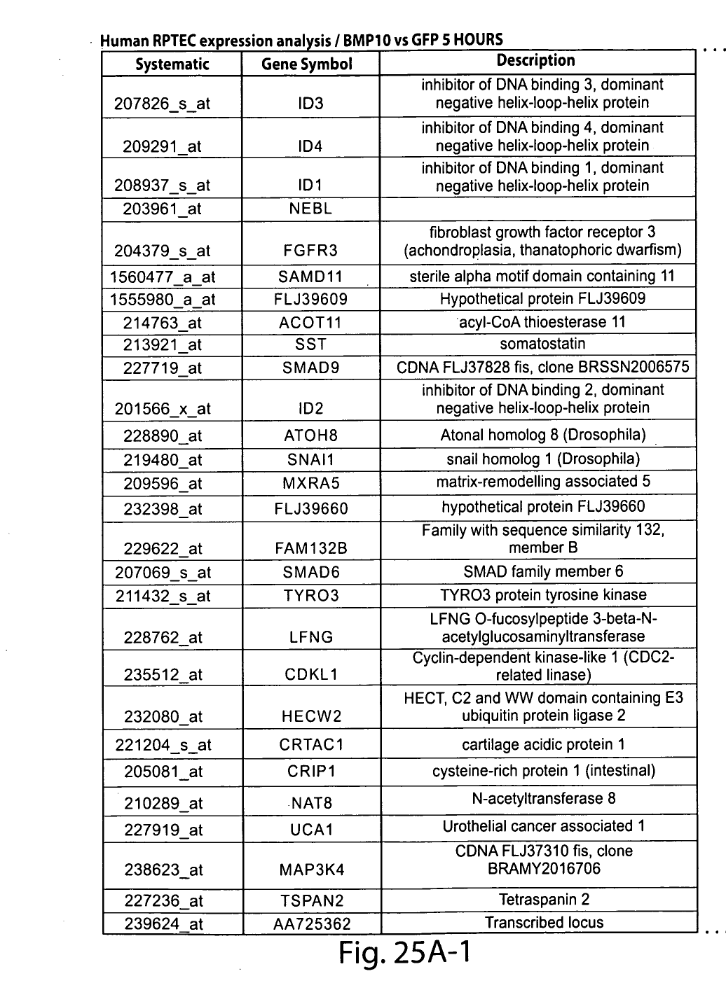

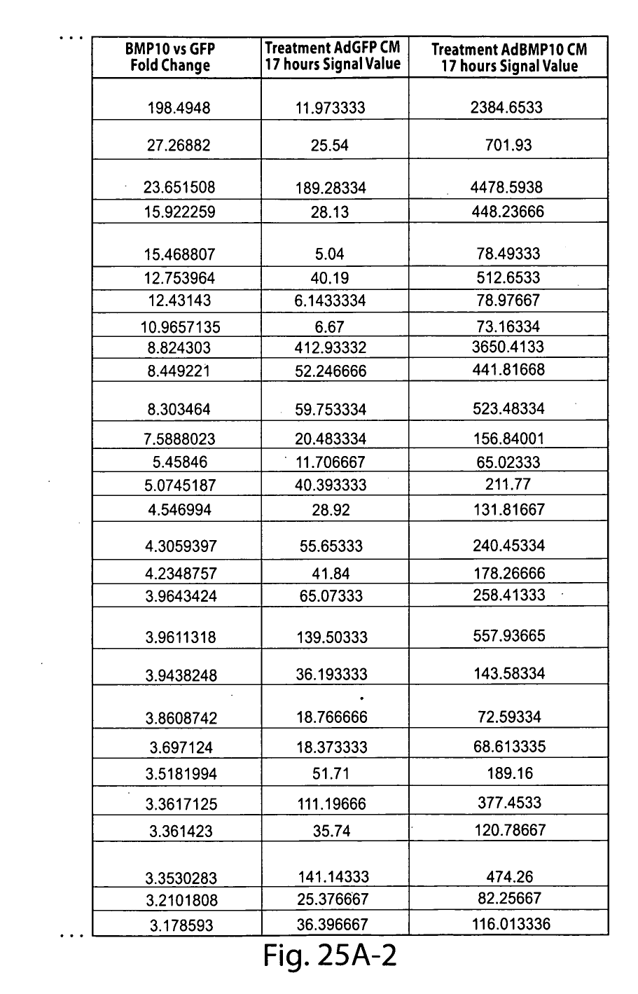

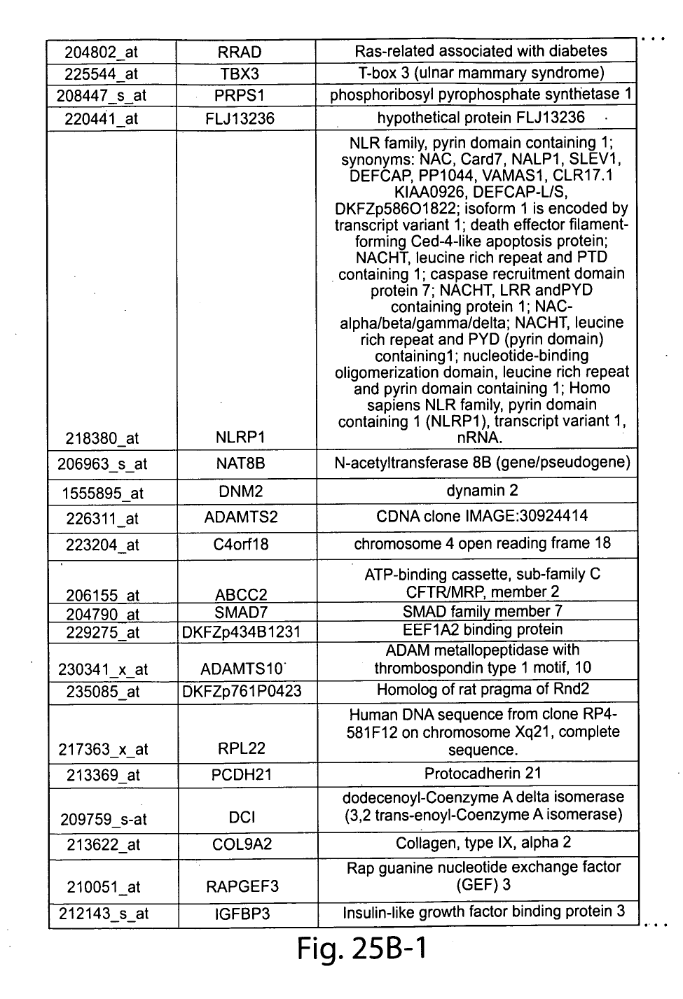

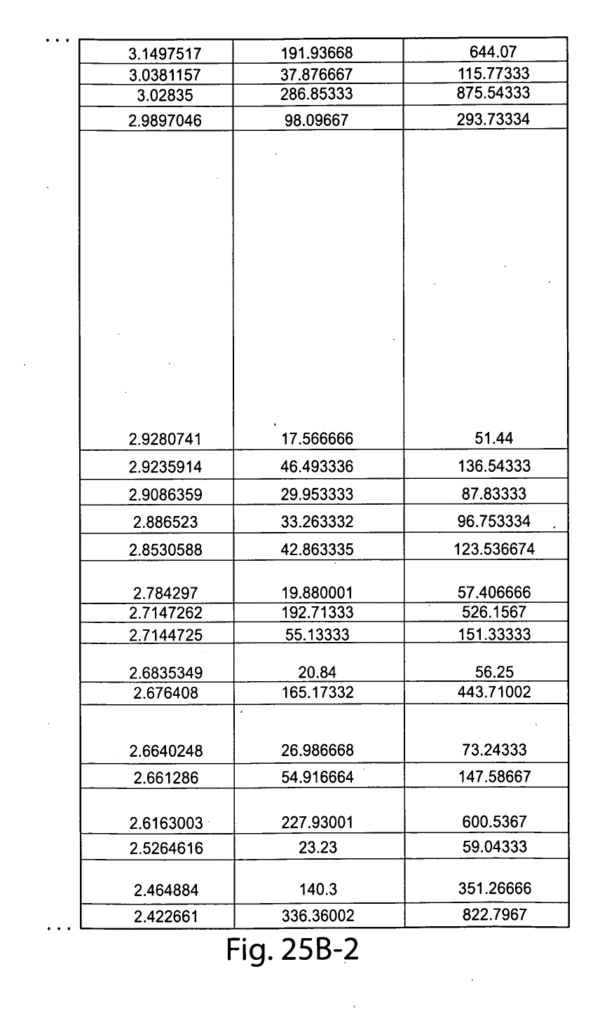

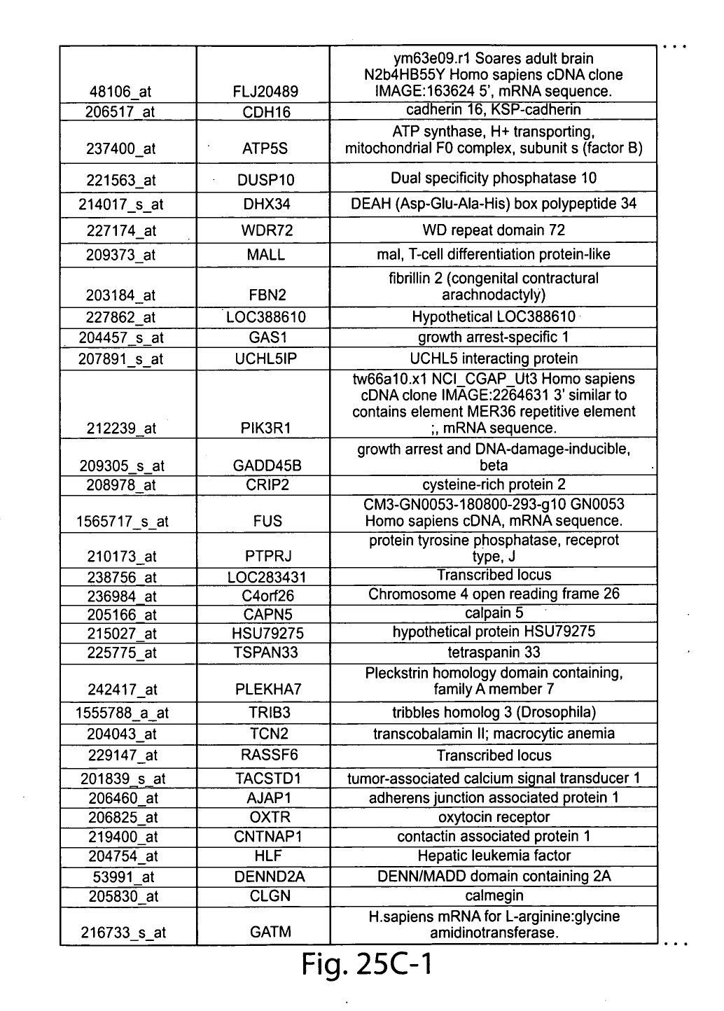

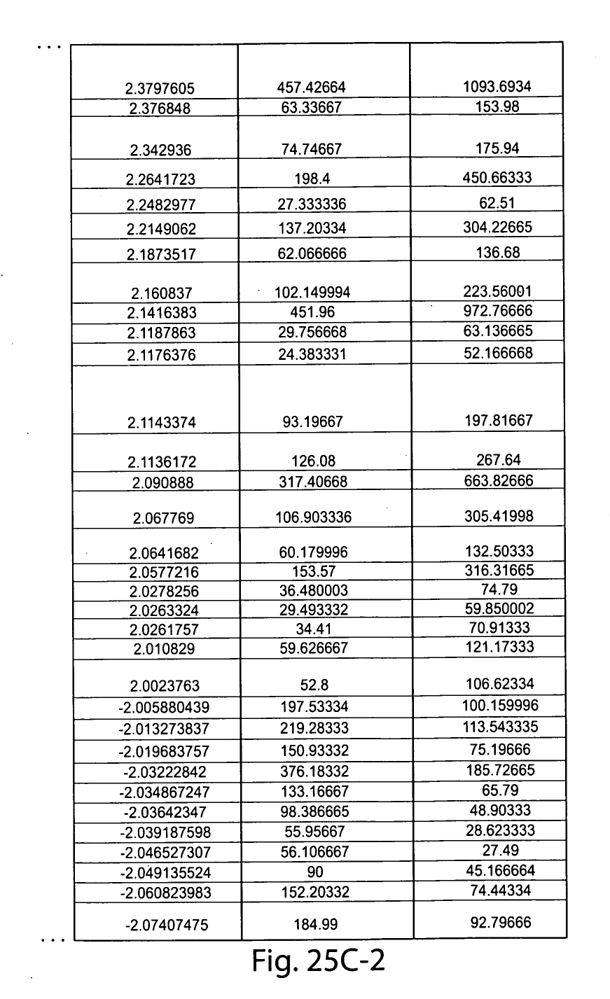

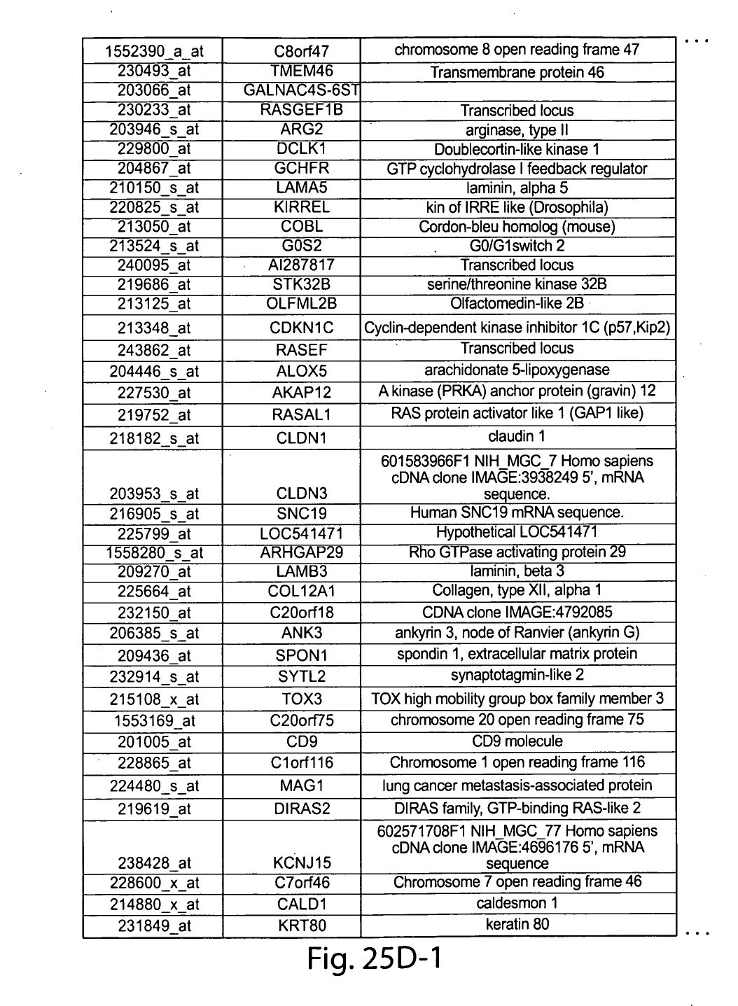

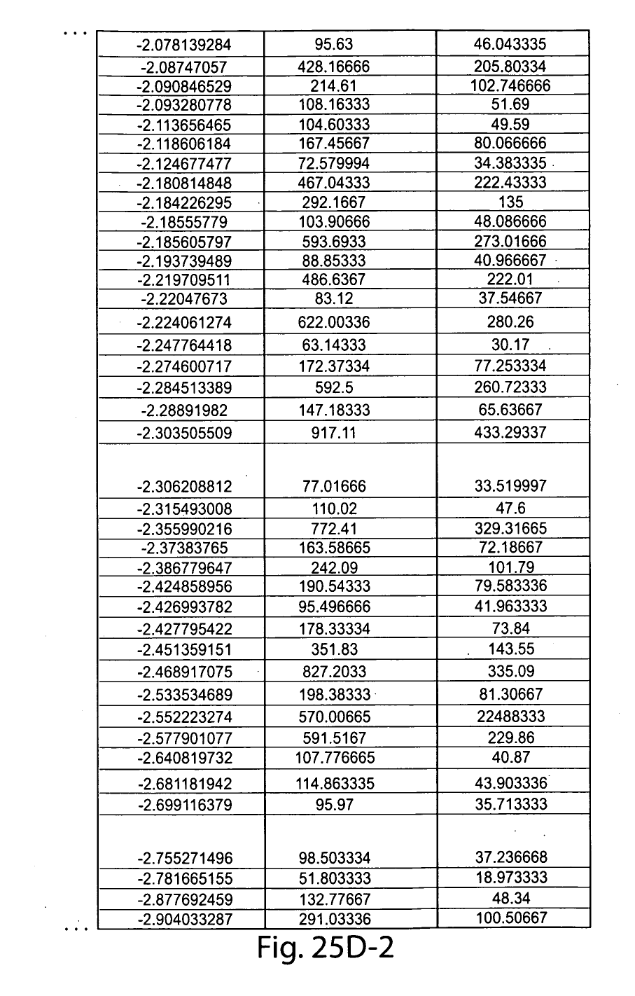

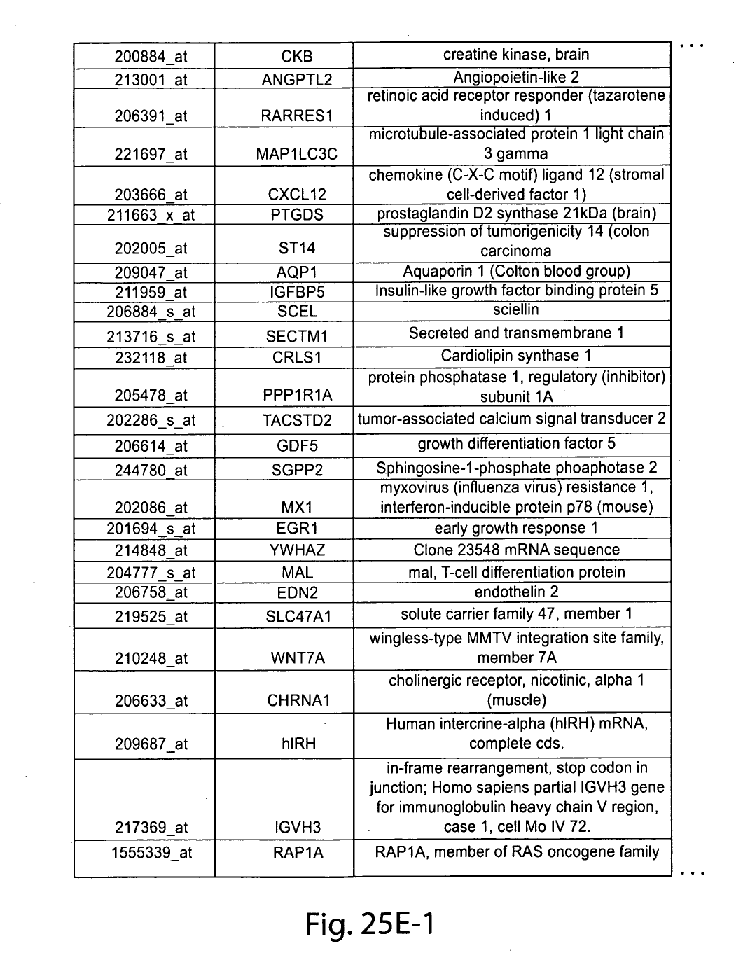

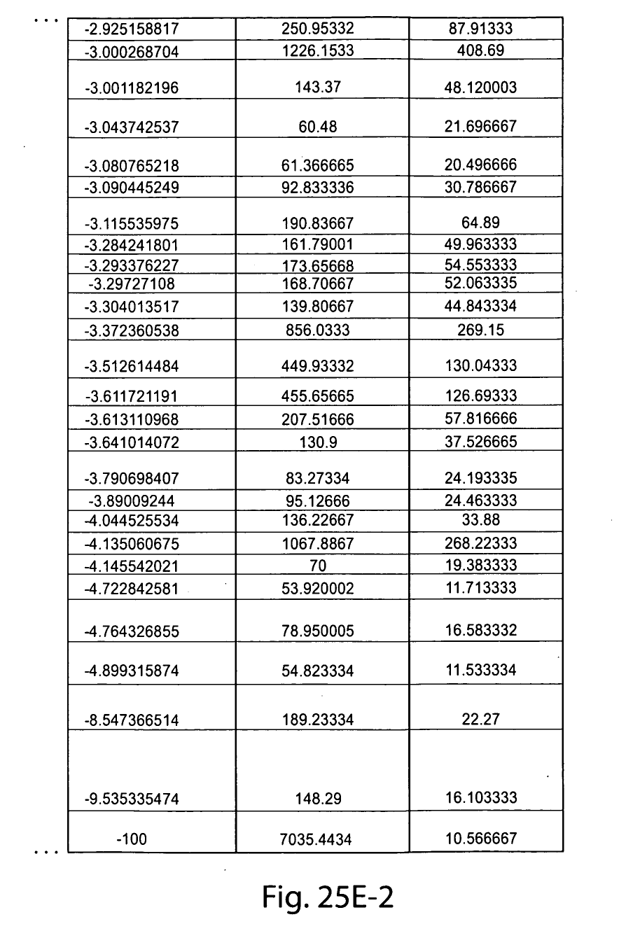

Results for global gene expression analysis for 5 hours of BMP-10 treatment are shown in FIGS. 24A-24D, and results for 17 hours of BMP-10 treatment are shown in FIGS. 25A-25E. Signal values were calculated and data was filtered as follows: transcripts determined to be increasing in expression with AdBMP-10 CM treatment had to be considered “present” (signal value ≧43) with a change in expression of ≧2 fold relative to the AdGFP CM treated control sample and a p-value ≦0.05. Genes determined to be decreasing with AdBMP-10 CM treatment had to be considered “present” (signal value ≧43) in the AdGFP CM control sample with a change in expression of ≦2 fold relative to the AdGFP CM treated control sample and a p-value ≦0.05.

### EXAMPLE 10

### In Vivo Global Gene Expression Analysis of Genes Differentially Expressed in the Heart, Muscle, and Fat Tissues of AdBMP-10 Mice Versus Control Mice

Experiment A. In Vivo Global Gene Expression Analysis Using Affymetrix GeneChip MU11k Array with RNA from Heart Tissue of AdBMP-10 Mice.

### Methods.

C57BL/6 mice were injected intravenously with replication deficient recombinant adenovirus encoding human BMP-10 (AdBMP-10) or control virus (5×10¹⁰ particles), and were sacrificed at day 3 or day 7 after injection. Total RNA was prepared from the heart tissue (as in this experiment; and from muscle and fat tissues for the following two experiments as follows. Frozen tissues (muscle, fat and heart) were pulverized, in liquid nitrogen, with the use of a mortar and pestle and lysed in tissue lysis buffer (RNAgents Kit Catalog Number Z5110, Promega, Madison, Wis.). Total RNA was isolated with a modification of the manufacturers recommendations. Briefly, RNA was precipitated with the addition of isopropanol and washed twice with cold 75% ethanol. The pellet was dissolved in RNeasy minikit sample lysis buffer and RNA was purified according to the manufacturers recommendations (Qiagen, Hidden, Germany). Total RNA was quantitated from a measure of UV absorption at 260 nm. An aliquot of total RNA was resolved with the use of agarose gel electrophoresis and RNA integrity was assessed from a visual comparison of the relative intensities of the 18S and 28S rRNA bands. For all samples, the intensity of the 28S rRNA band exceeded that of the 18S band.

Total RNA was prepared for hybridization by denaturing 5 μg of total RNA isolated from various tissues for 10 minutes at 70° C. with 100 pM T7/T24-tagged oligo-dt primer and cooled on ice. First strand cDNA synthesis was performed under the following buffer conditions; 1× first strand buffer (Invitrogen Life Technologies, Carlsbad, Calif.), 10 mM DTT (GIBCO/Invitrogen), 500 μm of each dNTP (Invitrogen Life Technologies), 400 units of Superscript RT II (Invitrogen Life Technologies) and 40 units RNAse inhibitor (Ambion, Austin, Tex.). Reaction proceeded at 47° C. for 1 hour. Second strand cDNA was synthesized by the addition of the following reagents at the final concentrations listed, 1× second strand buffer (Invitrogen Life Technologies), an additional 200 μm of each dNTP (Invitrogen Life Technologies), 40 units of E. coli DNA polymerase I (Invitrogen Life Technologies), 2 units E. coli RNaseH (Invitrogen Life Technologies), and 10 units of E. coli DNA ligase. The reaction was incubated at 15.80 C for 2 hours and 6 units of T4 DNA polymerase (New England Biolabs, Beverly, Mass.) was added. The reaction was incubated at 15.8° C. for an additional 5 minutes. The resulting double stranded cDNA was purified using BioMag carboxyl terminated particles as follows; 0.2 mg of BioMag particles (Polysciences Inc., Warrington, Pa.) were equilibrated by washing three times with 0.5M EDTA and resuspended at a concentration of 22.2 mg/ml in 0.5M EDTA. The double stranded cDNA reaction was diluted to a final concentration of 10% PEG/1.25M NaCl and bead suspension was added resulting in a final bead concentration of 0.614 mg/ml. The reaction was incubated at room temperature for 10 minutes. The cDNA/bead complexes were washed with 300 μl of 70% ethanol, the ethanol was removed and the tubes were allowed to air dry. The cDNA was eluted with the addition of 20 μl of 10 mM Tris-acetate, pH 7.8, incubated for 2-5 minutes and the cDNA containing supernatant was removed.

10 μl of purified double stranded cDNA was added to an in vitro transcription (IVT) reaction containing, 1×IVT buffer (Ambion, Austin, Tex.) 5,000 units T7 RNA polymerase (Epicentre Technologies, Madison, Wis.), 3 mM GTP, 1.5 mM ATP, 1.2 mM CTP and 1.2 mM UTP (Amersham/Pharmacia, Uppsala, Sweden), 0.4 mM each bio-16 UTP and bio-11 CTP (Enzo Diagnostics, Farmingdale, N.Y.), and 80 units RNase inhibitor (Ambion, Austin, Tex.). The reaction was incubated at 37° C. for 16 hours. Labeled RNA was purified over a column using the Qiagen RNeasy minikit RNA cleanup procedure and RNA yield was quantitated by measuring absorbance at 260 nm.

Hybridization to Affymetrix oligonucleotide arrays was performed as follows. To improve hybridization efficiencies, 15 μg of cRNA was fragmented as previously described. The fragmented cRNA probes were used to create a GeneChip hybridization solution as suggested by the manufacturer (Affymetrix, Santa Clara, Calif.). Hybridization solutions were pre-hybridized to two glass beads (Fisher Scientific, Pittsburgh, Pa.) at 45° C. overnight. The hybridization solution was removed to a clean tube and heated for 1-2 min at 95° C. and microcentrifuged on high for 2 minutes to pellet insoluble debris. These solutions were then hybridized to Affymetrix microarrays. Microarrays were hybridized with 180 ul of the hybridization solution at 45° C. and rotating at 45-60 rpm overnight. After overnight incubation the hybridization solutions were recovered and chips were washed and prepared for scanning according to the manufacturer's protocols (Affymetrix, Santa Clara, Calif.). Raw fluorescence data was collected and reduced with the use of the GeneChip 3.1 application (Affymetrix, Santa Clara, Calif.) to create the primary data, i.e., the calculated gene values.

### Results.

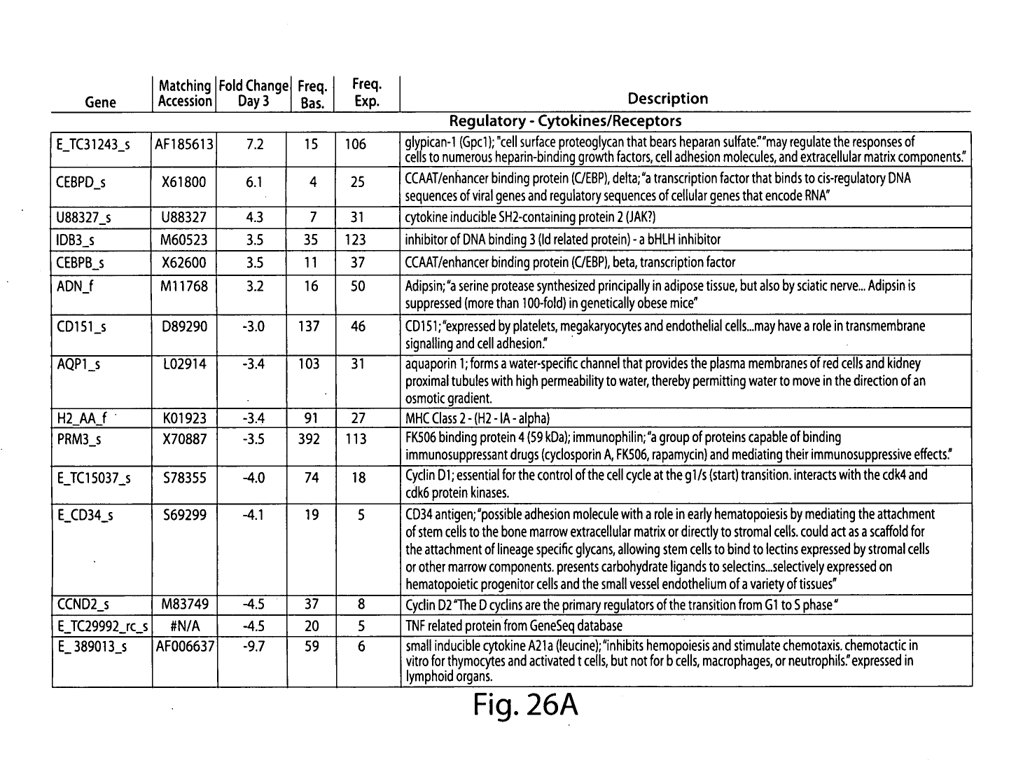

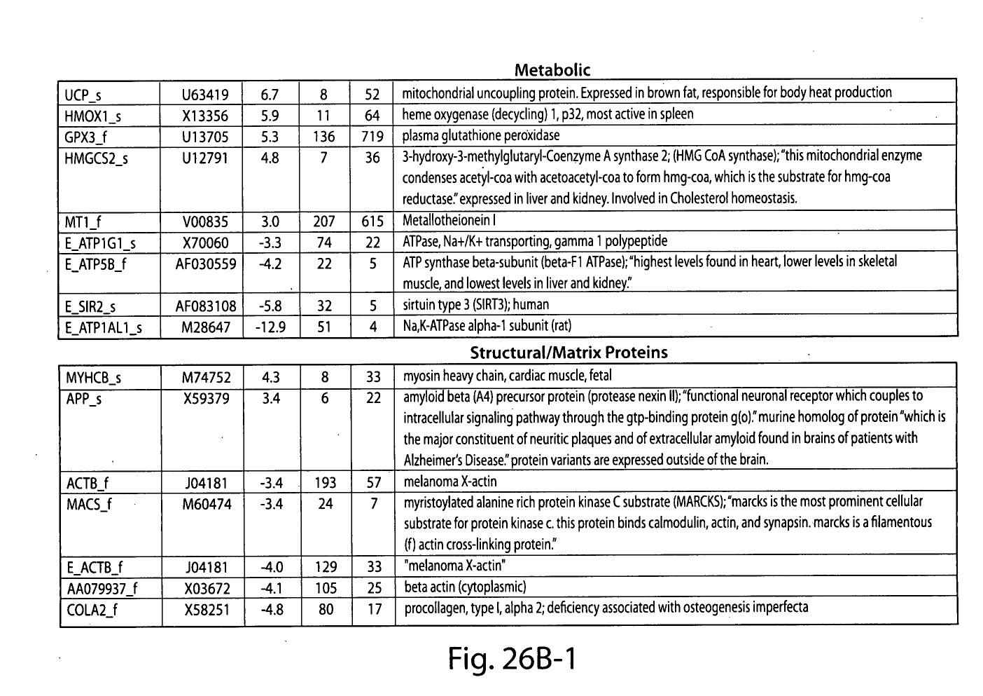

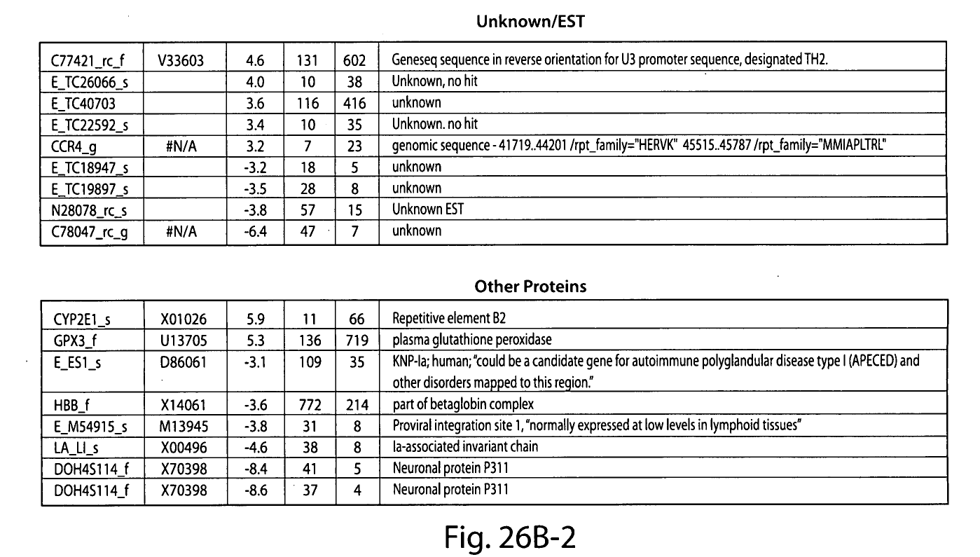

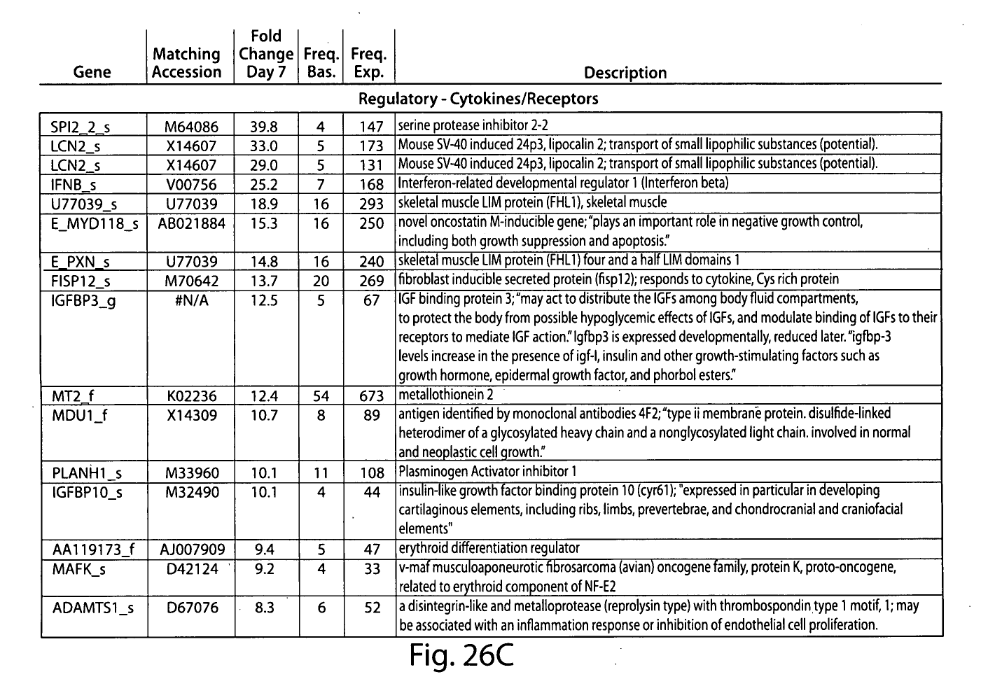

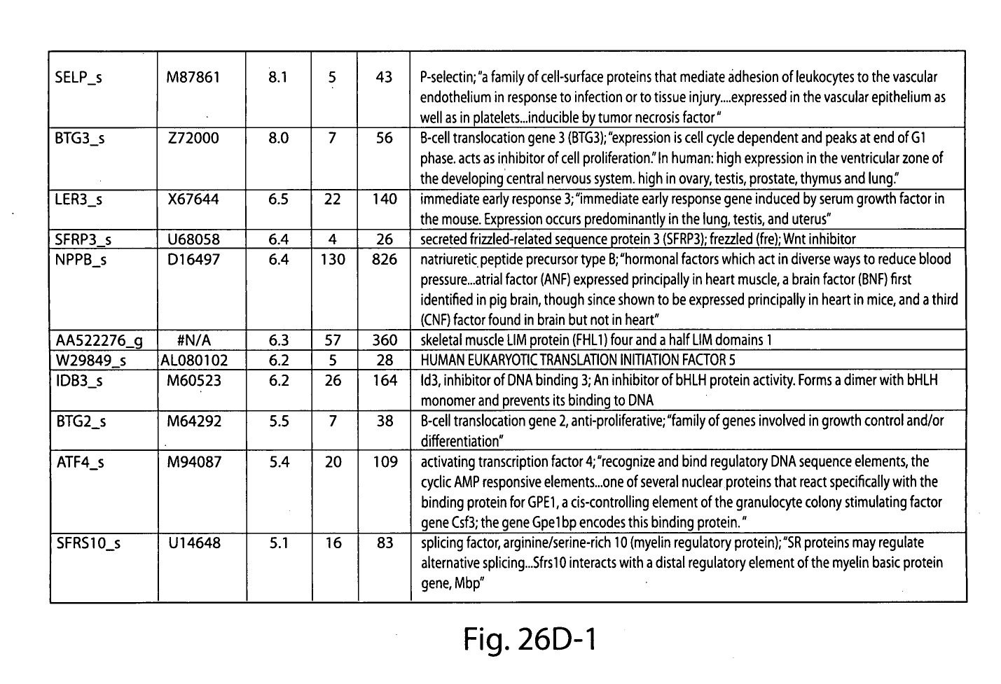

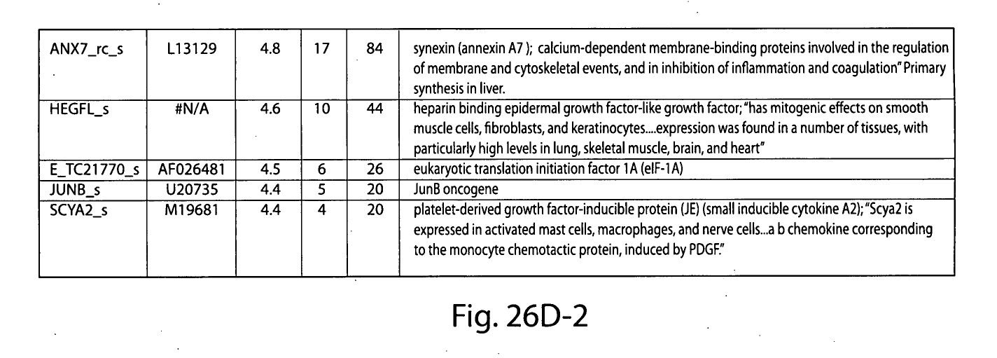

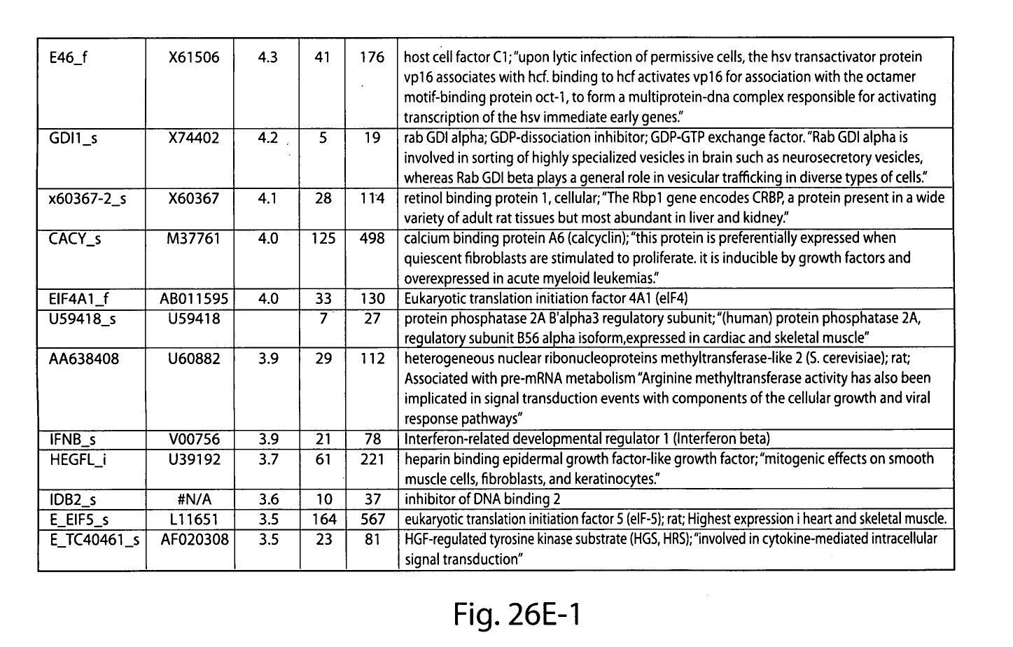

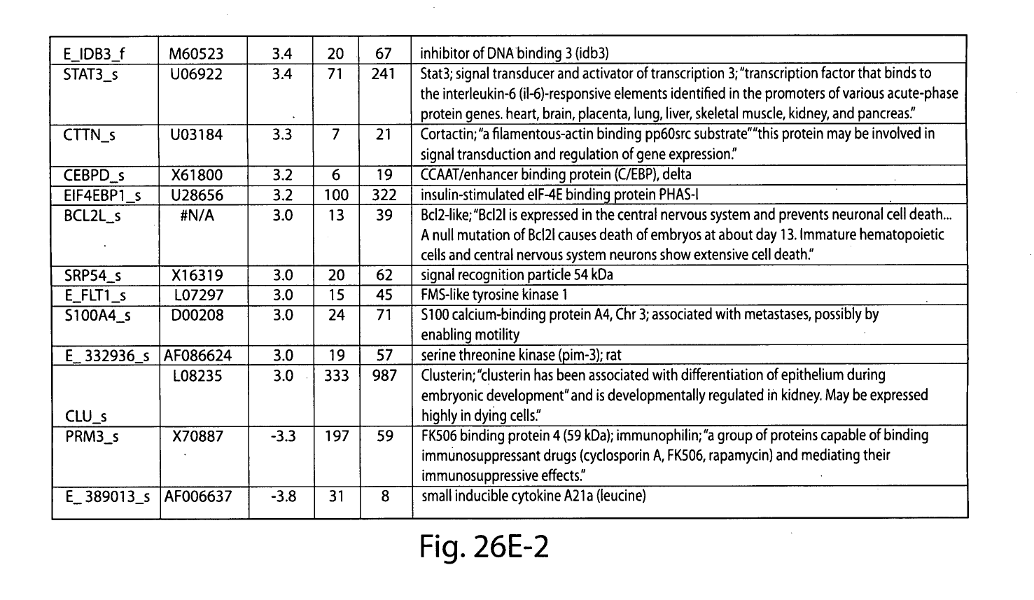

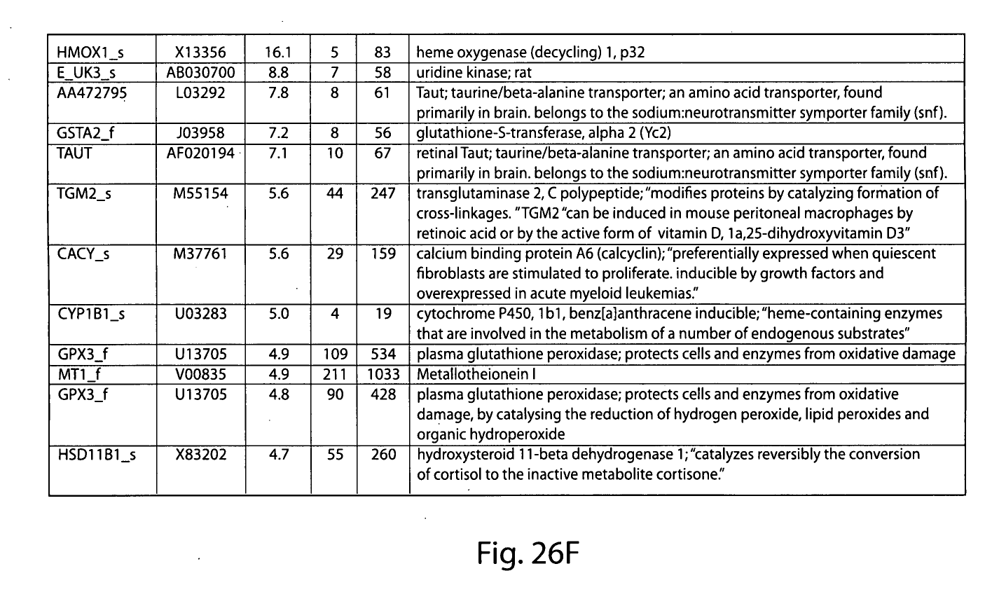

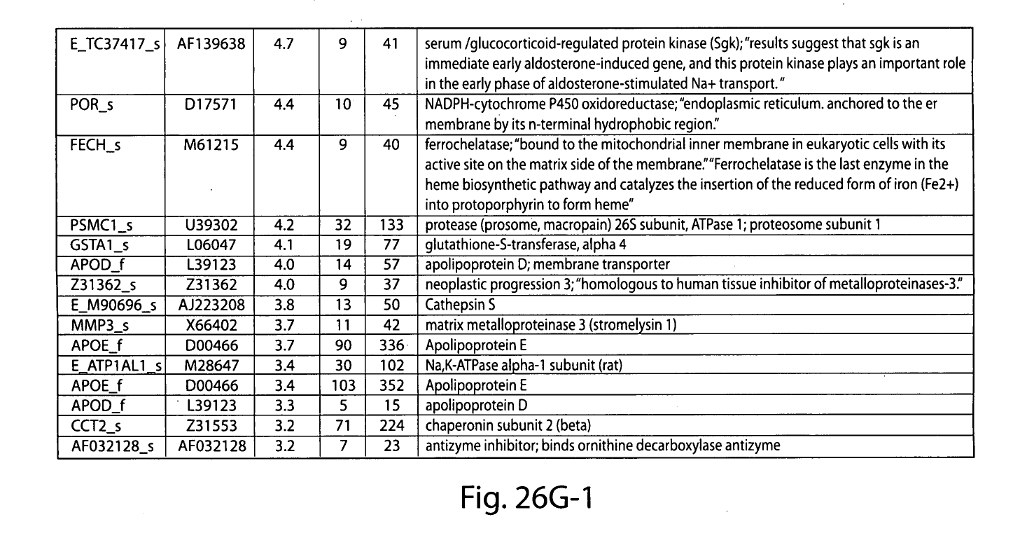

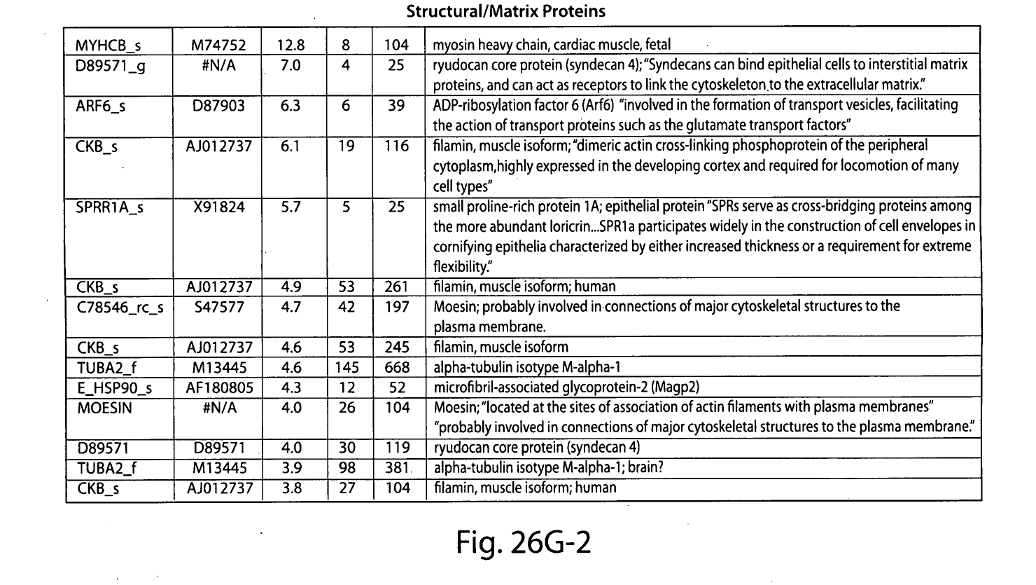

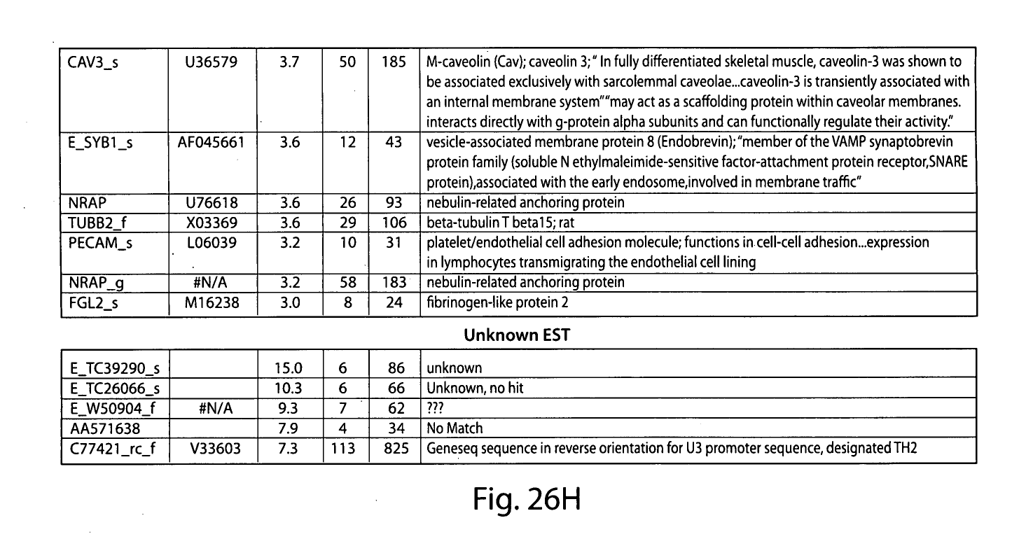

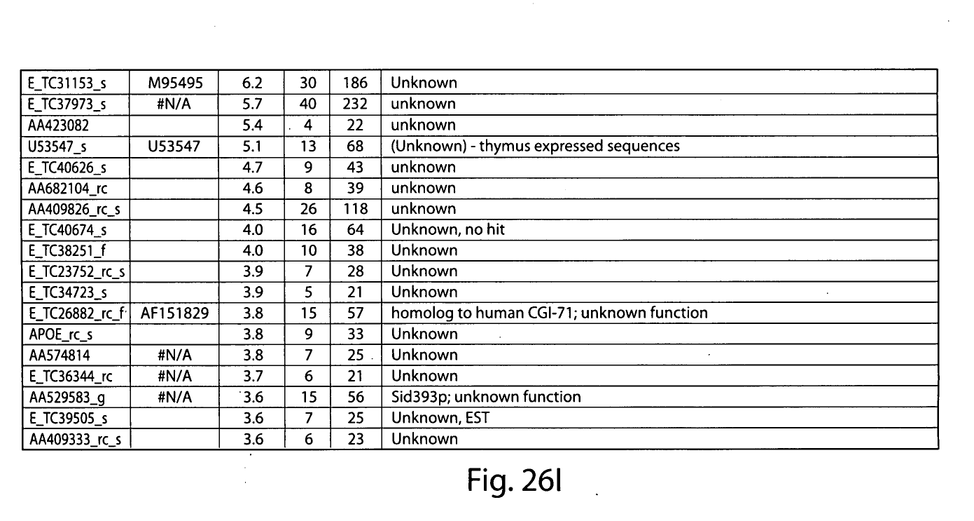

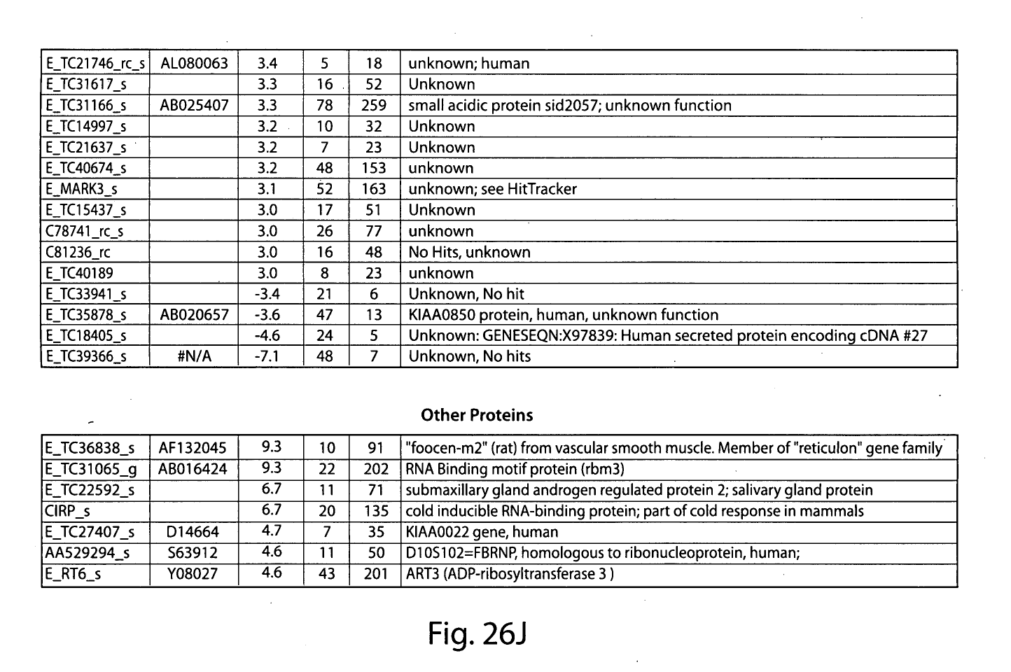

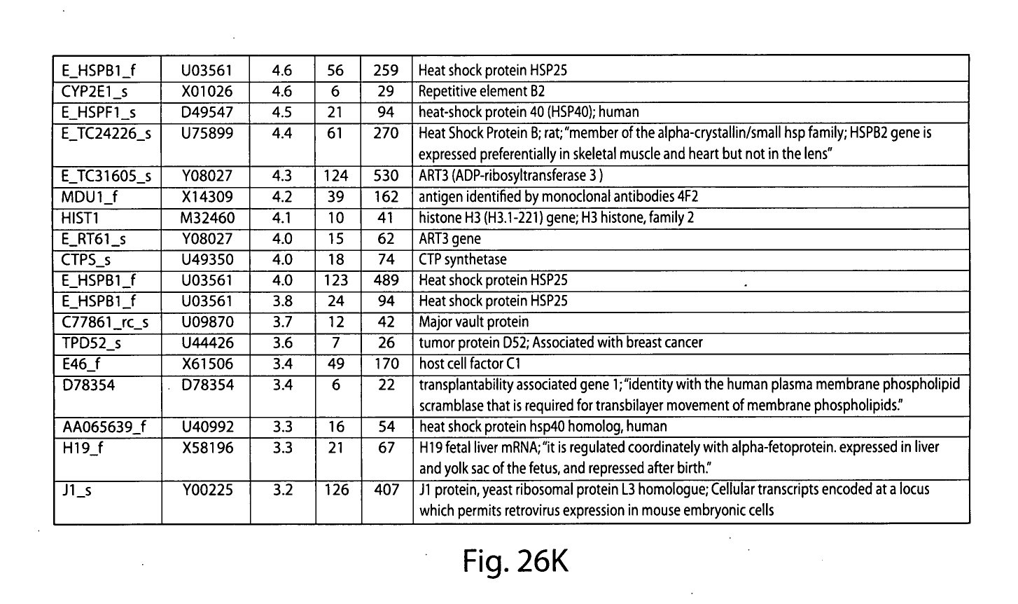

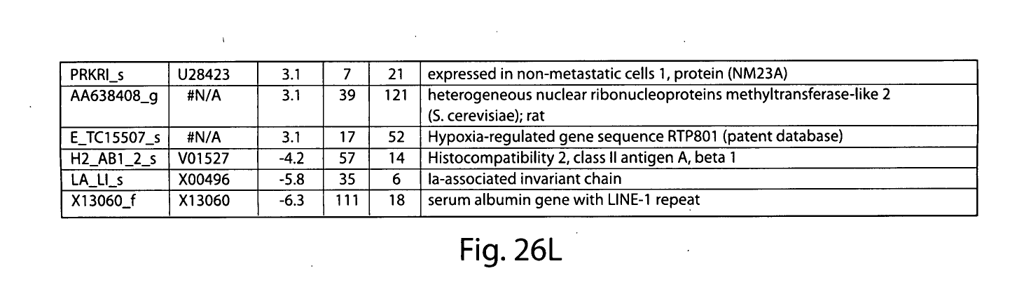

Analysis of RNA isolated from the heart tissue of mice three days after injection with AdBMP-10 as measured with the Affymetrix® GeneChip MU11k was compared to the control; results are shown in FIG. 26A-26B. Global gene expression analysis results for seven days post-injection are shown in FIG. 26C-26L.

Experiment B. In Vivo Transcriptional Profiling Using Affymetrix GeneChip MU11k Murine Array with RNA from Muscle Tissue of AdBMP-10 Mice

### Methods.

Muscle tissue from AdBMP-10 mice, injected as described above, was prepared as described above from mice at sacrificed three and seven days post-injection. Hybridization to Affymetrix oligonucleotide arrays was performed as described above.

### Results.

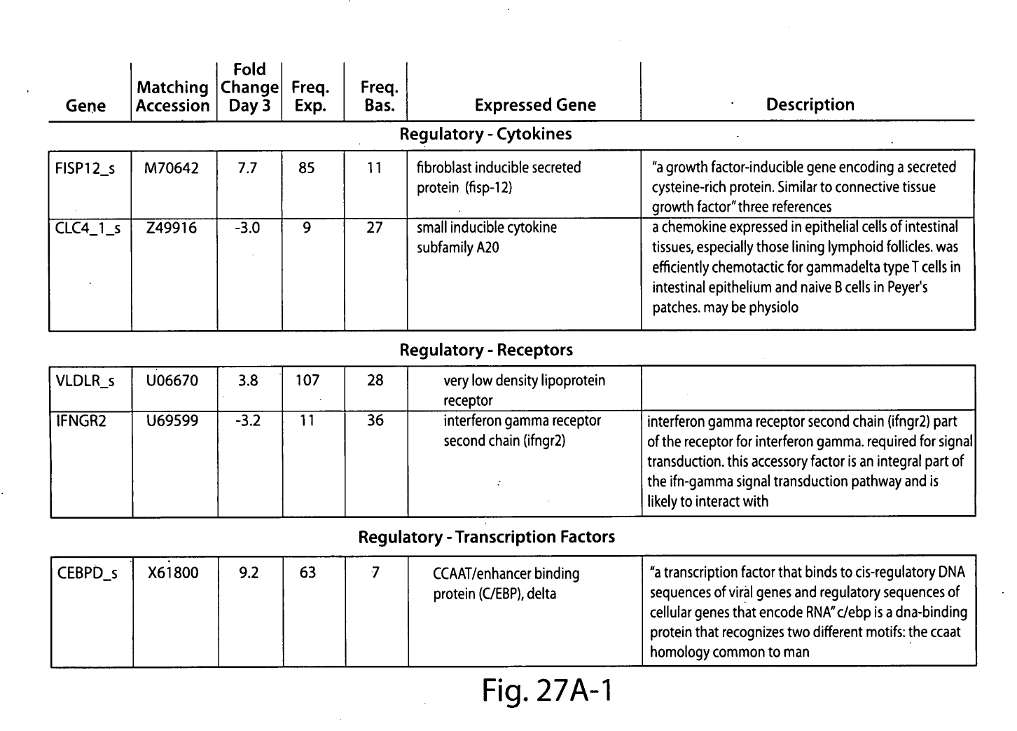

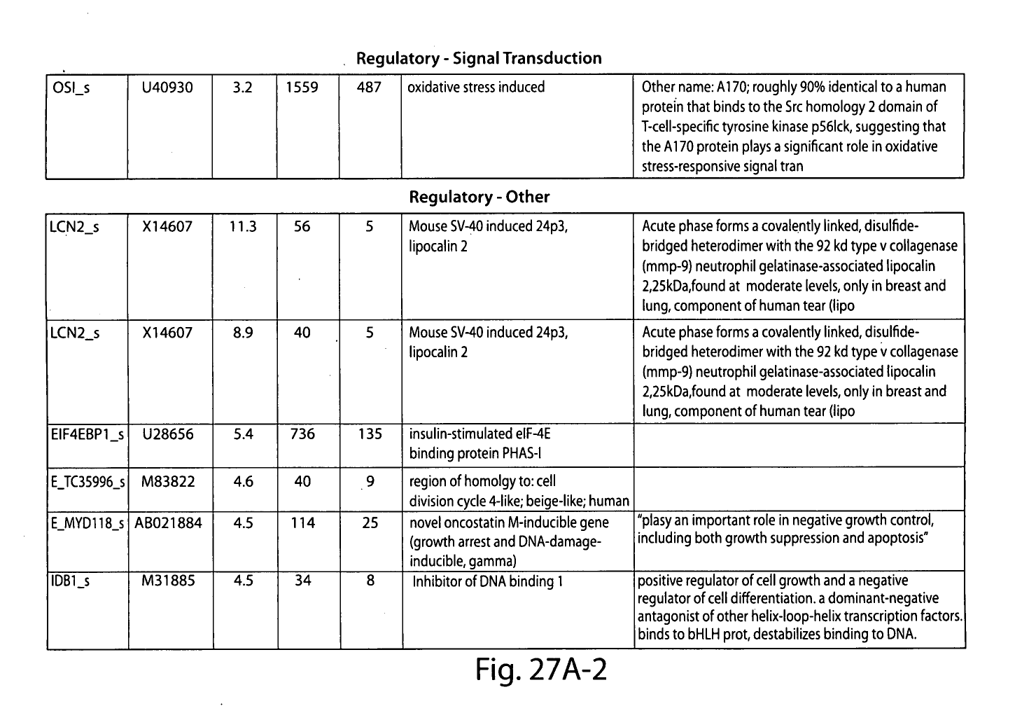

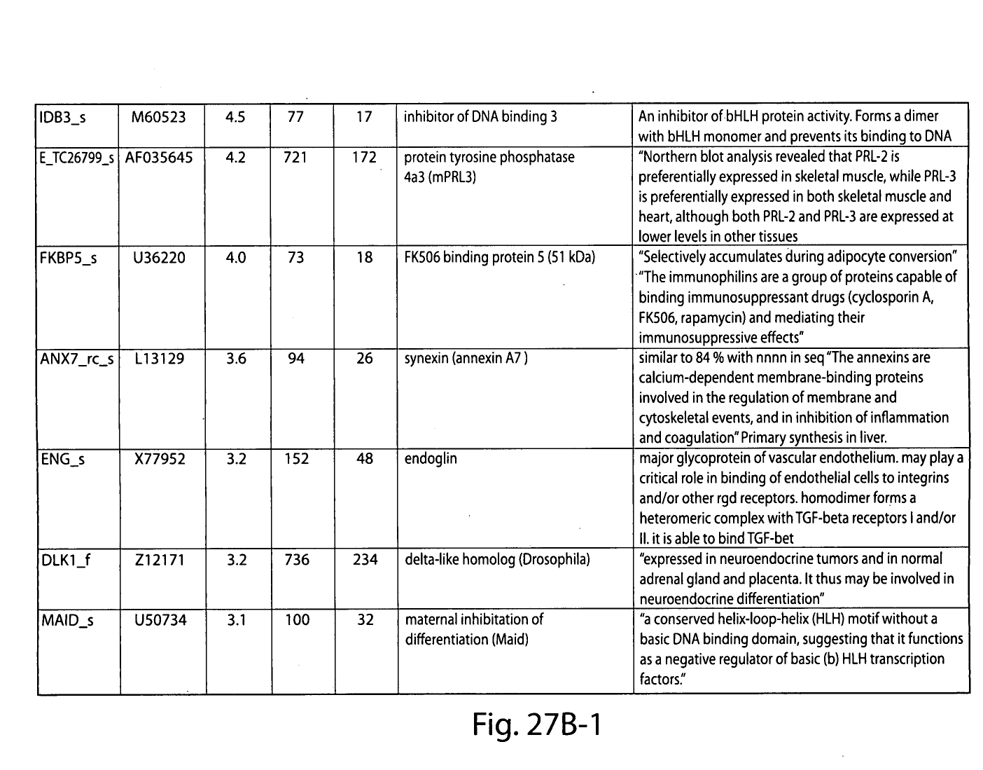

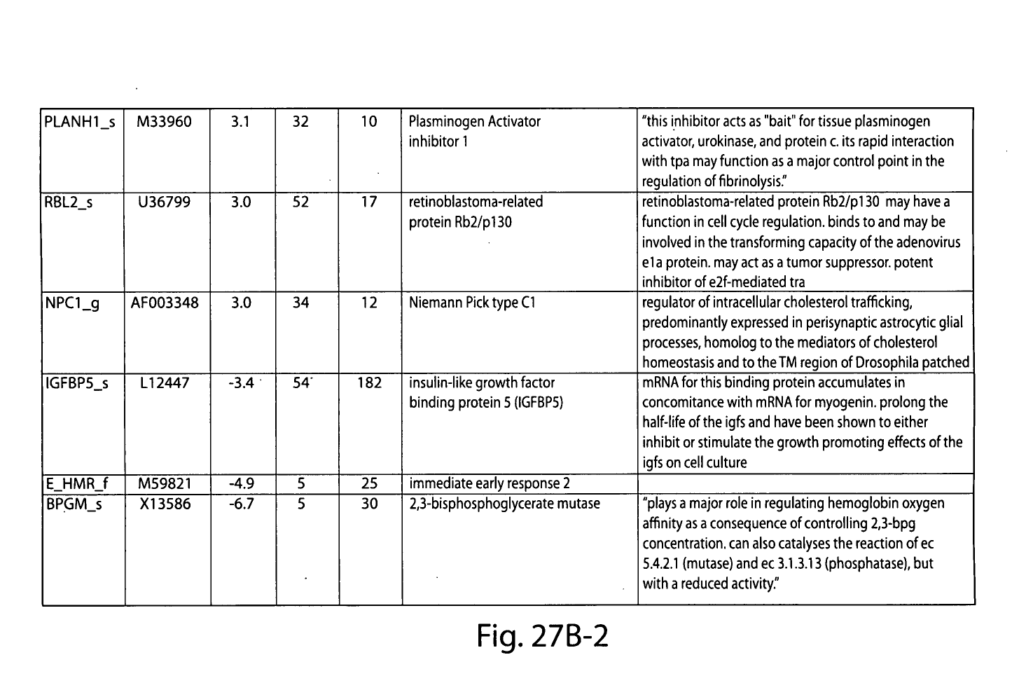

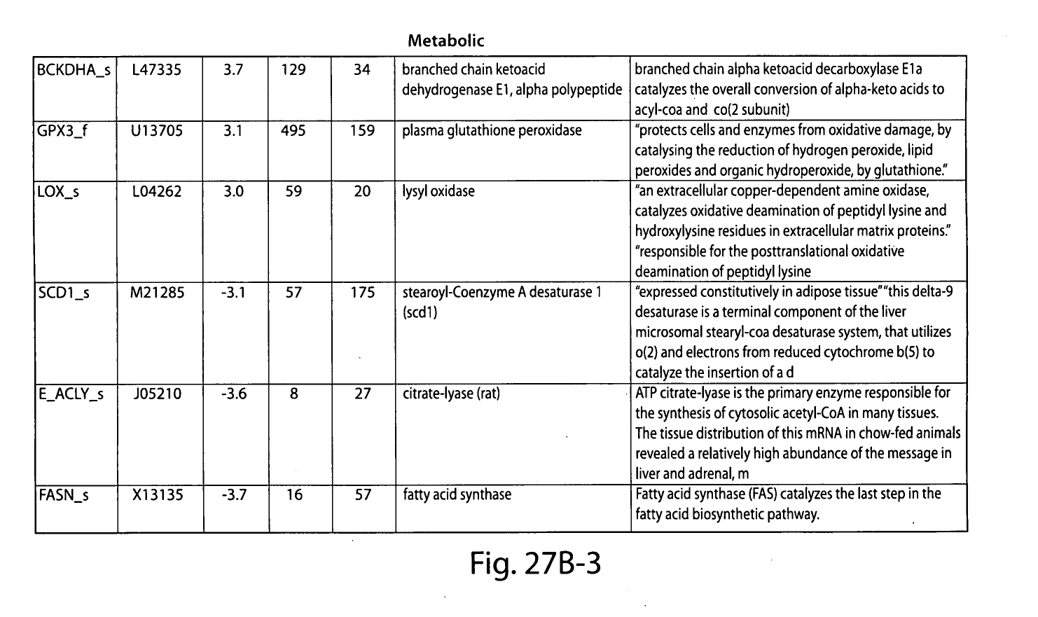

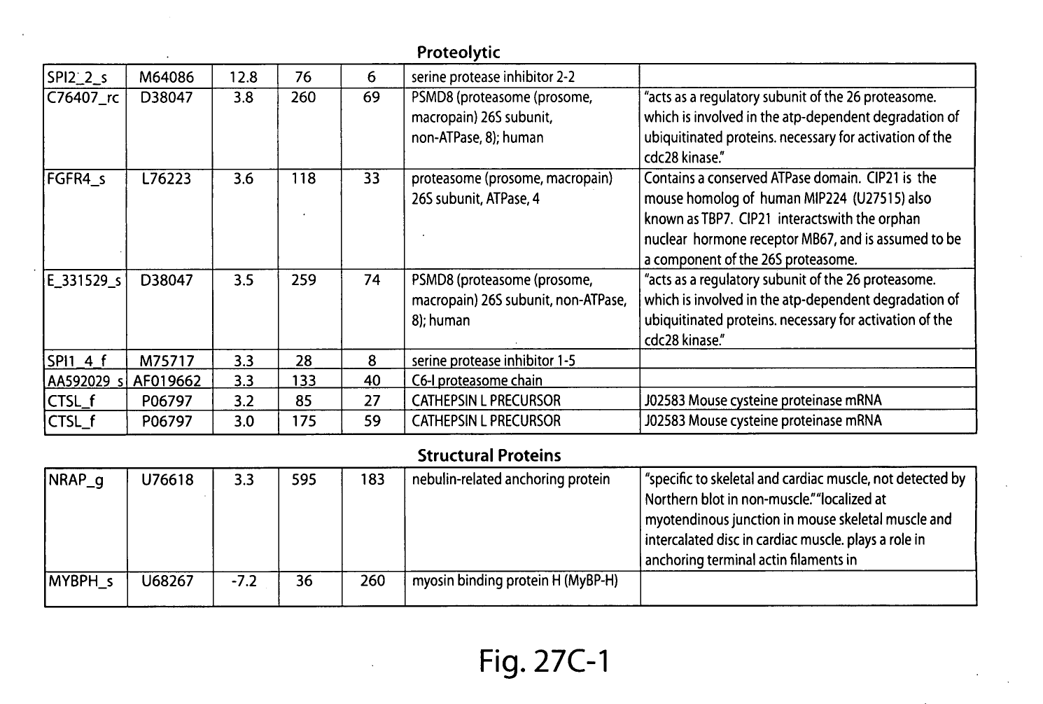

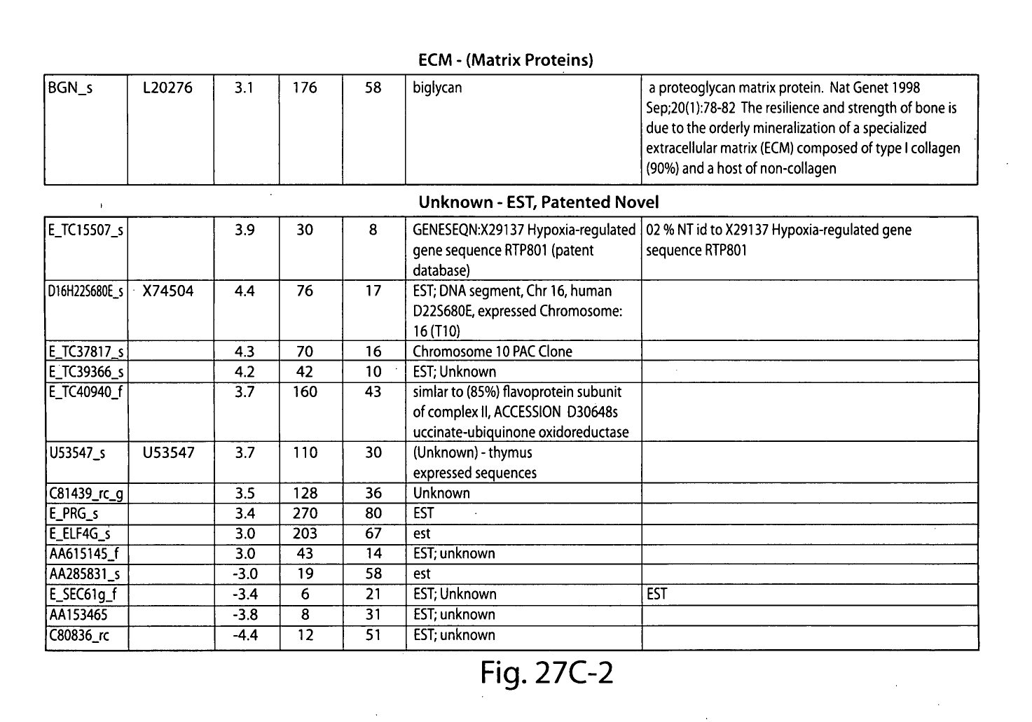

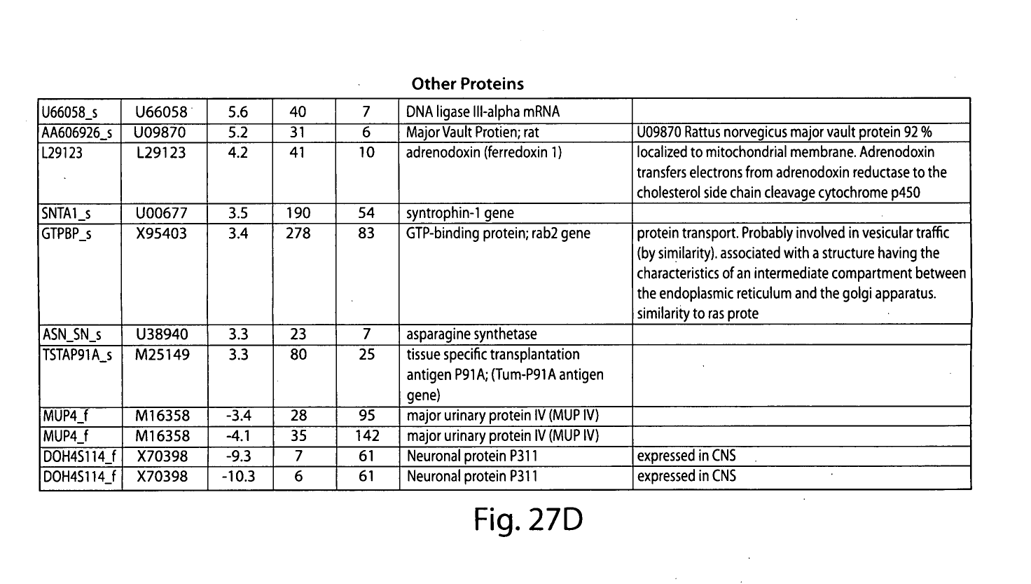

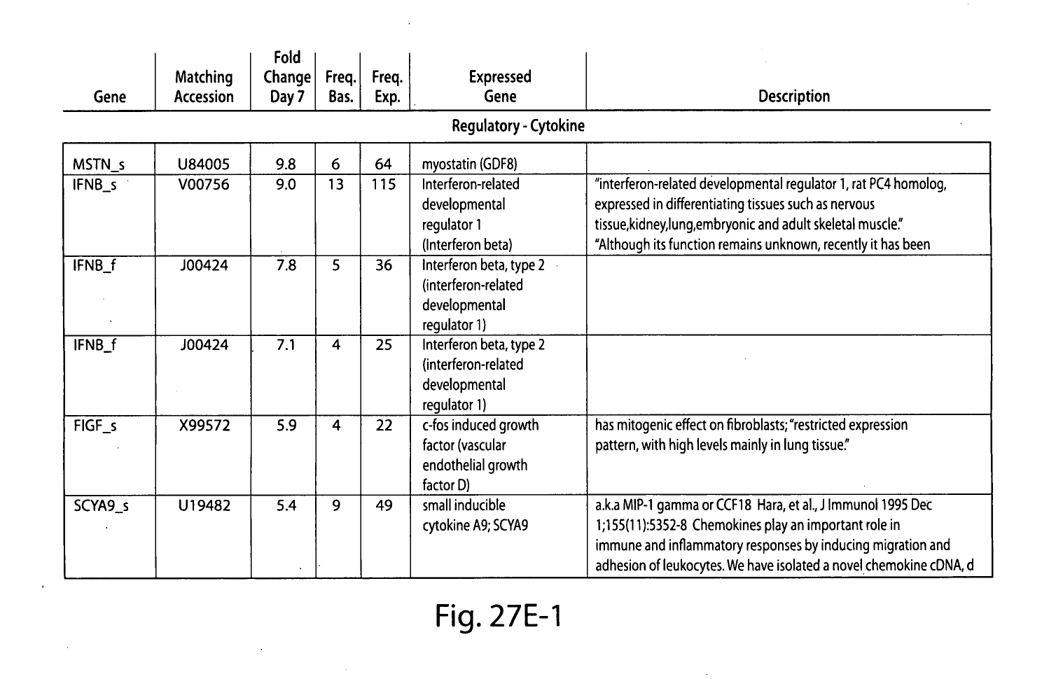

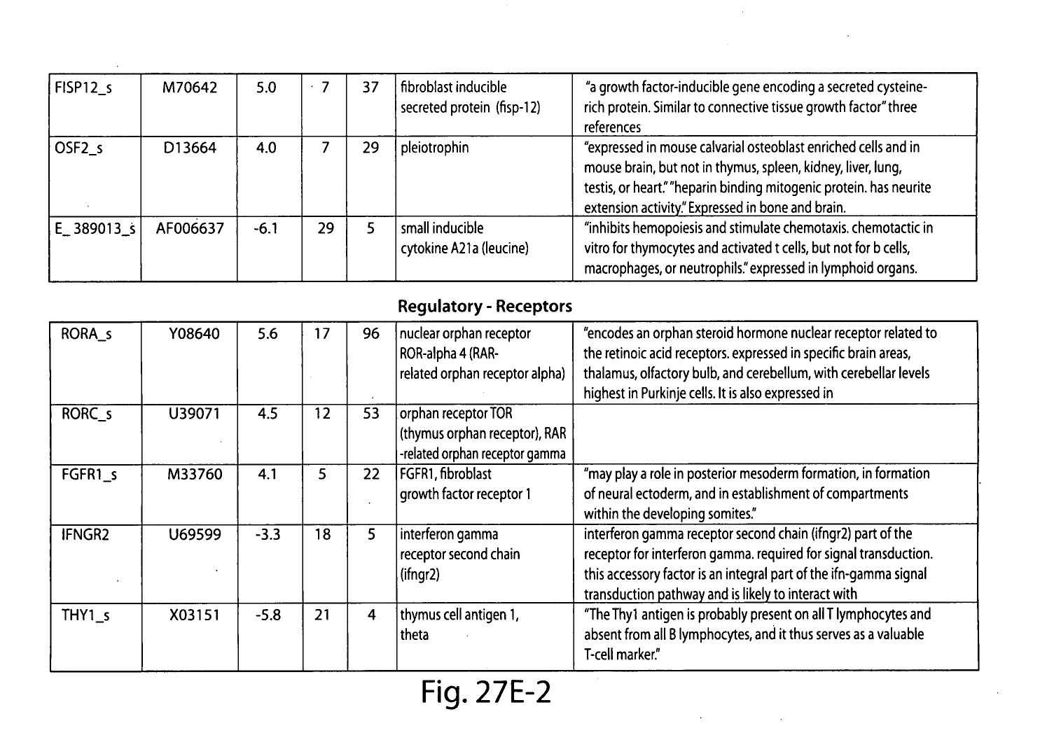

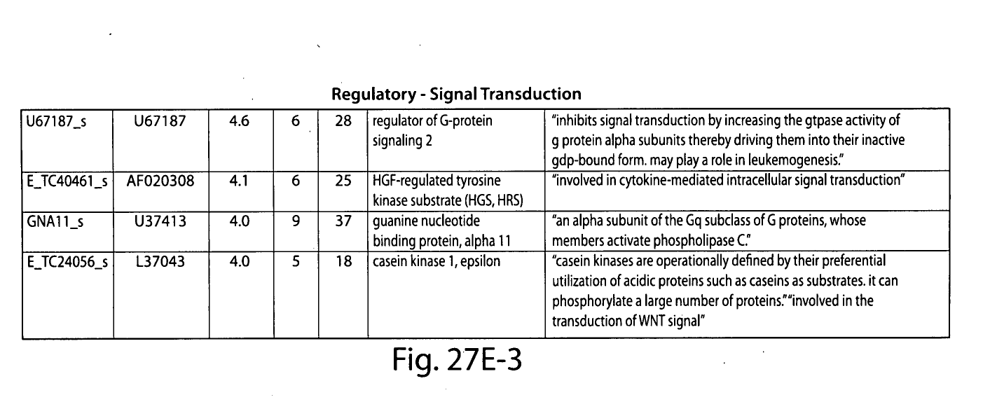

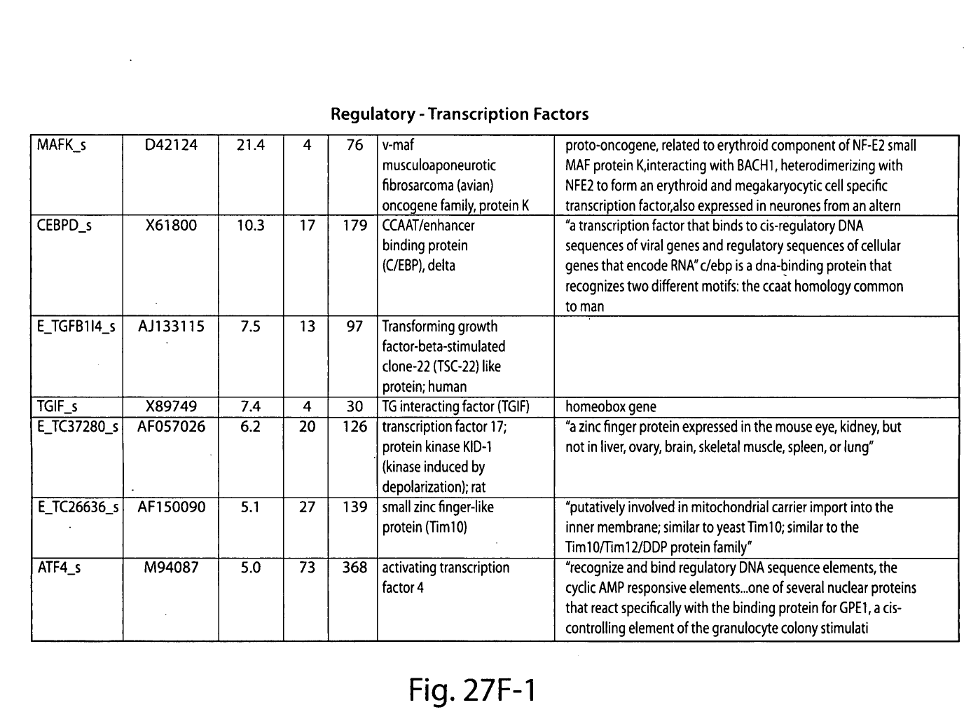

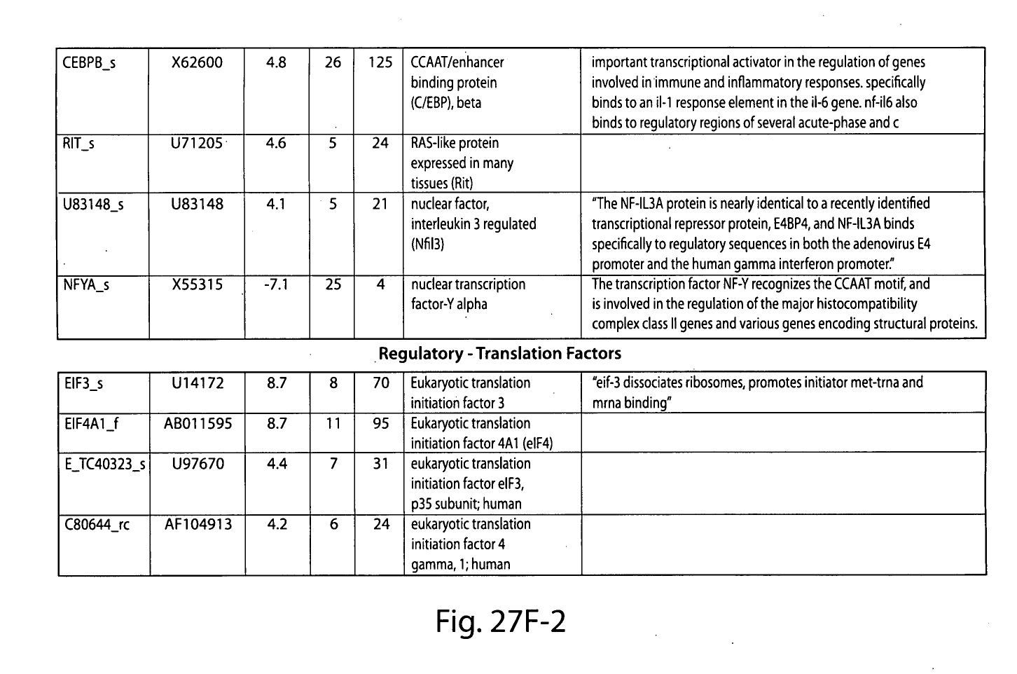

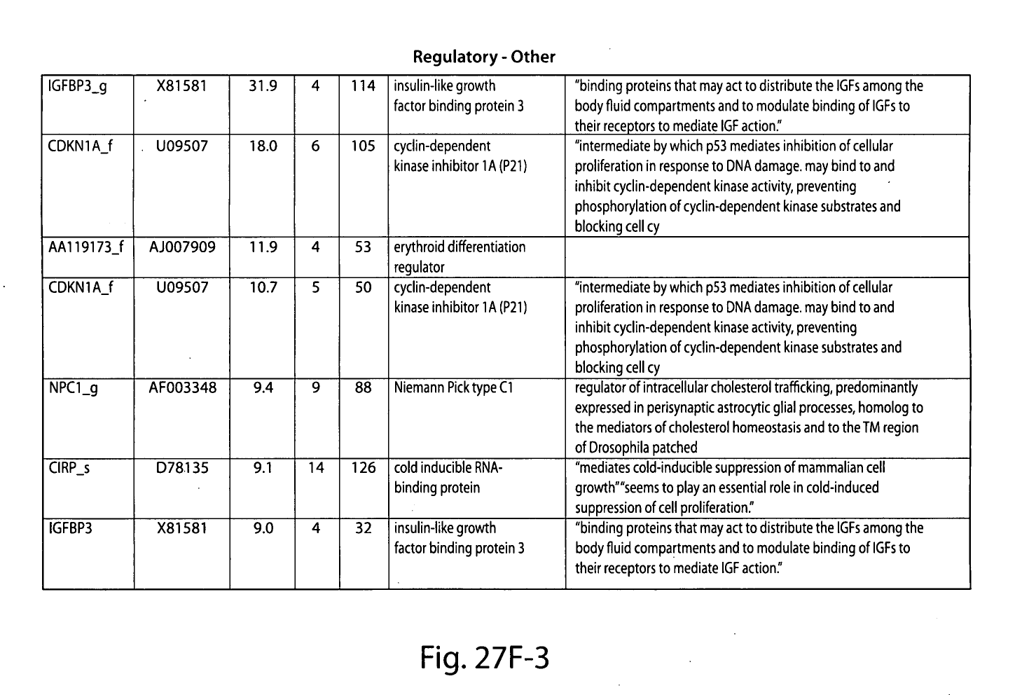

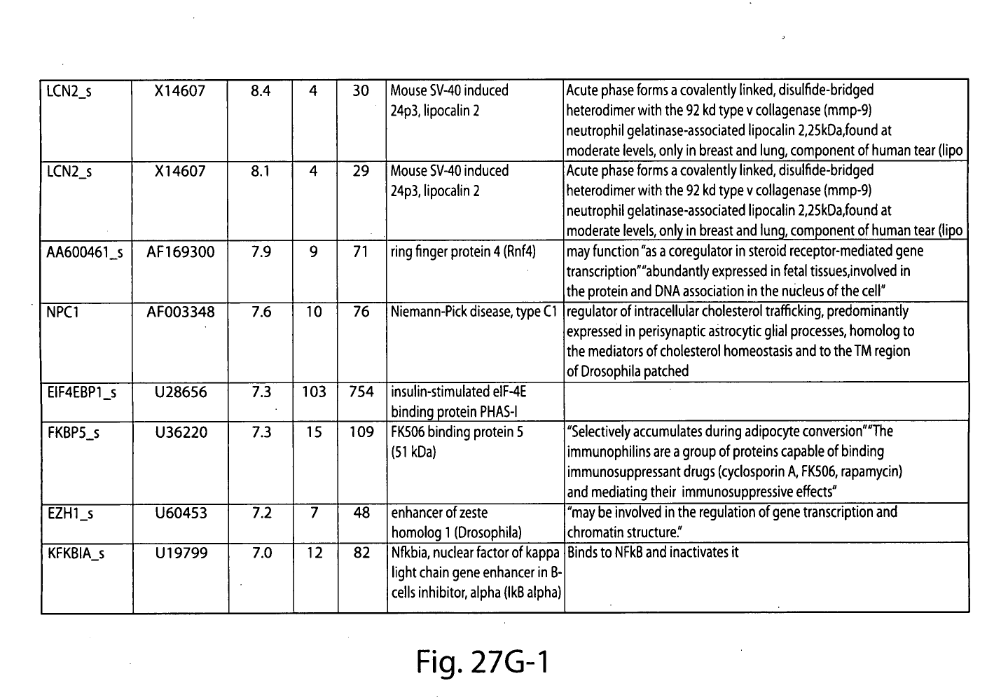

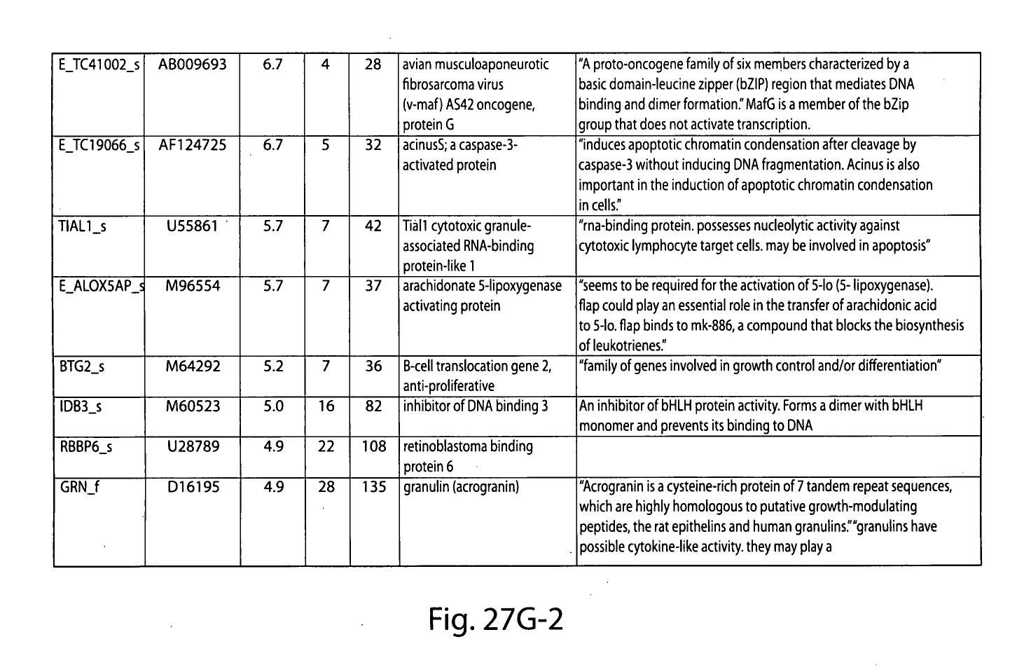

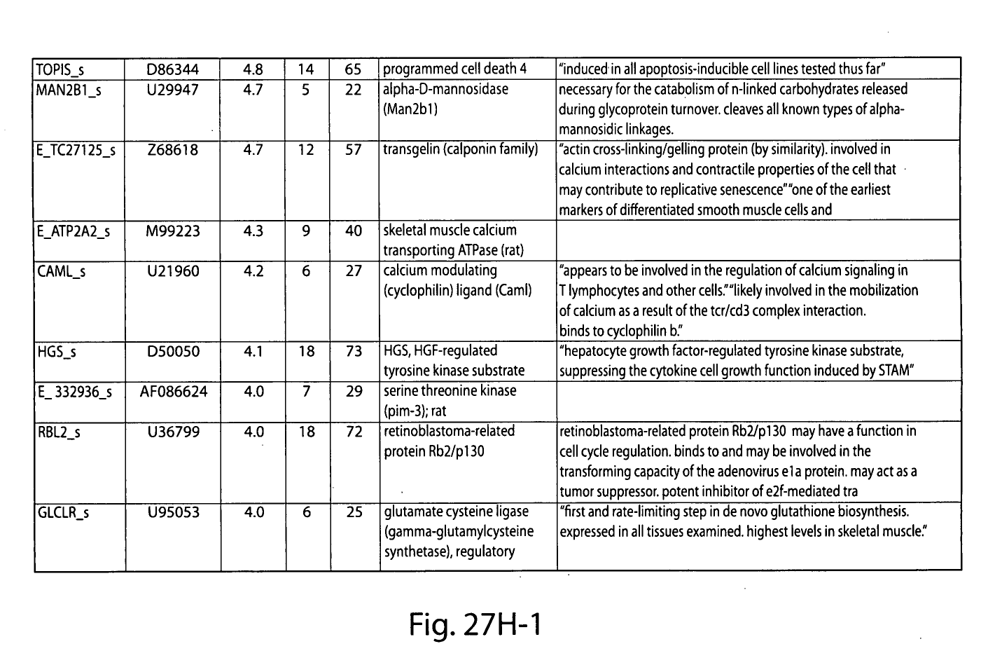

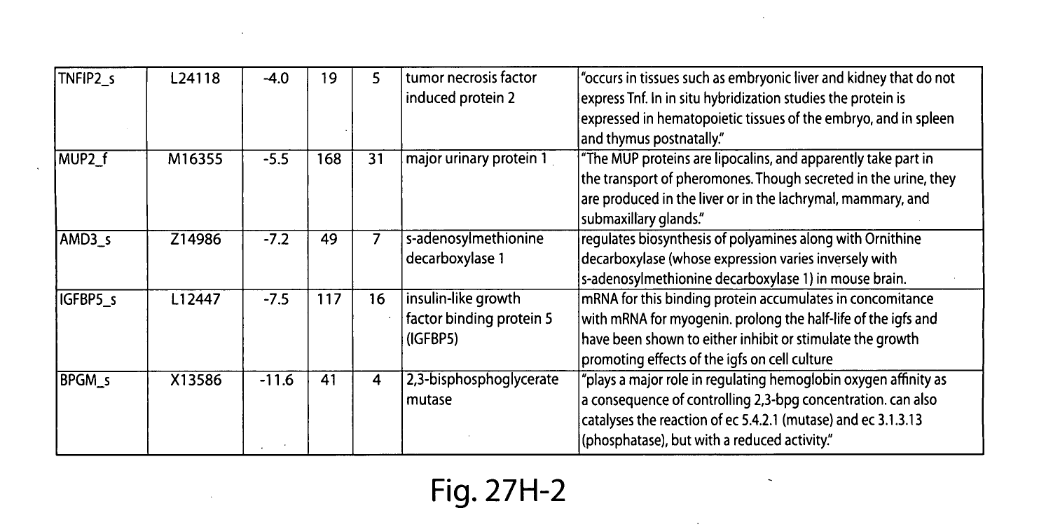

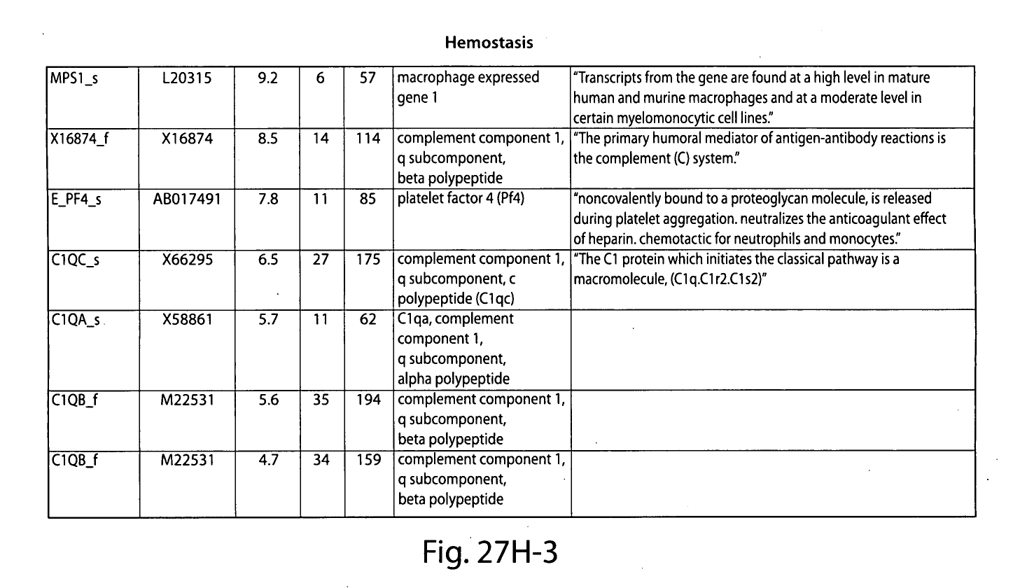

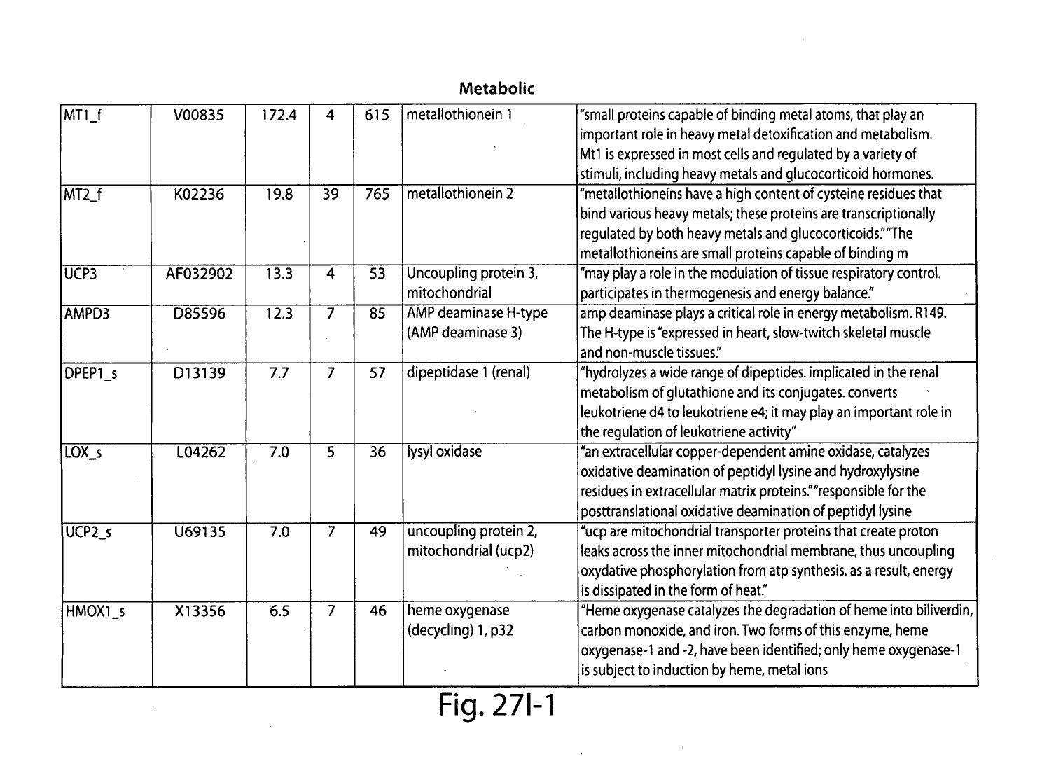

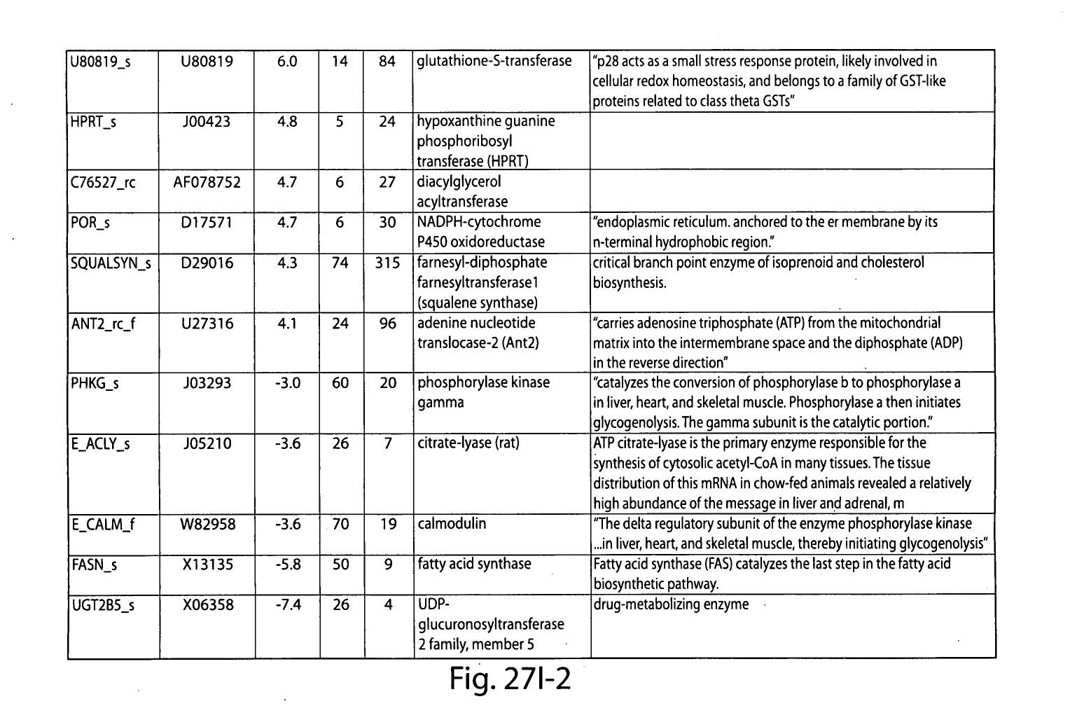

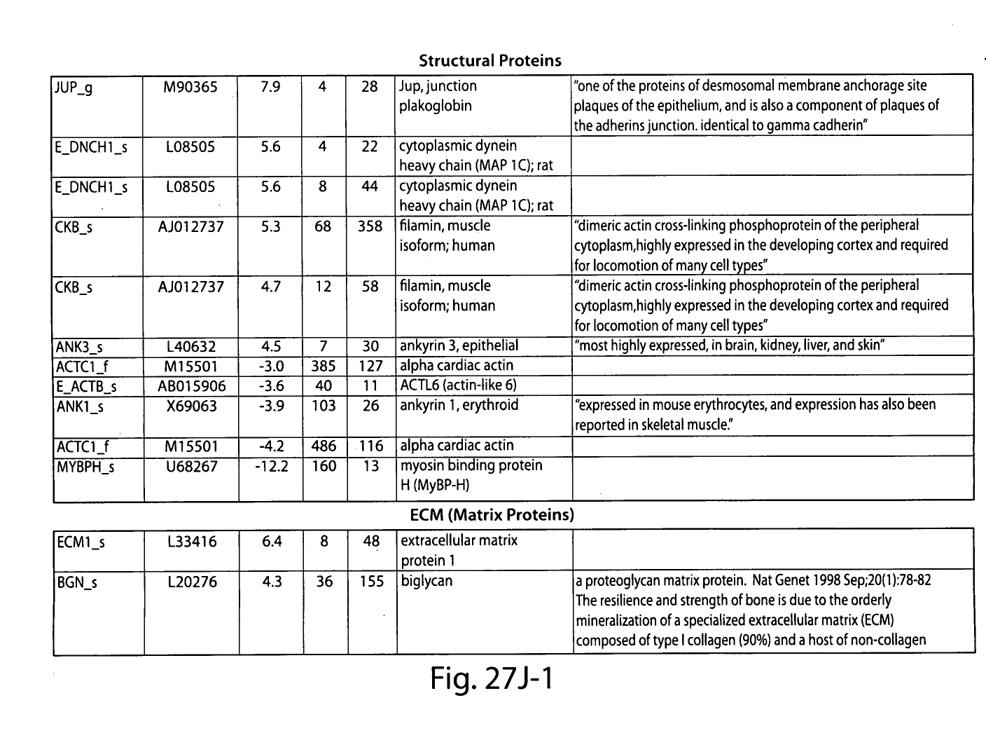

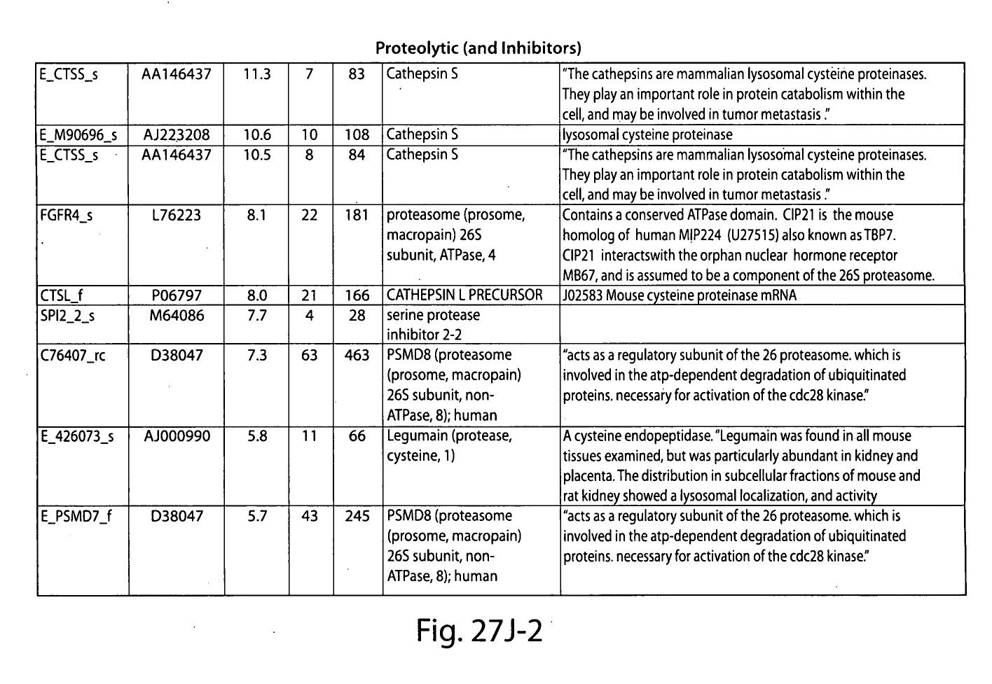

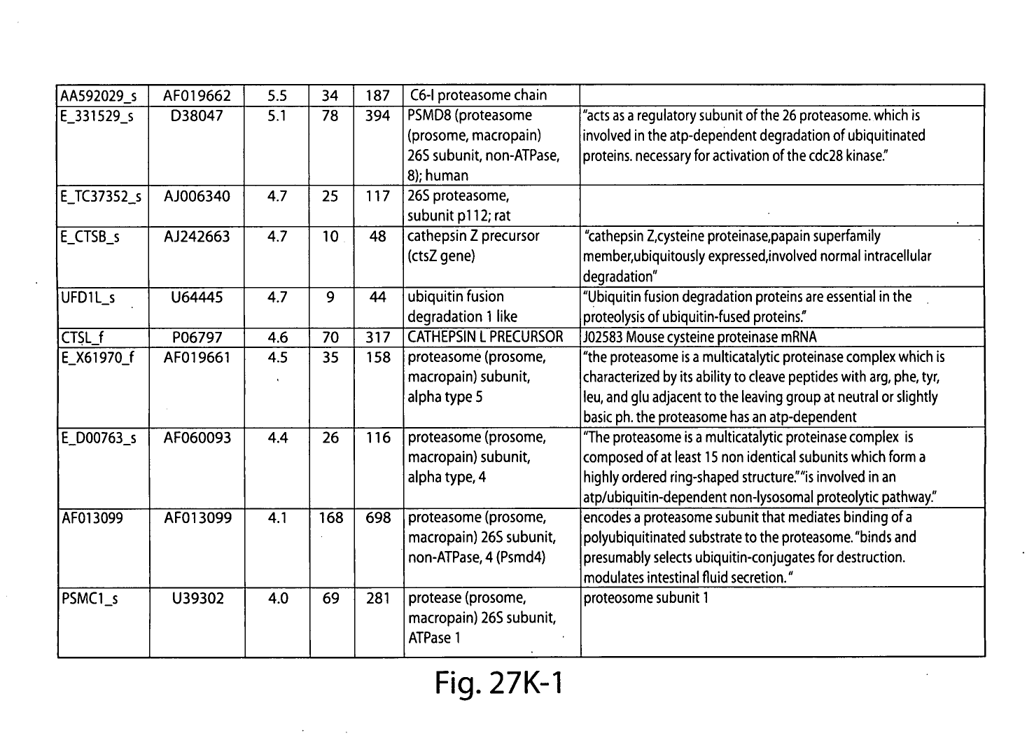

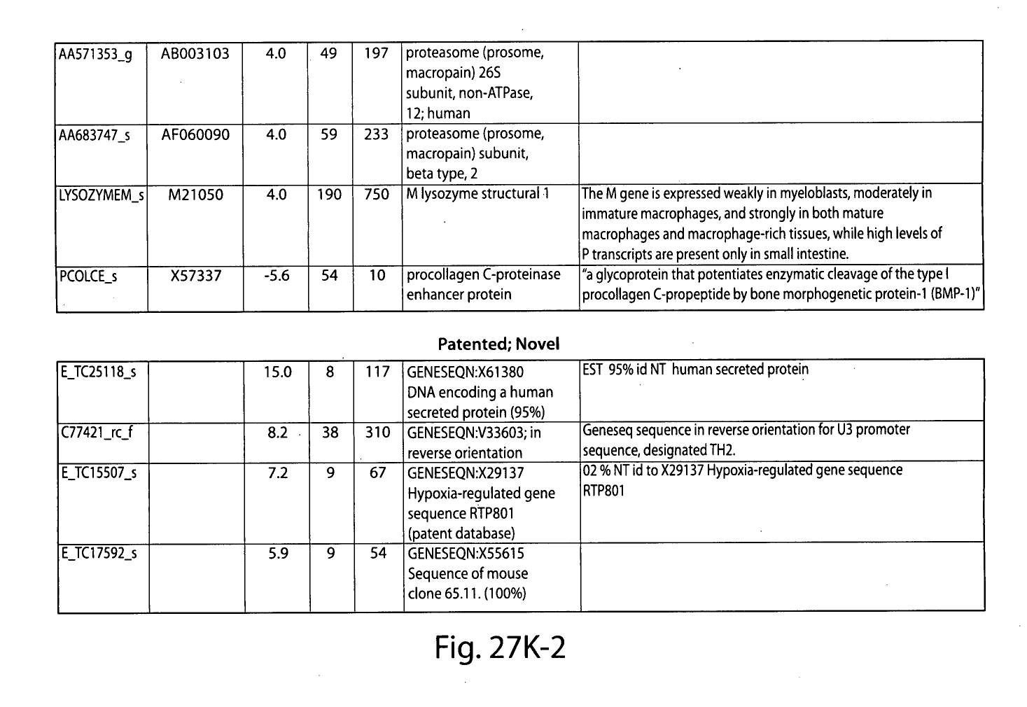

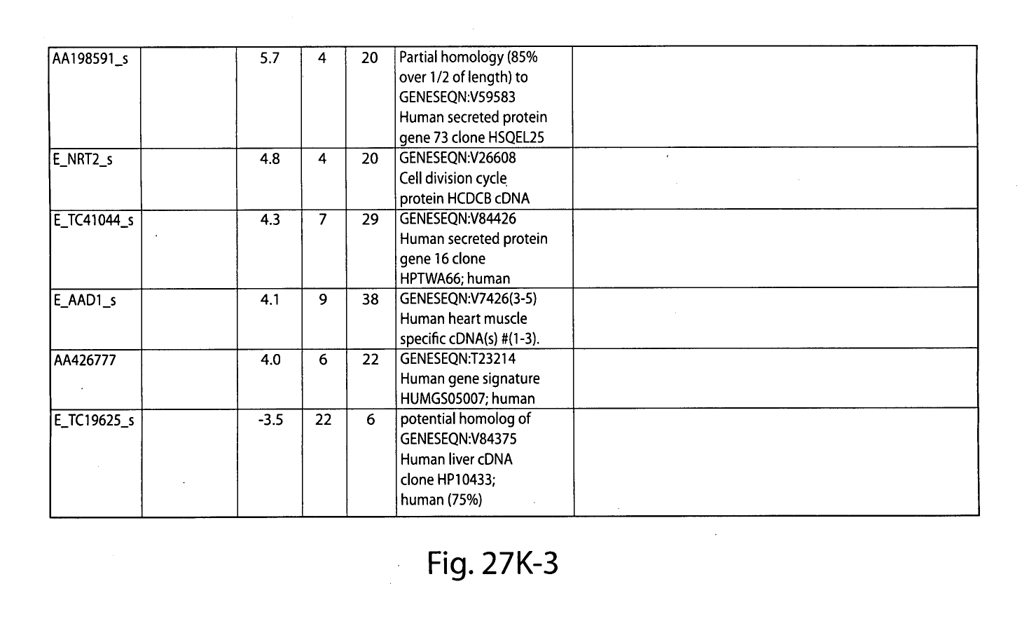

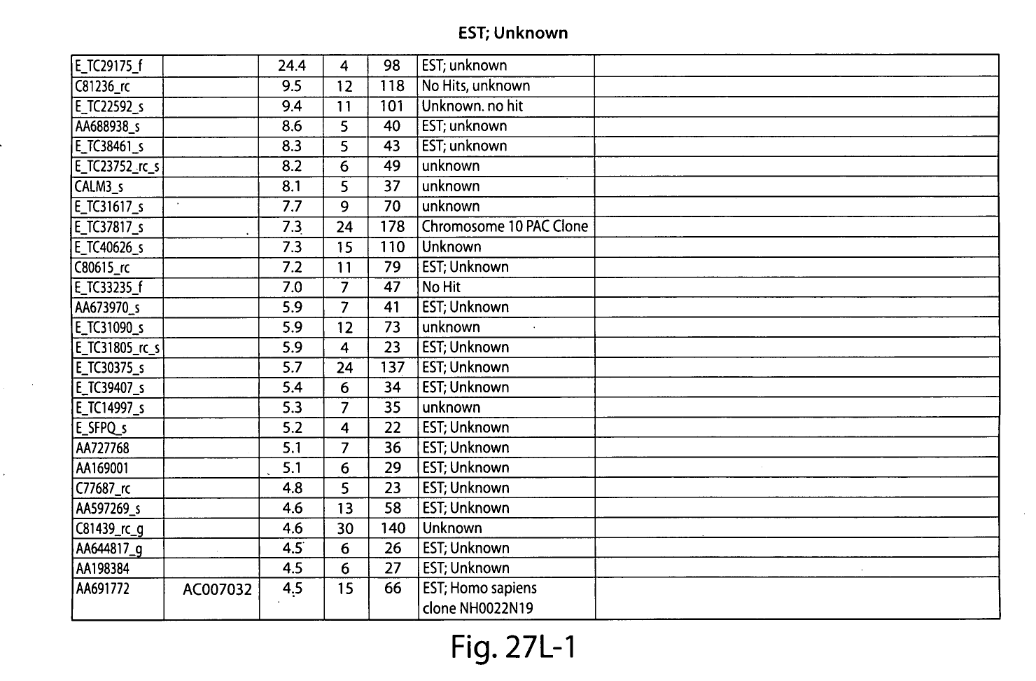

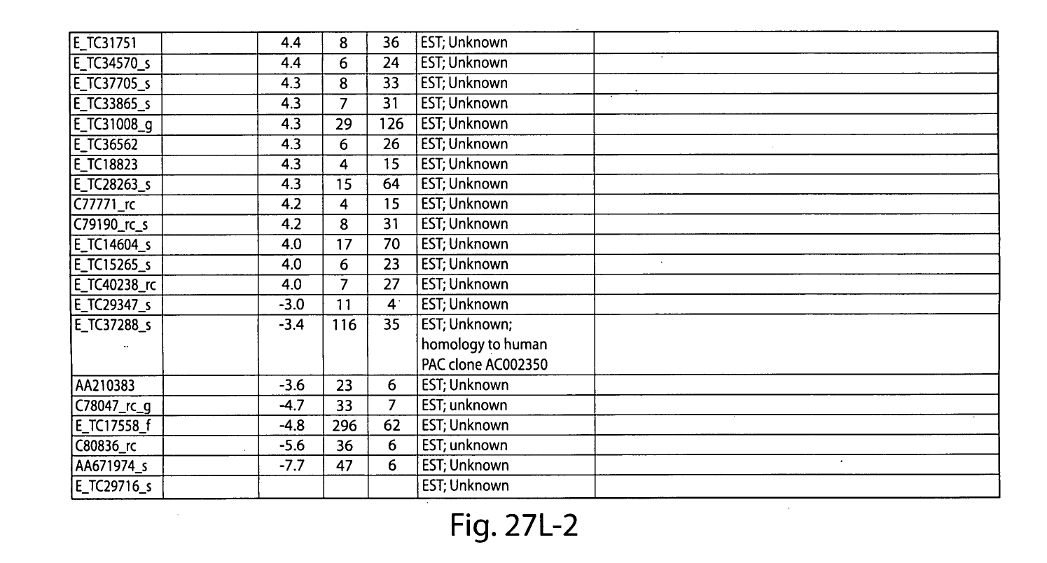

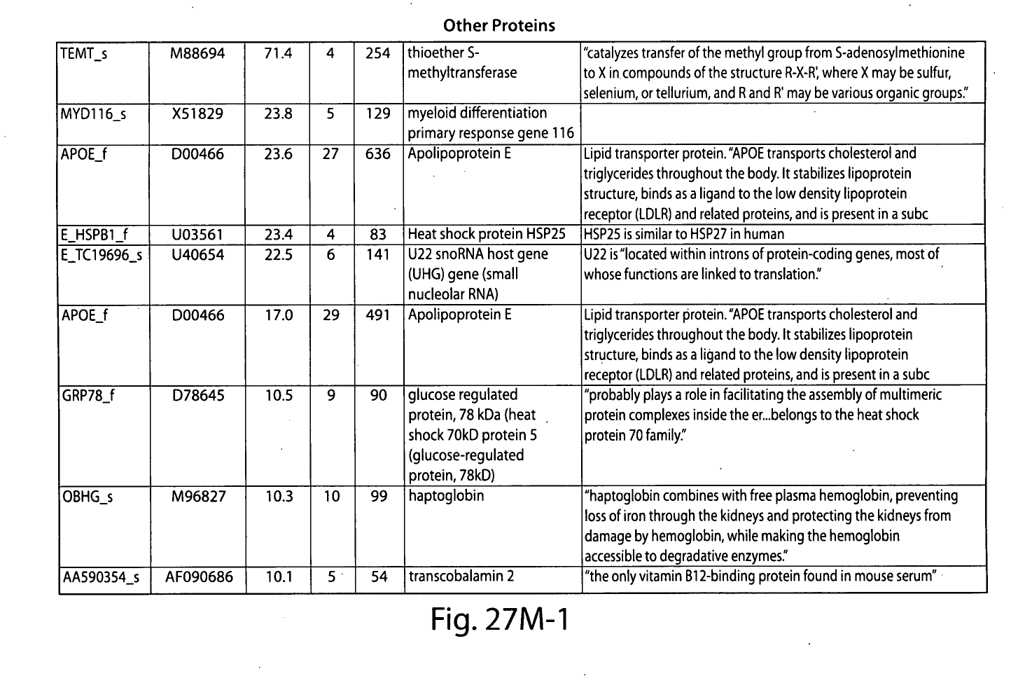

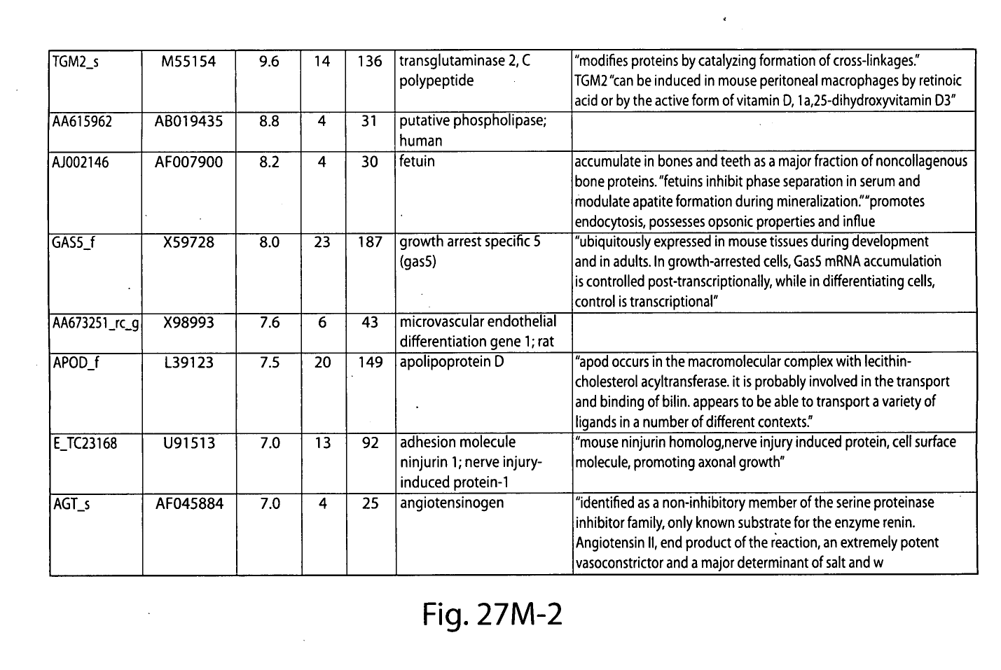

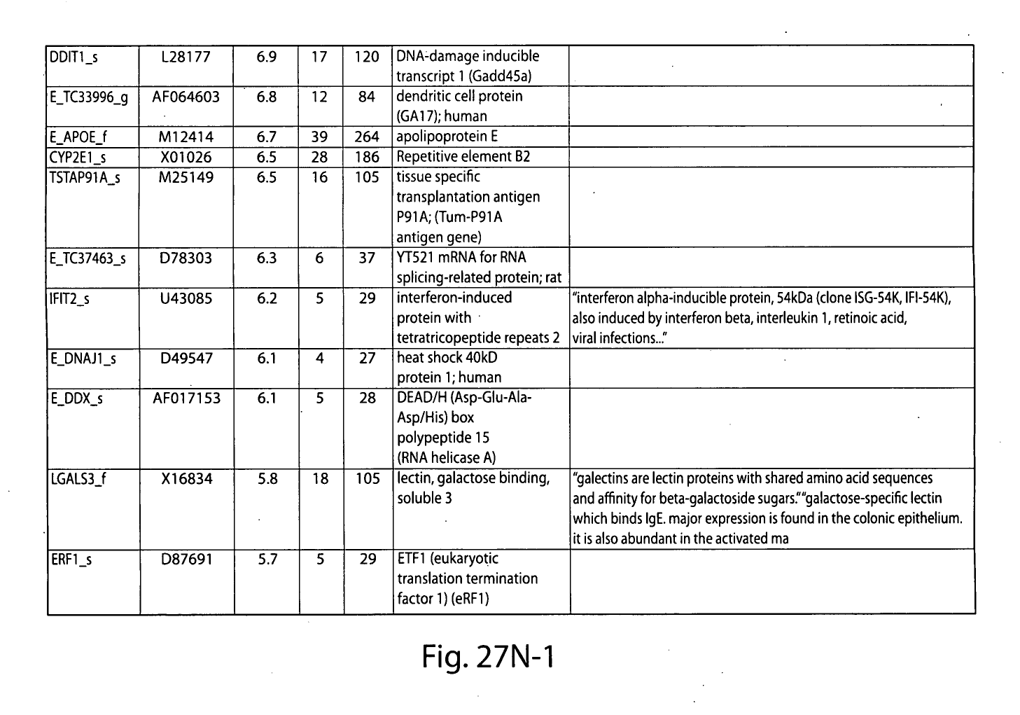

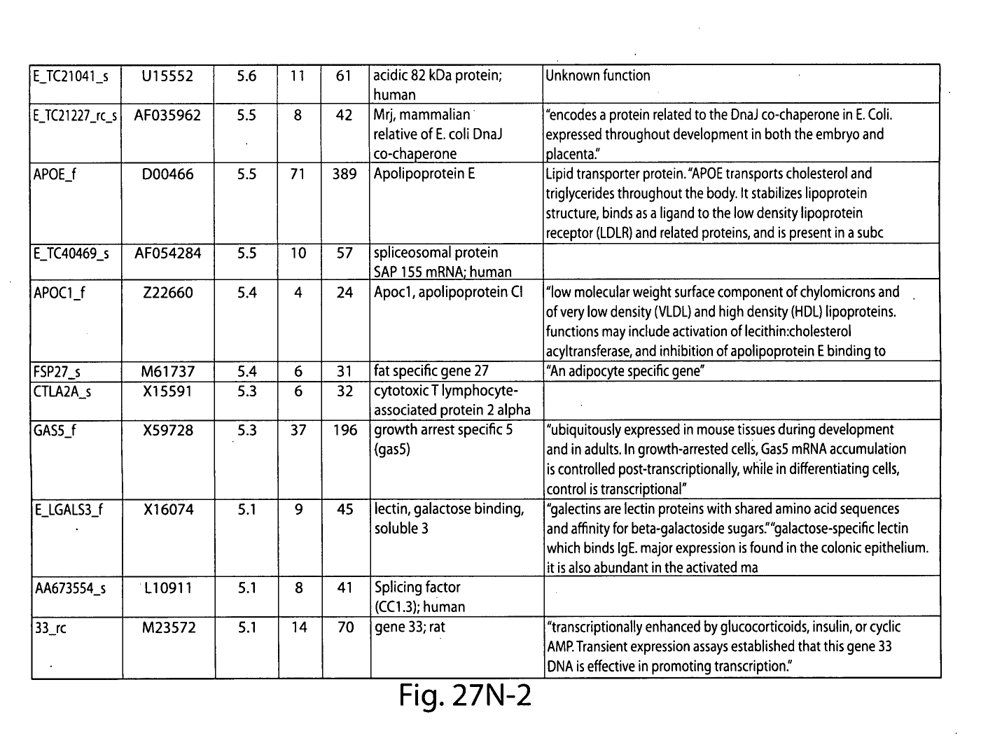

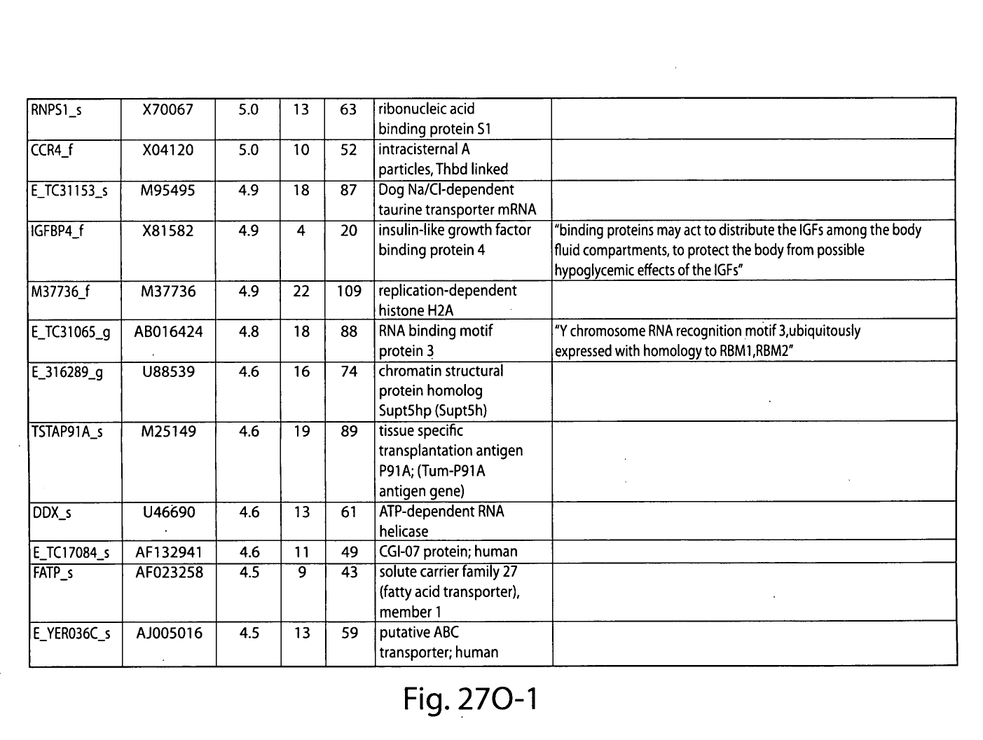

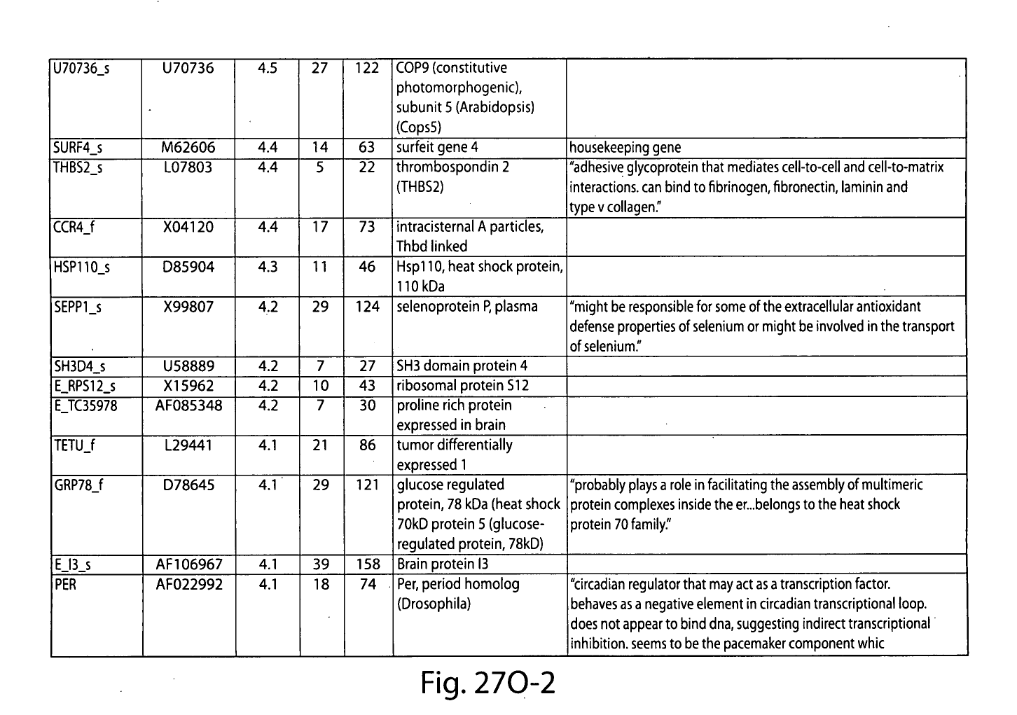

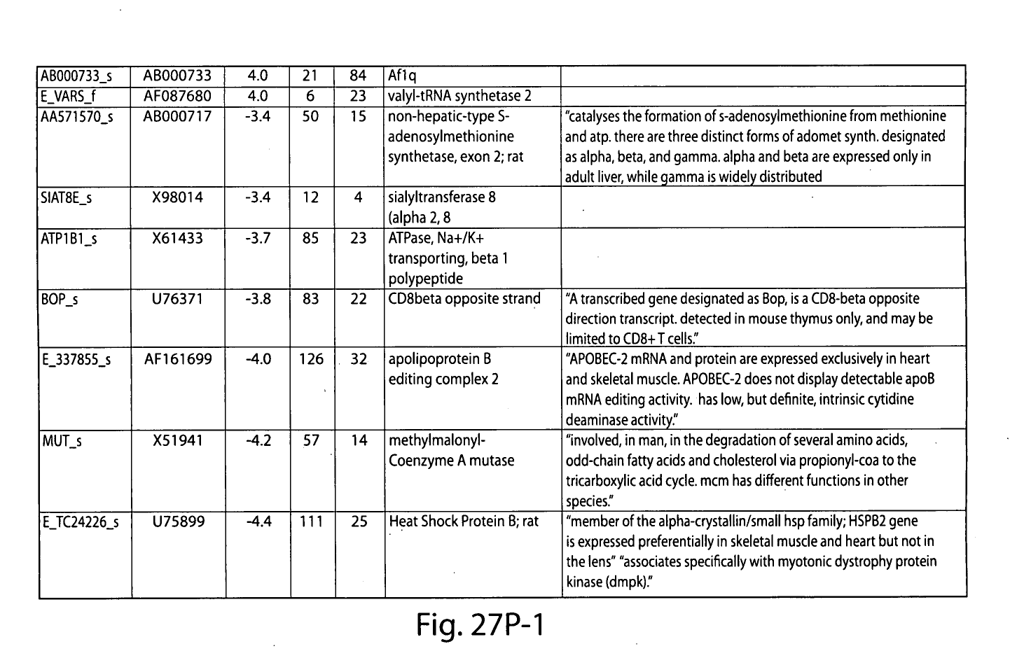

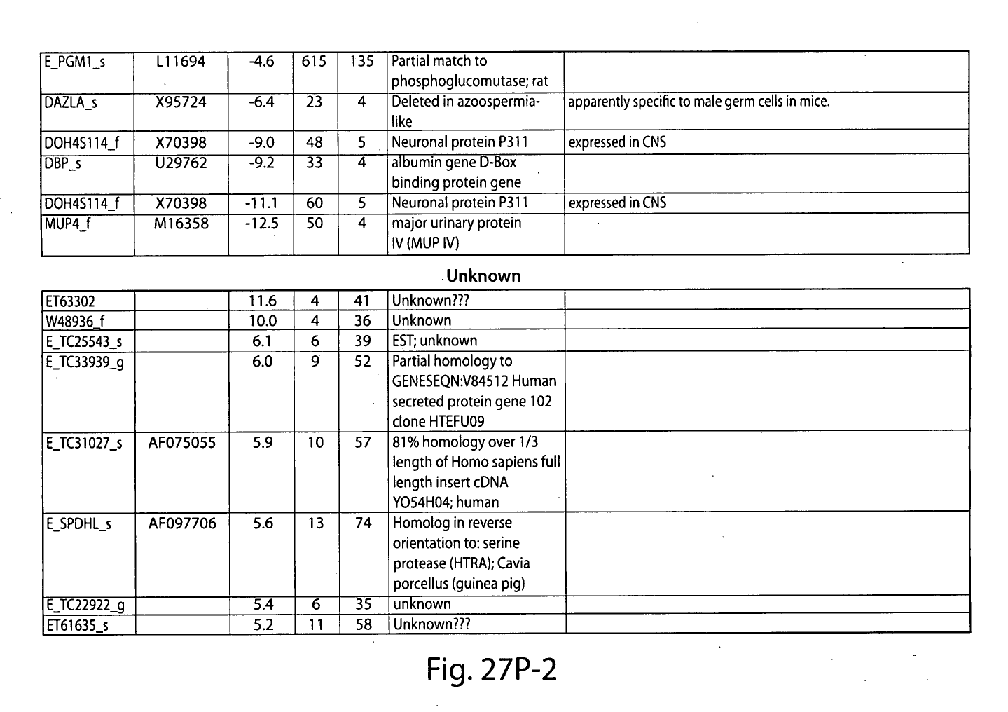

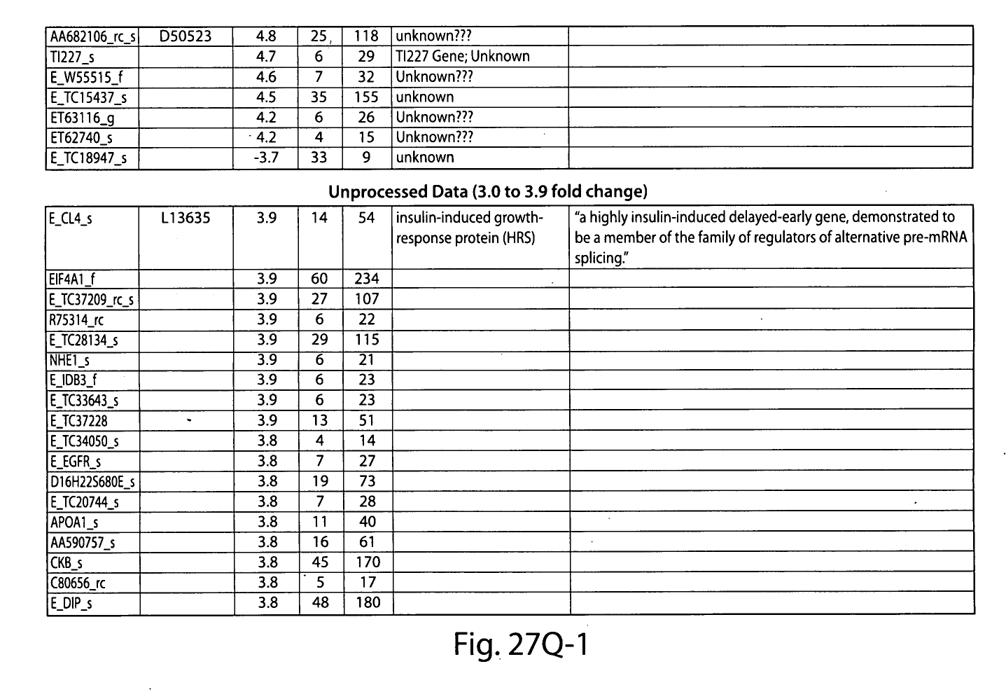

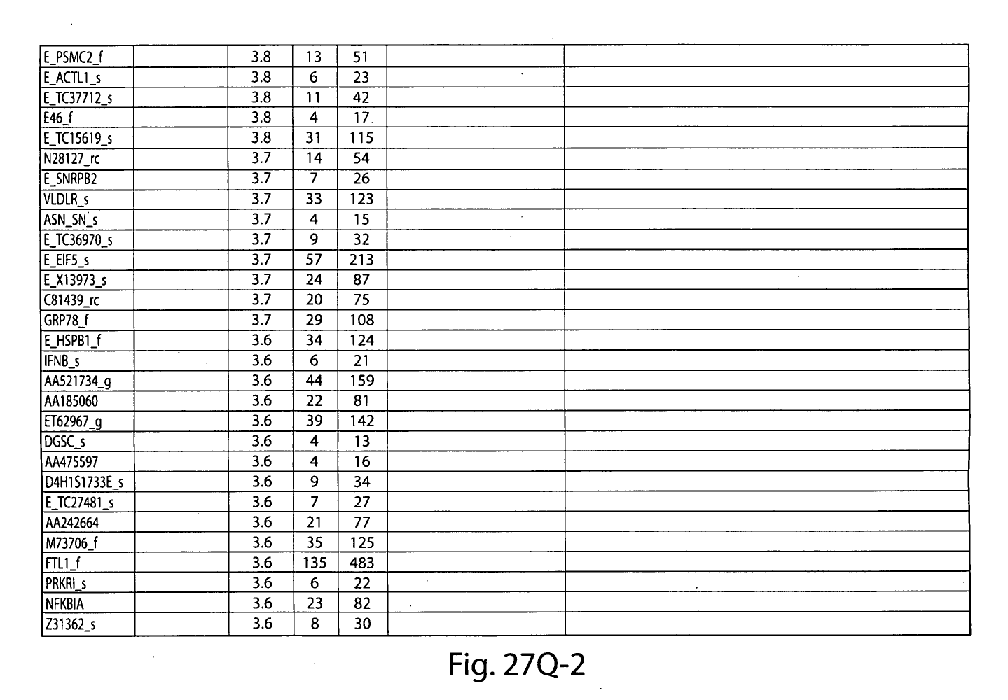

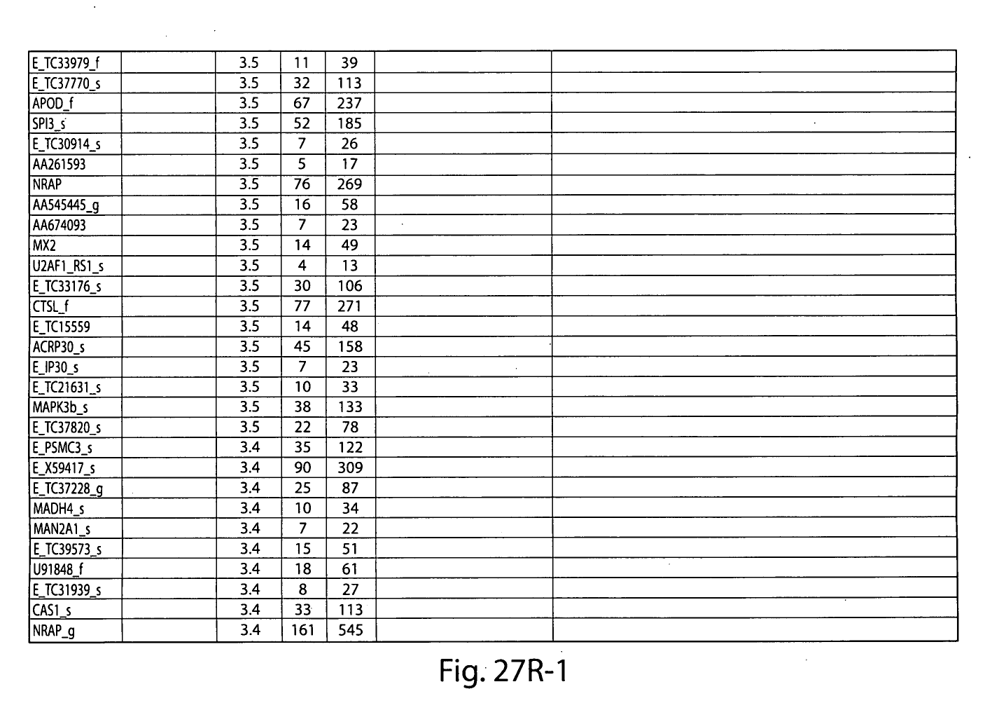

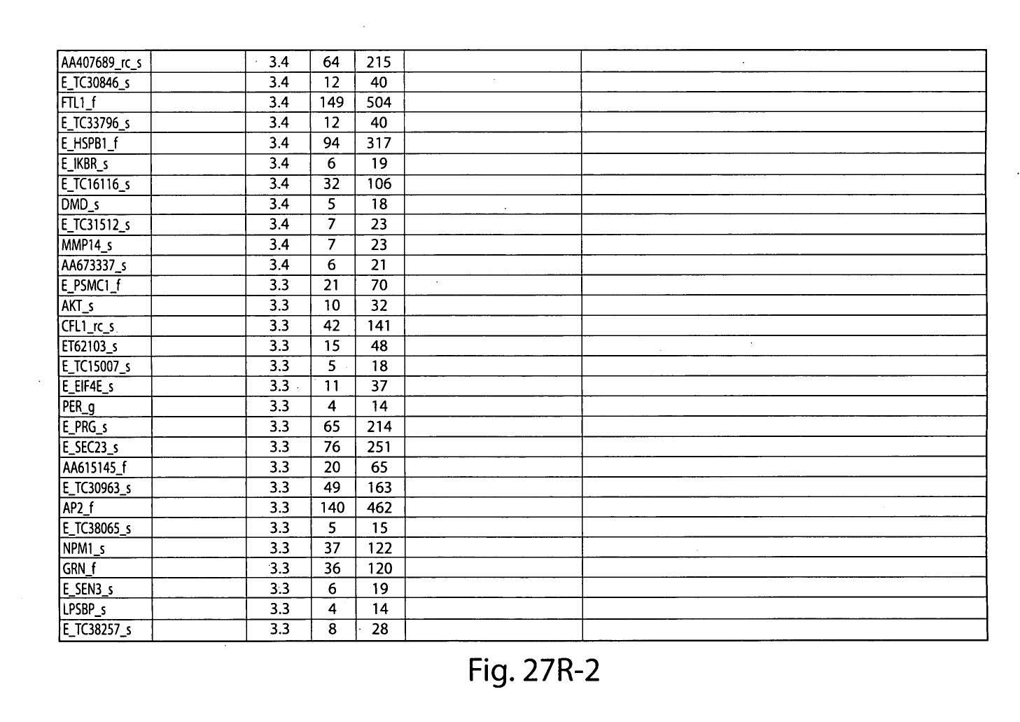

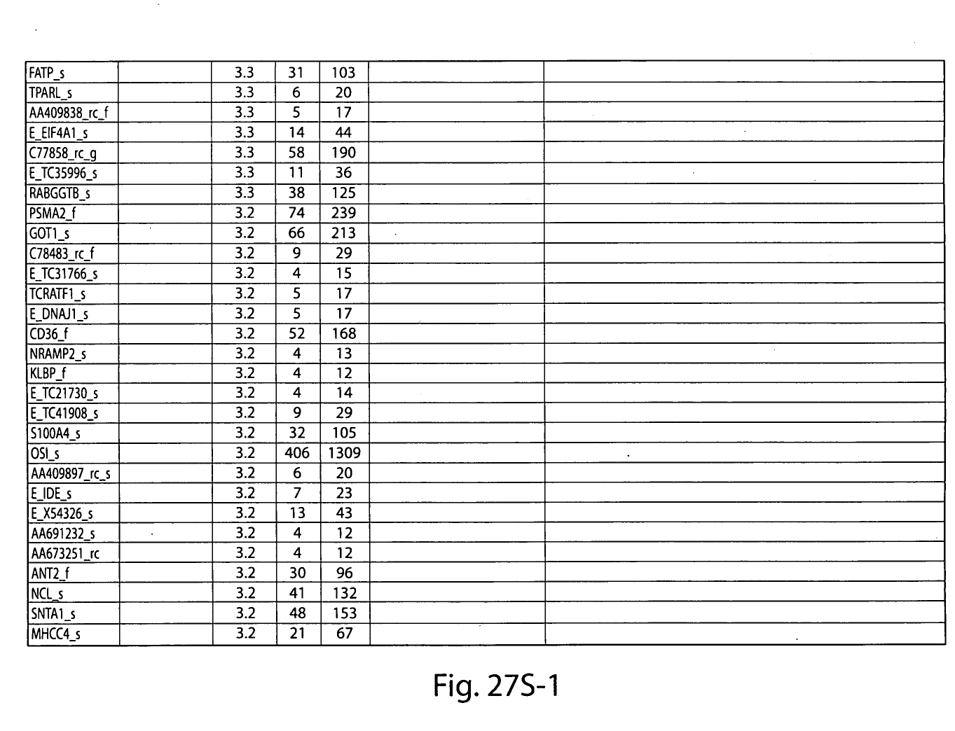

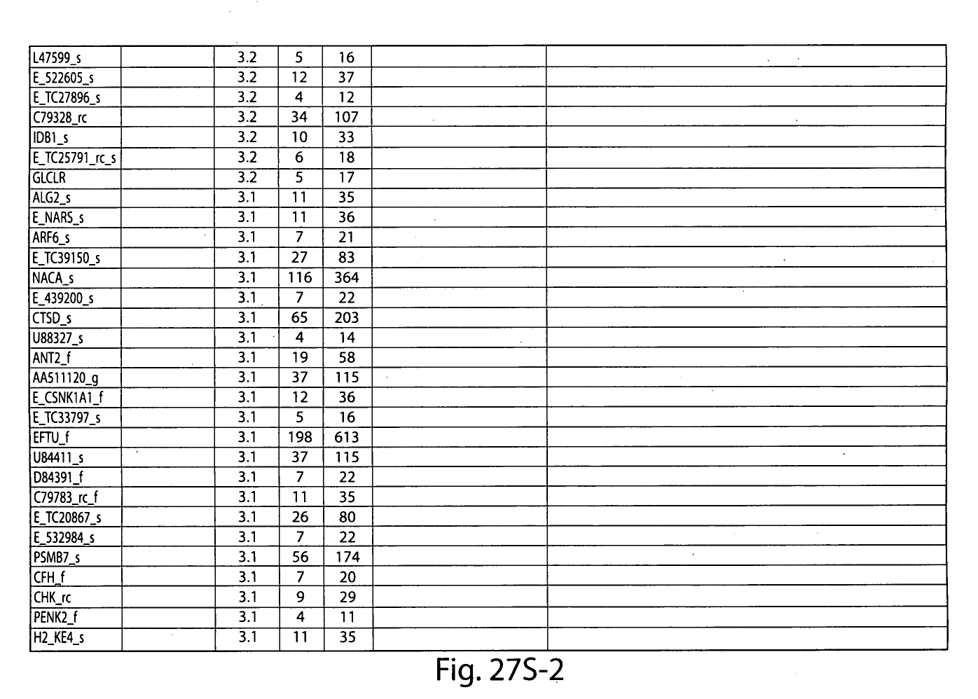

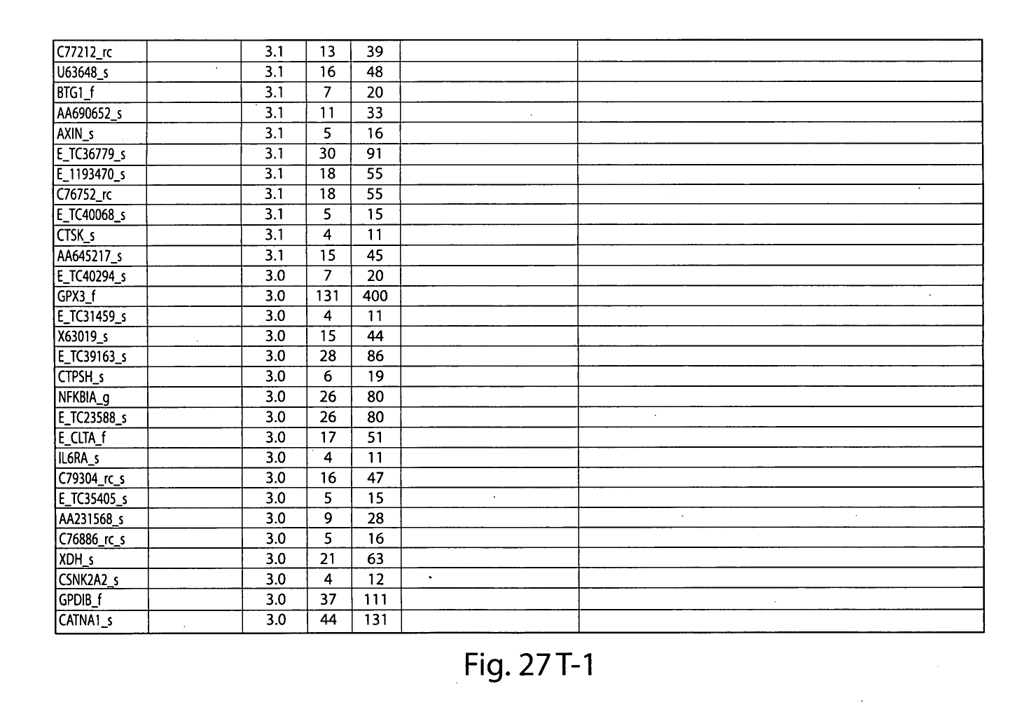

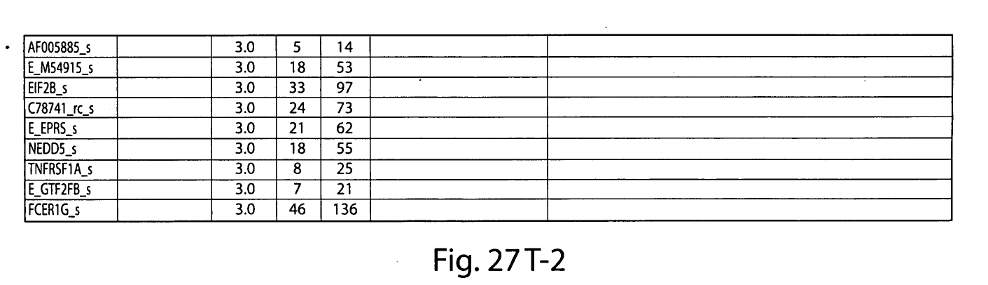

Global gene expression analysis of muscle tissue of mice three and seven days after injection with AdBMP-10 as measured with the Affymetrix® GeneChip MU11k murine array was compared to control. Results for three days post-injection are shown in FIGS. 27A-27D; results for seven days post-injection are shown in FIGS. 27E-27T.

Experiment C. In Vivo Global Gene Expression Analysis Using Affymetrix GeneChip MU11k Murine Array with RNA from Fat Tissue of AdBMP-10 Mice.

### Methods.

RNA from fat tissue from AdBMP-10 mice, injected as described above, was prepared as described above from mice sacrificed at day 3. Hybridization to Affymetrix oligonucleotide arrays was performed as described above.

### Results.

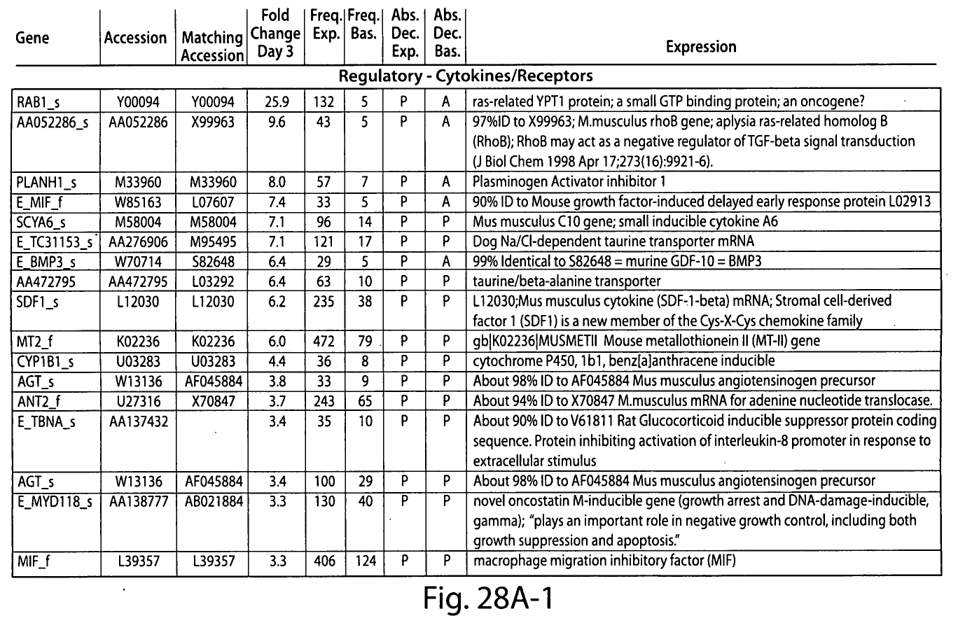

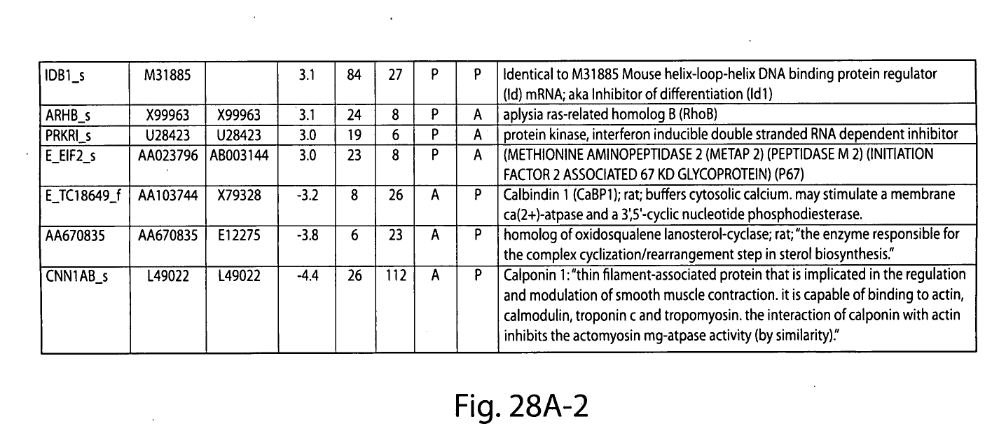

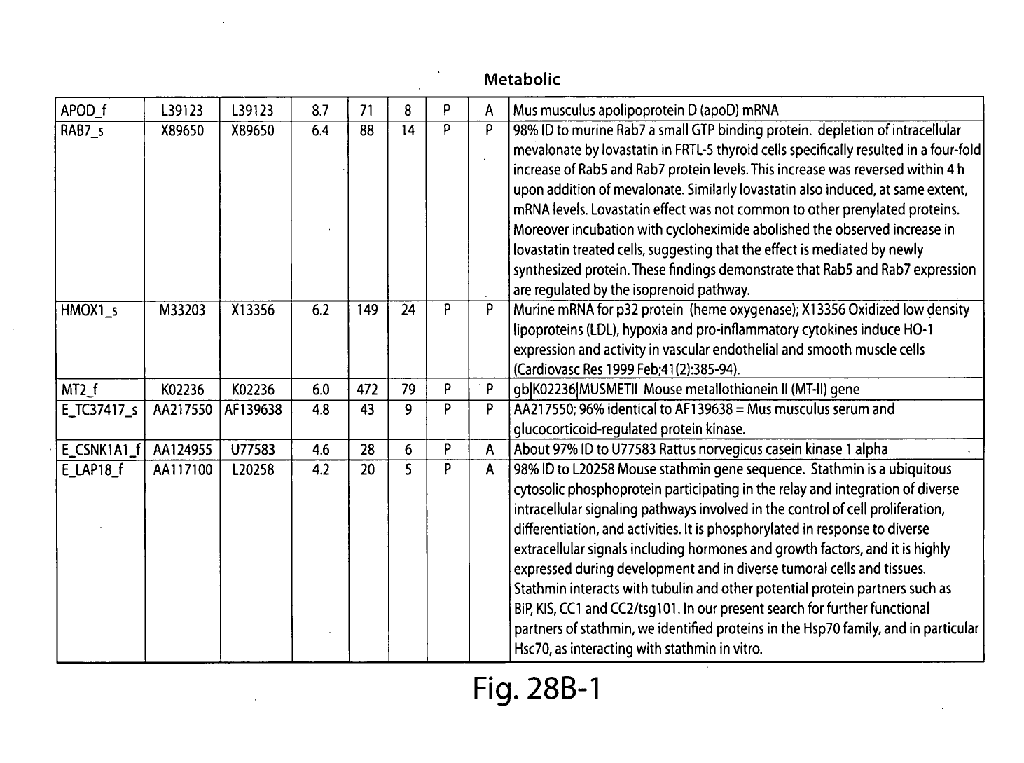

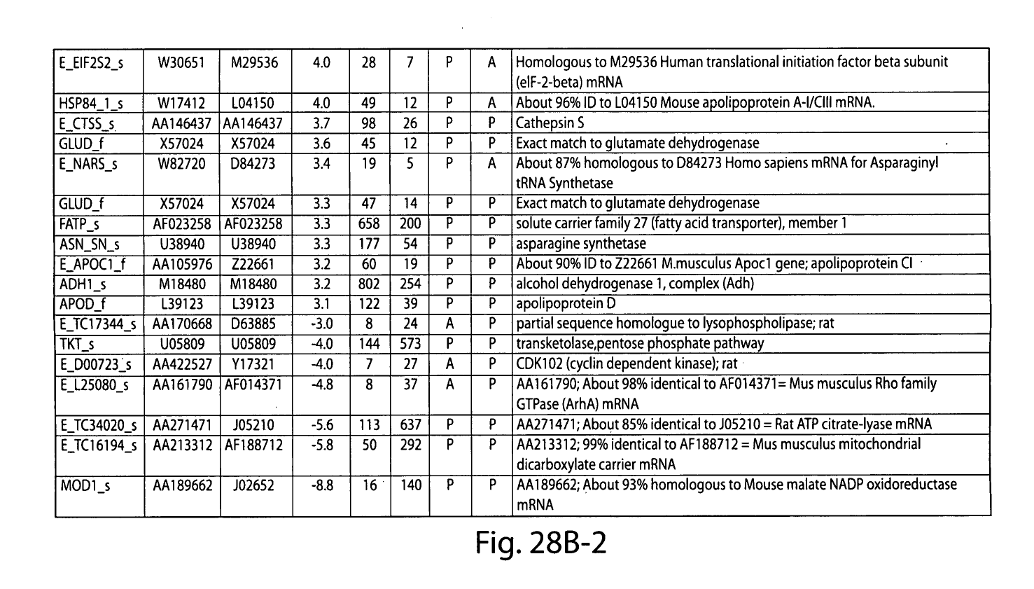

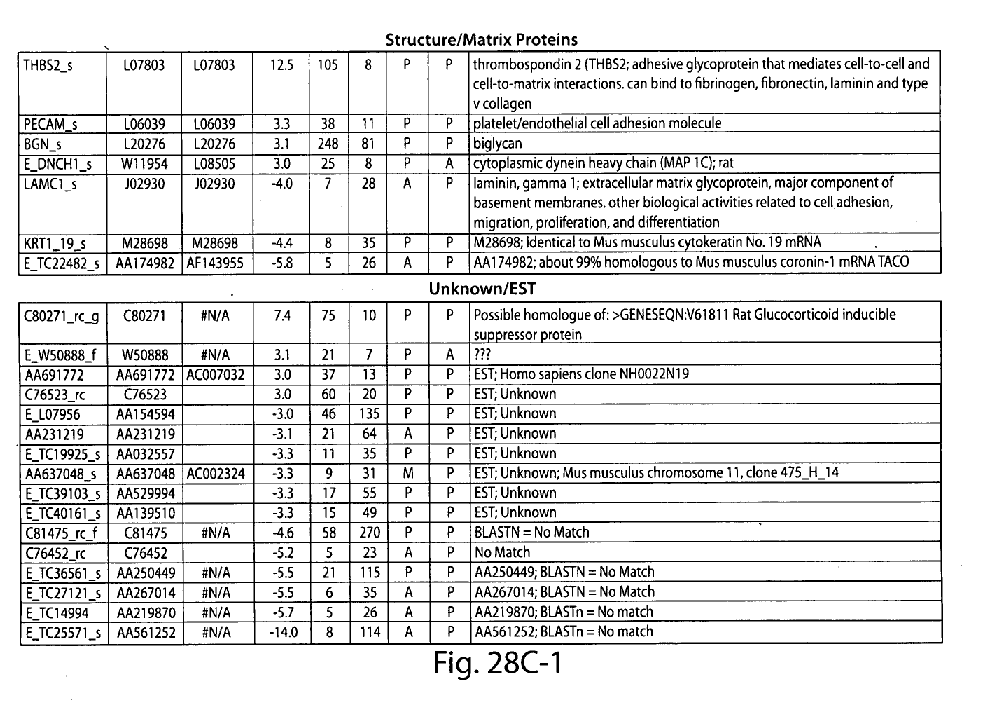

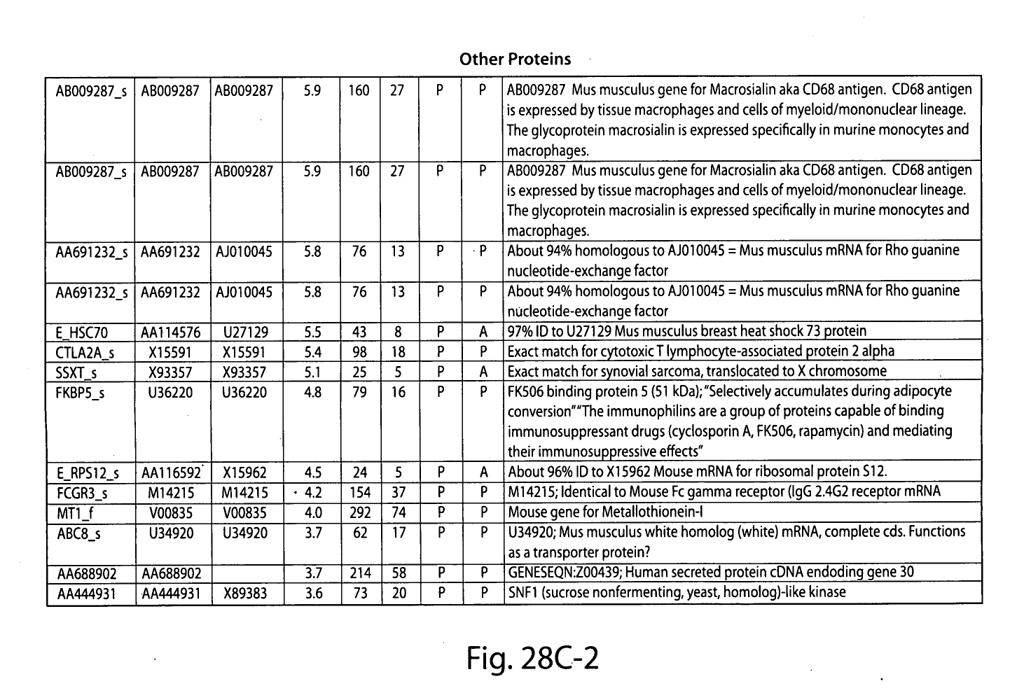

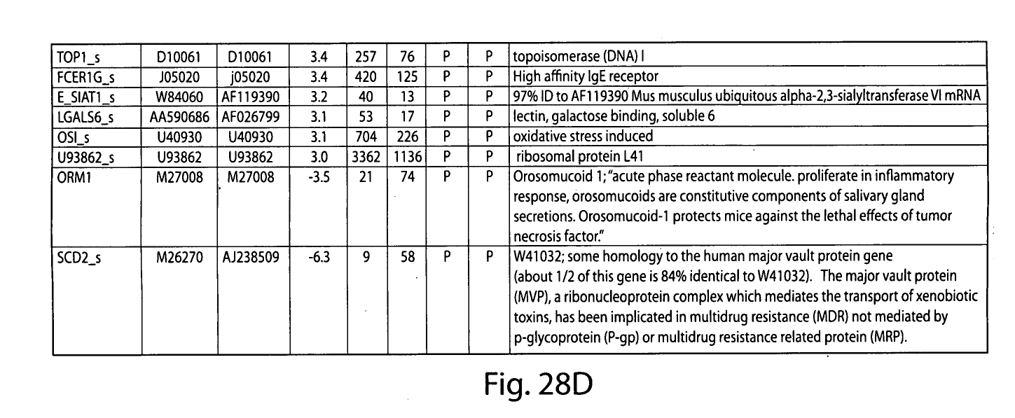

Global gene expression analysis of fat tissue of mice three days after injection with AdBMP-10 as measured with the Affymetrix® GeneChip MU11k murine arrays was compared to control. Results are shown in FIGS. 28A-28D.

In summary, notable gene expression changes (illustrative, not exhaustive) in tissues of AdBMP-10 treated mice were as follows. GDF8 mRNA expression was upregulated in muscle; also known as myostatin, GDF8 is a negative regulator of muscle growth. Expression of known BMP responsive genes such as Smad6, Smad7, and inhibitors of DNA binding Id1, Id2, and Id4 were also increased. Smad6 and Id1 mRNA were increased in fat and heart tissue. Id proteins are indicative of endothelial cell activation. Smad6 is an inhibitor of BMP and TGFθ signaling. Endoglin, a TGFΣ-family Type III co-receptor was increased in the muscle of AdBMP-10 treated mice. Transcriptional profiling of AdBMP-10 mice revealed decreased expression of an important regulator of integrins and cell-cell adhesion, the Ras-related protein-1a (Rap1a).

### EXAMPLE 11

### Serum Levels of Stromal Cell-Derived Factor 1 (SDF-1) and Matrix Metallopeptidase 9 (MMP9) in AdBMP-10 Mice

### Methods.

ELISA analyses demonstrated increased levels of Stromal Cell-derived factor 1 (SDF-1) and matrix metallopeptidase 9 (MMP9) in serum of AdBMP-10 injected mice 1 day post injection. A single dose of 5×10¹⁰ particles of recombinant adenovirus encoding hBMP-10 was injected into the tail vein of female C57BL/6J mice, age 7-8 weeks (n=3). Control mice received an adenovirus encoding GFP or PBS/10% glycerol (n=3). Animals were sacrificed 24 hours post-injection and blood was collected by cardiac puncture into and spun for 10 minutes at 3000 rpm to separate serum. Serum was transferred to a clean tube and frozen at −80° C. Serum was sent to Pierce Biotechnology, Inc. for analysis using a custom designed SearchLight Multiplex Assay. Analytes tested included SDF-1 and MMP9.

### Results.

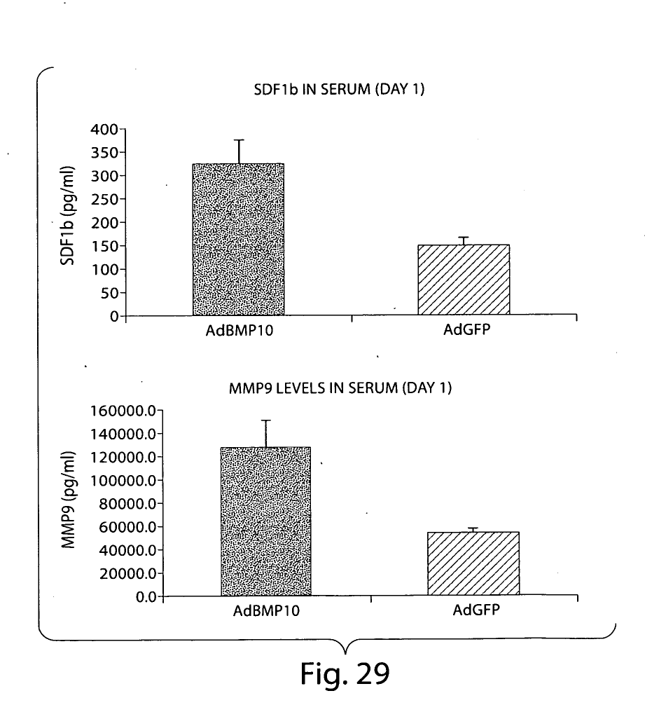

Serum levels of SDF-1 and MMP9 were increased in AdBMP-10 treated mice compared to controls at day 1 (n=3, p<0.05). Results from the ELISA assay are shown in FIG. 29.

What is claimed is:

1. A method of decreasing one or more biological activities of Bone Morphogenic Protein-10 (BMP-10) in a BMP-10-responsive cell or tissue, comprising:
contacting the BMP-10-responsive cell or tissue, said cell or tissue having an increase in one or more BMP-10 biological activities, with a BMP-10 antagonist in an amount sufficient to decrease one or more BMP-10 biological activities in the cell or tissue,
wherein the BMP-10-responsive cell or tissue is a vascular or a renal cell or tissue, or a fibrotic tissue; and
wherein the BMP-10 antagonist is selected from the group consisting of: an anti-BMP-10 antibody molecule, an anti-BMP-10 receptor antibody molecule, a soluble BMP-10 receptor, a BMP-10 nucleic acid inhibitor, a BMP-10 receptor nucleic acid inhibitor, a BMP-10 antagonistic propeptide, a BMP-10 binding domain fusion variant and a BMP-10 receptor binding domain fusion variant.

2. The method of claim 1, wherein the BMP-10 antagonist reduces one or more of the BMP-10 biological activities chosen from:
(i) phosphorylation of a Smad protein;
(ii) induction of gene expression of myostatin, endoglin or an inhibitory Smad;
(iii) increased expression of pro-angiogenic genes;
(iv) decreased expression of Ras-related protein-1a (Rap1a);
(v) modulation of expression of one or more genes in response to BMP-10 stimulation of endothelial cells in vitro or in vivo identified in FIGS. 22-28;
(vi) increased serum levels of stromal-derived differentiation factor (SDF-1) or matrix 25 metallopeptidase 9 (MMP-9); or
(vii) increased abnormalities in blood vessels, such as vascular dysplasia, hemorrhaging, telangiectasias, and/or arteriovenous malformations.

3. The method of claim 1, wherein the BMP-10 antagonist alters one or more of: vascular homeostasis, renal function, or formation or accumulation of fibrous tissue.

4. The method of claim 1, wherein the vascular cell or tissue is an endothelial or a smooth muscle cell or tissue.

5. The method of claim 1, wherein the contacting step occurs on the BMP-10-responsive cells or tissue present in a cell culture, wherein said cell culture is previously or simultaneously exposed to BMP-10.

6. The method of claim 1, wherein the contacting step occurs on the BMP-10-responsive cells or tissue present in a subject.

7. The method of claim 6, wherein the subject is a human patient having, or at risk of having, a vascular, renal or a fibrotic condition or disorder.

8. A method of treating or preventing a vascular, renal or fibrotic condition or disorder in a mammalian subject, comprising:
administering to the mammalian subject a BMP-10 antagonist, in an amount sufficient to inhibit or reduce one or more BMP-10 biological activities in a vascular or renal cell or tissue, or a fibrotic tissue, in the subject, wherein the BMP-10 antagonist is selected from the group consisting of: an anti-BMP-10 antibody molecule, an anti-BMP-10 receptor antibody molecule, a soluble BMP-10 receptor, a BMP-10 nucleic acid inhibitor, a BMP-10 receptor nucleic acid inhibitor, a BMP-10 antagonistic propeptide, a BMP-10 binding domain fusion variant and a BMP-10 receptor binding domain fusion variant.

9. The method of claim 7 or 8, wherein the vascular condition or disorder is characterized by endothelial or smooth muscle cell dysfunction.

10. The method of claim 7 or 8, wherein the subject is a human at risk of, or having, a disorder chosen from one or more of: Hereditary Hemorrhagic Telangiectasia (HHT), nephritic syndrome, nephropathy, diabetic nephropathy, retinopathy, stroke, atherosclerosis, arteriosclerosis, peripheral artery disease, hypertension, hyperlipidemia, thrombosis or restenosis.

11. The method of claim 7 or 8, wherein the subject is a human at risk of, or having, a neoplastic disorder selected from the group consisting of colorectal carcinoma, gastric carcinoma, breast carcinoma, lung carcinoma, esophageal carcinoma and liver carcinomas.

12. The method of claim 7 or 8, wherein the fibrotic condition or disorder is characterized by formation or accumulation of fibrous tissue in the liver, lung, kidney or heart.

13. The method of claim 1 or 8, wherein the BMP-10 is a mature or pro-peptide human BMP-10.

14. The method of claim 1 or 8, wherein the BMP-10 antagonist is an antibody molecule that binds to human BMP-10 or human BMP-10 receptor.

15. The method of claim 14, wherein the antibody molecule is a human, humanized, chimeric, camelid, shark or in vitro generated antibody, or antigen-binding fragment thereof, to human BMP-10 or human BMP-10 receptor polypeptide.

16. The method of claim 1 or 8, wherein the BMP-10 antagonist is a soluble fragment of a BMP-10 receptor or a BMP-10 antagonistic propeptide.

17. The method of claim 16, wherein the soluble fragment is fused to an immunoglobulin Fc region.

18. The method of claim 1 or 8, wherein the BMP-1 antagonist is a BMP-10 receptor or fragment thereof selected from the group consisting of an endoglin, an ALK receptor, a chordin, USAG-1, sclerosin and activin receptor IIB.

19. The method of claim 1 or 8, wherein the BMP-10 nucleic acid inhibitor or BMP-10 receptor nucleic acid inhibitor is selected from the group consisting of an antisense molecule, a ribozyme, RNAi, and a triple helix molecule.

20. A method of treating a disorder characterized by underactive or disrupted vascular or cardiac cell proliferation or activity, comprising:
administering to the subject a BMP-10 polypeptide, or a functional fragment thereof, in an amount sufficient to increase or stimulate one or more BMP-10 biological activities in a vascular or cardiac cell or tissue, thereby treating or preventing the disorder, wherein the disorder is a disorder or condition following endothelial cell injury.

21. A method of evaluating, diagnosing, or monitoring the progression of, a BMP-10 associated vascular, renal or fibrotic disorder or condition in a test sample, comprising:
evaluating the expression or activity of a nucleic acid or polypeptide chosen from BMP-10 or a BMP-10-associated gene, such that, a difference in the level of the nucleic acid or polypeptide relative to a reference sample is indicative of the presence or progression of the disorder or condition.

22. The method of claim 21, wherein the BMP-10-associated genes comprise one or more of GDF-8, GDF-10, endoglin, inhibitory Smad, and a pro-angiogenic gene.

23. A method, or an assay, for identifying a test compound that modulates vascular or renal function, comprising:
(i) providing or identifying a test agent that interacts binds to BMP-10 or a BMP-10 receptor polypeptide or nucleic acid; and
(ii) evaluating a change in an activity of a vascular or renal cell or tissue in the presence of the test agent, relative to a reference sample,
wherein the test compound is selected from the group consisting of an antibody molecule, BMP-10 peptide, a soluble BMP-10 receptor, a fusion of a soluble BMP-10 receptor, a small molecule, a naturally-occurring BMP-10 antagonist, an antisense molecule, a ribozyme, an RNAi and a triple helix molecule.

24. The method of claim 26, wherein the evaluating step comprises:
contacting one or more of: a BMP-10 or BMP-10 receptor polypeptide, or a nucleic acid encoding the BMP-10 or BMP-10 receptor, with the test compound; and
detecting a change in one or more activities of the BMP-10 or the BMP-10 receptor polypeptide or nucleic acid, in the presence of the test compound, relative to a control sample without the test compound, wherein the change in an activity of the vascular or renal cell or tissue is detected by measuring a change, in the presence of the test compound, relative to a reference sample in one or more of:
(i) phosphorylation of a Smad protein;
(ii) gene expression of myostatin, endoglin or an inhibitory Smad;
(iii) expression of one or more pro-angiogenic genes;
(iv) expression of Ras-related protein-1a (Rap1a);
(v) expression of one or more genes in response to BMP-10 stimulation of endothelial cells in vitro or in vivo identified in FIGS. 22-28;
(vi) serum levels of stromal-derived differentiation factor (SDF-1) or matrix metallopeptidase 9 (MMP-9); or
(vii) abnormalities in blood vessels, such as vascular dysplasia, hemorrhaging, telangiectasias or arteriovenous malformations,
wherein a decrease in one or more of (i)-(iii) and (vi), and an increase in (iv), is indicative of an antagonist of BMP-10 function; and
wherein an increase in one or more of (i)-(iii) and (vi), and a decrease in (iv) is indicative of an agonist of BMP-10 function.


SEQUENCE LISTING (21 sequences)

SEQ ID NO: 1 - DNA, 1584 bp, Homo sapiens
ggggagaggaagagtggtagggggagggagagagagaggaagagtttccaaacttgtctccagtgacaggagacatttacgttccacaagataaaactgccacttagagcccagggaagctaaaccttcctggcttggcctaggagctcgagcggagtcatgggctctctggtcctgacactgtgcgctcttttctgcctggcagcttacttggtttctggcagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaaggaacgccaaaggaaactactgtaagaggaccccgctctacatcgacttcaaggagattgggtgggactcctggatcatcgctccgcctggatacgaagcctatgaatgccgtggtgtttgtaactaccccctggcagagcatctcacacccacaaagcatgcaattatccaggccttggtccacctcaagaattcccagaaagcttccaaagcctgctgtgtgcccacaaagctagagcccatctccatcctctatttagacaaaggcgtcgtcacctacaagtttaaatacgaaggcatggccgtctccgaatgtggctgtagatagaagaagagtcctatggcttatttaataactgtaaatgtgtatatttggtgttcctatttaatgagattatttaataagggtgtacagtaatagaggcttgctgccttcaggaaatggacaggtcagtttgttgtaggaaatgcatatttt

SEQ ID NO: 2 - protein, 424 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 3 - DNA, 4263 bp, Homo sapiens
aggaaacggtttattaggagggagtggtggagctgggccaggcaggaagacgctggaataagaaacatttttgctccagcccccatcccagtcccgggaggctgccgcgccagctgcgccgagcgagcccctccccggctccagcccggtccggggccgcgcccggaccccagcccgccgtccagcgctggcggtgcaactgcggccgcgcggtggaggggaggtggccccggtccgccgaaggctagcgccccgccacccgcagagcgggcccagagggaccatgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacagcatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtgttgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaatagcccaggagcacctgattcctttctgcctgcagggggctgggggggtggggggcagtggatggtgccctatctgggtagaggtagtgtgagtgtggtgtgtgctggggatgggcagctgcgcctgcctgctcggcccccagcccacccagccaaaaatacagctgggctgaaacctgatcccctgctgtctggcctgctcaaagcggcaggctccctgacgcctggctctctccccacccctatggccagcatggtgcaccccctaccactcccgggacaggatgcaaaagaggctccagagtcagagtgccaagccagggaatcccagtcccagactcagagcccgggcctgcactttgccccctgcccttgatcaaccccactgccccaccagagctgccagggtggcacagggccctgtccagcccctggcacacacttccctgccaggcctcagcctctagcataagctccagagagccagggcccatcagtttctctctgtggatttgtatctcagctccatgatgccttgggctttctgtctcctcaacaagagtgcagcttgctgaatgtcagctgcctgagagagctggggcctgacttactagggcattaaatcctaagaggtcctactgaggtgtggcaggatcacaggccagtggaaaaagggcaggtcagatgggcaaggcccaggactttcagattaactgagaggatatcgaggccaagcatggcagggggaaggtcagtgggtgtcaagagacccaggtctgaccccggatgtttgctccatgtgacaaaagcaggcctgtctcaggaccttttcttttcttttttccttctttttttttttgacacggagtttcgctcttgttgtccaggctagagtgcaatggcatgatcccagctcaccgcaacgtctacctcccaggttcaaatcattctcttgcctcagactcccgagtagctgggattacaggcacatgccaccatgcctggctaattttgtatatttagtagaaacagggtttcaccatgctggccatgctggtctcgaactcctgacctcaggtgttccacctacctcagcctcccaaagtgctggggttacaggtgtgagccatcgcgcctggccaggacctttgtttcttatctacatattggaagatttggtcctgatgtcctttgaggcttctttagctctagttctctgacacttcagcctatatcacagctaacttcttcagtctcatctattccttatgctccagcccctggcaatttgcctcaagatgggggtttgaaaataactttacctgactcaaggagtgtctggagcacctcctagtctaagtctgcaagctccagttcttgcctaaaaccatgccagtggccacccttgggctcagacagctctgggccttttgaccacaagccagcccctcgccctctctgtggcatagtcttctctgccccaggactgcagggcggcttcctccaaggcttccaaggctcaaaagaaatttggctccatccaagaaggctccagctcccctactggcccctggctcaggcccacacccctggccaggcccagagagtgtgtctcaggagaattcaatggctctagagagacacacagaaagtttggcatttggaaatttcaaggatgtatgtatgctcacgtatggagcaggttgtcctggtccctgggtgcagggaagtgggctgcagggaagtggattggaggggagcttgaggaatataaggagcgggggtggagactcaggctatggacaaggacagccccaaggttgggaagacctggccttagtcgtcctcagcctaggggcagggcagtgaagaaagctctccccgctcctgctgtaatgacccagagtagcctccccaggccggcatcttatgtgtgtcttccaccatcctcatggtggcacttttctaggcctgtctcccagcattgtgcaaggctcggaagagaaccaggaagtgaaactgggtgaaaacagaaagctcaatggatgggctaggttcccagatcattagggcagagtttgcacgtcctctggtcactggaatccacccagcccacgaatcatctccctcttgaaggattttatttctactgggttttggaacaaactcctgctgagaccccacagccagaaactgaaagcagcagctccccaaagcctggaaaatccctaagagaaggcctggggcaggaagtggagtgacaggggacaggtagagagaagggggcccaatggccagggagtgaaggaggtggcgttgctgagagcagtctgcacatgcttctgtctgagtgcaggaaggtgttccagggtcgaaattacacttctcgtacctggagacgctgtttgtgggagcactgggctcatgcctggcacacaataggtctgcaataaaccatggttaaatcctga

SEQ ID NO: 4 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 5 - DNA, 3631 bp, Homo sapiens
gcggccgctgcagagattggaatccgcctgccgggcttggcgaaggagaagggaggaggcaggagcgaggagggaggagggccaagggcgggcaggaaggcttaggctcggcgcgtccgtccgcgcgcggcgaagatcgcacggcccgatcgaggggcgaccgggtcggggccgctgcacgccaagggcgaaggccgattcgggccccacttcgccccggcggctcgccgcgcccacccgctccgcgccgagggctggaggatgcgttccctggggtccggacttatgaaaatatgcatcagtttaatactgtcttggaattcatgagatggaagcataggtcaaagctgtttggagaaaatcagaagtacagttttatctagccacatcttggaggagtcgtaagaaagcagtgggagttgaagtcattgtcaagtgcttgcgatcttttacaagaaaatctcactgaatgatagtcatttaaattggtgaagtagcaagaccaattattaaaggtgacagtacacaggaaacattacaattgaacaatgcctcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctgatggttaaaccatcggaggagaaactctagactgcaagaactgtttttacccatggcatgggtggaattagagtggaataaggatgttaacttggttctcagactctttcttcactacgtgttcacaggctgctaatattaaacctttcagtactcttattaggatacaagctgggaacttctaaacacttcattctttatatatggacagctttattttaaatgtggtttttgatgcctttttttaagtgggtttttatgaactgcatcaagacttcaatcctgattagtgtctccagtcaagctctgggtactgaattgcctgttcataaaacggtgctttctgtgaaagccttaagaagataaatgagcgcagcagagatggagaaatagactttgccttttacctgagacattcagttcgtttgtattctacctttgtaaaacagcctatagatgatgatgtgtttgggatactgcttattttatgatagtttgtcctgtgtccttagtgatgtgtgtgtgtctccatgcacatgcacgccgggattcctctgctgccatttgaattagaagaaaataatttatatgcatgcacaggaagatattggtggccggtggttttgtgctttaaaaatgcaatatctgaccaagattcgccaatctcatacaagccatttactttgcaagtgagatagcttccccaccagctttattttttaacatgaaagctgatgccaaggccaaaagaagtttaaagcatctgtaaatttggactgttttccttcaaccaccattttttttgtggttattatttttgtcacggaaagcatcctctccaaagttggagcttctattgccatgaaccatgcttacaaagaaagcacttcttattgaagtgaattcctgcatttgatagcaatgtaagtgcctataaccatgttctatattctttattctcagtaacttttaaaagggaagttatttatattttgtgtataatgtgctttatttgcaaatcacccactcctttacaaccatactttatatatgtacatacattcatactgtagaaaccagctcatgtgtacctcatatcccatccttaagagaagaaatgttataaagtagaactaaatataaattttcagaattaatgcattcaaagtaatatatcaaatccaggactttgttaacttcaggtaaaaacttcattagggtaatatcatctcaattttttcaaatgaaaggattctctaattagaaatttatatgtcagagctgttataaatttatcaactgtcaaatatgttctggacagctaaatcatttgagatttttggttttttgatttctattccctaacttgtgaagacaatgaaaaatcaggcagaaatatttagtatctagtcagtatctgtagctacactgtataactgttcttcaataaaatggttcatattttatagaaaaaaaaaaaaaaaa

SEQ ID NO: 6 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 7 - DNA, 20 bp, Homo sapiens
gccactggatctgtccgatt

SEQ ID NO: 8 - DNA, 19 bp, Homo sapiens
cacccggagcagtgatgag

SEQ ID NO: 9 - DNA, 34 bp, Homo sapiens
acattgtcttacactgaaacggaggctaccaact

SEQ ID NO: 10 - DNA, 20 bp, Homo sapiens
cagaaggtgcggagcaaaat

SEQ ID NO: 11 - DNA, 21 bp, Homo sapiens
tgtacacccacacaccatcca

SEQ ID NO: 12 - DNA, 20 bp, Homo sapiens
ctgcggcatccagctgacgc

SEQ ID NO: 13 - DNA, 20 bp, Homo sapiens
ccacatcgctcagacaccat

SEQ ID NO: 14 - DNA, 18 bp, Homo sapiens
gcgcccaatacgaccaaa

SEQ ID NO: 15 - DNA, 25 bp, Homo sapiens
cgttgactccgaccttcaccttccc

SEQ ID NO: 16 - DNA, 2912 bp, Homo sapiens
tgaggagaacaaaaaagagagagagagccgagcgggggagcgatcgagggagctgagccgagagaaagagccgccgggcgctgcctcgccagacctcgctgggaccccggggccaccgggaggcacttttgtggaggggggagggggggcgacctcggcagcctcggcgcacgaagcgtccgagggcagcgtggggcgggctgcgacctctgcatcggtggactgcatttttaattaaggattcccagcagctctttgggatttttacagcttccactcatgtgttgacacccgcgtccaggagaaactcgctccaagtgcatctagcgcctgggacctgagacggcgttggcctttcgtgcatgcaaatccagggatttaggttttgtttgggatttccttttctttctttccttttttttttctttttgcagggagtaagaagggagctgggggtatcaacaagcctgcctttcggatcctgcgggaaaagcccatgtagttaagcgctttggtttaaaaaaaaggcaaggtaaaggcagggctttccagacacatttaggggttcgcgcgagcgctttgtgctcatggaccagccgcacaacttttgaaggctcgccggcccatgtggggtctttctggcggcgcgccgcctgcagcccccctaaagcgcgggggctggagttgttgagcagccccgccgctgtggtccatgtagccgctggccgcgcgcggactgcggctcggcgtgcgcgtgttcccggccgtcccgcctcggcgagctccctcatgttgtcgccctgcggcgccccttcgacgacaggctgtgcgcggtctgcacggcgctccgcggcggagcttcatgtggggctgcgacccgcgcagccggcgcctcgctgagggaacggacccccggtaaccggagaccgcctcccccccacccctggcgccaaaggatatcgtatgttcaggtccaaacgctcggggctggtgcggcgactttggcgaagtcgtgtggtccccgaccgggaggaaggcggcagcggcggcggcggtggcggcgacgaggatgggagcttgggcagccgagctgagccggccccgcgggcaagagagggcggaggctgcggccgctccgaagtccgcccggtagccccgcggcggccccgggacgcagtgggacagcgaggcgcccagggcgcggggaggcgccggcgcgcagggggccccccgaggcccatgtcggagccaggggccggcgctgggagctccctgctggacgtggcggagccgggaggcccgggctggctgcccgagagtgactgcgagacggtgacctgctgtctcttttcggagcgggacgccgccggcgcgccccgggacgccagcgaccccctggccggggcggccctggagccggcgggcggcgggcggagtcgcgaagcgcgctcgcggctgctgctgctggagcaggaactcaaaaccgtcacgtactcgctgctgaagcggctcaaggagcgctcgctggacacgctgctggaggcggtggagtcccgcggcggcgtgccgggcggctgcgtgctggtgccgcgcgccgacctccgcctgggcggccagcccgcgccgccgcagctgctgctcggccgcctctttcgctggcccgacctgcagcacgccgtggagctgaagcccctgtgcggctgccacagcttcgccgccgccgccgacggccctaccgtgtgctgcaacccctaccacttcagccggctctgcgggcccgaatctccgccacctccctactctcggctgtctcctcgcgacgagtacaagccactggatctgtccgattccacattgtcttacactgaaacggaggctaccaactccctcatcactgctccgggtgaattctcagacgccagcatgtctccggacgccaccaagccgagccactggtgcagcgtggcgtactgggagcaccggacgcgcgtgggccgcctctatgcggtgtacgaccaggccgtcagcatcttctacgacctacctcagggcagcggcttctgcctgggccagctcaacctggagcagcgcagcgagtcggtgcggcgaacgcgcagcaagatcggcttcggcatcctgctcagcaaggagcccgacggcgtgtgggcctacaaccgcggcgagcaccccatcttcgtcaactccccgacgctggacgcgcccggcggccgcgccctggtcgtgcgcaaggtgccccccggctactccatcaaggtgttcgacttcgagcgctcgggcctgcagcacgcgcccgagcccgacgccgccgacggcccctacgaccccaacagcgtccgcatcagcttcgccaagggctgggggccctgctactcccggcagttcatcacctcctgcccctgctggctggagatcctcctcaacaaccccagatagtggcggccccggcgggaggggcgggtgggaggccgcggccaccgccacctgccggcctcgagaggggccgatgcccagagacacagcccccacggacaaaaccccccagatatcatctacctagatttaatataaagttttatatattatatggaaatatatattatacttgtaattatggagtcatttttacaatgtaattatttatgtatggtgcaatgtgtgtatatggacaaaacaagaaagacgcactttggcttataattctttcaatacagatatattttctttctcttcctccttcctcttccttactttttatatatatatataaagaaaatgatacagcagagctaggtggaaaagcctgggtttggtgtatggtttttgagatattaatgcccagacaaaaagctaataccagtcactcgataataaagtattcgcattataaaaaa

SEQ ID NO: 17 - DNA, 1080 bp, Homo sapiens
atgcctctcctcctcttgctgctcctgctgccaagccccttacacgcgggcagcggcagaggcgaggccgagaccagagagtgcatctactacaacgccaactgggagctggagaggaccaaccagagcggcctggagagatgcgagggcgagcaggacaagaggctgcactgctacgccagctggcgcaacagcagcggcaccatcgagctggtgaagaagggctgctggctggacgacttcaactgctacgacaggcaggagtgcgtcgccaccgaggagaacccccaggtgtacttctgctgctgcgagggcaacttctgcaacgagaggttcacccacctgcccgaggctggcggccctgaggtgacctacgagccccctcccaccgcccccaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 18 - protein, 360 aa, Homo sapiens
MPLLLLLLLLPSPLHAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 40 aa, Artificial Sequence
  Description of Artificial Sequence Synthetic Peptide
SGGGGSGGGGSGGGGSGGGGSGGGGSGGGGSGGGGSGGGG

SEQ ID NO: 20 - protein, 6 aa, Artificial Sequence
  Description of Artificial Sequence Synthetic six-his peptide
HHHHHH

SEQ ID NO: 21 - protein, 12 aa, Artificial Sequence
  Description of Artificial Sequence Synthetic Peptide
DKGVVTYKFKYE